SEQUENCE LISTING (623 sequences)

SEQ ID NO: 1 - DNA, 18 bp, Artificial sequence
  Synthetic primer
aagaccgatgggcccttg

SEQ ID NO: 2 - DNA, 18 bp, Artificial sequence
  Synthetic primer
gaagaccttggggctggt

SEQ ID NO: 3 - DNA, 20 bp, Artificial sequence
  Synthetic primer
gggaattctcacaggagacg

SEQ ID NO: 4 - DNA, 19 bp, Artificial sequence
  Synthetic primer
gaagacggatgggctctgt

SEQ ID NO: 5 - DNA, 19 bp, Artificial sequence
  Synthetic primer
gggtgtctgcaccctgata

SEQ ID NO: 6 - DNA, 18 bp, Artificial sequence
  Synthetic primer
gacctcgggtgggaacac

SEQ ID NO: 7 - DNA, 18 bp, Artificial sequence
  Synthetic primer
ggtacacggcagggtcag

SEQ ID NO: 8 - DNA, 63 bp, Artificial sequence
  Synthetic primer
  misc_feature (34)..(45): n is a, c, g, or t
acactctttccctacacgacgctcttccgatctnnnnnnnnnnnnaagaccgatgggcccttg

SEQ ID NO: 9 - DNA, 63 bp, Artificial sequence
  Synthetic primer
  misc_feature (34)..(45): n is a, c, g, or t
acactctttccctacacgacgctcttccgatctnnnnnnnnnnnngaagaccttggggctggt

SEQ ID NO: 10 - DNA, 65 bp, Artificial sequence
  Synthetic primer
  misc_feature (34)..(45): n is a, c, g, or t
acactctttccctacacgacgctcttccgatctnnnnnnnnnnnngggaattctcacaggagacg

SEQ ID NO: 11 - DNA, 64 bp, Artificial sequence
  Synthetic primer
  misc_feature (34)..(45): n is a, c, g, or t
acactctttccctacacgacgctcttccgatctnnnnnnnnnnnngaagacggatgggctctgt

SEQ ID NO: 12 - DNA, 64 bp, Artificial sequence
  Synthetic primer
  misc_feature (34)..(45): n is a, c, g, or t
acactctttccctacacgacgctcttccgatctnnnnnnnnnnnngggtgtctgcaccctgata

SEQ ID NO: 13 - DNA, 39 bp, Artificial sequence
  Synthetic primer
gacgtgtgctcttccgatctcgcagaccctctcactcac

SEQ ID NO: 14 - DNA, 40 bp, Artificial sequence
  Synthetic primer
gacgtgtgctcttccgatcttggagctgaggtgaagaagc

SEQ ID NO: 15 - DNA, 40 bp, Artificial sequence
  Synthetic primer
gacgtgtgctcttccgatcttgcaatctgggtctgagttg

SEQ ID NO: 16 - DNA, 39 bp, Artificial sequence
  Synthetic primer
gacgtgtgctcttccgatctggctcaggactggtgaagc

SEQ ID NO: 17 - DNA, 40 bp, Artificial sequence
  Synthetic primer
gacgtgtgctcttccgatcttggagcagaggtgaaaaagc

SEQ ID NO: 18 - DNA, 39 bp, Artificial sequence
  Synthetic primer
gacgtgtgctcttccgatctggtgcagctgttggagtct

SEQ ID NO: 19 - DNA, 40 bp, Artificial sequence
  Synthetic primer
gacgtgtgctcttccgatctactgttgaagccttcggaga

SEQ ID NO: 20 - DNA, 40 bp, Artificial sequence
  Synthetic primer
gacgtgtgctcttccgatctaaacccacacagaccctcac

SEQ ID NO: 21 - DNA, 40 bp, Artificial sequence
  Synthetic primer
gacgtgtgctcttccgatctagtctggggctgaggtgaag

SEQ ID NO: 22 - DNA, 38 bp, Artificial sequence
  Synthetic primer
gacgtgtgctcttccgatctggcccaggactggtgaag

SEQ ID NO: 23 - DNA, 38 bp, Artificial sequence
  Synthetic primer
gacgtgtgctcttccgatctggtgcagctggtggagtc

SEQ ID NO: 24 - DNA, 20 bp, Artificial sequence
  Synthetic primer
acactctttccctacacgac

SEQ ID NO: 25 - DNA, 45 bp, Artificial sequence
  Synthetic primer
aatgatacggcgaccaccgagatctacactctttccctacacgac

SEQ ID NO: 26 - DNA, 66 bp, Artificial sequence
  Synthetic primer
caagcagaagacggcatacgagataacgaaacgtgactggagttcagacgtgtgctcttccgatct

SEQ ID NO: 27 - DNA, 66 bp, Artificial sequence
  Synthetic primer
caagcagaagacggcatacgagataacgtacggtgactggagttcagacgtgtgctcttccgatct

SEQ ID NO: 28 - DNA, 66 bp, Artificial sequence
  Synthetic primer
caagcagaagacggcatacgagataaccactcgtgactggagttcagacgtgtgctcttccgatct

SEQ ID NO: 29 - DNA, 66 bp, Artificial sequence
  Synthetic primer
caagcagaagacggcatacgagataaatcagtgtgactggagttcagacgtgtgctcttccgatct

SEQ ID NO: 30 - DNA, 66 bp, Artificial sequence
  Synthetic primer
caagcagaagacggcatacgagataagctcatgtgactggagttcagacgtgtgctcttccgatct

SEQ ID NO: 31 - DNA, 66 bp, Artificial sequence
  Synthetic primer
caagcagaagacggcatacgagataaaggaatgtgactggagttcagacgtgtgctcttccgatct

SEQ ID NO: 32 - DNA, 66 bp, Artificial sequence
  Synthetic primer
caagcagaagacggcatacgagataacttttggtgactggagttcagacgtgtgctcttccgatct

SEQ ID NO: 33 - DNA, 20 bp, Artificial sequence
  Synthetic primer
cagatctcagctggaccaca

SEQ ID NO: 34 - DNA, 20 bp, Artificial sequence
  Synthetic primer
tcatagaggatggtggcaga

SEQ ID NO: 35 - DNA, 20 bp, Artificial sequence
  Synthetic primer
cagatctcagctggaccaca

SEQ ID NO: 36 - DNA, 20 bp, Artificial sequence
  Synthetic primer
tcatagaggatggtggcaga

SEQ ID NO: 37 - DNA, 25 bp, Artificial sequence
  Synthetic primer
gcacccacatttctktcttacaatg

SEQ ID NO: 38 - DNA, 25 bp, Artificial sequence
  Synthetic primer
atgtgcaccaagactccttgttaaa

SEQ ID NO: 39 - DNA, 21 bp, Artificial sequence
  Synthetic primer
gcagctatggctttgaagctg

SEQ ID NO: 40 - DNA, 21 bp, Artificial sequence
  Synthetic primer
aavggytttgaggctgaattt

SEQ ID NO: 41 - DNA, 26 bp, Artificial sequence
  Synthetic primer
caagacaaaagttacaaacgaagtgg

SEQ ID NO: 42 - DNA, 25 bp, Artificial sequence
  Synthetic primer
tggacatgaaacaagaccaaagact

SEQ ID NO: 43 - DNA, 24 bp, Artificial sequence
  Synthetic primer
aaaaaggaaagaaagactgaaggt

SEQ ID NO: 44 - DNA, 25 bp, Artificial sequence
  Synthetic primer
tcagctggatatgagaagcagaaag

SEQ ID NO: 45 - DNA, 25 bp, Artificial sequence
  Synthetic primer
aagggaagsaacaaaggttttgaag

SEQ ID NO: 46 - DNA, 25 bp, Artificial sequence
  Synthetic primer
agaacacaaagtcgaacggaagata

SEQ ID NO: 47 - DNA, 25 bp, Artificial sequence
  Synthetic primer
ttgtgtctttgaccttaattcaatc

SEQ ID NO: 48 - DNA, 23 bp, Artificial sequence
  Synthetic primer
tcartgttccagagggagccayt

SEQ ID NO: 49 - DNA, 22 bp, Artificial sequence
  Synthetic primer
ctgagtgtccaggagggwgaca

SEQ ID NO: 50 - DNA, 24 bp, Artificial sequence
  Synthetic primer
agcagtggggaaatgatttttctt

SEQ ID NO: 51 - DNA, 25 bp, Artificial sequence
  Synthetic primer
tctagagagagcatcaaaggcttca

SEQ ID NO: 52 - DNA, 25 bp, Artificial sequence
  Synthetic primer
cgttcaaatgaaagagagaaacaca

SEQ ID NO: 53 - DNA, 25 bp, Artificial sequence
  Synthetic primer
cctgaaaagttcagaaaaccaggag

SEQ ID NO: 54 - DNA, 25 bp, Artificial sequence
  Synthetic primer
ccttattcgtcggaactcttttgat

SEQ ID NO: 55 - DNA, 25 bp, Artificial sequence
  Synthetic primer
ctggggaagaaaaggagaaagaaag

SEQ ID NO: 56 - DNA, 25 bp, Artificial sequence
  Synthetic primer
cagagagagcaaacaagtggaagac

SEQ ID NO: 57 - DNA, 25 bp, Artificial sequence
  Synthetic primer
catcaacctgttttacattccctca

SEQ ID NO: 58 - DNA, 25 bp, Artificial sequence
  Synthetic primer
gcattattgatagccatacgtccag

SEQ ID NO: 59 - DNA, 25 bp, Artificial sequence
  Synthetic primer
taaatggggatgaaaagaagaaagg

SEQ ID NO: 60 - DNA, 20 bp, Artificial sequence
  Synthetic primer
ctggtggacatcccgttttt

SEQ ID NO: 61 - DNA, 23 bp, Artificial sequence
  Synthetic primer
attggtatcgacagmttcmctcc

SEQ ID NO: 62 - DNA, 25 bp, Artificial sequence
  Synthetic primer
cctgtcctcctggtgacagtagtta

SEQ ID NO: 63 - DNA, 25 bp, Artificial sequence
  Synthetic primer
ggacccctcatgtccttatttaaca

SEQ ID NO: 64 - DNA, 25 bp, Artificial sequence
  Synthetic primer
tgctgaaggtcctacattcctgata

SEQ ID NO: 65 - DNA, 25 bp, Artificial sequence
  Synthetic primer
cccgtcttcctgatgatattactga

SEQ ID NO: 66 - DNA, 26 bp, Artificial sequence
  Synthetic primer
gaagattattttcctcatttatcagc

SEQ ID NO: 67 - DNA, 25 bp, Artificial sequence
  Synthetic primer
gggaaggccctaatatcttaatgga

SEQ ID NO: 68 - DNA, 25 bp, Artificial sequence
  Synthetic primer
cccagtgaagagatggttttcctta

SEQ ID NO: 69 - DNA, 26 bp, Artificial sequence
  Synthetic primer
tgaaggtcttatcttcttgatgatgc

SEQ ID NO: 70 - DNA, 25 bp, Artificial sequence
  Synthetic primer
aggtcctgtcctcttgatagcctta

SEQ ID NO: 71 - DNA, 25 bp, Artificial sequence
  Synthetic primer
ggaaaagaaagctcccacatttcta

SEQ ID NO: 72 - DNA, 25 bp, Artificial sequence
  Synthetic primer
cctcatttccctgatacaaatgcta

SEQ ID NO: 73 - DNA, 25 bp, Artificial sequence
  Synthetic primer
agcaggcagatgattctcgttattc

SEQ ID NO: 74 - DNA, 25 bp, Artificial sequence
  Synthetic primer
gtctggaatctctgtttgtgttgct

SEQ ID NO: 75 - DNA, 25 bp, Artificial sequence
  Synthetic primer
tgcagcttcttcagagagagacaat

SEQ ID NO: 76 - DNA, 25 bp, Artificial sequence
  Synthetic primer
gcattgtttccttgtttatgctgag

SEQ ID NO: 77 - DNA, 25 bp, Artificial sequence
  Synthetic primer
aagaaatccctggagttcatgtttt

SEQ ID NO: 78 - DNA, 25 bp, Artificial sequence
  Synthetic primer
gtacagacaaatcttggggcagaaa

SEQ ID NO: 79 - DNA, 25 bp, Artificial sequence
  Synthetic primer
tctgggccatratrctatgtattgg

SEQ ID NO: 80 - DNA, 22 bp, Artificial sequence
  Synthetic primer
agtgtgccaagtcgcttctcac

SEQ ID NO: 81 - DNA, 24 bp, Artificial sequence
  Synthetic primer
gggccccagtttatctttcagtat

SEQ ID NO: 82 - DNA, 24 bp, Artificial sequence
  Synthetic primer
cagytcctcctttggtatgacgag

SEQ ID NO: 83 - DNA, 25 bp, Artificial sequence
  Synthetic primer
gagggtaccactgacaaaggagaag

SEQ ID NO: 84 - DNA, 25 bp, Artificial sequence
  Synthetic primer
actcagttggtgagggtacaactgc

SEQ ID NO: 85 - DNA, 24 bp, Artificial sequence
  Synthetic primer
aggtaccactggcaaaggagaagt

SEQ ID NO: 86 - DNA, 24 bp, Artificial sequence
  Synthetic primer
tcagttggtgctggtatcactgay

SEQ ID NO: 87 - DNA, 25 bp, Artificial sequence
  Synthetic primer
tgctctcactgacaaaggagaagtt

SEQ ID NO: 88 - DNA, 25 bp, Artificial sequence
  Synthetic primer
tgctgctggtactactgacaaagaa

SEQ ID NO: 89 - DNA, 25 bp, Artificial sequence
  Synthetic primer
gctggtatcactgacaaaggagaag

SEQ ID NO: 90 - DNA, 24 bp, Artificial sequence
  Synthetic primer
caggtcatamtgccctttaytggt

SEQ ID NO: 91 - DNA, 25 bp, Artificial sequence
  Synthetic primer
gacttactcccagagtgatgctcaa

SEQ ID NO: 92 - DNA, 25 bp, Artificial sequence
  Synthetic primer
agggccmagagtttctgacttmctt

SEQ ID NO: 93 - DNA, 25 bp, Artificial sequence
  Synthetic primer
gccagagtttctgacttatttccag

SEQ ID NO: 94 - DNA, 25 bp, Artificial sequence
  Synthetic primer
tgctcagattaggaaccattattca

SEQ ID NO: 95 - DNA, 24 bp, Artificial sequence
  Synthetic primer
aacagtgttctgatatcgacagga

SEQ ID NO: 96 - DNA, 25 bp, Artificial sequence
  Synthetic primer
gtactggtaccaacagagcctggac

SEQ ID NO: 97 - DNA, 23 bp, Artificial sequence
  Synthetic primer
ggtatcgacaagaccygggrcat

SEQ ID NO: 98 - DNA, 25 bp, Artificial sequence
  Synthetic primer
acagttgcctaaggatcgattttct

SEQ ID NO: 99 - DNA, 25 bp, Artificial sequence
  Synthetic primer
cagggactggaattgctgarttact

SEQ ID NO: 100 - DNA, 24 bp, Artificial sequence
  Synthetic primer
tctggtacagacagaccatgatgc

SEQ ID NO: 101 - DNA, 25 bp, Artificial sequence
  Synthetic primer
ttcgttttatgaaaagatgcagagc

SEQ ID NO: 102 - DNA, 25 bp, Artificial sequence
  Synthetic primer
atcgattcttagctgaaaggactgg

SEQ ID NO: 103 - DNA, 25 bp, Artificial sequence
  Synthetic primer
agacacccctgataacttccaatcc

SEQ ID NO: 104 - DNA, 25 bp, Artificial sequence
  Synthetic primer
aaacaggtatgcccaaggaaagatt

SEQ ID NO: 105 - DNA, 25 bp, Artificial sequence
  Synthetic primer
aaacattgcagttgattcagggatg

SEQ ID NO: 106 - DNA, 25 bp, Artificial sequence
  Synthetic primer
catagatgagtcaggaatgccaaag

SEQ ID NO: 107 - DNA, 24 bp, Artificial sequence
  Synthetic primer
aacagtgttctgatatcgacagga

SEQ ID NO: 108 - DNA, 27 bp, Artificial sequence
  Synthetic primer
tcagaaaggagatatagctgaagggta

SEQ ID NO: 109 - DNA, 25 bp, Artificial sequence
  Synthetic primer
caaggccacatacgagcaaggcgtc

SEQ ID NO: 110 - DNA, 26 bp, Artificial sequence
  Synthetic primer
tcagaaagcagaaataatcaatgagc

SEQ ID NO: 111 - DNA, 26 bp, Artificial sequence
  Synthetic primer
gaggagatctaactgaaggctacgtg

SEQ ID NO: 112 - DNA, 24 bp, Artificial sequence
  Synthetic primer
caagaaacggagatgcacaagaag

SEQ ID NO: 113 - DNA, 27 bp, Artificial sequence
  Synthetic primer
cggttgatctattactcctttgatgtc

SEQ ID NO: 114 - DNA, 25 bp, Artificial sequence
  Synthetic primer
aattccacagagaagggagatcttt

SEQ ID NO: 115 - DNA, 25 bp, Artificial sequence
  Synthetic primer
actgggagcactgaaaaaggagata

SEQ ID NO: 116 - DNA, 25 bp, Artificial sequence
  Synthetic primer
ttcaatgaatgttgaggtgactgat

SEQ ID NO: 117 - DNA, 27 bp, Artificial sequence
  Synthetic primer
cggctgatctatttctcatatgatgtt

SEQ ID NO: 118 - DNA, 22 bp, Artificial sequence
  Synthetic primer
gacactgatcgcaactgcaaat

SEQ ID NO: 119 - DNA, 23 bp, Artificial sequence
  Synthetic primer
gcctccagctgctcttctactcc

SEQ ID NO: 120 - DNA, 62 bp, Artificial sequence
  Synthetic primer
  misc_feature (34)..(34): n is a, c, g, or t
  misc_feature (36)..(36): n is a, c, g, or t
  misc_feature (38)..(44): n is a, c, g, or t
acactctttccctacacgacgctcttccgatctnhnhnnnnnnnggtacacggcagggtcag

SEQ ID NO: 121 - DNA, 62 bp, Artificial sequence
  Synthetic primer
  misc_feature (34)..(34): n is a, c, g, or t
  misc_feature (36)..(36): n is a, c, g, or t
  misc_feature (38)..(44): n is a, c, g, or t
acactctttccctacacgacgctcttccgatctnhnhnnnnnnngacctcgggtgggaacac

SEQ ID NO: 122 - DNA, 45 bp, Artificial sequence
  Synthetic primer
gacgtgtgctcttccgatctgamaggtcgtttttcttcattcctt

SEQ ID NO: 123 - DNA, 45 bp, Artificial sequence
  Synthetic primer
gacgtgtgctcttccgatctagggacgatacaacatgacctatga

SEQ ID NO: 124 - DNA, 40 bp, Artificial sequence
  Synthetic primer
gacgtgtgctcttccgatcttccttccacctgavgaaacc

SEQ ID NO: 125 - DNA, 44 bp, Artificial sequence
  Synthetic primer
gacgtgtgctcttccgatctttyaatctgaggaaaccctctgtg

SEQ ID NO: 126 - DNA, 44 bp, Artificial sequence
  Synthetic primer
gacgtgtgctcttccgatctgacagaaagtccagcactctgagc

SEQ ID NO: 127 - DNA, 45 bp, Artificial sequence
  Synthetic primer
gacgtgtgctcttccgatctggataaacatctgtctctgcgcatt

SEQ ID NO: 128 - DNA, 45 bp, Artificial sequence
  Synthetic primer
gacgtgtgctcttccgatctcacctttgataccacccttaamcag

SEQ ID NO: 129 - DNA, 45 bp, Artificial sequence
  Synthetic primer
gacgtgtgctcttccgatctttactgaagaatggaagcagcttgt

SEQ ID NO: 130 - DNA, 45 bp, Artificial sequence
  Synthetic primer
gacgtgtgctcttccgatctcgtaargaaaccacttctttccact

SEQ ID NO: 131 - DNA, 43 bp, Artificial sequence
  Synthetic primer
gacgtgtgctcttccgatctaagcaaagctctctgcacatcac

SEQ ID NO: 132 - DNA, 46 bp, Artificial sequence
  Synthetic primer
gacgtgtgctcttccgatctgcttggaaaagaraarttttatagtg

SEQ ID NO: 133 - DNA, 43 bp, Artificial sequence
  Synthetic primer
gacgtgtgctcttccgatctgaagatggaaggtttacagcaca

SEQ ID NO: 134 - DNA, 47 bp, Artificial sequence
  Synthetic primer
gacgtgtgctcttccgatcttyattatagacattcgttcaaatrtgg

SEQ ID NO: 135 - DNA, 45 bp, Artificial sequence
  Synthetic primer
gacgtgtgctcttccgatctttgaatttccagaaggcaagaaaat

SEQ ID NO: 136 - DNA, 45 bp, Artificial sequence
  Synthetic primer
gacgtgtgctcttccgatctgaccttaacaaaggcgagacatctt

SEQ ID NO: 137 - DNA, 45 bp, Artificial sequence
  Synthetic primer
gacgtgtgctcttccgatctcttgacacttccaagaaaagcagtt

SEQ ID NO: 138 - DNA, 45 bp, Artificial sequence
  Synthetic primer
gacgtgtgctcttccgatctttttcaggccagtcctatcaagagt

SEQ ID NO: 139 - DNA, 45 bp, Artificial sequence
  Synthetic primer
gacgtgtgctcttccgatcttgaaataagtggtcggtattcttgg

SEQ ID NO: 140 - DNA, 45 bp, Artificial sequence
  Synthetic primer
gacgtgtgctcttccgatctagccacattaacaaagaaggaaagc

SEQ ID NO: 141 - DNA, 44 bp, Artificial sequence
  Synthetic primer
gacgtgtgctcttccgatctttaatgcctcgctggataaatcat

SEQ ID NO: 142 - DNA, 44 bp, Artificial sequence
  Synthetic primer
gacgtgtgctcttccgatctgctacggaacgctacagcttattg

SEQ ID NO: 143 - DNA, 47 bp, Artificial sequence
  Synthetic primer
gacgtgtgctcttccgatcttgagtgaaaagaaagaaggaagattca

SEQ ID NO: 144 - DNA, 45 bp, Artificial sequence
  Synthetic primer
gacgtgtgctcttccgatcttaccaaggagggttacagctatttg

SEQ ID NO: 145 - DNA, 45 bp, Artificial sequence
  Synthetic primer
gacgtgtgctcttccgatcttggagaagtgaagaagcagaaaaga

SEQ ID NO: 146 - DNA, 45 bp, Artificial sequence
  Synthetic primer
gacgtgtgctcttccgatctaagacagaaagtccagyaccttgat

SEQ ID NO: 147 - DNA, 45 bp, Artificial sequence
  Synthetic primer
gacgtgtgctcttccgatcttggagaagtgaagaagctgaagaga

SEQ ID NO: 148 - DNA, 45 bp, Artificial sequence
  Synthetic primer
gacgtgtgctcttccgatctgaagactaaaatccgcagtcaaagc

SEQ ID NO: 149 - DNA, 47 bp, Artificial sequence
  Synthetic primer
gacgtgtgctcttccgatcttccattaaggataaaaatgaagatgga

SEQ ID NO: 150 - DNA, 45 bp, Artificial sequence
  Synthetic primer
gacgtgtgctcttccgatctaagcrgcaaagctccctgtacctta

SEQ ID NO: 151 - DNA, 44 bp, Artificial sequence
  Synthetic primer
gacgtgtgctcttccgatctaatgcgacacagggtcaatattct

SEQ ID NO: 152 - DNA, 45 bp, Artificial sequence
  Synthetic primer
gacgtgtgctcttccgatcttgtggatagaaaacaggacagaagg

SEQ ID NO: 153 - DNA, 45 bp, Artificial sequence
  Synthetic primer
gacgtgtgctcttccgatcttaagtcaaatgcaaagcctgtgaac

SEQ ID NO: 154 - DNA, 45 bp, Artificial sequence
  Synthetic primer
gacgtgtgctcttccgatctggggaagagaaaagtcatgaaaaga

SEQ ID NO: 155 - DNA, 45 bp, Artificial sequence
  Synthetic primer
gacgtgtgctcttccgatctggaagactgactgctcagtttggta

SEQ ID NO: 156 - DNA, 45 bp, Artificial sequence
  Synthetic primer
gacgtgtgctcttccgatcttggaattgaaaagaagtcaggaaga

SEQ ID NO: 157 - DNA, 45 bp, Artificial sequence
  Synthetic primer
gacgtgtgctcttccgatctagaagatcagtggaagattcacagc

SEQ ID NO: 158 - DNA, 45 bp, Artificial sequence
  Synthetic primer
gacgtgtgctcttccgatctagaaagcagccaaatccttcagtct

SEQ ID NO: 159 - DNA, 45 bp, Artificial sequence
  Synthetic primer
gacgtgtgctcttccgatctgacgattaatggcctcacttgatac

SEQ ID NO: 160 - DNA, 45 bp, Artificial sequence
  Synthetic primer
gacgtgtgctcttccgatctggaggcggaaatattaaagacaaaa

SEQ ID NO: 161 - DNA, 45 bp, Artificial sequence
  Synthetic primer
gacgtgtgctcttccgatctgcatggaagattaattgccacaata

SEQ ID NO: 162 - DNA, 45 bp, Artificial sequence
  Synthetic primer
gacgtgtgctcttccgatctctgacagctctcgcttataccttca

SEQ ID NO: 163 - DNA, 45 bp, Artificial sequence
  Synthetic primer
gacgtgtgctcttccgatctgcctgatggatcaaatttcactctg

SEQ ID NO: 164 - DNA, 45 bp, Artificial sequence
  Synthetic primer
gacgtgtgctcttccgatctaatgaaacagttccaaatcgmttct

SEQ ID NO: 165 - DNA, 42 bp, Artificial sequence
  Synthetic primer
gacgtgtgctcttccgatctccaagtcgcttctcacctgaat

SEQ ID NO: 166 - DNA, 44 bp, Artificial sequence
  Synthetic primer
gacgtgtgctcttccgatctcgccagttctctaactctcgctct

SEQ ID NO: 167 - DNA, 45 bp, Artificial sequence
  Synthetic primer
gacgtgtgctcttccgatctttactgagtcaaacacggagctagg

SEQ ID NO: 168 - DNA, 45 bp, Artificial sequence
  Synthetic primer
gacgtgtgctcttccgatctctctgagatgaatgtgagtgccttg

SEQ ID NO: 169 - DNA, 43 bp, Artificial sequence
  Synthetic primer
gacgtgtgctcttccgatctctgagctgaatgtgaacgccttg

SEQ ID NO: 170 - DNA, 45 bp, Artificial sequence
  Synthetic primer
gacgtgtgctcttccgatcttctccagattaaacaaacgggagtt

SEQ ID NO: 171 - DNA, 45 bp, Artificial sequence
  Synthetic primer
gacgtgtgctcttccgatctctgatggctacaatgtctccagatt

SEQ ID NO: 172 - DNA, 45 bp, Artificial sequence
  Synthetic primer
gacgtgtgctcttccgatctagtgtctccagagcaaacacagatg

SEQ ID NO: 173 - DNA, 45 bp, Artificial sequence
  Synthetic primer
gacgtgtgctcttccgatctgtctccagatcaamcacagaggatt

SEQ ID NO: 174 - DNA, 44 bp, Artificial sequence
  Synthetic primer
gacgtgtgctcttccgatctaaacacagaggatttcccrctcag

SEQ ID NO: 175 - DNA, 43 bp, Artificial sequence
  Synthetic primer
gacgtgtgctcttccgatctgtctgagggatccatctccactc

SEQ ID NO: 176 - DNA, 44 bp, Artificial sequence
  Synthetic primer
gacgtgtgctcttccgatcttcgcttctctgcagagaggactgg

SEQ ID NO: 177 - DNA, 45 bp, Artificial sequence
  Synthetic primer
gacgtgtgctcttccgatctctgagggatccgtctctactctgaa

SEQ ID NO: 178 - DNA, 43 bp, Artificial sequence
  Synthetic primer
gacgtgtgctcttccgatctctgagrgatccgtctccactctg

SEQ ID NO: 179 - DNA, 43 bp, Artificial sequence
  Synthetic primer
gacgtgtgctcttccgatctggtctgaggatctttctccacct

SEQ ID NO: 180 - DNA, 43 bp, Artificial sequence
  Synthetic primer
gacgtgtgctcttccgatctgagggatccatctccactctgac

SEQ ID NO: 181 - DNA, 43 bp, Artificial sequence
  Synthetic primer
gacgtgtgctcttccgatctctgcagagaggcctaagggatct

SEQ ID NO: 182 - DNA, 43 bp, Artificial sequence
  Synthetic primer
gacgtgtgctcttccgatctaagctcaagcattttccctcaac

SEQ ID NO: 183 - DNA, 45 bp, Artificial sequence
  Synthetic primer
gacgtgtgctcttccgatctatgtcacagaggggtactgtgtttc

SEQ ID NO: 184 - DNA, 43 bp, Artificial sequence
  Synthetic primer
gacgtgtgctcttccgatctacagttccctgacttgcactctg

SEQ ID NO: 185 - DNA, 45 bp, Artificial sequence
  Synthetic primer
gacgtgtgctcttccgatctacaaaggagaagtctcagatggcta

SEQ ID NO: 186 - DNA, 44 bp, Artificial sequence
  Synthetic primer
gacgtgtgctcttccgatcttgtctccagatccaagacagagaa

SEQ ID NO: 187 - DNA, 44 bp, Artificial sequence
  Synthetic primer
gacgtgtgctcttccgatctctgcagagaggctcaaaggagtag

SEQ ID NO: 188 - DNA, 45 bp, Artificial sequence
  Synthetic primer
gacgtgtgctcttccgatctatcattctcyactctgaggatccar

SEQ ID NO: 189 - DNA, 45 bp, Artificial sequence
  Synthetic primer
gacgtgtgctcttccgatctactctgargatccagccctcagaac

SEQ ID NO: 190 - DNA, 46 bp, Artificial sequence
  Synthetic primer
gacgtgtgctcttccgatctcagctcaacagttcagtgactatcat

SEQ ID NO: 191 - DNA, 45 bp, Artificial sequence
  Synthetic primer
gacgtgtgctcttccgatctgaaaggactggagggacgtattcta

SEQ ID NO: 192 - DNA, 44 bp, Artificial sequence
  Synthetic primer
gacgtgtgctcttccgatctgccgaacacttctttctgctttct

SEQ ID NO: 193 - DNA, 45 bp, Artificial sequence
  Synthetic primer
gacgtgtgctcttccgatctattttcagctaagtgcctcccaaat

SEQ ID NO: 194 - DNA, 44 bp, Artificial sequence
  Synthetic primer
gacgtgtgctcttccgatctcacagctgaaagacctaacggaac

SEQ ID NO: 195 - DNA, 45 bp, Artificial sequence
  Synthetic primer
gacgtgtgctcttccgatctattttctgctgaatttcccaaagag

SEQ ID NO: 196 - DNA, 43 bp, Artificial sequence
  Synthetic primer
gacgtgtgctcttccgatctgtctctcgggagaagaaggaatc

SEQ ID NO: 197 - DNA, 45 bp, Artificial sequence
  Synthetic primer
gacgtgtgctcttccgatctgacaagtttctcatcaaccatgcaa

SEQ ID NO: 198 - DNA, 44 bp, Artificial sequence
  Synthetic primer
gacgtgtgctcttccgatctcaatgctccaaaaactcatcctgt

SEQ ID NO: 199 - DNA, 45 bp, Artificial sequence
  Synthetic primer
gacgtgtgctcttccgatctaggagaaggggctatttcttctcag

SEQ ID NO: 200 - DNA, 45 bp, Artificial sequence
  Synthetic primer
gacgtgtgctcttccgatctattctcatctcaatgccccaagaac

SEQ ID NO: 201 - DNA, 43 bp, Artificial sequence
  Synthetic primer
gacgtgtgctcttccgatctgacaggcacaggctaaattctcc

SEQ ID NO: 202 - DNA, 45 bp, Artificial sequence
  Synthetic primer
gacgtgtgctcttccgatctagtctccagaataaggacggagcat

SEQ ID NO: 203 - DNA, 45 bp, Artificial sequence
  Synthetic primer
gacgtgtgctcttccgatctctctgaggggtatcatgtttcttga

SEQ ID NO: 204 - DNA, 46 bp, Artificial sequence
  Synthetic primer
gacgtgtgctcttccgatctcaaagtctctcgaaaagagaagagga

SEQ ID NO: 205 - DNA, 44 bp, Artificial sequence
  Synthetic primer
gacgtgtgctcttccgatctaagaaggagcgcttctccctgatt

SEQ ID NO: 206 - DNA, 42 bp, Artificial sequence
  Synthetic primer
gacgtgtgctcttccgatctcgcccaaacctaacattctcaa

SEQ ID NO: 207 - DNA, 43 bp, Artificial sequence
  Synthetic primer
gacgtgtgctcttccgatctccagaatctctcagcctccagac

SEQ ID NO: 208 - DNA, 45 bp, Artificial sequence; identical to SEQ ID NO: 25
  Synthetic primer

SEQ ID NO: 209 - DNA, 66 bp, Artificial sequence
  Synthetic primer
  misc_feature (27)..(32): n is a, c, g, or t
caagcagaagacggcatacgagataannnnnngtgactggagttcagacgtgtgctcttccgatct

SEQ ID NO: 210 - DNA, 60 bp, Artificial sequence
  Synthetic primer
  misc_feature (24)..(28): n is a, c, g, or t
  misc_feature (60)..(60): n is a, c, g, or t
aagcagtggtatcaacgcagagtnnnnnttttttttttttttttttttttttttttttvn

SEQ ID NO: 211 - DNA, 30 bp, Artificial sequence
  Synthetic primer
aagcagtggtatcaacgcagagtcgacggg

SEQ ID NO: 212 - DNA, 23 bp, Artificial sequence
  Synthetic primer
aagcagtggtatcaacgcagagt

SEQ ID NO: 213 - DNA, 23 bp, Artificial sequence
  Synthetic primer
aagcagtggtatcaacgcagagt

SEQ ID NO: 214 - DNA, 25 bp, Artificial sequence
  Synthetic primer
gcacccacatttctktcttacaatg

SEQ ID NO: 215 - DNA, 25 bp, Artificial sequence
  Synthetic primer
atgtgcaccaagactccttgttaaa

SEQ ID NO: 216 - DNA, 21 bp, Artificial sequence
  Synthetic primer
gcagctatggctttgaagctg

SEQ ID NO: 217 - DNA, 21 bp, Artificial sequence
  Synthetic primer
aavggytttgaggctgaattt

SEQ ID NO: 218 - DNA, 26 bp, Artificial sequence
  Synthetic primer
caagacaaaagttacaaacgaagtgg

SEQ ID NO: 219 - DNA, 25 bp, Artificial sequence
  Synthetic primer
tggacatgaaacaagaccaaagact

SEQ ID NO: 220 - DNA, 24 bp, Artificial sequence
  Synthetic primer
aaaaaggaaagaaagactgaaggt

SEQ ID NO: 221 - DNA, 25 bp, Artificial sequence
  Synthetic primer
tcagctggatatgagaagcagaaag

SEQ ID NO: 222 - DNA, 25 bp, Artificial sequence
  Synthetic primer
aagggaagsaacaaaggttttgaag

SEQ ID NO: 223 - DNA, 25 bp, Artificial sequence
  Synthetic primer
agaacacaaagtcgaacggaagata

SEQ ID NO: 224 - DNA, 25 bp, Artificial sequence
  Synthetic primer
ttgtgtctttgaccttaattcaatc

SEQ ID NO: 225 - DNA, 23 bp, Artificial sequence
  Synthetic primer
tcartgttccagagggagccayt

SEQ ID NO: 226 - DNA, 22 bp, Artificial sequence
  Synthetic primer
ctgagtgtccaggagggwgaca

SEQ ID NO: 227 - DNA, 24 bp, Artificial sequence
  Synthetic primer
agcagtggggaaatgatttttctt

SEQ ID NO: 228 - DNA, 25 bp, Artificial sequence
  Synthetic primer
tctagagagagcatcaaaggcttca

SEQ ID NO: 229 - DNA, 25 bp, Artificial sequence
  Synthetic primer
cgttcaaatgaaagagagaaacaca

SEQ ID NO: 230 - DNA, 25 bp, Artificial sequence
  Synthetic primer
cctgaaaagttcagaaaaccaggag

SEQ ID NO: 231 - DNA, 25 bp, Artificial sequence
  Synthetic primer
ccttattcgtcggaactcttttgat

SEQ ID NO: 232 - DNA, 25 bp, Artificial sequence
  Synthetic primer
ctggggaagaaaaggagaaagaaag

SEQ ID NO: 233 - DNA, 25 bp, Artificial sequence
  Synthetic primer
cagagagagcaaacaagtggaagac

SEQ ID NO: 234 - DNA, 25 bp, Artificial sequence
  Synthetic primer
catcaacctgttttacattccctca

SEQ ID NO: 235 - DNA, 25 bp, Artificial sequence
  Synthetic primer
gcattattgatagccatacgtccag

SEQ ID NO: 236 - DNA, 25 bp, Artificial sequence
  Synthetic primer
taaatggggatgaaaagaagaaagg

SEQ ID NO: 237 - DNA, 20 bp, Artificial sequence
  Synthetic primer
ctggtggacatcccgttttt

SEQ ID NO: 238 - DNA, 23 bp, Artificial sequence
  Synthetic primer
attggtatcgacagmttcmctcc

SEQ ID NO: 239 - DNA, 25 bp, Artificial sequence
  Synthetic primer
cctgtcctcctggtgacagtagtta

SEQ ID NO: 240 - DNA, 25 bp, Artificial sequence
  Synthetic primer
ggacccctcatgtccttatttaaca

SEQ ID NO: 241 - DNA, 25 bp, Artificial sequence
  Synthetic primer
tgctgaaggtcctacattcctgata

SEQ ID NO: 242 - DNA, 25 bp, Artificial sequence
  Synthetic primer
cccgtcttcctgatgatattactga

SEQ ID NO: 243 - DNA, 26 bp, Artificial sequence
  Synthetic primer
gaagattattttcctcatttatcagc

SEQ ID NO: 244 - DNA, 25 bp, Artificial sequence
  Synthetic primer
gggaaggccctaatatcttaatgga

SEQ ID NO: 245 - DNA, 25 bp, Artificial sequence
  Synthetic primer
cccagtgaagagatggttttcctta

SEQ ID NO: 246 - DNA, 26 bp, Artificial sequence
  Synthetic primer
tgaaggtcttatcttcttgatgatgc

SEQ ID NO: 247 - DNA, 25 bp, Artificial sequence
  Synthetic primer
aggtcctgtcctcttgatagcctta

SEQ ID NO: 248 - DNA, 25 bp, Artificial sequence
  Synthetic primer
ggaaaagaaagctcccacatttcta

SEQ ID NO: 249 - DNA, 25 bp, Artificial sequence
  Synthetic primer
cctcatttccctgatacaaatgcta

SEQ ID NO: 250 - DNA, 25 bp, Artificial sequence
  Synthetic primer
agcaggcagatgattctcgttattc

SEQ ID NO: 251 - DNA, 25 bp, Artificial sequence
  Synthetic primer
gtctggaatctctgtttgtgttgct

SEQ ID NO: 252 - DNA, 25 bp, Artificial sequence
  Synthetic primer
tgcagcttcttcagagagagacaat

SEQ ID NO: 253 - DNA, 25 bp, Artificial sequence
  Synthetic primer
gcattgtttccttgtttatgctgag

SEQ ID NO: 254 - DNA, 25 bp, Artificial sequence
  Synthetic primer
aagaaatccctggagttcatgtttt

SEQ ID NO: 255 - DNA, 25 bp, Artificial sequence
  Synthetic primer
gtacagacaaatcttggggcagaaa

SEQ ID NO: 256 - DNA, 25 bp, Artificial sequence
  Synthetic primer
tctgggccatratrctatgtattgg

SEQ ID NO: 257 - DNA, 22 bp, Artificial sequence
  Synthetic primer
agtgtgccaagtcgcttctcac

SEQ ID NO: 258 - DNA, 24 bp, Artificial sequence
  Synthetic primer
gggccccagtttatctttcagtat

SEQ ID NO: 259 - DNA, 24 bp, Artificial sequence
  Synthetic primer
cagytcctcctttggtatgacgag

SEQ ID NO: 260 - DNA, 25 bp, Artificial sequence
  Synthetic primer
gagggtaccactgacaaaggagaag

SEQ ID NO: 261 - DNA, 25 bp, Artificial sequence
  Synthetic primer
actcagttggtgagggtacaactgc

SEQ ID NO: 262 - DNA, 24 bp, Artificial sequence
  Synthetic primer
aggtaccactggcaaaggagaagt

SEQ ID NO: 263 - DNA, 24 bp, Artificial sequence
  Synthetic primer
tcagttggtgctggtatcactgay

SEQ ID NO: 264 - DNA, 25 bp, Artificial sequence
  Synthetic primer
tgctctcactgacaaaggagaagtt

SEQ ID NO: 265 - DNA, 25 bp, Artificial sequence
  Synthetic primer
tgctgctggtactactgacaaagaa

SEQ ID NO: 266 - DNA, 25 bp, Artificial sequence
  Synthetic primer
gctggtatcactgacaaaggagaag

SEQ ID NO: 267 - DNA, 24 bp, Artificial sequence
  Synthetic primer
caggtcatamtgccctttaytggt

SEQ ID NO: 268 - DNA, 25 bp, Artificial sequence
  Synthetic primer
gacttactcccagagtgatgctcaa

SEQ ID NO: 269 - DNA, 25 bp, Artificial sequence
  Synthetic primer
agggccmagagtttctgacttmctt

SEQ ID NO: 270 - DNA, 25 bp, Artificial sequence
  Synthetic primer
gccagagtttctgacttatttccag

SEQ ID NO: 271 - DNA, 25 bp, Artificial sequence
  Synthetic primer
tgctcagattaggaaccattattca

SEQ ID NO: 272 - DNA, 25 bp, Artificial sequence
  Synthetic primer
gtactggtaccaacagagcctggac

SEQ ID NO: 273 - DNA, 23 bp, Artificial sequence
  Synthetic primer
ggtatcgacaagaccygggrcat

SEQ ID NO: 274 - DNA, 25 bp, Artificial sequence
  Synthetic primer
acagttgcctaaggatcgattttct

SEQ ID NO: 275 - DNA, 25 bp, Artificial sequence
  Synthetic primer
cagggactggaattgctgarttact

SEQ ID NO: 276 - DNA, 24 bp, Artificial sequence
  Synthetic primer
tctggtacagacagaccatgatgc

SEQ ID NO: 277 - DNA, 25 bp, Artificial sequence
  Synthetic primer
ttcgttttatgaaaagatgcagagc

SEQ ID NO: 278 - DNA, 25 bp, Artificial sequence
  Synthetic primer
atcgattcttagctgaaaggactgg

SEQ ID NO: 279 - DNA, 25 bp, Artificial sequence
  Synthetic primer
agacacccctgataacttccaatcc

SEQ ID NO: 280 - DNA, 25 bp, Artificial sequence
  Synthetic primer
aaacaggtatgcccaaggaaagatt

SEQ ID NO: 281 - DNA, 25 bp, Artificial sequence
  Synthetic primer
aaacattgcagttgattcagggatg

SEQ ID NO: 282 - DNA, 25 bp, Artificial sequence
  Synthetic primer
catagatgagtcaggaatgccaaag

SEQ ID NO: 283 - DNA, 27 bp, Artificial sequence
  Synthetic primer
tcagaaaggagatatagctgaagggta

SEQ ID NO: 284 - DNA, 25 bp, Artificial sequence
  Synthetic primer
caaggccacatacgagcaaggcgtc

SEQ ID NO: 285 - DNA, 26 bp, Artificial sequence
  Synthetic primer
tcagaaagcagaaataatcaatgagc

SEQ ID NO: 286 - DNA, 26 bp, Artificial sequence
  Synthetic primer
gaggagatctaactgaaggctacgtg

SEQ ID NO: 287 - DNA, 24 bp, Artificial sequence
  Synthetic primer
caagaaacggagatgcacaagaag

SEQ ID NO: 288 - DNA, 27 bp, Artificial sequence
  Synthetic primer
cggttgatctattactcctttgatgtc

SEQ ID NO: 289 - DNA, 25 bp, Artificial sequence
  Synthetic primer
aattccacagagaagggagatcttt

SEQ ID NO: 290 - DNA, 25 bp, Artificial sequence
  Synthetic primer
actgggagcactgaaaaaggagata

SEQ ID NO: 291 - DNA, 25 bp, Artificial sequence
  Synthetic primer
ttcaatgaatgttgaggtgactgat

SEQ ID NO: 292 - DNA, 27 bp, Artificial sequence
  Synthetic primer
cggctgatctatttctcatatgatgtt

SEQ ID NO: 293 - DNA, 22 bp, Artificial sequence
  Synthetic primer
gacactgatcgcaactgcaaat

SEQ ID NO: 294 - DNA, 23 bp, Artificial sequence
  Synthetic primer
gcctccagctgctcttctactcc

SEQ ID NO: 295 - DNA, 23 bp, Artificial sequence
  Synthetic primer
aagcagtggtatcaacgcagagt

SEQ ID NO: 296 - DNA, 45 bp, Artificial sequence; identical to SEQ ID NO: 122
  Synthetic primer

SEQ ID NO: 297 - DNA, 45 bp, Artificial sequence; identical to SEQ ID NO: 123
  Synthetic primer

SEQ ID NO: 298 - DNA, 40 bp, Artificial sequence; identical to SEQ ID NO: 124
  Synthetic primer

SEQ ID NO: 299 - DNA, 44 bp, Artificial sequence; identical to SEQ ID NO: 125
  Synthetic primer

SEQ ID NO: 300 - DNA, 44 bp, Artificial sequence; identical to SEQ ID NO: 126
  Synthetic primer

SEQ ID NO: 301 - DNA, 45 bp, Artificial sequence; identical to SEQ ID NO: 127
  Synthetic primer

SEQ ID NO: 302 - DNA, 45 bp, Artificial sequence; identical to SEQ ID NO: 128
  Synthetic primer

SEQ ID NO: 303 - DNA, 45 bp, Artificial sequence; identical to SEQ ID NO: 129
  Synthetic primer

SEQ ID NO: 304 - DNA, 45 bp, Artificial sequence; identical to SEQ ID NO: 130
  Synthetic primer

SEQ ID NO: 305 - DNA, 43 bp, Artificial sequence; identical to SEQ ID NO: 131
  Synthetic primer

SEQ ID NO: 306 - DNA, 46 bp, Artificial sequence; identical to SEQ ID NO: 132
  Synthetic primer

SEQ ID NO: 307 - DNA, 43 bp, Artificial sequence; identical to SEQ ID NO: 133
  Synthetic primer

SEQ ID NO: 308 - DNA, 47 bp, Artificial sequence; identical to SEQ ID NO: 134
  Synthetic primer

SEQ ID NO: 309 - DNA, 45 bp, Artificial sequence; identical to SEQ ID NO: 135
  Synthetic primer

SEQ ID NO: 310 - DNA, 45 bp, Artificial sequence; identical to SEQ ID NO: 136
  Synthetic primer

SEQ ID NO: 311 - DNA, 45 bp, Artificial sequence; identical to SEQ ID NO: 137
  Synthetic primer

SEQ ID NO: 312 - DNA, 45 bp, Artificial sequence; identical to SEQ ID NO: 138
  Synthetic primer

SEQ ID NO: 313 - DNA, 45 bp, Artificial sequence; identical to SEQ ID NO: 139
  Synthetic primer

SEQ ID NO: 314 - DNA, 45 bp, Artificial sequence; identical to SEQ ID NO: 140
  Synthetic primer

SEQ ID NO: 315 - DNA, 44 bp, Artificial sequence; identical to SEQ ID NO: 141
  Synthetic primer

SEQ ID NO: 316 - DNA, 44 bp, Artificial sequence; identical to SEQ ID NO: 142
  Synthetic primer

SEQ ID NO: 317 - DNA, 47 bp, Artificial sequence; identical to SEQ ID NO: 143
  Synthetic primer

SEQ ID NO: 318 - DNA, 45 bp, Artificial sequence; identical to SEQ ID NO: 144
  Synthetic primer

SEQ ID NO: 319 - DNA, 45 bp, Artificial sequence; identical to SEQ ID NO: 145
  Synthetic primer

SEQ ID NO: 320 - DNA, 45 bp, Artificial sequence; identical to SEQ ID NO: 146
  Synthetic primer

SEQ ID NO: 321 - DNA, 45 bp, Artificial sequence; identical to SEQ ID NO: 147
  Synthetic primer

SEQ ID NO: 322 - DNA, 45 bp, Artificial sequence; identical to SEQ ID NO: 148
  Synthetic primer

SEQ ID NO: 323 - DNA, 47 bp, Artificial sequence; identical to SEQ ID NO: 149
  Synthetic primer

SEQ ID NO: 324 - DNA, 45 bp, Artificial sequence; identical to SEQ ID NO: 150
  Synthetic primer

SEQ ID NO: 325 - DNA, 44 bp, Artificial sequence; identical to SEQ ID NO: 151
  Synthetic primer

SEQ ID NO: 326 - DNA, 45 bp, Artificial sequence; identical to SEQ ID NO: 152
  Synthetic primer

SEQ ID NO: 327 - DNA, 45 bp, Artificial sequence; identical to SEQ ID NO: 153
  Synthetic primer

SEQ ID NO: 328 - DNA, 45 bp, Artificial sequence; identical to SEQ ID NO: 154
  Synthetic primer

SEQ ID NO: 329 - DNA, 45 bp, Artificial sequence; identical to SEQ ID NO: 155
  Synthetic primer

SEQ ID NO: 330 - DNA, 45 bp, Artificial sequence; identical to SEQ ID NO: 156
  Synthetic primer

SEQ ID NO: 331 - DNA, 45 bp, Artificial sequence; identical to SEQ ID NO: 157
  Synthetic primer

SEQ ID NO: 332 - DNA, 45 bp, Artificial sequence; identical to SEQ ID NO: 158
  Synthetic primer

SEQ ID NO: 333 - DNA, 45 bp, Artificial sequence; identical to SEQ ID NO: 159
  Synthetic primer

SEQ ID NO: 334 - DNA, 45 bp, Artificial sequence; identical to SEQ ID NO: 160
  Synthetic primer

SEQ ID NO: 335 - DNA, 45 bp, Artificial sequence; identical to SEQ ID NO: 161
  Synthetic primer

SEQ ID NO: 336 - DNA, 45 bp, Artificial sequence; identical to SEQ ID NO: 162
  Synthetic primer

SEQ ID NO: 337 - DNA, 45 bp, Artificial sequence; identical to SEQ ID NO: 163
  Synthetic primer

SEQ ID NO: 338 - DNA, 45 bp, Artificial sequence; identical to SEQ ID NO: 164
  Synthetic primer

SEQ ID NO: 339 - DNA, 42 bp, Artificial sequence; identical to SEQ ID NO: 165
  Synthetic primer

SEQ ID NO: 340 - DNA, 44 bp, Artificial sequence; identical to SEQ ID NO: 166
  Synthetic primer

SEQ ID NO: 341 - DNA, 45 bp, Artificial sequence; identical to SEQ ID NO: 167
  Synthetic primer

SEQ ID NO: 342 - DNA, 45 bp, Artificial sequence; identical to SEQ ID NO: 168
  Synthetic primer

SEQ ID NO: 343 - DNA, 43 bp, Artificial sequence; identical to SEQ ID NO: 169
  Synthetic primer

SEQ ID NO: 344 - DNA, 45 bp, Artificial sequence; identical to SEQ ID NO: 170
  Synthetic primer

SEQ ID NO: 345 - DNA, 45 bp, Artificial sequence; identical to SEQ ID NO: 171
  Synthetic primer

SEQ ID NO: 346 - DNA, 45 bp, Artificial sequence; identical to SEQ ID NO: 172
  Synthetic primer

SEQ ID NO: 347 - DNA, 45 bp, Artificial sequence; identical to SEQ ID NO: 173
  Synthetic primer

SEQ ID NO: 348 - DNA, 44 bp, Artificial sequence; identical to SEQ ID NO: 174
  Synthetic primer

SEQ ID NO: 349 - DNA, 43 bp, Artificial sequence; identical to SEQ ID NO: 175
  Synthetic primer

SEQ ID NO: 350 - DNA, 44 bp, Artificial sequence; identical to SEQ ID NO: 176
  Synthetic primer

SEQ ID NO: 351 - DNA, 45 bp, Artificial sequence; identical to SEQ ID NO: 177
  Synthetic primer

SEQ ID NO: 352 - DNA, 43 bp, Artificial sequence; identical to SEQ ID NO: 178
  Synthetic primer

SEQ ID NO: 353 - DNA, 43 bp, Artificial sequence; identical to SEQ ID NO: 179
  Synthetic primer

SEQ ID NO: 354 - DNA, 43 bp, Artificial sequence; identical to SEQ ID NO: 180
  Synthetic primer

SEQ ID NO: 355 - DNA, 43 bp, Artificial sequence; identical to SEQ ID NO: 181
  Synthetic primer

SEQ ID NO: 356 - DNA, 43 bp, Artificial sequence; identical to SEQ ID NO: 182
  Synthetic primer

SEQ ID NO: 357 - DNA, 45 bp, Artificial sequence; identical to SEQ ID NO: 183
  Synthetic primer

SEQ ID NO: 358 - DNA, 43 bp, Artificial sequence; identical to SEQ ID NO: 184
  Synthetic primer

SEQ ID NO: 359 - DNA, 45 bp, Artificial sequence; identical to SEQ ID NO: 185
  Synthetic primer

SEQ ID NO: 360 - DNA, 44 bp, Artificial sequence; identical to SEQ ID NO: 186
  Synthetic primer

SEQ ID NO: 361 - DNA, 44 bp, Artificial sequence; identical to SEQ ID NO: 187
  Synthetic primer

SEQ ID NO: 362 - DNA, 45 bp, Artificial sequence; identical to SEQ ID NO: 188
  Synthetic primer

SEQ ID NO: 363 - DNA, 45 bp, Artificial sequence; identical to SEQ ID NO: 189
  Synthetic primer

SEQ ID NO: 364 - DNA, 46 bp, Artificial sequence; identical to SEQ ID NO: 190
  Synthetic primer

SEQ ID NO: 365 - DNA, 45 bp, Artificial sequence; identical to SEQ ID NO: 191
  Synthetic primer

SEQ ID NO: 366 - DNA, 44 bp, Artificial sequence; identical to SEQ ID NO: 192
  Synthetic primer

SEQ ID NO: 367 - DNA, 45 bp, Artificial sequence; identical to SEQ ID NO: 193
  Synthetic primer

SEQ ID NO: 368 - DNA, 44 bp, Artificial sequence; identical to SEQ ID NO: 194
  Synthetic primer

SEQ ID NO: 369 - DNA, 45 bp, Artificial sequence; identical to SEQ ID NO: 195
  Synthetic primer

SEQ ID NO: 370 - DNA, 43 bp, Artificial sequence; identical to SEQ ID NO: 196
  Synthetic primer

SEQ ID NO: 371 - DNA, 45 bp, Artificial sequence; identical to SEQ ID NO: 197
  Synthetic primer

SEQ ID NO: 372 - DNA, 44 bp, Artificial sequence; identical to SEQ ID NO: 198
  Synthetic primer

SEQ ID NO: 373 - DNA, 45 bp, Artificial sequence; identical to SEQ ID NO: 199
  Synthetic primer

SEQ ID NO: 374 - DNA, 45 bp, Artificial sequence; identical to SEQ ID NO: 200
  Synthetic primer

SEQ ID NO: 375 - DNA, 43 bp, Artificial sequence; identical to SEQ ID NO: 201
  Synthetic primer

SEQ ID NO: 376 - DNA, 45 bp, Artificial sequence; identical to SEQ ID NO: 202
  Synthetic primer

SEQ ID NO: 377 - DNA, 45 bp, Artificial sequence; identical to SEQ ID NO: 203
  Synthetic primer

SEQ ID NO: 378 - DNA, 46 bp, Artificial sequence; identical to SEQ ID NO: 204
  Synthetic primer

SEQ ID NO: 379 - DNA, 44 bp, Artificial sequence; identical to SEQ ID NO: 205
  Synthetic primer

SEQ ID NO: 380 - DNA, 42 bp, Artificial sequence; identical to SEQ ID NO: 206
  Synthetic primer

SEQ ID NO: 381 - DNA, 43 bp, Artificial sequence; identical to SEQ ID NO: 207
  Synthetic primer

SEQ ID NO: 382 - DNA, 66 bp, Artificial sequence; identical to SEQ ID NO: 209
  Synthetic primer
  misc_feature (27)..(32): n is a, c, g, or t

SEQ ID NO: 383 - DNA, 86 bp, Artificial sequence
  Synthetic primer
  misc_feature (59)..(59): n is a, c, g, or t
  misc_feature (61)..(61): n is a, c, g, or t
  misc_feature (63)..(63): n is a, c, g, or t
aatgatacggcgaccaccgagatctacactctttccctacacgacgctcttccgatctnhnhnaagcagtggtatcaacgcagagt

SEQ ID NO: 384 - DNA, 63 bp, Artificial sequence
  Synthetic primer
  misc_feature (34)..(45): n is a, c, g, or t
acactctttccctacacgacgctcttccgatctnnnnnnnnnnnngacctcgggtgggaacac

SEQ ID NO: 385 - DNA, 20 bp, Artificial sequence
  Synthetic primer
acactctttccctacacgac

SEQ ID NO: 386 - DNA, 45 bp, Artificial sequence; identical to SEQ ID NO: 162
  Synthetic primer

SEQ ID NO: 387 - DNA, 45 bp, Artificial sequence; identical to SEQ ID NO: 163
  Synthetic primer

SEQ ID NO: 388 - DNA, 45 bp, Artificial sequence; identical to SEQ ID NO: 164
  Synthetic primer

SEQ ID NO: 389 - DNA, 42 bp, Artificial sequence; identical to SEQ ID NO: 165
  Synthetic primer

SEQ ID NO: 390 - DNA, 44 bp, Artificial sequence; identical to SEQ ID NO: 166
  Synthetic primer

SEQ ID NO: 391 - DNA, 45 bp, Artificial sequence; identical to SEQ ID NO: 167
  Synthetic primer

SEQ ID NO: 392 - DNA, 45 bp, Artificial sequence; identical to SEQ ID NO: 168
  Synthetic primer

SEQ ID NO: 393 - DNA, 43 bp, Artificial sequence; identical to SEQ ID NO: 169
  Synthetic primer

SEQ ID NO: 394 - DNA, 45 bp, Artificial sequence; identical to SEQ ID NO: 170
  Synthetic primer

SEQ ID NO: 395 - DNA, 45 bp, Artificial sequence; identical to SEQ ID NO: 171
  Synthetic primer

SEQ ID NO: 396 - DNA, 45 bp, Artificial sequence; identical to SEQ ID NO: 172
  Synthetic primer

SEQ ID NO: 397 - DNA, 45 bp, Artificial sequence; identical to SEQ ID NO: 173
  Synthetic primer

SEQ ID NO: 398 - DNA, 44 bp, Artificial sequence; identical to SEQ ID NO: 174
  Synthetic primer

SEQ ID NO: 399 - DNA, 43 bp, Artificial sequence; identical to SEQ ID NO: 175
  Synthetic primer

SEQ ID NO: 400 - DNA, 44 bp, Artificial sequence; identical to SEQ ID NO: 176
  Synthetic primer

SEQ ID NO: 401 - DNA, 45 bp, Artificial sequence; identical to SEQ ID NO: 177
  Synthetic primer

SEQ ID NO: 402 - DNA, 43 bp, Artificial sequence; identical to SEQ ID NO: 178
  Synthetic primer

SEQ ID NO: 403 - DNA, 43 bp, Artificial sequence; identical to SEQ ID NO: 179
  Synthetic primer

SEQ ID NO: 404 - DNA, 43 bp, Artificial sequence; identical to SEQ ID NO: 180
  Synthetic primer

SEQ ID NO: 405 - DNA, 43 bp, Artificial sequence; identical to SEQ ID NO: 181
  Synthetic primer

SEQ ID NO: 406 - DNA, 43 bp, Artificial sequence; identical to SEQ ID NO: 182
  Synthetic primer

SEQ ID NO: 407 - DNA, 45 bp, Artificial sequence; identical to SEQ ID NO: 183
  Synthetic primer

SEQ ID NO: 408 - DNA, 43 bp, Artificial sequence; identical to SEQ ID NO: 184
  Synthetic primer

SEQ ID NO: 409 - DNA, 45 bp, Artificial sequence; identical to SEQ ID NO: 185
  Synthetic primer

SEQ ID NO: 410 - DNA, 44 bp, Artificial sequence; identical to SEQ ID NO: 186
  Synthetic primer

SEQ ID NO: 411 - DNA, 44 bp, Artificial sequence; identical to SEQ ID NO: 187
  Synthetic primer

SEQ ID NO: 412 - DNA, 45 bp, Artificial sequence; identical to SEQ ID NO: 188
  Synthetic primer

SEQ ID NO: 413 - DNA, 45 bp, Artificial sequence; identical to SEQ ID NO: 189
  Synthetic primer

SEQ ID NO: 414 - DNA, 46 bp, Artificial sequence; identical to SEQ ID NO: 190
  Synthetic primer

SEQ ID NO: 415 - DNA, 45 bp, Artificial sequence; identical to SEQ ID NO: 191
  Synthetic primer

SEQ ID NO: 416 - DNA, 44 bp, Artificial sequence; identical to SEQ ID NO: 192
  Synthetic primer

SEQ ID NO: 417 - DNA, 45 bp, Artificial sequence; identical to SEQ ID NO: 193
  Synthetic primer

SEQ ID NO: 418 - DNA, 44 bp, Artificial sequence; identical to SEQ ID NO: 194
  Synthetic primer

SEQ ID NO: 419 - DNA, 45 bp, Artificial sequence; identical to SEQ ID NO: 195
  Synthetic primer

SEQ ID NO: 420 - DNA, 43 bp, Artificial sequence; identical to SEQ ID NO: 196
  Synthetic primer

SEQ ID NO: 421 - DNA, 45 bp, Artificial sequence; identical to SEQ ID NO: 197
  Synthetic primer

SEQ ID NO: 422 - DNA, 44 bp, Artificial sequence; identical to SEQ ID NO: 198
  Synthetic primer

SEQ ID NO: 423 - DNA, 45 bp, Artificial sequence; identical to SEQ ID NO: 199
  Synthetic primer

SEQ ID NO: 424 - DNA, 45 bp, Artificial sequence; identical to SEQ ID NO: 200
  Synthetic primer

SEQ ID NO: 425 - DNA, 43 bp, Artificial sequence; identical to SEQ ID NO: 201
  Synthetic primer

SEQ ID NO: 426 - DNA, 45 bp, Artificial sequence; identical to SEQ ID NO: 202
  Synthetic primer

SEQ ID NO: 427 - DNA, 45 bp, Artificial sequence; identical to SEQ ID NO: 203
  Synthetic primer

SEQ ID NO: 428 - DNA, 46 bp, Artificial sequence; identical to SEQ ID NO: 204
  Synthetic primer

SEQ ID NO: 429 - DNA, 44 bp, Artificial sequence; identical to SEQ ID NO: 205
  Synthetic primer

SEQ ID NO: 430 - DNA, 42 bp, Artificial sequence; identical to SEQ ID NO: 206
  Synthetic primer

SEQ ID NO: 431 - DNA, 43 bp, Artificial sequence; identical to SEQ ID NO: 207
  Synthetic primer

SEQ ID NO: 432 - DNA, 45 bp, Artificial sequence; identical to SEQ ID NO: 25
  Synthetic primer

SEQ ID NO: 433 - DNA, 66 bp, Artificial sequence; identical to SEQ ID NO: 209
  Synthetic primer
  misc_feature (27)..(32): n is a, c, g, or t

SEQ ID NO: 434 - DNA, 63 bp, Artificial sequence
  Synthetic primer
  misc_feature (34)..(45): n is a, c, g, or t
acactctttccctacacgacgctcttccgatctnnnnnnnnnnnnggtacacggcagggtcag

SEQ ID NO: 435 - DNA, 20 bp, Artificial sequence
  Synthetic primer
acactctttccctacacgac

SEQ ID NO: 436 - DNA, 45 bp, Artificial sequence; identical to SEQ ID NO: 122
  Synthetic primer

SEQ ID NO: 437 - DNA, 45 bp, Artificial sequence; identical to SEQ ID NO: 123
  Synthetic primer

SEQ ID NO: 438 - DNA, 40 bp, Artificial sequence; identical to SEQ ID NO: 124
  Synthetic primer

SEQ ID NO: 439 - DNA, 44 bp, Artificial sequence; identical to SEQ ID NO: 125
  Synthetic primer

SEQ ID NO: 440 - DNA, 44 bp, Artificial sequence; identical to SEQ ID NO: 126
  Synthetic primer

SEQ ID NO: 441 - DNA, 45 bp, Artificial sequence; identical to SEQ ID NO: 127
  Synthetic primer

SEQ ID NO: 442 - DNA, 45 bp, Artificial sequence; identical to SEQ ID NO: 128
  Synthetic primer

SEQ ID NO: 443 - DNA, 45 bp, Artificial sequence; identical to SEQ ID NO: 129
  Synthetic primer

SEQ ID NO: 444 - DNA, 45 bp, Artificial sequence; identical to SEQ ID NO: 130
  Synthetic primer

SEQ ID NO: 445 - DNA, 43 bp, Artificial sequence; identical to SEQ ID NO: 131
  Synthetic primer

SEQ ID NO: 446 - DNA, 46 bp, Artificial sequence; identical to SEQ ID NO: 132
  Synthetic primer

SEQ ID NO: 447 - DNA, 43 bp, Artificial sequence; identical to SEQ ID NO: 133
  Synthetic primer

SEQ ID NO: 448 - DNA, 47 bp, Artificial sequence; identical to SEQ ID NO: 134
  Synthetic primer

SEQ ID NO: 449 - DNA, 45 bp, Artificial sequence; identical to SEQ ID NO: 135
  Synthetic primer

SEQ ID NO: 450 - DNA, 45 bp, Artificial sequence; identical to SEQ ID NO: 136
  Synthetic primer

SEQ ID NO: 451 - DNA, 45 bp, Artificial sequence; identical to SEQ ID NO: 137
  Synthetic primer

SEQ ID NO: 452 - DNA, 45 bp, Artificial sequence; identical to SEQ ID NO: 138
  Synthetic primer

SEQ ID NO: 453 - DNA, 45 bp, Artificial sequence; identical to SEQ ID NO: 139
  Synthetic primer

SEQ ID NO: 454 - DNA, 45 bp, Artificial sequence; identical to SEQ ID NO: 140
  Synthetic primer

SEQ ID NO: 455 - DNA, 44 bp, Artificial sequence; identical to SEQ ID NO: 141
  Synthetic primer

SEQ ID NO: 456 - DNA, 44 bp, Artificial sequence; identical to SEQ ID NO: 142
  Synthetic primer

SEQ ID NO: 457 - DNA, 47 bp, Artificial sequence; identical to SEQ ID NO: 143
  Synthetic primer

SEQ ID NO: 458 - DNA, 45 bp, Artificial sequence; identical to SEQ ID NO: 144
  Synthetic primer

SEQ ID NO: 459 - DNA, 45 bp, Artificial sequence; identical to SEQ ID NO: 145
  Synthetic primer

SEQ ID NO: 460 - DNA, 45 bp, Artificial sequence; identical to SEQ ID NO: 146
  Synthetic primer

SEQ ID NO: 461 - DNA, 45 bp, Artificial sequence; identical to SEQ ID NO: 147
  Synthetic primer

SEQ ID NO: 462 - DNA, 45 bp, Artificial sequence; identical to SEQ ID NO: 148
  Synthetic primer

SEQ ID NO: 463 - DNA, 47 bp, Artificial sequence; identical to SEQ ID NO: 149
  Synthetic primer

SEQ ID NO: 464 - DNA, 45 bp, Artificial sequence; identical to SEQ ID NO: 150
  Synthetic primer

SEQ ID NO: 465 - DNA, 44 bp, Artificial sequence; identical to SEQ ID NO: 151
  Synthetic primer

SEQ ID NO: 466 - DNA, 45 bp, Artificial sequence; identical to SEQ ID NO: 152
  Synthetic primer

SEQ ID NO: 467 - DNA, 45 bp, Artificial sequence; identical to SEQ ID NO: 153
  Synthetic primer

SEQ ID NO: 468 - DNA, 45 bp, Artificial sequence; identical to SEQ ID NO: 154
  Synthetic primer

SEQ ID NO: 469 - DNA, 45 bp, Artificial sequence; identical to SEQ ID NO: 155
  Synthetic primer

SEQ ID NO: 470 - DNA, 45 bp, Artificial sequence; identical to SEQ ID NO: 156
  Synthetic primer

SEQ ID NO: 471 - DNA, 45 bp, Artificial sequence; identical to SEQ ID NO: 157
  Synthetic primer

SEQ ID NO: 472 - DNA, 45 bp, Artificial sequence; identical to SEQ ID NO: 158
  Synthetic primer

SEQ ID NO: 473 - DNA, 45 bp, Artificial sequence; identical to SEQ ID NO: 159
  Synthetic primer

SEQ ID NO: 474 - DNA, 45 bp, Artificial sequence; identical to SEQ ID NO: 160
  Synthetic primer

SEQ ID NO: 475 - DNA, 45 bp, Artificial sequence; identical to SEQ ID NO: 161
  Synthetic primer

SEQ ID NO: 476 - DNA, 45 bp, Artificial sequence; identical to SEQ ID NO: 25
  Synthetic primer

SEQ ID NO: 477 - DNA, 66 bp, Artificial sequence; identical to SEQ ID NO: 209
  Synthetic primer
  misc_feature (27)..(32): n is a, c, g, or t

SEQ ID NO: 478 - DNA, 66 bp, Artificial sequence
  Synthetic primer
  misc_feature (34)..(45): n is a, c, g, or t
acactctttccctacacgacgctcttccgatctnnnnnnnnnnnnagcaggttctgggttctggat

SEQ ID NO: 479 - DNA, 43 bp, Artificial sequence
  Synthetic primer
gacgtgtgctcttccgatctcagttacctgcttctgacagagc

SEQ ID NO: 480 - DNA, 42 bp, Artificial sequence
  Synthetic primer
gacgtgtgctcttccgatctaaagccaaacgattctccctgc

SEQ ID NO: 481 - DNA, 41 bp, Artificial sequence
  Synthetic primer
gacgtgtgctcttccgatctagatgctaagcacagcacgct

SEQ ID NO: 482 - DNA, 41 bp, Artificial sequence
  Synthetic primer
gacgtgtgctcttccgatcttccataagagcagcagctcct

SEQ ID NO: 483 - DNA, 43 bp, Artificial sequence
  Synthetic primer
gacgtgtgctcttccgatctgctctttgcacatttcctcctcc

SEQ ID NO: 484 - DNA, 43 bp, Artificial sequence
  Synthetic primer
gacgtgtgctcttccgatctgctctttgactatatcctcctcc

SEQ ID NO: 485 - DNA, 43 bp, Artificial sequence
  Synthetic primer
gacgtgtgctcttccgatcttctccttgcacatyrhagactct

SEQ ID NO: 486 - DNA, 43 bp, Artificial sequence
  Synthetic primer
gacgtgtgctcttccgatcttccatcagccttrtcatttcarc

SEQ ID NO: 487 - DNA, 45 bp, Artificial sequence
  Synthetic primer
gacgtgtgctcttccgatctgcakaacttagaacatsttcacagg

SEQ ID NO: 488 - DNA, 43 bp, Artificial sequence
  Synthetic primer
gacgtgtgctcttccgatctagttccatcggactcatcatcac

SEQ ID NO: 489 - DNA, 42 bp, Artificial sequence
  Synthetic primer
gacgtgtgctcttccgatcttcaacctgaagaaatccccagc

SEQ ID NO: 490 - DNA, 40 bp, Artificial sequence
  Synthetic primer
gacgtgtgctcttccgatctgctccctgttcatcgccaga

SEQ ID NO: 491 - DNA, 41 bp, Artificial sequence
  Synthetic primer
gacgtgtgctcttccgatctaacaaaagyggcaaacactkc

SEQ ID NO: 492 - DNA, 40 bp, Artificial sequence
  Synthetic primer
gacgtgtgctcttccgatctcggaagctcagcactctgag

SEQ ID NO: 493 - DNA, 42 bp, Artificial sequence
  Synthetic primer
gacgtgtgctcttccgatctgcgtctccttacatataacagc

SEQ ID NO: 494 - DNA, 42 bp, Artificial sequence
  Synthetic primer
gacgtgtgctcttccgatctctgacagaaagtcaagcaccty

SEQ ID NO: 495 - DNA, 43 bp, Artificial sequence
  Synthetic primer
gacgtgtgctcttccgatctgctcttttccctgctcacaaagg

SEQ ID NO: 496 - DNA, 40 bp, Artificial sequence
  Synthetic primer
gacgtgtgctcttccgatcttgcacttctcccctgcactt

SEQ ID NO: 497 - DNA, 44 bp, Artificial sequence
  Synthetic primer
gacgtgtgctcttccgatcttctctctatctgaacatcacagca

SEQ ID NO: 498 - DNA, 42 bp, Artificial sequence
  Synthetic primer
gacgtgtgctcttccgatctactctctctgaacctcacagct

SEQ ID NO: 499 - DNA, 38 bp, Artificial sequence
  Synthetic primer
gacgtgtgctcttccgatctdctacagcacccygcaca

SEQ ID NO: 500 - DNA, 42 bp, Artificial sequence
  Synthetic primer
gacgtgtgctcttccgatctttctccctgcacawcacagaca

SEQ ID NO: 501 - DNA, 42 bp, Artificial sequence
  Synthetic primer
gacgtgtgctcttccgatctacccttctccctacacatcata

SEQ ID NO: 502 - DNA, 43 bp, Artificial sequence
  Synthetic primer
gacgtgtgctcttccgatctacacctttccctgcacattacag

SEQ ID NO: 503 - DNA, 44 bp, Artificial sequence
  Synthetic primer
gacgtgtgctcttccgatctctggataagaaaggcaaacacatc

SEQ ID NO: 504 - DNA, 40 bp, Artificial sequence
  Synthetic primer
gacgtgtgctcttccgatcttccttccacttrcrgaaagc

SEQ ID NO: 505 - DNA, 42 bp, Artificial sequence
  Synthetic primer
gacgtgtgctcttccgatctttccttccacttgcagaaaacc

SEQ ID NO: 506 - DNA, 41 bp, Artificial sequence
  Synthetic primer
gacgtgtgctcttccgatctgctacacatcagagactccca

SEQ ID NO: 507 - DNA, 41 bp, Artificial sequence
  Synthetic primer
gacgtgtgctcttccgatctcctgcacatcaragactccca

SEQ ID NO: 508 - DNA, 41 bp, Artificial sequence
  Synthetic primer
gacgtgtgctcttccgatctcctacacatcagagarccrca

SEQ ID NO: 509 - DNA, 40 bp, Artificial sequence
  Synthetic primer
gacgtgtgctcttccgatctcctgcacatcagagagtcgc

SEQ ID NO: 510 - DNA, 41 bp, Artificial sequence
  Synthetic primer
gacgtgtgctcttccgatctccttgacacytccagccarag

SEQ ID NO: 511 - DNA, 43 bp, Artificial sequence
  Synthetic primer
gacgtgtgctcttccgatctctgagttcagcaagagyractct

SEQ ID NO: 512 - DNA, 45 bp, Artificial sequence; identical to SEQ ID NO: 25
  Synthetic primer

SEQ ID NO: 513 - DNA, 66 bp, Artificial sequence; identical to SEQ ID NO: 209
  Synthetic primer
  misc_feature (27)..(32): n is a, c, g, or t

SEQ ID NO: 514 - DNA, 67 bp, Artificial sequence
  Synthetic primer
  misc_feature (34)..(45): n is a, c, g, or t
acactctttccctacacgacgctcttccgatctnnnnnnnnnnnngggtggagtcacatttctcaga

SEQ ID NO: 515 - DNA, 20 bp, Artificial sequence
  Synthetic primer
acactctttccctacacgac

SEQ ID NO: 516 - DNA, 41 bp, Artificial sequence
  Synthetic primer
gacgtgtgctcttccgatcttcactgatacggagctgaggc

SEQ ID NO: 517 - DNA, 43 bp, Artificial sequence
  Synthetic primer
gacgtgtgctcttccgatctgctttcccctgacattagagtca

SEQ ID NO: 518 - DNA, 44 bp, Artificial sequence
  Synthetic primer
gacgtgtgctcttccgatcttcctactctattctgaagacccag

SEQ ID NO: 519 - DNA, 43 bp, Artificial sequence
  Synthetic primer
gacgtgtgctcttccgatctctctgaratgaacatgagtgcct

SEQ ID NO: 520 - DNA, 43 bp, Artificial sequence
  Synthetic primer
gacgtgtgctcttccgatctaatccaacagttcaacgactttt

SEQ ID NO: 521 - DNA, 41 bp, Artificial sequence
  Synthetic primer
gacgtgtgctcttccgatctgacttcttcctcctgctggaa

SEQ ID NO: 522 - DNA, 42 bp, Artificial sequence
  Synthetic primer
gacgtgtgctcttccgatctttctcyctcattctggagttgg

SEQ ID NO: 523 - DNA, 43 bp, Artificial sequence
  Synthetic primer
gacgtgtgctcttccgatctctccactctcaagatccagtctg

SEQ ID NO: 524 - DNA, 44 bp, Artificial sequence
  Synthetic primer
gacgtgtgctcttccgatctccttctccactctgaagattcaac

SEQ ID NO: 525 - DNA, 43 bp, Artificial sequence
  Synthetic primer
gacgtgtgctcttccgatctgtcgcactcaactctgaagatcc

SEQ ID NO: 526 - DNA, 44 bp, Artificial sequence
  Synthetic primer
gacgtgtgctcttccgatcttctgctctctctacattggctctg

SEQ ID NO: 527 - DNA, 42 bp, Artificial sequence
  Synthetic primer
gacgtgtgctcttccgatctggaacccaacatcctaaagtgg

SEQ ID NO: 528 - DNA, 42 bp, Artificial sequence
  Synthetic primer
gacgtgtgctcttccgatcttctctcactgtgacatctgccc

SEQ ID NO: 529 - DNA, 44 bp, Artificial sequence
  Synthetic primer
gacgtgtgctcttccgatctccatttagaccttcagatcacagc

SEQ ID NO: 530 - DNA, 44 bp, Artificial sequence
  Synthetic primer
gacgtgtgctcttccgatctcatcagtcatcccaacttatcctt

SEQ ID NO: 531 - DNA, 44 bp, Artificial sequence
  Synthetic primer
gacgtgtgctcttccgatctatgtaccatagagatccagtccag

SEQ ID NO: 532 - DNA, 42 bp, Artificial sequence
  Synthetic primer
gacgtgtgctcttccgatctgcagcttggaaatcagttcctc

SEQ ID NO: 533 - DNA, 41 bp, Artificial sequence
  Synthetic primer
gacgtgtgctcttccgatctctgggaatcagaacgtgcgaa

SEQ ID NO: 534 - DNA, 43 bp, Artificial sequence
  Synthetic primer
gacgtgtgctcttccgatctgcatcctggaaatcctatcctct

SEQ ID NO: 535 - DNA, 43 bp, Artificial sequence
  Synthetic primer
gacgtgtgctcttccgatctctcatccttcatcttggaaatgc

SEQ ID NO: 536 - DNA, 43 bp, Artificial sequence
  Synthetic primer
gacgtgtgctcttccgatctcagcctagaaattcagtcctctg

SEQ ID NO: 537 - DNA, 44 bp, Artificial sequence
  Synthetic primer
gacgtgtgctcttccgatctgaatcctacctcatgttaagcaca

SEQ ID NO: 538 - DNA, 42 bp, Artificial sequence
  Synthetic primer
gacgtgtgctcttccgatctaaatcttccagcatcgaccagg

SEQ ID NO: 539 - DNA, 42 bp, Artificial sequence
  Synthetic primer
gacgtgtgctcttccgatctagcatttctccctgattctgga

SEQ ID NO: 540 - DNA, 42 bp, Artificial sequence
  Synthetic primer
gacgtgtgctcttccgatctctctgaaaatccaacccacagc

SEQ ID NO: 541 - DNA, 43 bp, Artificial sequence
  Synthetic primer
gacgtgtgctcttccgatctcgttgacagtgaacaatgcaagg

SEQ ID NO: 542 - DNA, 42 bp, Artificial sequence
  Synthetic primer
gacgtgtgctcttccgatctttcatcctaagcacggagaagc

SEQ ID NO: 543 - DNA, 46 bp, Artificial sequence
  Synthetic primer
gacgtgtgctcttccgatcttcagataaagctcatttgaatcttcg

SEQ ID NO: 544 - DNA, 44 bp, Artificial sequence
  Synthetic primer
gacgtgtgctcttccgatctagacagctccaagctacttttaca

SEQ ID NO: 545 - skipped

SEQ ID NO: 546 - DNA, 44 bp, Artificial sequence
  Synthetic primer
gacgtgtgctcttccgatctggattgttctccactctgaagatt

SEQ ID NO: 547 - DNA, 44 bp, Artificial sequence
  Synthetic primer
gacgtgtgctcttccgatctcaatttggtgactagcatcctgaa

SEQ ID NO: 548 - DNA, 43 bp, Artificial sequence
  Synthetic primer
gacgtgtgctcttccgatctcacagaggacttcaccttcactg

SEQ ID NO: 549 - DNA, 43 bp, Artificial sequence
  Synthetic primer
gacgtgtgctcttccgatctctccttctccatgttgaagagcc

SEQ ID NO: 550 - DNA, 45 bp, Artificial sequence; identical to SEQ ID NO: 25
  Synthetic primer

SEQ ID NO: 551 - DNA, 66 bp, Artificial sequence; identical to SEQ ID NO: 209
  Synthetic primer
  misc_feature (27)..(32): n is a, c, g, or t

SEQ ID NO: 552 - DNA, 67 bp, Artificial sequence
  Synthetic primer
  misc_feature (34)..(45): n is a, c, g, or t
acactctttccctacacgacgctcttccgatctnnnnnnnnnnnngatgacttcagtgttgttctgg

SEQ ID NO: 553 - DNA, 62 bp, Artificial sequence
  Synthetic primer
  misc_feature (34)..(45): n is a, c, g, or t
acactctttccctacacgacgctcttccgatctnnnnnnnnnnnncagggatccagagttcc

SEQ ID NO: 554 - DNA, 63 bp, Artificial sequence
  Synthetic primer
  misc_feature (34)..(45): n is a, c, g, or t
acactctttccctacacgacgctcttccgatctnnnnnnnnnnnncaggtcacattcatcgtg

SEQ ID NO: 555 - DNA, 61 bp, Artificial sequence
  Synthetic primer
  misc_feature (34)..(45): n is a, c, g, or t
acactctttccctacacgacgctcttccgatctnnnnnnnnnnnnagtggctgacttccaa

SEQ ID NO: 556 - DNA, 64 bp, Artificial sequence
  Synthetic primer
  misc_feature (34)..(45): n is a, c, g, or t
acactctttccctacacgacgctcttccgatctnnnnnnnnnnnncacagtgctcatgttcagg

SEQ ID NO: 557 - DNA, 40 bp, Artificial sequence
  Synthetic primer
gacgtgtgctcttccgatctagrtycagctgcarcagtct

SEQ ID NO: 558 - DNA, 39 bp, Artificial sequence
  Synthetic primer
gacgtgtgctcttccgatctaggtccaactgcagcagcc

SEQ ID NO: 559 - DNA, 40 bp, Artificial sequence
  Synthetic primer
gacgtgtgctcttccgatcttctgcctggtgacwttccca

SEQ ID NO: 560 - DNA, 39 bp, Artificial sequence
  Synthetic primer
gacgtgtgctcttccgatctgtgcagcttcaggagtcag

SEQ ID NO: 561 - DNA, 40 bp, Artificial sequence
  Synthetic primer
gacgtgtgctcttccgatctgaggtgaagcttctcgagtc

SEQ ID NO: 562 - DNA, 40 bp, Artificial sequence
  Synthetic primer
gacgtgtgctcttccgatctgaagtgaagctggtggagtc

SEQ ID NO: 563 - DNA, 41 bp, Artificial sequence
  Synthetic primer
gacgtgtgctcttccgatctatgkacttgggactgarctgt

SEQ ID NO: 564 - DNA, 40 bp, Artificial sequence
  Synthetic primer
gacgtgtgctcttccgatctcagtgtgaggtgaagctggt

SEQ ID NO: 565 - DNA, 41 bp, Artificial sequence
  Synthetic primer
gacgtgtgctcttccgatctccaggttactctgaaagagtc

SEQ ID NO: 566 - DNA, 41 bp, Artificial sequence
  Synthetic primer
gacgtgtgctcttccgatcttgtggaccttgctattcctga

SEQ ID NO: 567 - DNA, 41 bp, Artificial sequence
  Synthetic primer
gacgtgtgctcttccgatcttgttggggctgaagtgggttt

SEQ ID NO: 568 - DNA, 41 bp, Artificial sequence
  Synthetic primer
gacgtgtgctcttccgatctatggagtgggaactgagctta

SEQ ID NO: 569 - DNA, 39 bp, Artificial sequence
  Synthetic primer
gacgtgtgctcttccgatctagcttcaggagtcaggacc

SEQ ID NO: 570 - DNA, 40 bp, Artificial sequence
  Synthetic primer
gacgtgtgctcttccgatctcaggtgcagcttgtagagac

SEQ ID NO: 571 - DNA, 41 bp, Artificial sequence
  Synthetic primer
gacgtgtgctcttccgatctatgcagctgggtcatcttctt

SEQ ID NO: 572 - DNA, 41 bp, Artificial sequence
  Synthetic primer
gacgtgtgctcttccgatctgactggatttggatcackctc

SEQ ID NO: 573 - DNA, 41 bp, Artificial sequence
  Synthetic primer
gacgtgtgctcttccgatcttggagtttggacttagttggg

SEQ ID NO: 574 - DNA, 20 bp, Artificial sequence
  Synthetic primer
acactctttccctacacgac

SEQ ID NO: 575 - DNA, 45 bp, Artificial sequence; identical to SEQ ID NO: 25
  Synthetic primer

SEQ ID NO: 576 - DNA, 66 bp, Artificial sequence; identical to SEQ ID NO: 209
  Synthetic primer
  misc_feature (27)..(32): n is a, c, g, or t

SEQ ID NO: 577 - DNA, 63 bp, Artificial sequence
  Synthetic primer
  misc_feature (34)..(45): n is a, c, g, or t
acactctttccctacacgacgctcttccgatctnnnnnnnnnnnnagtccttgaccaggcagc

SEQ ID NO: 578 - DNA, 64 bp, Artificial sequence
  Synthetic primer
  misc_feature (34)..(45): n is a, c, g, or t
acactctttccctacacgacgctcttccgatctnnnnnnnnnnnngaygaccacgttcccatct

SEQ ID NO: 579 - DNA, 65 bp, Artificial sequence; identical to SEQ ID NO: 10
  Synthetic primer
  misc_feature (34)..(45): n is a, c, g, or t

SEQ ID NO: 580 - DNA, 64 bp, Artificial sequence; identical to SEQ ID NO: 11
  Synthetic primer
  misc_feature (34)..(45): n is a, c, g, or t

SEQ ID NO: 581 - DNA, 64 bp, Artificial sequence; identical to SEQ ID NO: 12
  Synthetic primer
  misc_feature (34)..(45): n is a, c, g, or t

SEQ ID NO: 582 - DNA, 39 bp, Artificial sequence; identical to SEQ ID NO: 13
  Synthetic primer

SEQ ID NO: 583 - DNA, 40 bp, Artificial sequence; identical to SEQ ID NO: 14
  Synthetic primer

SEQ ID NO: 584 - DNA, 40 bp, Artificial sequence; identical to SEQ ID NO: 15
  Synthetic primer

SEQ ID NO: 585 - DNA, 39 bp, Artificial sequence; identical to SEQ ID NO: 16
  Synthetic primer

SEQ ID NO: 586 - DNA, 40 bp, Artificial sequence; identical to SEQ ID NO: 17
  Synthetic primer

SEQ ID NO: 587 - DNA, 39 bp, Artificial sequence; identical to SEQ ID NO: 18
  Synthetic primer

SEQ ID NO: 588 - DNA, 40 bp, Artificial sequence; identical to SEQ ID NO: 19
  Synthetic primer

SEQ ID NO: 589 - DNA, 40 bp, Artificial sequence; identical to SEQ ID NO: 20
  Synthetic primer

SEQ ID NO: 590 - DNA, 40 bp, Artificial sequence; identical to SEQ ID NO: 21
  Synthetic primer

SEQ ID NO: 591 - DNA, 38 bp, Artificial sequence; identical to SEQ ID NO: 22
  Synthetic primer

SEQ ID NO: 592 - DNA, 38 bp, Artificial sequence; identical to SEQ ID NO: 23
  Synthetic primer

SEQ ID NO: 593 - DNA, 20 bp, Artificial sequence
  Synthetic primer
acactctttccctacacgac

SEQ ID NO: 594 - DNA, 45 bp, Artificial sequence; identical to SEQ ID NO: 25
  Synthetic primer

SEQ ID NO: 595 - DNA, 66 bp, Artificial sequence; identical to SEQ ID NO: 209
  Synthetic primer
  misc_feature (27)..(32): n is a, c, g, or t

SEQ ID NO: 596 - DNA, 26 bp, Artificial sequence
  Synthetic primer
  misc_feature (26)..(26): n is a, c, g, or t
ttttttttttttttttttttttttvn

SEQ ID NO: 597 - DNA, 21 bp, Artificial sequence
  Synthetic primer
gagccatcagaagcagagatc

SEQ ID NO: 598 - DNA, 19 bp, Artificial sequence
  Synthetic primer
ctccttcccattcacccac

SEQ ID NO: 599 - DNA, 22 bp, Artificial sequence
  Synthetic primer
ccacacccaaaaggccacactg

SEQ ID NO: 600 - DNA, 160 bp, Artificial sequence
  Synthetic oligonucleotide
agtggtggttactactggagctggatccgccagcacccagggaagggcctggagtggattgggtacatctattacagtgggagcacctactacaacccgtccctcaagagtcgagttaccatatcagtagacacgtctaagaaccagttctccctgaagc

SEQ ID NO: 601 - DNA, 211 bp, Artificial sequence
  Synthetic oligonucleotide
tgggtgcgacaggcccctggacaagggcttgagtggatgggatggatcagcgcttacaatggtaacacaaactatgcacagaagctccagggcagagtcaccatgaccacagacacatccacgagacacagcctacatggagctgaggagcctgagatctgacgacacggccgtgtattacggccagggaaccctggtcaccgtctcctca

SEQ ID NO: 602 - DNA, 217 bp, Artificial sequence
  Synthetic oligonucleotide
cactgggtcgccaggcttccgggaaagggctggagtgggtggccgtattagaagcaaagctaacagttacgcgacagcatatgctgcgtcggtgaaaggcaggttcaccatctccagagatgattcaaagaacacggcgtatctgcaaatgaacagcctgaaaaccgaggacacggccgtgtattacggccagggaaccctggtcaccgtctcctca

SEQ ID NO: 603 - DNA, 46 bp, Artificial sequence
  Synthetic oligonucleotide
gtgtggcaggaagctcctacgagcagtacttcgggccgggcaccag

SEQ ID NO: 604 - DNA, 46 bp, Artificial sequence; identical to SEQ ID NO: 603
  Synthetic oligonucleotide

SEQ ID NO: 605 - DNA, 46 bp, Artificial sequence; identical to SEQ ID NO: 603
  Synthetic oligonucleotide

SEQ ID NO: 606 - DNA, 46 bp, Artificial sequence; identical to SEQ ID NO: 603
  Synthetic oligonucleotide

SEQ ID NO: 607 - DNA, 46 bp, Artificial sequence
  Synthetic oligonucleotide
gtgtggcaggaagctcctacgagcagtacttcgggccgggctgcag

SEQ ID NO: 608 - DNA, 46 bp, Artificial sequence
  Synthetic oligonucleotide
gtgtggcaggaagctcctacgagcagtacttcgggccgggcacctg

SEQ ID NO: 609 - DNA, 46 bp, Artificial sequence; identical to SEQ ID NO: 603
  Synthetic oligonucleotide

SEQ ID NO: 610 - DNA, 46 bp, Artificial sequence; identical to SEQ ID NO: 603
  Synthetic oligonucleotide

SEQ ID NO: 611 - DNA, 46 bp, Artificial sequence; identical to SEQ ID NO: 603
  Synthetic oligonucleotide

SEQ ID NO: 612 - DNA, 46 bp, Artificial sequence
  Synthetic oligonucleotide
tggacaggggaagcacagatacgcagtattttggcccaggcacccg

SEQ ID NO: 613 - DNA, 46 bp, Artificial sequence; identical to SEQ ID NO: 612
  Synthetic oligonucleotide

SEQ ID NO: 614 - DNA, 46 bp, Artificial sequence; identical to SEQ ID NO: 612
  Synthetic oligonucleotide

SEQ ID NO: 615 - DNA, 46 bp, Artificial sequence
  Synthetic oligonucleotide
gtgtggcgggaagctcctacgagcagtacttcgggccgggcaccag

SEQ ID NO: 616 - DNA, 37 bp, Artificial sequence
  Synthetic oligonucleotide
cacaccctgcagccagaagactcggccctgtatctct

SEQ ID NO: 617 - DNA, 37 bp, Artificial sequence
  Synthetic oligonucleotide
accctgcagccagaagactcggccctgtatctctgcg

SEQ ID NO: 618 - DNA, 37 bp, Artificial sequence
  Synthetic oligonucleotide
gagttggctgctccctcccagacatctgtgtacttct

SEQ ID NO: 619 - DNA, 37 bp, Artificial sequence
  Synthetic oligonucleotide
gacttgctgcctccagactagtcgtctttgtacttct

SEQ ID NO: 620 - protein, 13 aa, Artificial sequence
  Synthetic peptide
CASSVGTPYEQYF

SEQ ID NO: 621 - DNA, 39 bp, Artificial sequence
  Synthetic oligonucleotide
tgtgccagcagcaggacagaaaatcagccccagcatttt

SEQ ID NO: 622 - DNA, 39 bp, Artificial sequence
  Synthetic oligonucleotide
tgtgccagcagtagaacagagaatcagccccagcatttt

SEQ ID NO: 623 - DNA, 39 bp, Artificial sequence
  Synthetic oligonucleotide
tgtgccagcagtaggacagagaatcagccccagcatttt

(19) United States
(12) Patent Application Publication
(10) Pub. No.: US 2020/0131564 A1
(43) Pub. Date: Apr. 30, 2020

(54) HIGH-COVERAGE AND ULTRA-ACCURATE IMMUNE REPERTOIRE SEQUENCING USING MOLECULAR IDENTIFIERS

(71) Applicant: BOARD OF REGENTS, THE UNIVERSITY OF TEXAS SYSTEM, Austin, TX (US)

(72) Inventors: Ning JIANG, Austin, TX (US); Keyue MA, Austin, TX (US); Ben S. WENDEL, Austin, TX (US); Chenfeng HE, Austin, TX (US); Mingjuan QU, Austin, TX (US)

(21) Appl. No.: 16/628,828

(22) PCT Filed: Jul. 9, 2018
(86) PCT No.: PCT/US2018/041261

§ 371 Date: Jan. 6, 2020

Related U.S. Application Data
(60) Provisional application No. 62/529,859, filed on Jul. 7, 2017.
(60) Provisional application No. 62/620,820, filed on Jan. 23, 2018.

(51) Int. Cl. C12Q 1/6806; C12Q 1/6881; G16B 40/10; G16B 40/20; G16B 30/10

(52) U.S. Cl. CPC C12Q 1/6806; C12Q 1/6881; G16B 40/10; G16B 40/20; G16B 30/10; C12Q 2565/514; C12Q 2521/107; C12Q 2525/161; C12Q 2535/122; C12Q 2563/179; C12Q 2521/101

(57) ABSTRACT

The present disclosure provides methods for the amplification and sequencing of the immune repertoire using barcoded oligonucleotides with molecular identifiers (MIDs). Further provided are methods for clustering-based data analysis of the sequencing reads to determine the immune repertoire.

The present application claims the priority benefit of U.S. Provisional Application Ser. No. 62/529,859, filed Jul. 7, 2017, and 62/620,820, filed Jan. 23, 2018, the entire contents of which are hereby incorporated by reference.

The invention was made with government support under Grant Nos. R00 AG040149 and S10 OD020072 awarded by the National Institutes of Health. The government has certain rights in the invention.

### INCORPORATION OF SEQUENCE LISTING

The sequence listing that is contained in the file named “UTFB1098WO.txt”, which is 123 KB (as measured in Microsoft Windows) and was created on Jul. 9, 2018, is filed herewith by electronic submission and is incorporated by reference herein.

### BACKGROUND

### 1. Field

The present invention relates generally to the fields of molecular biology and immunology. More particularly, it concerns sequencing of the immune repertoire.

### 2. Description of Related Art

The body generates millions of T cells and B cells, each bearing a unique T cell receptor (TCR) or secreting unique antibodies respectively. Through V(D)J recombination, millions of different TCR or antibodies are generated. In general, they are collectively referred to as the immune repertoire. The signature of the immune repertoire can be used to differentiate between healthy immune systems and disease-related immune systems. Due to the nature of recombination and somatic hypermutation accurate recovery of immune repertoire sequence information is essential, however, this is prone to being affected by PCR and sequencing error.

Immune repertoire sequencing (IR-seq) has become a useful tool to quantify the composition of the various antigen receptor repertoires, such as antibody (Georgiou et al., 2014) and TCR (Robins, 2013). However, early versions of IR-seq suffer from high amplification bias and high sequencing error rates. Although studies have focused on ways to control these artifacts through data analysis (Weinstein et al., 2009; Jiang et al., 2011; Bolotin et al., 2012; Michaeli et al., 2012; Jiang et al., 2013; Zhu et al., 2013), accurate sequencing information was not possible until recent applications using molecular identifiers (Vollmers et al., 2013; Shugay et al., 2014; Vander Heiden et al., 2014). However, there is an unmet need for a general framework for the use of molecular identifiers, including the efficient use of molecular identifiers to tag each transcript, methods for grouping reads to generate consensus sequences, and quality metrics to analyze IR-seq methods. Answers to these questions are important for overall repertoire diversity estimates and controlling the accuracy of the sequence information obtained.

### SUMMARY

In certain embodiments, the present disclosure provides methods and compositions for analyzing the immune repertoire (e.g., antibody and TCR sequencing). In a first embodiment, there is provided a method of amplifying variable immune sequences comprising producing cDNA from a plurality of RNA molecules using barcoded oligonucleotides, wherein the barcoded oligonucleotides comprise a molecular identifier (MID) and a gene-specific primer, thereby generating a plurality of MID-tagged cDNAs; and amplifying the MID-tagged cDNAs using nested PCR, thereby producing a plurality of MID-tagged variable immune sequences.

In some aspects, the gene-specific primer hybridizes to the constant region of an immunological receptor. In certain aspects, the immunological receptor is an immunoglobulin, T cell receptor (TCR), major histocompatibility receptor, NK cell receptor, complement receptor, Fc receptor or fragment thereof. In some aspects, the constant region is an immunoglobulin heavy chain, immunoglobulin light chain, TCR α chain or TCR β chain. In particular aspects, the gene-specific primer comprises SEQ ID NO:1 (AAGACCGATGGGCCCTTG), SEQ ID NO:2 (GAAGACCTTGGGGCTGGT), SEQ ID NO:3 (GGGAATTCTCACAGGAGACG), SEQ ID NO:4 (GAAGACGGATGGGCTCTGT), or SEQ ID NO:5 (GGGTGTCTGCACCCTGATA). In some aspects, the gene-specific primer is gene-specific primer is SEQ ID NO:6 (GACCTCGGGTGGGAACAC) or SEQ ID NO:7 (GGTACACGGCAGGGTCAG).

In certain aspects, the plurality of MID-tagged variable immune sequences are further defined as nucleic acids which encode for the variable region of an immunoglobulin, T cell receptor (TCR), major histocompatibility receptor, NK cell receptor, complement receptor, Fc receptor, or fragment thereof.

In some aspects, the method further comprises isolating a plurality of RNA molecules from a sample prior to step (a). In certain aspects, the plurality of RNA molecules comprises an input RNA of 10%, 20%, 30%, or higher (e.g., 0.1, 0.2, 0.3, 0.4, 0.5, 1, 2, 5, 10, or more μg). In certain aspects, the sample is blood, lymph, sputum, or tissue. In particular aspects, the sample is a blood sample. In some aspects, the sample comprises peripheral blood mononuclear cells, B cells, T cells, or plasmablasts. In certain aspects, the samples comprises 1,000 to 10,000,000 cells, such as about 1,000,000 cells. In one particular aspects, the sample comprises less than 1,000 cells. In other aspects, the sample comprises more than 10,000,000 cells. In certain aspects, the sample is obtained from a subject having an autoimmune disease, an infectious disease, or cancer. In some aspects, the sample is obtained from a transplant recipient or vaccine recipient. In some aspects, the sample is obtained from a subject being treated with an immunosuppressive therapy.

In particular aspects, the MID comprises 8-16 nucleotides, such as 8-12 nucleotides, such as 8, 9, 10, 11, or 12 nucleotides. In specific aspects, the MID comprises 9 nucleotides. In other aspects, the MID comprises 12 nucleotides.

In additional aspects, the method further comprises digesting the barcoded oligonucleotides with an enzyme prior to step (b). In particular aspects, the enzyme is exonuclease I.

In some aspects, steps (a) and (b) are performed in the same reaction container, such as a tube. In particular aspects, the mixture from step (a) is not transferred to a different reaction tube for step (b). In some aspects, the sample comprises more than 1,000 cells (e.g., 1,000,000 cells) and is aliquoted into multiple tubes for step (a) which are not switched for step (b). In particular aspects, the cDNA of step (a) is not subjected to a purification prior to step (b). In some aspects, there is no purification of cDNA by size exclusion chromatography.

In certain aspects, the nested PCR comprises using a first set of primers specific to the leader region of an immunoglobulin or TCR. In some aspects, the first set of primers specific to the leader region of an immunoglobulin or TCR are selected from the primers listed in Table 1.

In some aspects, the method further comprises sequencing the plurality of MID-tagged immune variable sequences to obtain sequencing reads and analyzing the sequencing reads to determine the immune repertoire of the sample. In certain aspects, analyzing comprises performing clustering data analysis. In some aspects, clustering data analysis comprises merging paired-end raw reads, identifying immunological receptor reads, and grouping sequence reads with identical MIDs.

In particular aspects, the method further comprises applying a threshold clustering process to cluster reads with identical MIDs into subgroups. In some aspects, the clustering threshold is 1 to 20% of the read length. In certain aspects, the clustering threshold is 4 to 6% of the read length. In particular aspects, the clustering threshold is 14 to 15% of the read length.

In some aspects, the method further comprises building a consensus sequence for each cluster to produce a collection of consensus sequences. In certain aspects, the collection of consensus sequences is used to determine the diversity and/or abundance of the immune repertoire.

In certain aspects, the method further comprises calculating the sequencing error rate. In some aspects, the error rate is less than 0.005%. In particular aspects, the error rate is less than 0.004%.

In some aspects, the method further comprises counting RNA molecule copy number (e.g., TCR transcript number). In certain aspects, the immune sequences are TCRs. In some aspects, the counting is based on input cell number, percentage of RNA input, and sequencing depth. In certain aspects, counting comprises performing digital PCR, such as using primers of Table 1. In certain aspects, TCR RNA molecule copy number is determined for a single cell. In particular aspects, single cell counting comprises fitting distribution of reads under each MID sub-group into two binomial distributions.

In another embodiment, there is provided a method for monitoring T cell clonal expansion in a subject comprising obtaining a population of T cells from the subject; determining the TCR sequence by the method of the embodiments; and quantifying T cell clonal expansion. In some aspects, the T cells are effector T cells. In certain aspects, the subject has a viral infection, such as CMV. In some aspects, the subject has cancer, an infectious disease, or autoimmune disease. In certain aspects, the sample subject is a transplant or vaccine recipient. In further aspects, the method further comprises using T cell expansion quantification to predict response to a treatment or vaccine.

Another embodiment provides a method of producing a cDNA library for immune repertoire analysis comprising obtaining a plurality of RNA molecules; hybridizing the plurality of RNA molecules to oligo(dT)-containing primers; performing reverse transcription using template switching oligonucleotides comprising a molecular identifier (MID) and a poly-uracil region, thereby generating a plurality of cDNAs; and PCR amplifying the plurality of cDNAs, thereby producing a cDNA library for immune repertoire analysis. In certain aspects, steps (c) and (d) comprise performing rapid amplification of cDNA ends (RACE). In some aspects, the method further comprises the addition of carrier RNA to the cells.

In some aspects, the poly-uracil region comprises 2, 3, 4, 5, or 6 uracils. In certain aspects, the method further comprises contacting the template switching oligonucleotides with uracil-specific excision reagent (USER) enzyme prior to step (d), thereby degrading the template switching oligonucleotides.

In certain aspects, obtaining in step (a) comprises isolating a plurality of RNA molecules from a sample. In certain aspects, the plurality of RNA molecules comprises an input RNA of 10%, 20%, 30%, or higher (e.g., 0.1, 0.2, 0.3, 0.4, 0.5, 1, 2, 5, 10, or more μg). In some aspects, the sample is blood, lymph, sputum, or tissue. In particular aspects, the sample is a blood sample. In certain aspects, the sample comprises peripheral blood mononuclear cells, B cells, T cells, or plasmablasts. In some aspects, the sample comprises 1,000 to 10,000,000 cells, such as 1,000 to 1,000,000 cells. In some aspects, the sample comprises less than 1,000 cells. In particular aspects, the sample comprises less than 100 cells. In some aspects, the sample comprises more than 10,000,000 cells. In some aspects, the sample is obtained from a subject having an autoimmune disease, an infectious disease or cancer. In some aspects, the sample is obtained from a transplant recipient or vaccine recipient. In particular aspects, the sample is obtained from a subject being treated with an immunosuppressive therapy.

In particular aspects, the MID comprises 8-16 nucleotides, such as 8, 9, 10, 11, or 12 nucleotides. In specific aspects, the MID comprises 9 nucleotides. In other aspects, the MID comprises 12 nucleotides.

In some aspects, steps (b) to (d) are performed in the same reaction tube(s). In certain aspects, the cDNA of step (c) is not subjected to a purification prior to step (d).

In some aspects, the method further comprises performing immune repertoire analysis. In certain aspects, performing immune repertoire analysis comprises performing whole transcriptome sequencing of the cDNA library. In some aspects, performing immune repertoire analysis comprises immunoglobulin and/or TCR amplification prior to sequencing of the cDNA library.

In certain aspects, the method further comprises performing clustering data analysis. In some aspects, clustering data analysis comprises merging paired-end raw reads, identifying immunological receptor reads, and grouping sequence reads with identical MIDs. In certain aspects, the method further comprises applying a threshold clustering process to cluster reads with identical MIDs into subgroups. In some aspects, the clustering threshold is 1 to 20% of the read length. In particular aspects, the clustering threshold is 4 to 6% of the read length. In some aspects, the clustering threshold is 14 to 15% of the read length. In certain aspects, the method further comprises building a consensus sequence for each cluster to produce a collection of consensus sequences. In some aspects, the collection of consensus sequences is used to determine the diversity of the immune repertoire. In certain aspects, the method further comprises calculating the sequencing error rate. In some aspects, the error rate is less than 0.005%. In particular aspects, the error rate is less than 0.004%.

A further embodiment provides a composition comprising T cell primers listed in Table 1. In some aspects, the T cells primers are further defined as single cell TCR sequencing primers, bulk TCR repertoire sequencing primers (MIDCIRS-TCR), or single cell TCR with single cell RNA-sequencing primer. Further provided are methods of using the T cells primer for TCR sequencing.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1B: Overview of molecular identifier (MID, also referred to as UMI) clustering-based IR-seq (MIDCRS). (A) Schematics of tagging single Ig transcripts with MIDs. (B) Schematics of the informatics pipeline of MID clustering-based IR-seq which includes joining two reads, performing clustering to generate MID sub-groups, and building consensus.

FIGS. 2A-2B: Antibody repertoire diversity estimate using naïve B cells as input materials (A) Total RNA sampling depth (5%, 10% or 30%) and diversity coverage for a range of samples with different amount of naïve B cells. Naïve B cells were sorted into different amounts. Either 5% or 30% of total RNA was used as input material in generating the amplicon libraries. Slope of the correlation curves indicates the estimated diversity. (B) Rarefaction analysis of optimum sequencing depth for each sample in library 3. Reads from library that was made with 30% RNA input was sub-sampled to different depths, and the number of unique consensus was calculated.

FIGS. 3A-3D: Robustness of MID clustering-based IR-seq method. (A) Comparison of diversity estimates obtained by analyzing antibody heavy chain sequences using two different lengths to show the appropriateness of our sub-clustering threshold. Reads from library 3 were used in this analysis. (B) Types of read lengths in each MID sub-groups after analyzing reads from library 3 following the schematics in FIG. 1. (C) Reduction of artificial diversity using MID clustering-based IR-seq. Two sequencing depths were compared, which were 5× or 100× of the cell number. (D) Comparison between raw error rate and improved error rate after using MID clustering-based IR-seq for three run with different library loading density.

FIGS. 4A-4C: Ultra-accurate high-coverage of antibody repertoire with a large dynamic range of input cells for MIDCIRS. (A) Correlation between number of cells and number of unique RNA molecules after using MIDCIRS. RNA from as few as 1,000 to as many as 1,000,000 NBCs was used as input material in generating the amplicon libraries. Slope indicates the estimated diversity coverage. (B, C) Rarefaction analysis of optimum sequencing depth for each sample with (B) and without (C) using MIDCIRS.

FIGS. 5A-5C: Infants and toddlers are separated into two stages based on SHM load. (A) Distribution of SHM number for infants (N=6) and toddlers (N=9), from whom we had paired pre- and acute malaria samples, weighted by unique RNA molecules. Long vertical lines represent the number of mutations above which 10% of sequences fall for the respective samples. * and † demarcate samples derived from the same individuals followed for 2 malaria seasons. (B) Age-related average number of mutations in pre- (circle, N=24, N_{infant}=11, N_{Toddler}=13) and acute malaria (triangle, N=15, N_{infant}=6, N_{Toddler}=9) samples, weighted by RNA molecules. Dashed line indicates the age boundary for infants (<12 months old) and toddlers (12-47 months old). (C) Comparison of average number of mutations for paired infants and toddlers. Pre- and acute malaria samples separated by isotype; lines connect paired samples (N_{Infant,paired}=6, N_{Toddler,paired}=9). Bars indicate means. *P<0.05, **P<0.01, N.S. indicates no significant difference by two-tailed Mann-Whitney U test (between age groups, dashed lines) or two-tailed Wilcoxon Signed-Rank test (between paired timepoints, solid lines). Differences in variance were not significant by squared ranks test.

FIGS. 6A-6J: Decrease of naïve B cell and increase of memory B cell percentages show a two-stage trend and correlate with SHM load. (A) NaïB percentages of total B cells from the pre-malaria samples (N=22) vary with age. Dashed vertical line depicts the cutoff between infants and toddlers. (B) NaïB percentages of total B cells compared between infants (N=9) and toddlers (N=13). (C-E) NaïB percentages correlate with average number of mutations (SHM load) in IgM (C), IgG (D), and IgA (E) sequences from bulk PBMCs in pre-malaria samples (N=22). (F) MemB percentages of total B cells from the pre-malaria samples (N=22) vary with age. Dashed vertical line depicts the cutoff between infants and toddlers. (G) MemB percentages of total B cells compared between infants (N=9) and toddlers (N=13). (H-J) MemB percentages correlate with average number of mutations (SHM load) in IgM (H), IgG (I), and IgA (j=J) sequences from bulk PBMCs in pre-malaria samples (N=22). (B and G) Bars indicate means; **P<0.01, ***P<0.001, two-tailed Mann-Whitney U test. (C to E and H-J) p and P values determined by Spearman's rank correlation listed in each panel.

FIGS. 7A-7F: Antigen selection strength comparisons between infants and toddlers. Selection strength distributions, as determined by BASELINe (Yaari et al., 2012), were compared between infants and toddlers for PBMCs from pre- (A-C) (N_{infant}=6, N_{toddler}=9) and acute (D-F) (N_{infant}=6, N_{toddler}=9) malaria timepoints, separated by isotype: (A,D) IgM, (B,E) IgG, and (C,F) IgA. Selection strength on CDR (CDR1 and 2, top half of each panel) and FWR (FWR2 and 3, bottom half of each panel) for unique RNA molecules was calculated. CDR3 and FWR4 were omitted due to the difficulty in determining the germline sequence. FWR1 for all sequences was also omitted because it was not covered entirely by some of the primers. P value calculated as previously described (Yaari et al., 2012).

FIGS. 8A-8E: B cell lineage complexity change under malaria stimulation. (A) Diversity and size of B cell lineages for infants (N=6) and toddlers (N=9) from whom paired PBMC samples at pre- and acute malaria were obtained. Each circle represents an individual lineage. The area of each circle is proportional to the SHM load. Labeled arrows indicate representative lineages whose intra-lineage structures were shown in detail in (B) and (C). Each circle's x and y coordinates were determined by its diversity (the number of unique RNA molecules in a lineage) and size (the number of total RNA molecules in a lineage), respectively. Blue and pink dashed lines represent the linear fit for pre- and acute malaria lineages, respectively. Black dashed lines indicate y=x parity, such that lineages lying on the parity line are comprised entirely of unique RNA molecules with minimum clonal expansion, such as lineage in (C). On the other hand, lineages comprised of clonally expanded RNA molecules are close to they axis, such as lineage (C). (B,C) Each node is a unique RNA molecule species. The height of the node corresponds to the number of RNA molecules of the same species, the color corresponds to number of nucleotide mutations, and the distance between nodes is proportional to the Levenshtein distance between the node sequences, as indicated in the legend above each lineage. All unlabeled nodes share the isotype with the root. (D) The non-singleton lineage percent (lineages comprised of at least 2 RNA molecules) between infants and toddlers at pre- and acute malaria. *P<0.05 by two-tailed Wilcoxon Signed-Rank test (between timepoints, solid lines); N.S. indicates no significant difference by two-tailed Mann-Whitney U test (between age groups, dashed lines). (E) The difference of linear regression slopes (angles), or degree of diversity change, between pre- and acute malaria for infants and toddlers. N.S. indicates no significant difference by two-tailed Mann-Whitney U test. Bars indicate means. Differences in variance were not significant by squared ranks test.

FIGS. 9A-9F: Two-timepoint-shared lineage analysis reveals SHM increment during acute malaria infection. (A) Average SHM for sequences from pre- and acute malaria timepoints within lineages containing sequences from both timepoints for infants (N=6) and toddlers (N=9). (B) Average SHM increase upon acute malaria infection for infants and toddlers from (A). (C) Flow diagram for two-timepoint-shared lineage containing pre-malaria MemB identification and acute progeny analysis. Percentages represent the average percent of unique sequences classified by the indicated slice, range in brackets. (D) Average SHM load for pre-malaria MemBs with acute progeny and their acute progenies for malaria-experienced toddlers with FACS sorted pre-malaria MemBs (N=8). (E) Isotype distribution of pre-malaria MemBs with acute progeny. (F) Isotype fate of acute progenies stemming from IgM pre-malaria MemBs. Lines connect the same subjects. Bars indicate means. (A, D-F) *P<0.05, N.S. indicates not significant by two-tailed Wilcoxon Signed-Rank test. (B) *P<0.05 by two-tailed Mann-Whitney U test.

FIG. 10: Cumulative distribution of reads as a function of Levenshtein distance between RNA control templates and sequencing reads. The lengths of control templates and reads were 150 bp. More than 99% of reads are similar to control templates under the Levenshtein distance of 23. Therefore we set the sub-group clustering threshold as 15% of the read length.

FIG. 11: Comparison between raw error rate and improved error rate after using MIDCIRS. Raw reads error rates (top) and MIDCIRS consensus error rates (bottom) for 3 Miseq runs.

FIG. 12: Sample collection timeline. All pre-malaria blood draws were taken in May, just before the start of the rainy season. Acute malaria blood draws were taken 7 days after the onset of acute febrile malaria. Unless otherwise indicated (^{a}), all samples were collected during 2011. Average precipitation was estimated from the neighboring city of Bamako, Mali (climatemps.com). * Same individual; † Same individual; ^{a} Drawn in 2012.

FIGS. 13A-B: Rarefaction analysis of paired PBMC malaria cohort sequencing libraries. (A) Pre-malaria PBMC rarefaction curves (N=15). (B) Acute malaria PBMC rarefaction curves (N=15). Raw reads were subsampled to varying depths, and MIDCIRS was used to determine the number of unique RNA molecules. All single-read sequences that occurred before subsampling were discarded. Single-read sequences that occurred as a results of subsampling were included as unique RNA molecules. The number of unique RNA molecules discovered saturated for all samples, indicating adequate sequencing depth.

FIGS. 14A-B: Antibody isotype distribution for infants and toddlers. Antibody isotypes were assigned based on the portion of the constant region sequenced for infants (A) and toddlers (B). Isotype distribution was weighted on the number of RNA molecules.

FIGS. 15A-B: Correlation between VDJ usage in paired PBMCs samples (N=15 pairs of pre-malaria and acute malaria). Correlations weighted by reads (A) or by lineage (B). The color bar left of each panel as well as in figure legend indicates the sample group: infant pre-malaria, toddler pre-malaria, infant acute malaria, and toddler acute malaria. The diagonal lines in each panel indicate same sample self-correlation; two shorter off-diagonal lines indicate correlations from two timepoints of the same individual.

FIG. 16: CDR3 amino acid lengths of infants (N=6) and toddlers (N=9) at pre-malaria (top) and acute malaria (bottom) timepoints, separated by isotype.

FIG. 17: Correlation between average number of mutations and age for initial, paired pre- and acute malaria samples. Initial samples (N=15) suggested a step-wise increase in SHM load around 12 months which prompted us to divide our cohort into two age groups and delve further into the antibody repertoire properties. We have since added 9 pre-malaria samples around the transition, 11 months to 17 months, which were shown in FIG. 5.

FIG. 18: Flow cytometry B cell gating and atypical memory percentage. B cells were first gated by scatter, then live, dump (CD4, CD8, CD14, CD56) negative, and then CD19⁺. Conventional memory B cells (CD20⁺CD27⁺), plasmablasts (CD27^{bright}CD38^{bright}), and naïve B cells (CD20⁺CD27⁻CD38^{low}) were gated for further analysis. Atypical memory B cells (CD20⁺CD27⁻CD38^{low}IgD⁻) make up a minor portion of the naïve-like B cells. Percentage of total B cells is displayed for each subpopulation.

FIGS. 19A-D: Comparison between pre-malaria plasmablast percentage of total B cells and average number of mutations. (A) Plasmablast percentages of total B cells compared with age. (B-D) Plasmablast percentages of total B cells compared with average number of mutations of IgM (B), IgG (C), and IgA (D) sequences from bulk PBMCs in pre-malaria samples from infants (N=9) and toddlers (N=13). p and P values determined by Spearman's rank correlation have been listed in the figure.

FIG. 20: Lineage structure visualization. Lineage distribution structures for pre-malaria and acute malaria samples for all individuals with corresponding pre-malaria and acute malaria PBMC samples. A 24 year old adult malaria patient was also included. Lineages composed of only a single unique RNA molecule were excluded. Clonal lineages shown in FIG. 8 are densely packed here. Therefore, it is not intended to show intra-lineage structure for all individual lineages in each panel; rather, each panel provides an overview of all lineages for one individual at one timepoint. The darker the cluster in each oval-shaped global lineage map, the more densely packed lineages there are.

FIG. 21: Comparison between different thresholds for lineage formation. 90% and 95% nucleotide similarities of the CDR3 region were used as the threshold to generate lineages. The distribution of the size vs diversity of lineages and the linear regressions (dashed lines) of the lineage distributions generated by the two thresholds were compared. The area of the circle corresponds to the average SHM within the lineage. Black dotted line depicts y=x parity.

FIG. 22: Pre-malaria lineage diversification between infants and toddlers. Pre-malaria lineage size/diversity linear regression slopes (FIG. 9A, dashed lines) were compared between infants and toddlers. N.S. indicates not significant by Mann Whitney U test, two-tailed. Bars indicate means.

FIG. 23: Adult B cell lineage. Size and diversity of B cell lineages between pre-malaria and acute malaria samples for a 24 year old adult malaria patient. Area of the circles corresponds to the average number of mutations within that lineage. Dashed lines represent the linear fit for pre- and acute lineages; black dotted line depicts y=x parity. Both axes were trimmed to be consistent with the main figures.

FIG. 24: Multi-timepoint shared lineage example. Intra-lineage structure for a representative lineage from FIG. 9. Blue dashed curve encompasses the pre-malaria timepoint derived sequence, and pink dashed curve encompasses the acute malaria timepoint derived sequences. Each node is a unique RNA molecule species. The height of the node corresponds to the number of RNA molecules of the same species, the color corresponds to the SHM load, and the distance between nodes is proportional to the Levenshtein distance between the node sequences, as indicated in the legend above the lineage. Unlabeled node shares the isotype with the root.

FIG. 25: Pre-malaria memory B cells' acute progeny RNA abundance. Shared lineages containing sequences from pre-malaria memory B cells and acute malaria PBMCs were formed as in FIG. 9c-f and FIG. 25. Acute sequences from these lineages were classified as direct progeny if they can be traced directly back to a pre-malaria memory B cell sequence or indirect progeny if they cannot (i.e. they stem from a separate branch in the lineage tree). The RNA abundance distribution for these sequences were split by isotype and compared to the bulk acute PBMCs from the same individuals (N=8 toddlers, Tod5 was not included because there were insufficient cells for FACS sorting). Vertical dashed line indicates 10 RNA molecule cutoff, with the percentage of unique RNA molecules larger than this cutoff displayed in the top right corner of each panel.

FIGS. 26A-C: Sequence alignment for illustrated lineages. The CDR3 region has been highlighted. The top row displays the IMGT germline allele sequence, and dashes indicate where the sequences are identical to the germline. (A) Corresponds to the lineage in FIG. 9B (germline=SEQ ID NO: 600), (B) corresponds to the lineage in FIG. 9C (germline=SEQ ID NO: 601), and (C) corresponds to the lineage in FIG. 25 (germline=SEQ ID NO: 602).

FIGS. 27A-D: MIDCIRS improves accuracy of TCR diversity estimation with sub-clustering. (A) The percentage of observed MIDs containing sub-clusters is linearly dependent on RNA input, which is defined as cell number multiplied by percentage of RNA (e.g. 20,000 cells with 10% RNA is equivalent to 2,000 RNA input). Line represents linear regression fit, F-test on the slope, p<10⁻⁹. (B) The theoretical percentage of MIDs with sub-clusters is approximately linearly dependent on copies of target molecules when copies of target molecules are less than 5,000,000 (bottom right insert). The theoretical percentage of MIDs with sub-clusters was calculated by equation (2). (C) Rarefaction curve of unique CDR3s with or without sub-clustering. Number of unique CDR3s in three libraries made with three different RNA inputs from sorted one million naïve CD8⁺ T cells are shown here. Data from other cell inputs are in FIG. 33. (D) Illustration of consensus TCR sequence building without (top) and with (bottom) sub-clustering. Top: without sub-clustering, chimera sequences are generated when different TCR RNA molecules are tagged with the same MID; bottom: TCR RNA molecules that are tagged with same MID are sub-clustered to reveal truly represented TCR sequences. Short vertical black lines indicate nucleotide differences between two TCR sequences.

FIGS. 28A-D: MIDCIRS is capable of accurate digital counting of TCR RNA molecules. (A) Rarefaction curve of detected TCR RNA molecules before and after error correction on MIDs in 20,000 naïve CD8⁺ T cells for three RNA input amounts. Data from other cell inputs are in FIG. 35. (B) Comparison of rarefaction curve of detected RNA molecules and unique CDR3s in 20,000 naïve CD8⁺ T cells for three RNA input amounts. (C) Rarefaction curve of number of unique CDR3s with single RNA copy in 20,000 naïve CD8⁺ T cells for three RNA input amounts. Sequencing reads were subsampled to different depth and unique CDR3s were tallied. Data from other cell inputs are in FIG. 37A. (D) The percentage of overlapping clones with single RNA copy at different sequencing depths by sub-sampling in 20,000 naïve CD8⁺ T cells for three RNA input amounts. The overlapping clones were compared between two adjacent sub-samplings and overlap percentage was calculated by dividing the number of overlapping clones by the total number of clones observed in the deeper sub-sampling. Data from other cell input are in FIG. 37B.

FIGS. 29A-C: TCR RNA copy number per cell estimation and experimental validation. (A) Diversity coverage of unique productive CDR3s with different RNA inputs and cell numbers (Line represents linear regression fit, F-test on the slope, R²>0.99 and p<10⁻³ for all different RNA inputs). (B) Diversity coverages with different RNA inputs using 3 as a predicted TCR RNA molecule copy number per cell. Dashed line is the theoretical prediction; dots are diversity coverages observed in libraries with different RNA inputs as illustrated in (A), assuming diversity coverage at 90% RNA input is 1. (C) Digital PCR results of TCR RNA molecule copies per cell in different CD8⁺ T cell subset. (N, naïve; CM, central memory; EM, effector memory; E, effector; NTC, no template control; n.s., not significant by Mann-Whitney U test; n.s: p-value>0.05 by Mann-Whitney U test).

FIGS. 30A-C: MIDCIRS is sensitive to detect both low copy and highly clonal expanded TCRs. (A) Number of RNA molecules detected by sequencing for each spike-in TCR control sequences (the numbers in the legend denote copies of each TCR spike-in control sequence added). (B) Comparison of clone size distribution in naïve CD8⁺ T cells and CMVpp65-specific effector CD8⁺ T cells (dashed line indicates TCR sequences with 20 copies of RNA molecules). (C) The percentage of RNA molecules that varying degree of clonally expanded CDR3 account for.

FIG. 31: CDR3 length differences within multi-RNA containing MIDs before and after sub-clustering. The number of different CDR3 lengths within multi-RNA containing MIDs from one million naïve CD8⁺ T cells (50% RNA input) was plotted before sub-clustering (orange) and within the sub-clusters (green).

FIG. 32: Rarefaction curve of unique CDR3s with or without sub-clustering. Number of unique CDR3s in libraries made using three different RNA inputs (10%, 30% and 50%) from sorted 20,000, 100,000 and 200,000 naïve CD8⁺ T cells are shown here.

FIGS. 33A-B: Representative demonstration of chimera consensus sequences generated without sub-clustering (chimera TCR sequence in FIG. 27C). (A). Two different TCR RNAs (RNA2-TCR1 and RNA2-TCR2) were tagged with the same MID (RNA2), while one of the TCRs (TCR1) has a sister RNA tagged by another MID (RNA1). After building consensus sequence weighted by quality score and number of reads at each nucleotide position, a chimera consensus sequence was generated from RNA2-tagged TCR sequences (Top box, TCR1 tagged with RNA1; bottom box, two TCR sequences tagged with same MID; *, sequencing or PCR errors that are removed in the consensus building; sequence outside the top box, true TCR1 consensus sequence; sequence outside the bottom box, chimera consensus sequence; arrow, chimera nucleotide base that differs from the rest of consensus sequence was generated by weighing read number and quality score at each nucleotide). (top to bottom, SEQ ID NOs: 603-615) (B) Multiple singleton TCR RNAs were tagged with the same MID (RNA1) that were generated by either sequencing or PCR errors. Without sub-clustering, these singletons failed to be removed and a chimera consensus sequence was generated. (top to bottom, SEQ ID NOs: 616-619)

FIG. 34: Rarefaction curve of detected TCR RNA molecules before and after MID correction in 100,000, 200,000 and 1,000,000 naïve CD8⁺ T cells for three RNA input amounts.

FIG. 35: Distribution of reads under each MID sub-group. Top expressed unique CDR3 in eight naïve CD8⁺ T cell libraries were first separated into MID sub-groups, then the histograms of read numbers under each MID sub-group were plotted here (Blue line) (Green line is the final fitting of two negative binomial distributions of the blue line; red line is the fitting of individual negative binomial distributions).

FIGS. 36A-B: MIDCIRS is capable of accurate digital counting of TCR RNA molecules. (A) Rarefaction curve of number of unique CDR3s with single-copy RNA in 100,000, 200,000 and 1,000,000 naïve CD8⁺ T cells for three RNA input amounts. The 10% RNA had the lowest number of single-copy clones and the 50% had the highest. (B) The percentage of overlapping clones with single-copy of transcript at different sequencing depths by sub-sampling in 100,000, 200,000 and 1,000,000 naïve CD8⁺ T cells for three RNA input amounts. The overlapping clones were compared between two adjacent sub-samplings and the overlap percentage was calculated by dividing the number of overlapping clones by the total number of clones observed in the deeper sub-sampling. For the 100,000 and 200,000 naïve T cells, the 10% RNA had the lowest overlap percentage which it had the highest in the 1,000,000 naïve T cells.

FIG. 37: Curve fitting of diversity coverages as a function of different RNA inputs using 3 as a predicted TCR RNA molecule copy number per cell. Dashed line is the theoretical prediction; red dots are diversity coverages observed in libraries with different RNA inputs (20%, pseudo-40%, pseudo-60% and pseudo-80%), assuming diversity coverage at pseudo-80% RNA input is 1.

FIG. 38: Comparison of diversity coverage between MIDCIRS and MIGEC pipelines on the same set of data presented in this study. P-value was determined by paired Wilcoxon test.

FIG. 39: CDR3 clone size distribution of 20,000, 100,000, 200,000 and 1,000,000 naïve CD8⁺ T cells. Red dashed line is the fitted power law distribution.

FIGS. 40A-40D: RPs undergo distinct CD4 count decline within 1 year of infection. (A) Study design and sample collection timeline. (B-D) CD4 count (B), viral load (C), and CD4/CD8 ratio (D) comparison for RP (circles, n=5) and TP (triangles, n=5) between visit 1 and visit 2. *P<0.05, two-tailed paired t test (solid lines) or two-tailed Whitney Mann U test (dashed lines). Bars indicate means.

FIGS. 41A-41D: Global IgG SHM reduces with declining CD4 count. (A) Average SHM load comparisons for RP (circles, n=5) and TP (triangles, n=5) between visit 1 and visit 2, split by isotype: IgM (top), IgG (middle), and IgA (bottom). *P<0.05, two-tailed paired t test. Bars indicate means. (B,C) Average SHM load (B) and unmutated percentage of unique sequences (C) correlations with CD4 count, split by isotype: IgM (top), IgG (middle), and IgA (bottom). Spearman's p and corresponding P-value indicated in each panel. (D) BASELINe (Yaari et al., 2012) selection strength comparisons for RP (solid curves) and TP (dotted curves) for visit 1 and visit 2, split by isotype: IgM (top), IgG (middle), and IgA (bottom). Selection strength for CDR (top half of each panel) and FWR (bottom half of each panel) calculated separately. See Table 17 for P-values for pairwise comparisons. For IgG, the most discussed isotype in this figure, all comparisons for the FWR are statistically significant, and all comparisons but one (RP visit 2 vs TP visit 2) for the CDR are statistically significant.

FIGS. 42A-42F: Antibody lineage tracking within one year reveals strong ongoing SHM in RP and to a lesser extent TP with decreased antigen selection strength in both groups. (A) SHM load comparison for RP (circles, n=5) and TP (triangles, n=5) between visit 1 and visit 2 sequences within the same lineages. *P<0.05; ** P<0.01, two-tailed paired t test. Bars indicate means. (B) Average SHM increase between visit 1 and visit 2 sequences within the same lineages. *P<0.05, two-tailed Whitney Mann U test. Bars indicate means. (C) Correlations between SHM increase and CD4 count at visit 1. Spearman's p and corresponding P-value indicated in panel. (D) BASELINe (Yaari et al., 2012) selection strength comparisons for RP (solid curves) and TP (dotted curves) for visit 1 and visit 2 sequences from two-timepoint lineages. Selection strength for CDR (top half) and FWR (bottom half) calculated separately. See Table 18 for P-values for pairwise comparisons. All comparisons but two (RP visit 1 vs TP visit 2 and TP visit 1 vs TP visit 2) are significant for the FWR, and all comparisons but one (RP visit 2 vs TP visit 2) are significant for the CDR. (E) Density contour plot of SHM increase for two-timepoint lineages by visit 1 average SHM load for RP (top) and TP (bottom). Grey dashed box indicates lineages lowly mutated at visit 1 (≤10 SHM) that increase by visit 2 (≥5 SHM increase) analyzed in F; number indicates percent of lineages falling within the box. (F) BASELINe selection strength analysis of lineages lowly mutated at visit 1 (blue) that increase by visit 2 (magenta) for RP (left) and TP (right). *P<0.05; *** P<0.0005, calculated as previously described (Yaari et al., 2012).

FIG. 43: IgG SHM load negatively correlates with viral load. Average SHM load correlations with viral load, split by isotype: IgM (top), IgG (middle), and IgA (bottom). Spearman's ρ and corresponding P-value indicated in each panel.

FIG. 44: Higher IgG SMH load is associated with lower activation of CD8+ T cells. Average SHM load correlations with the percent of CD8⁺ T cells expressing CD38, split by isotype: IgM (top), IgG (middle), and IgA (bottom). Spearman's ρ and corresponding P-value indicated in each panel.

FIGS. 45A-45C: Increase in unmutated sequences partially accounts for IgG SHM decrease. (A) Correlations between unmutated percentage of unique sequences and viral load, split by isotype: IgM (top), IgG (middle), and IgA (bottom). (B,C) Correlations between average SHM load excluding unmutated sequences and CD4 count (B) and viral load (C), split by isotype: IgM (top), IgG (middle), and IgA (bottom). Spearman's ρ and corresponding P-value indicated in each panel.

FIG. 46: SHM increase within two-timepoint lineages correlates with viral load. Correlation between SHM increase and viral load at visit 1. Spearman's ρ and corresponding P-value indicated in plot.

FIGS. 47A-47C: GC TFH cells become clonally expanded. (A) Representative plots showing sorting strategy to identify naïve, memory, and GC TFH cells. (B) Breakdown of the proportion of the TCR repertoire represented by clones of different sizes for sorted naïve, memory, and GC TFH cells from HIV+LNs. TCR clone size was normalized by the total number of TCR transcripts on nucleotide sequences. (C) NSE of the TCR repertoire of sorted naïve, memory, and GC TFH cells. Gray lines link the same patient. Bars indicate means. *P<0.05 by two-tailed Wilcoxon signed-rank test (n=8 HIV-infected LNs).

FIGS. 48A-C: Antigen-driven clonal selection signature in GC TFH cells of HIV-infected LNs. (A) Representative degeneracy plot from sample H2. Coding degeneracy level [number of unique TCR nucleotide (nt) sequences encoding a common CDR3 amino acid sequence] of each CDR3 amino acid sequence is plotted against their frequency (measured as percentage of total TCR transcripts) in naïve, memory, and GC TFH cells. Each dot is a unique CDR3 amino acid sequence. Red dashed lines indicate cutoffs for degenerate (two or more nucleotide sequences coding for the same amino acid sequence; horizontal) and expanded (0.1% or more of TCR transcripts; vertical) clones. Arrow points to example degenerate clone in (B). (B) Example of CDR3 amino acid degeneracy. Amino acid (top row, SEQ ID NO: 620) and nucleotide (bottom row, SEQ ID NOs: 621, 622, and 623) sequences for three distinct nucleotide sequences (0.41% of total TCR transcripts) that code for the same amino acid sequence as indicated by arrow in (A): Y=3 and X=0.41%. Boxes and highlights indicate redundant codons. (C) Comparison of Q1 degenerate-abundant clone percentage in naïve, memory, and GC TFH cells. Gray lines link the same patient. Bars indicate means. *P<0.05 by two-tailed Wilcoxon signed-rank test (n=8 HIV-infected LNs).

FIGS. 49A-49D: GC TFH cells exhibit HIV antigen-driven clonal expansion and selection. (A) Gag-specific TCR clones overlap with HIV+LN CD4+ T cell populations. Each thin slice of the arc represents a unique TCR sequence, ordered by the clone size (inner circle). Gray curves indicate Gag-specific TCR nucleotide sequences found in naïve (outer circle), memory (outer circle), and GC TFH (outer circle) populations. No Gag overlapping clones were detected for one individual, H8. (B) Number of Gag-specific TCR clones observed in naïve, memory, and GC TFH populations. Gray lines link the same patient. Bars indicate means (P values by two-tailed paired t test). (C) Mean clone size of Gag-specific T cells, HA-specific T cells, and bulk clones of unknown specificity from the GC TFH population. (D) Number of distinct nucleotide (nt) sequences per CDR3 amino acid (aa) sequence for Gag-specific T cells, HA-specific T cells, or bulk GC TFH cells. Data from all four individuals were aggregated for (C) and (D). Error bars indicate SEM. N.S., not significant. ***P<0.001 by two-tailed t test.

FIG. 50: GC TFH cells are clonally expanded. Breakdown of the proportion of the TCR repertoire represented by clones of different sizes for sorted naïve, memory, and GC TFH cells from HIV+LNs for each individual. TCR clone size was normalized by the total number of TCR transcripts on nucleotide (nt) sequences.

FIG. 51: Antigen-driven clonal selection signature in GC TFH cells of HIV-infected LNs. Coding degeneracy level (number of unique TCR nucleotide (nt) sequences encoding a common CDR3 amino acid (aa) sequence) of each CDR3 aa sequence is plotted against their frequency (measured as % of total TCR transcript) in naïve, memory, and GC TFH cells. Each dot is a unique CDR3 aa sequence. Red dashed lines indicate cutoffs for degenerate (2 or more nt sequences coding for the same aa sequence, horizontal) and expanded (0.1% or more of TCR transcripts, vertical) clones. Each panel is broken into 4 quadrants: Q1: degenerate-abundant clones; Q2: degenerate-rare clones; Q3: nondegenerate-rare clones; Q4: nondegenerate-abundant clones.

FIGS. 52A-52B: HA-specific CD4 T cell clones detected in HIV-infected LNs. (A) HA-specific TCR clones overlap with HIV+LN CD4+ T cell populations. Each thin slice of the arc represents a unique TCR sequence, ordered by the clone size (inner circle). Gray curves indicate HA-specific TCR nucleotide sequences found in naïve (outer circle), memory (outer circle), and GC TFH (outer circle) populations. No HA-overlapping clones were detected for one subject, H2. (B) Number of HA-specific TCR clones observed in naïve, memory, and GC TFH populations. Gray lines connect samples from the same patient. Bars indicate means. Indicated P-value by two-tailed paired t test.

### DESCRIPTION OF ILLUSTRATIVE EMBODIMENTS

Immune repertoire sequencing (IR-seq) has become a useful tool to quantify the composition of the various antigen receptor repertoires, such as antibody and T cell receptor. Early versions of IR-seq suffer from high amplification bias and high sequencing errors. However, the use of molecular identifiers (MIDs) can improve immune repertoire sequencing (IR-seq) accuracy. Accordingly, in certain embodiments, the present disclosure provides methods to use MIDs to group reads, build consensus, and estimate diversity.

One method of the present disclosure uses a barcoding strategy to provide error-free immune repertoire sequencing. In particular, the barcodes are unique molecular identifiers (e.g., 9-12 nucleotides in length) which label RNA molecules and are then used to group reads into MID groups. Barcoded oligonucleotides comprising a MID and a gene-specific primer are used as primers for reverse transcription to produce MID-tagged cDNA. The barcoded oligonucleotides are then degraded by the addition of an enzyme, such as exonuclease I, prior to performing PCR amplification. Importantly, the reverse transcription and amplification are performed in a single tube as no cDNA purification is required. A quality threshold clustering process is then applied to cluster reads with same MID into subgroups. This clustering-based analysis method separates different molecules (e.g., RNA) tagged with the same MID sequence. This clustering threshold was experimentally validated to ensure accuracy of clusters generated. An algorithm can be used to optimize and speed up the clustering process. A consensus sequence may then be built from each sub-group by considering the number of reads in each subgroup and their sequencing quality score. The multiple consensus with the exact sequences may then be combined and considered as the unique consensus. The use of MIDs reduces the bias and error introduced by PCR and sequencing, rescues sequencing reads, and estimates the immune repertoire diversity more accurately. This technology, referred to herein as the MID clustering-based IR-seq (MIDCIRS) method, has a lower error rate compared with current technology, and the error rate is not affected by the raw sequencing quality that often fluctuates.

The MIDCIRS method may be used to quantitatively study TCR RNA molecule copy number and clonality in T cells. In the present studies, MIDCIRS was applied to TCR (MIDCIRS TCR-seq) and CD5⁺ T cells were used as a test bed to build a model to count TCR RNA molecule copy number based on input cell numbers, percentage of RNA input, and sequencing depth. The studies also demonstrated a significant improvement in detection sensitivity. Thus, the present studies demonstrated accuracy, sensitivity, and the wide dynamic range of MIDCIRS TCR-seq. Therefore, MIDCIRS may be used for sensitive detection of a single cell in as many as one million naïve T cells and an accurate estimation of the degree of T cell clonal expression, such as the ability to detect one unique T cell clone in 1,000,000 T cells.

In another method, there is provided a modified SMART™-Seq protocol to analyze the immune repertoire with a very low error rate. In this method, the template switching oligonucleotide comprises a MID sequence and a poly-uracil region. The amplified full-length cDNA may then be used for sequencing to analyze the immune repertoire. The poly-U cleavage site is used to digest the barcoded oligonucleotides after reverse transcription to prevent false barcodes which can be generated in PCR steps. Thus, the immune repertoire sequencing methods provided herein can be used to achieve higher RNA capture efficiency from a low RNA input amount compared with current technologies.

In further aspects, the immune sequencing methods provided herein can be used for accurately measuring antibody repertoire sequence composition, diversity, and abundance to aide in the understanding of the repertoire response to infections and vaccinations. Studying the antibody repertoire in young children or limited tissue or sample or sorted cell populations is challenging in several regards: 1) lack of analytical tools to exhaustively study the antibody repertoire from small volumes of blood, 2) lack of informatic analysis tools to turn high-throughput data into knowledge, 3) the rarity of a large set of samples from young children obtained before and at the time of a natural infection, and 4) the small amount of sample, such as pediatric blood draw, limited tissue sample, or sorted small amount of cells are extremely prone to errors generated in PCR because they need to have a high number of PCR cycles to generate enough material to make library. While analysis of the repertoire response is challenging when studying a small amount of blood obtained from infants, the highly accurate and high-coverage repertoire sequencing method provided herein can be applied to as few as 1,000 naïve B cells (NBCs). The high accuracy, coverage, and large dynamic range on input cell numbers allowed for the study of age-related antibody repertoire development and diversification before and during acute malaria in infants (<12 months old) and toddlers (12-42 months old) using 4-8 ml of blood draws. Unexpectedly, it was discovered that high levels of somatic hypermutation (SMH) were present in infants as young as three months old. SHM levels gradually increased with age in infants and stabilized in toddlers. Despite differences in SHM levels between infants and toddlers, SHMs in both age groups were similarly selected, and the degree of repertoire diversification was also similar. Unexpectedly, detailed analysis of memory B cells (MBCs) revealed a large fraction of IgM antibodies that retain SHM and isotype switch potential and gradually increase SHMs with each year of malaria exposure. These results highlight the vast potential of antibody repertoire diversification in infants and toddlers, which could have a profound impact on vaccination and immunization strategies in children.

### I. Definitions

“Subject” and “patient” refer to either a human or non-human, such as primates, mammals, and vertebrates. In particular embodiments, the subject is a human.

“Sample” means a material obtained or isolated from a fresh or preserved biological sample or synthetically-created source that contains immune nucleic acids of interest. In certain embodiments, a sample is the biological material that contains the variable immune region(s) for which data or information are sought. Samples can include at least one cell, fetal cell, cell culture, tissue specimen, blood, serum, plasma, saliva, urine, tear, vaginal secretion, sweat, lymph fluid, cerebrospinal fluid, mucosa secretion, peritoneal fluid, ascites fluid, fecal matter, body exudates, umbilical cord blood, chorionic villi, amniotic fluid, embryonic tissue, multicellular embryo, lysate, extract, solution, or reaction mixture suspected of containing immune nucleic acids of interest. Samples can also include non-human sources, such as non-human primates, rodents and other mammals.

The term “autoimmune disease” refers to conditions in which there is an undesirable immune response directed at endogenous molecules. Autoimmune diseases may be primarily T cell mediated, antibody mediated, or a combination of both. The following listing of specific conditions is intended to be exemplary, not comprehensive. Autoimmune diseases include rheumatoid arthritis, a chronic autoimmune inflammatory synovitis affecting 0.8% of the world population.

A subject's “immunosuppressive state” or “immunocompetence” as used herein refers to the ability of the subjects immune system to mount an immune response to a pathogen or tissue (e.g., such as a transplanted organ).

An “immunosuppressive drug”, “immunosuppressant” and the like refer to any drug that reduces the activity, proliferation and/or survival of one or more immune cell types. Such cell types include any T or B lymphocyte populations. A “T-helper cell suppressant” refers to any immunosuppressant that acts on T-helper cells. Examples of T-helper cell suppressants include but are not limited to cyclosporine, tacrolimus, sirolimus, myriocin, mycophenolate, and so forth.

An “immunosuppressive regimen” involves the administration or prescription of one or more immunosuppressive drugs to a subject. Adjustments to a drug regimen may include adjusting the dose, frequency of administration, level of a drug in the subject's blood, and/or which drugs are used in the regimen. The immunosuppressive regimen may include steroids and/or thymocyte depleting antibodies in addition to immunosuppressive drugs.

The term “antibody” herein is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity. The term “immunoglobulin” or “antibody” includes, but is not limited to, any antigen-binding protein product of a vertebrate, e.g. mammalian, immunoglobulin gene complex, including human immunoglobulin isotypes IgA, IgD, IgM, IgG and IgE. In general, an antibody (or immunoglobulin) is a protein that includes two molecules, each molecule having two different polypeptides, the shorter of which functions as the light chains of the antibody and the longer of which polypeptides function as the heavy chains of the antibody. Normally, as used herein, an antibody will include at least one variable region from a heavy or light chain. Additionally, the antibody may comprise combinations of variable regions. Through processes of genetic recombination, somatic hypermutation, and junctional changes a very large repertoire of different sequences can be generated encoding the variable regions of these proteins. In addition, isotype switching (also referred to as class switching and class switch recombination (CSR)), occurs after activation of the B-cell and results in a change in the sequence encoding the constant region of the antibody.

The term “primer” or “oligonucleotide primer” as used herein, refers to an oligonucleotide that hybridizes to the template strand of a nucleic acid and initiates synthesis of a nucleic acid strand complementary to the template strand when placed under conditions in which synthesis of a primer extension product is induced, i.e., in the presence of nucleotides and a polymerization-inducing agent such as a DNA or RNA polymerase and at suitable temperature, pH, metal concentration, and salt concentration. The primer is generally single-stranded for maximum efficiency in amplification, but may alternatively be double-stranded. If double-stranded, the primer can first be treated to separate its strands before being used to prepare extension products. This denaturation step is typically effected by heat, but may alternatively be carried out using alkali, followed by neutralization. Thus, a “primer” is complementary to a template, and complexes by hydrogen bonding or hybridization with the template to give a primer/template complex for initiation of synthesis by a polymerase, which is extended by the addition of covalently bonded bases linked at its 3′ end complementary to the template in the process of DNA or RNA synthesis.

“Polymerase chain reaction,” or “PCR,” means a reaction for the in vitro amplification of specific DNA sequences by the simultaneous primer extension of complementary strands of DNA. In other words, PCR is a reaction for making multiple copies or replicates of a target nucleic acid flanked by primer binding sites, such reaction comprising one or more repetitions of the following steps: (i) denaturing the target nucleic acid, (ii) annealing primers to the primer binding sites, and (iii) extending the primers by a nucleic acid polymerase in the presence of nucleoside triphosphates. Usually, the reaction is cycled through different temperatures optimized for each step in a thermal cycler instrument. Particular temperatures, durations at each step, and rates of change between steps depend on many factors well-known to those of ordinary skill in the art, e.g., exemplified by the references: McPherson et al., editors, PCR: A Practical Approach and PCR2: A Practical Approach (IRL Press, Oxford, 1991 and 1995, respectively).

“Nested PCR” refers to a two-stage PCR wherein the amplicon of a first PCR becomes the sample for a second PCR using a new set of primers, at least one of which binds to an interior location of the first amplicon. As used herein, “initial primers” or “first set of primers” in reference to a nested amplification reaction mean the primers used to generate a first amplicon, and “secondary primers” or “second set of primers” mean the one or more primers used to generate a second, or nested, amplicon. “Multiplexed PCR” means a PCR wherein multiple target sequences (or a single target sequence and one or more reference sequences) are simultaneously carried out in the same reaction mixture, e.g. Bernard et al, 1999) (two-color real-time PCR). Usually, distinct sets of primers are employed for each sequence being amplified.

The term “Rapid Amplification of cDNA Ends” (or “RACE”) as used herein refers to the PCR amplification of a cDNA strand from a known sequence to either the 3′ or 5′ end of the cDNA strand.

The methods utilize the ability of certain nucleic acid polymerases to “template switch,” using a first nucleic acid strand as a template for polymerization, and then switching to a second template nucleic acid strand while continuing the polymerization reaction. The term “template switching” reaction refers to a process of template-dependent synthesis of the complementary strand by a DNA polymerase using two templates in consecutive order and which are not covalently linked to each other by phosphodiester bonds. The synthesized complementary strand will be a single continuous strand complementary to both templates. Typically, the first template is polyA+RNA and the second template is a “template switching oligonucleotide.”

To “specifically hybridize” to a nucleic acid means, with respect to a first nucleic acid, that the first nucleic acid hybridizes to a second nucleic acid with greater affinity than to any other nucleic acid.

The terms “molecular identifier (MID)” and “unique molecular identifier (UMI)” are used interchangeably herein to refer to a unique nucleotide sequence that is used to identify a single cell or a subpopulation of cells. UMIs can be linked to a target nucleic acid of interest during amplification (e.g., reverse transcription or PCR) and used to trace back the amplicon to the cell from which the target nucleic acid originated. A UMI can be added to a target nucleic acid of interest during amplification by carrying out reverse transcription with a primer that contains a region comprising the barcode sequence and a region that is complementary to the target nucleic acid such that the barcode sequence is incorporated into the final amplified target nucleic acid product (i.e., amplicon). Barcodes can be included in either the forward primer or the reverse primer or both primers used in PCR to amplify a target nucleic acid. In particular aspects, each UMI corresponds to DNA sequences derived from the same RNA molecule. The UMI may be any number of nucleotides of sufficient length to distinguish the UMI from other UMIs. For example, a UMI may be anywhere from 8 to 20 nucleotides long, such as 8 to 11, or 12 to 20. In particular aspects, the UMI has a length of 9 random nucleotides. The term “unique molecular identifier,” “UMI,” “molecular identifier,” “MID,” and “barcode” are used interchangeably herein.

A “consensus sequence” is the sequence of an original RNA molecule as determined by clustering reads that share the same MID and have identical or near-identical sequences. The consensus sequence reduces error in the high throughput screens discussed herein.

### II. Immune Repertoire Sequencing

Embodiments of the present disclosure provides methods for analyzing the immune repertoire of a subject through amplification and sequencing of all or a portion of the molecules that make up the immune system, including, but not limited to immunoglobulins, T cells receptors, and MHC receptors. In particular aspects, the immune repertoire includes the antibody repertoire and/or TCR binding repertoire. In one method, the immune repertoire analysis is performed on RNA isolated from a biological sample. The isolated RNA is then reverse transcribed to cDNA using a barcoded oligonucleotide to attach a MID to the 3′end during the first strand synthesis. The cDNA is then amplified by two PCR reactions for preparation of a sequencing library including the addition of sequencing adaptors and indexes. These steps can be performed in a single tube and, thus, are highly amenable to multiplexing.

A. Nucleic Acid Sample

Certain embodiments of the present disclosure concern the amplification of a variable immune region from a starting sample. In some aspects, the sample is a peripheral whole blood sample from a subject. RNA is then isolated from the peripheral whole blood sample, or fraction thereof (e.g., peripheral blood mononuclear cells), prior to reverse transcription of the isolated RNA using immune repertoire (e.g., immunoglobulin heavy chain or TCR beta chain specific primers) to generate immunoglobulin (e.g., heavy chain or light chain) or TCR (e.g., alpha, beta, delta or gamma chain) cDNA transcripts.

The subject can be a patient, for example, a patient with an autoimmune disease, an infectious disease or cancer, or a transplant recipient. The subject can be a human or a non-human mammal. The subject can be a male or female subject of any age (e.g., a fetus, an infant, a child, or an adult).

Samples can include, for example, a bodily fluid from a subject, including amniotic fluid surrounding a fetus, aqueous humor, bile, blood and blood plasma, cerumen (earwax), Cowper's fluid or pre-ejaculatory fluid, chyle, chyme, female ejaculate, interstitial fluid, lymph, menses, breast milk, mucus (including snot and phlegm), pleural fluid, pus, saliva, sebum (skin oil), semen, serum, sweat, tears, urine, vaginal lubrication, vomit, feces, internal body fluids including cerebrospinal fluid surrounding the brain and the spinal cord, synovial fluid surrounding bone joints, intracellular fluid (the fluid inside cells), and vitreous humour (the fluids in the eyeball). In particular aspects, the sample is a blood sample, such as a peripheral whole blood sample, or a fraction thereof. Preferably, the sample is whole, unfractionated blood. The blood sample can be about 0.02, 0.03, 0.04, 0.05, 0.06, 0.07, 0.08 0.09, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, or more than 5 mL. The sample can be obtained by a health care provider, for example, a physician, physician assistant, nurse, veterinarian, dermatologist, rheumatologist, dentist, paramedic, or surgeon. The sample can be obtained by a research technician. More than one sample from a subject can be obtained.

For isolation of cells from tissue, an appropriate solution can be used for dispersion or suspension. Such solution will generally be a balanced salt solution, e.g. normal saline, PBS, Hank's balanced salt solution, conveniently supplemented with fetal calf serum or other naturally occurring factors, in conjunction with an acceptable buffer at low concentration, generally from 5-25 mM. Convenient buffers include HEPES, phosphate buffers, and lactate buffers. The separated cells can be collected in any appropriate medium that maintains the viability of the cells, usually having a cushion of serum at the bottom of the collection tube. Various media are commercially available and may be used according to the nature of the cells, including dMEM, HBSS, dPBS, RPMI, and Iscove's medium, frequently supplemented with fetal calf serum.

The sample can include immune cells. The immune cells can include T-cells and/or B-cells. T-cells (T lymphocytes) include, for example, cells that express T-cell receptors. T-cells include Helper T-cells (effector T-cells or Th cells), cytotoxic T-cells (CTLs), memory T-cells, and regulatory T-cells. The sample can include a single cell in some applications (e.g., a calibration test to define relevant T-cells) or more generally at least 1,000, at least 10,000, at least 100,000, at least 250,000, at least 500,000, at least 750,000, or at least 1,000,000 T-cells.

B-cells include, for example, plasma B cells, memory B cells, Bl cells, B2 cells, marginal-zone B cells, and follicular B cells. B-cells can express immunoglobulins (antibodies, B cell receptor). The sample can include a single cell in some applications (e.g., a calibration test to define relevant B cells) or more generally at least 1,000, at least 10,000, at least 100,000, at least 250,000, at least 500,000, at least 750,000, or at least 1,000,000 B-cells.

The sample can include nucleic acids, for example, DNA (e.g., genomic DNA or mitochondrial DNA) or RNA (e.g., messenger RNA or microRNA). The nucleic acid can be cell-free DNA or RNA. In the methods of the present disclosure, the amount of RNA or DNA from a subject that can be analyzed includes, for example, as low as a single cell in some applications (e.g., a calibration test) and as many as 10 million cells or more translating to a range of DNA of 6 pg-60 μg, and RNA of approximately 1 pg-10 μg. The input RNA can be 10%, 15%, 30% or higher and about 0.1, 0.2, 0.5, 1, 2, 3, 4, 5, 10, 15, or more pg.

B. Barcoded Oligonucleotides

The isolated RNA is then reverse transcribed to cDNA using barcoded oligonucleotides which comprise a molecular identifier (MID) attached to a primer, preferably a gene-specific primer (e.g. a primer to the constant region of the antibody heavy chain or TCR). The information in RNA in a sample can be converted to cDNA by using reverse transcription using techniques well known to those of ordinary skill in the art (see e.g., Sambrook, 1989). PolyA primers, random primers, and/or gene specific primers can be used in reverse transcription reactions. Polymerases that can be used for amplification in the methods of the present disclosure include, for example, Taq polymerase, AccuPrime polymerase, or Pfu. The choice of polymerase to use can be based on whether fidelity or efficiency is preferred.

Additionally, the barcoded oligonucleotide can comprise a poly-U region to facilitate subsequent digestion of the barcoded oligonucleotide to prevent PCR bias. The barcoded oligonucleotide can further comprise an adaptor or fragment thereof for a sequencing platform (e.g., a partial P5 or P7 adaptor for Illumina® sequencing). The order of the MID, gene-specific primer, and poly-U region can be varied. For example, the gene-specific primer can be positioned 3′ to the MID or 5′ to the MID. In some embodiments, the gene-specific primer is directly contiguous with the MID. In some embodiments, the gene-specific primer is separated from the MID by 1, 2, 3, 4, 5, 6, 7, 8, 9, 10, or more nucleotides. In some embodiments, the poly-U region is positioned between the gene-specific primer and MID, 3′ of the MID, or 5′ of the MID.

In some aspects, the barcoded oligonucleotide further comprises a sample barcode that can be used to identify a sample or source of the nucleic acid material. Thus, where nucleic acid samples are derived from multiple sources, the nucleic acids in each nucleic acid sample can be tagged with different nucleic acid tags such that the source of the sample can be identified. Barcodes, also commonly referred to indexes, tags, and the like, are well known to those of skill in the art. Any suitable barcode or set of barcodes can be used, as known in the art and as exemplified by the disclosures of U.S. Pat. No. 8,053,192 and PCT Publication No. WO05/068656, which are incorporated herein by reference in their entireties. Barcoding of single cells can be performed as described, for example in the disclosure of U.S. 2013/0274117, which is incorporated herein by reference in its entirety.

1. Unique Molecular Identifier

During the reverse transcription of the isolated RNA, a short MID sequence is added to at least one end of the cDNA as part of the barcoded oligonucleotide. The MID is an oligonucleotide of 8-20 nucleotides, particularly 8-12 nucleotides, such as 8, 9, 10, 11, or 12, nucleotides in length. In particular aspects, the MID is comprised of 12 or 9 random (e.g., degenerate) nucleotides. Because each cDNA molecule is labeled with a unique tag prior to amplification, the differential amplification of each cDNA molecule can be corrected for by counting each unique tag once, thereby providing a faithful measure of the abundance of each species in the repertoire. Sequence replicates of each cDNA molecule identified by the same molecular tag can be used to construct consensus sequences, therefore allowing correction for amplification and sequencing errors. The design, incorporation and application of MIDs can take place as known in the art, as exemplified by, for example, the disclosures of WO 2012/142213, Islam et al., 2014 (using a 5 or 6 bp MID, without clustering analysis), and Kivioja, T. et al., 2012, each of which is incorporated by reference in its entirety.

2. Poly-U Region

The barcoded oligonucleotide can further comprise a modified component such as, for example, a modified nucleotide or a modified bond. In one embodiment, the modified nucleotide or bond differs in at least one respect from deoxycytosine (dC), deoxyadenine (dA), deoxyguanine (dG) or deoxythymine (dT). Where the barcoded oligonucleotide is DNA, examples of modified nucleotides include ribonucleotides or derivatives thereof (for example: uracil (U), adenine (A), guanine (G) and cytosine(C)), and deoxyribonucleotides or derivatives thereof such as deoxyuracil (dU) and 8-oxo-guanine. Where the barcoded oligonucleotide is RNA, the modified nucleotide may be a dU, a modified ribonucleotide or deoxyribonucleotide. Examples of modified ribonucleotides and deoxyribonucleotides include abasic sugar phosphates, inosine, deoxyinosine, 2,6-diamino-4-hydroxy-5-formamidopyrimidine (foramidopyrimidine-guanine, (fapy)-guanine), 8-oxoadenine, 1,N6-ethenoadenine, 3-methyladenine, 4,6-diamino-5-formamidopyrimidine, 5,6-dihydrothymine, 5,6-dihydroxyuracil, 5-formyluracil, 5-hydroxy-5-methylhydanton, 5-hydroxycytosine, 5-hydroxymethylcystosine, 5-hydroxymethyluracil, 5-hydroxyuracil, 6-hydroxy-5,6-dihydrothymine, 6-methyladenine, 7,8-dihydro-8-oxoguanine (8-oxoguanine), 7-methylguanine, aflatoxin B1-fapy-guanine, fapy-adenine, hypoxanthine, methyl-fapy-guanine, methyltartonylurea and thymine glycol. Examples of modified bonds include any bond linking two nucleotides or modified nucleotides that is not a phosphodiester bond. An example of a modified bond is a phosphorothiolate linkage.

The barcoded oligonucleotide can be cleaved at or near a modified nucleotide or bond by enzymes or chemical reagents, collectively referred to herein as “cleaving agents.” Examples of cleaving agents include DNA repair enzymes, glycosylases, DNA cleaving endonucleases, ribonucleases and silver nitrate. Where the modified nucleotide is a ribonucleotide, the barcoded oligonucleotide can be cleaved with an endoribonuclease; and where the modified component is a phosphorothiolate linkage, the barcoded oligonucleotide can be cleaved by treatment with silver nitrate (Cosstick et al., 1990).

In some embodiments, the barcoded oligonucleotide is digested with an enzyme prior to amplification with PCR to digest the MID primer. The enzyme may be exonuclease I.

In particular embodiments, the barcoded oligonucleotide comprises a poly-U region, such as between the MID and gene-specific primer. The barcoded oligonucleotide can thus be cleaved at the poly-U region. This poly-U region can be used to digest the barcoded oligonucleotide after reverse transcription to prevent false barcodes which can be generated in PCR steps. For example, cleavage at dU may be achieved using uracil DNA glycosylase and endonuclease VIII (USER™, NEB, Ipswich, Mass.) (U.S. Pat. No. 7,435,572; incorporated herein by reference).

3. Gene-Specific Primer

The gene-specific primer is specific to a region on an immunoglobulin or TCR, particularly hybridizing to the constant region of the immunological receptor. Thus, the gene-specific primer can be designed to hybridize to the constant region of an immunoglobulin heavy chain or immunoglobulin light chain or TCR alpha chain or TCR beta chain. For example, the gene-specific primer can have a sequence for IgG: SEQ ID NO:1 (AAGACCGATGGGCCCTTG), IgA: SEQ ID NO:2 (GAAGACCTTGGGGCTGGT), IgM: SEQ ID NO:3 (GGGAATTCTCACAGGAGACG), IgE: SEQ ID NO:4 (GAAGACGGATGGGCTCTGT), or IgD: SEQ ID NO:5 (GGGTGTCTGCACCCTGATA). The gene-specific primer may have a sequence for TCR β: SEQ ID NO:6 (GACCTCGGGTGGGAACAC) or TCR α: SEQ ID NO:7 (GGTACACGGCAGGGTCAG).

TABLE 1
Primer Sequences

| | |
|---|---|
| MIDCIRS Ab | SEQ ID NO: |

RT primers

| | | |
|---|---|---|
| IgG | ACACTCTTTCCCTACACGACGCTCTTCCGATCTNNNNNNNNNNNNAAGA | 8 |
| | CCGATGGGCCCTTG | |
| IgA | ACACTCTTTCCCTACACGACGCTCTTCCGATCTNNNNNNNNNNNNGAAG | 9 |
| | ACCTTGGGGCTGGT | |
| IgM | ACACTCTTTCCCTACACGACGCTCTTCCGATCTNNNNNNNNNNNNGGGA | 10 |
| | ATTCTCACAGGAGACG | |
| IgE | ACACTCTTTCCCTACACGACGCTCTTCCGATCTNNNNNNNNNNNNGAAG | 11 |
| | ACGGATGGGCTCTGT | |
| IgD | ACACTCTTTCCCTACACGACGCTCTTCCGATCTNNNNNNNNNNNNGGGT | 12 |
| | GTCTGCACCCTGATA | |

1^{st} PCR forward primers

| | | |
|---|---|---|
| ILLUPE2LR1 | GACGTGTGCTCTTCCGATCTCGCAGACCCTCTCACTCAC | 13 |
| ILLUPE2LR2 | GACGTGTGCTCTTCCGATCTTGGAGCTGAGGTGAAGAAGC | 14 |
| ILLUPE2LR3 | GACGTGTGCTCTTCCGATCTTGCAATCTGGGTCTGAGTTG | 15 |
| ILLUPE2LR4 | GACGTGTGCTCTTCCGATCTGGCTCAGGACTGGTGAAGC | 16 |
| ILLUPE2LR5 | GACGTGTGCTCTTCCGATCTTGGAGCAGAGGTGAAAAAGC | 17 |
| ILLUPE2LR6 | GACGTGTGCTCTTCCGATCTGGTGCAGCTGTTGGAGTCT | 18 |
| ILLUPE2LR7 | GACGTGTGCTCTTCCGATCTACTGTTGAAGCCTTCGGAGA | 19 |
| ILLUPE2LR8 | GACGTGTGCTCTTCCGATCTAAACCCACACAGACCCTCAC | 20 |
| ILLUPE2LR9 | GACGTGTGCTCTTCCGATCTAGTCTGGGGCTGAGGTGAAG | 21 |
| ILLUPE2LR10 | GACGTGTGCTCTTCCGATCTGGCCCAGGACTGGTGAAG | 22 |
| ILLUPE2LR11 | GACGTGTGCTCTTCCGATCTGGTGCAGCTGGTGGAGTC | 23 |
| ILLUPE1adaptor_short | ACACTCTTTCCCTACACGAC | 24 |

2^{nd} PCR reverse primer

| | | |
|---|---|---|
| ILLUPE1adaptor | AATGATACGGCGACCACCGAGATCTACACTCTTTCCCTACACGAC | 25 |

2^{nd} PCR forward primers with 7 library barcodes

| | | |
|---|---|---|
| ILLUPE2TSBC2 | CAAGCAGAAGACGGCATACGAGATAACGAAACGTGACTGGAGTTCAGAC | 26 |
| 1 | GTGTGCTCTTCCGATCT | |
| ILLUPE2TSBC2 | CAAGCAGAAGACGGCATACGAGATAACGTACGGTGACTGGAGTTCAGAC | 27 |
| 2 | GTGTGCTCTTCCGATCT | |
| ILLUPE2TSBC2 | CAAGCAGAAGACGGCATACGAGATAACCACTCGTGACTGGAGTTCAGAC | 28 |
| 3 | GTGTGCTCTTCCGATCT | |
| ILLUPE2TSBC2 | CAAGCAGAAGACGGCATACGAGATAAATCAGTGTGACTGGAGTTCAGAC | 29 |
| 5 | GTGTGCTCTTCCGATCT | |
| ILLUPE2TSBC2 | CAAGCAGAAGACGGCATACGAGATAAGCTCATGTGACTGGAGTTCAGAC | 30 |
| 6 | GTGTGCTCTTCCGATCT | |
| ILLUPE2TSBC2 | CAAGCAGAAGACGGCATACGAGATAAAGGAATGTGACTGGAGTTCAGAC | 31 |
| 7 | GTGTGCTCTTCCGATCT | |
| ILLUPE2TSBC2 | CAAGCAGAAGACGGCATACGAGATAACTTTTGGTGACTGGAGTTCAGAC | 32 |
| 8 | GTGTGCTCTTCCGATCT | |

iTAST

| | | |
|---|---|---|
| RT | | |
| RT_TCRa | CAGATCTCAGCTGGACCACA | 33 |
| RT_TCRb | TCATAGAGGATGGTGGCAGA | 34 |

1st PCR:

| | | |
|---|---|---|
| 1st PCR | CAGATCTCAGCTGGACCACA | 35 |
| reverse_TCRa | | |
| 1st PCR | TCATAGAGGATGGTGGCAGA | 36 |
| reverse_TCRb | | |
| TRAV1-1/2 | GCACCCACATTTCTKTCTTACAATG | 37 |
| TRAV2 | ATGTGCACCAAGACTCCTTGTTAAA | 38 |
| TRAV3 | GCAGCTATGGCTTTGAAGCTG | 39 |
| TRAV8 | AAVGGYTTTGAGGCTGAATTT | 40 |
| TRAV4 | CAAGACAAAAGTTACAAACGAAGTGG | 41 |
| TRAV5 | TGGACATGAAACAAGACCAAAGACT | 42 |
| TRAV6 | AAAAAGGAAAGAAAGACTGAAGGT | 43 |
| TRAV7 | TCAGCTGGATATGAGAAGCAGAAAG | 44 |
| TRAV9 | AAGGGAAGSAACAAAGGTTTTGAAG | 45 |
| TRAV10 | AGAACACAAAGTCGAACGGAAGATA | 46 |
| TRAV11/15 | TTGTGTCTTTGACCTTAATTCAATC | 47 |
| TRAV12 | TCARTGTTCCAGAGGGAGCCAYT | 48 |
| TRAV13 | CTGAGTGTCCAGGAGGGWGACA | 49 |
| TRAV14 | AGCAGTGGGGAAATGATTTTTCTT | 50 |
| TRAV16 | TCTAGAGAGAGCATCAAAGGCTTCA | 51 |
| TRAV17 | CGTTCAAATGAAAGAGAGAAACACA | 52 |
| TRAV18 | CCTGAAAAGTTCAGAAAACCAGGAG | 53 |
| TRAV19 | CCTTATTCGTCGGAACTCTTTTGAT | 54 |
| TRAV20 | CTGGGGAAGAAAAGGAGAAAGAAAG | 55 |
| TRAV21 | CAGAGAGAGCAAACAAGTGGAAGAC | 56 |
| TRAV22 | CATCAACCTGTTTTACATTCCCTCA | 57 |
| TRAV23 | GCATTATTGATAGCCATACGTCCAG | 58 |
| TRAV24 | TAAATGGGGATGAAAAGAAGAAAGG | 59 |
| TRAV25 | CTGGTGGACATCCCGTTTTT | 60 |
| TRAV26 | ATTGGTATCGACAGMTTCMCTCC | 61 |
| TRAV27 | CCTGTCCTCCTGGTGACAGTAGTTA | 62 |
| TRAV28 | GGACCCCTCATGTCCTTATTTAACA | 63 |
| TRAV29 | TGCTGAAGGTCCTACATTCCTGATA | 64 |
| TRAV30 | CCCGTCTTCCTGATGATATTACTGA | 65 |
| TRAV31 | GAAGATTATTTTCCTCATTTATCAGC | 66 |
| TRAV32 | GGGAAGGCCCTAATATCTTAATGGA | 67 |
| TRAV33 | CCCAGTGAAGAGATGGTTTTCCTTA | 68 |
| TRAV34 | TGAAGGTCTTATCTTCTTGATGATGC | 69 |
| TRAV35 | AGGTCCTGTCCTCTTGATAGCCTTA | 70 |
| TRAV36 | GGAAAAGAAAGCTCCCACATTTCTA | 71 |
| TRAV37 | CCTCATTTCCCTGATACAAATGCTA | 72 |
| TRAV38 | AGCAGGCAGATGATTCTCGTTATTC | 73 |
| TRAV39 | GTCTGGAATCTCTGTTTGTGTTGCT | 74 |
| TRAV40 | TGCAGCTTCTTCAGAGAGAGACAAT | 75 |
| TRAV41 | GCATTGTTTCCTTGTTTATGCTGAG | 76 |
| TRBV1 | AAGAAATCCCTGGAGTTCATGTTTT | 77 |
| TRBV2 | GTACAGACAAATCTTGGGGCAGAAA | 78 |
| TRBV3 | TCTGGGCCATRATRCTATGTATTGG | 79 |
| TRBV4 | AGTGTGCCAAGTCGCTTCTCAC | 80 |
| TRBV5-1/2/3/4/5/6/7 | GGGCCCCAGTTTATCTTTCAGTAT | 81 |
| TRBV5-8 | CAGYTCCTCCTTTGGTATGACGAG | 82 |
| TRBV6-1 | GAGGGTACCACTGACAAAGGAGAAG | 83 |
| TRBV6-2/3 | ACTCAGTTGGTGAGGGTACAACTGC | 84 |
| TRBV6-4 | AGGTACCACTGGCAAAGGAGAAGT | 85 |
| TRBV6-5/6 | TCAGTTGGTGCTGGTATCACTGAY | 86 |
| TRBV6-7 | TGCTCTCACTGACAAAGGAGAAGTT | 87 |
| TRBV6-8 | TGCTGCTGGTACTACTGACAAAGAA | 88 |
| TRBV6-9 | GCTGGTATCACTGACAAAGGAGAAG | 89 |
| TRBV7-1/2/3 | CAGGTCATAMTGCCCTTTAYTGGT | 90 |
| TRBV7-4 | GACTTACTCCCAGAGTGATGCTCAA | 91 |
| TRBV7-5/6/7/9 | AGGGCCMAGAGTTTCTGACTTMCTT | 92 |
| TRBV7-8 | GCCAGAGTTTCTGACTTATTTCCAG | 93 |
| TRBV8-1 | TGCTCAGATTAGGAACCATTATTCA | 94 |
| TRBV8-2 | AACAGTGTTCTGATATCGACAGGA | 95 |
| TRBV9 | GTACTGGTACCAACAGAGCCTGGAC | 96 |
| TRBV10 | GGTATCGACAAGACCYGGGRCAT | 97 |
| TRBV11 | ACAGTTGCCTAAGGATCGATTTTCT | 98 |
| TRBV12-1/2 | CAGGGACTGGAATTGCTGARTTACT | 99 |
| TRVB12-3/4/5 | TCTGGTACAGACAGACCATGATGC | 100 |
| TRBV13 | TTCGTTTTATGAAAAGATGCAGAGC | 101 |
| TRBV14 | ATCGATTCTTAGCTGAAAGGACTGG | 102 |
| TRBV15 | AGACACCCCTGATAACTTCCAATCC | 103 |
| TRBV16 | AAACAGGTATGCCCAAGGAAAGATT | 104 |
| TRBV17 | AAACATTGCAGTTGATTCAGGGATG | 105 |
| TRBV18 | CATAGATGAGTCAGGAATGCCAAAG | 106 |
| TRBV19 | TCAGAAAGGAGATATAGCTGAAGGGTA | 108 |
| TRBV20-1 | CAAGGCCACATACGAGCAAGGCGTC | 109 |
| TRBV21-1 | TCAGAAAGCAGAAATAATCAATGAGC | 110 |
| TRBV22-1 | GAGGAGATCTAACTGAAGGCTACGTG | 111 |
| TRBV23-1 | CAAGAAACGGAGATGCACAAGAAG | 112 |
| TRBV24-1 | CGGTTGATCTATTACTCCTTTGATGTC | 113 |
| TRBV25-1 | AATTCCACAGAGAAGGGAGATCTTT | 114 |
| TRBV26 | ACTGGGAGCACTGAAAAAGGAGATA | 115 |
| TRBV27 | TTCAATGAATGTTGAGGTGACTGAT | 116 |
| TRBV28 | CGGCTGATCTATTTCTCATATGATGTT | 117 |
| TRBV29-1 | GACACTGATCGCAACTGCAAAT | 118 |
| TRBV30 | GCCTCCAGCTGCTCTTCTACTCC | 119 |

2nd PCR:

| | | |
|---|---|---|
| 2nd PCR | ACACTCTTTCCCTACACGACGCTCTTCCGATCT NHNHN XXXXXX | 120 |
| reverse_TCRa | GGTACACGGCAGGGTCAG | |
| 2nd PCR | ACACTCTTTCCCTACACGACGCTCTTCCGATCT NHNHN XXXXXX | 121 |
| reverse_TCRb | GACCTCGGGTGGGAACAC | |

2nd PCR forward:

| | | |
|---|---|---|
| TRAV1-1/2 | GACGTGTGCTCTTCCGATCTGAMAGGTCGTTTTTCTTCATTCCTT | 122 |
| TRAV2 | GACGTGTGCTCTTCCGATCTAGGGACGATACAACATGACCTATGA | 123 |
| TRAV3/8-2/4/5/6/7 | GACGTGTGCTCTTCCGATCTTCCTTCCACCTGAVGAAACC | 124 |
| TRAV8-1/2/3 | GACGTGTGCTCTTCCGATCTTTYAATCTGAGGAAACCCTCTGTG | 125 |
| TRAV4 | GACGTGTGCTCTTCCGATCTGACAGAAAGTCCAGCACTCTGAGC | 126 |
| TRAV5 | GACGTGTGCTCTTCCGATCTGGATAAACATCTGTCTCTGCGCATT | 127 |
| TRAV6 | GACGTGTGCTCTTCCGATCTCACCTTTGATACCACCCTTAAMCAG | 128 |
| TRAV7 | GACGTGTGCTCTTCCGATCTTTACTGAAGAATGGAAGCAGCTTGT | 129 |
| TRAV9 | GACGTGTGCTCTTCCGATCTCGTAARGAAACCACTTCTTTCCACT | 130 |
| TRAV10 | GACGTGTGCTCTTCCGATCTAAGCAAAGCTCTCTGCACATCAC | 131 |
| TRAV11/15 | GACGTGTGCTCTTCCGATCTGCTTGGAAAAGARAARTTTTATAGTG | 132 |
| TRAV12 | GACGTGTGCTCTTCCGATCTGAAGATGGAAGGTTTACAGCACA | 133 |
| TRAV13 | GACGTGTGCTCTTCCGATCTTYATTATAGACATTCGTTCAAATRTGG | 134 |
| TRAV14 | GACGTGTGCTCTTCCGATCTTTGAATTTCCAGAAGGCAAGAAAAT | 135 |
| TRAV16 | GACGTGTGCTCTTCCGATCTGACCTTAACAAAGGCGAGACATCTT | 136 |
| TRAV17 | GACGTGTGCTCTTCCGATCTCTTGACACTTCCAAGAAAAGCAGTT | 137 |
| TRAV18 | GACGTGTGCTCTTCCGATCTTTTTCAGGCCAGTCCTATCAAGAGT | 138 |
| TRAV19 | GACGTGTGCTCTTCCGATCTTGAAATAAGTGGTCGGTATTCTTGG | 139 |
| TRAV20 | GACGTGTGCTCTTCCGATCTAGCCACATTAACAAAGAAGGAAAGC | 140 |
| TRAV21 | GACGTGTGCTCTTCCGATCTTTAATGCCTCGCTGGATAAATCAT | 141 |
| TRAV22 | GACGTGTGCTCTTCCGATCTGCTACGGAACGCTACAGCTTATTG | 142 |
| TRAV23 | GACGTGTGCTCTTCCGATCTTGAGTGAAAAGAAAGAAGGAAGATTCA | 143 |
| TRAV24 | GACGTGTGCTCTTCCGATCTTACCAAGGAGGGTTACAGCTATTTG | 144 |
| TRAV25 | GACGTGTGCTCTTCCGATCTTGGAGAAGTGAAGAAGCAGAAAAGA | 145 |
| TRAV26 | GACGTGTGCTCTTCCGATCTAAGACAGAAAGTCCAGYACCTTGAT | 146 |
| TRAV27 | GACGTGTGCTCTTCCGATCTTGGAGAAGTGAAGAAGCTGAAGAGA | 147 |
| TRAV28 | GACGTGTGCTCTTCCGATCTGAAGACTAAAATCCGCAGTCAAAGC | 148 |
| TRAV29 | GACGTGTGCTCTTCCGATCTTCCATTAAGGATAAAAATGAAGATGGA | 149 |
| TRAV30 | GACGTGTGCTCTTCCGATCTAAGCRGCAAAGCTCCCTGTACCTTA | 150 |
| TRAV31 | GACGTGTGCTCTTCCGATCTAATGCGACACAGGGTCAATATTCT | 151 |
| TRAV32 | GACGTGTGCTCTTCCGATCTTGTGGATAGAAAACAGGACAGAAGG | 152 |
| TRAV33 | GACGTGTGCTCTTCCGATCTTAAGTCAAATGCAAAGCCTGTGAAC | 153 |
| TRAV34 | GACGTGTGCTCTTCCGATCTGGGGAAGAGAAAAGTCATGAAAAGA | 154 |
| TRAV35 | GACGTGTGCTCTTCCGATCTGGAAGACTGACTGCTCAGTTTGGTA | 155 |
| TRAV36 | GACGTGTGCTCTTCCGATCTTGGAATTGAAAAGAAGTCAGGAAGA | 156 |
| TRAV37 | GACGTGTGCTCTTCCGATCTAGAAGATCAGTGGAAGATTCACAGC | 157 |
| TRAV38 | GACGTGTGCTCTTCCGATCTAGAAAGCAGCCAAATCCTTCAGTCT | 158 |
| TRAV39 | GACGTGTGCTCTTCCGATCTGACGATTAATGGCCTCACTTGATAC | 159 |
| TRAV40 | GACGTGTGCTCTTCCGATCTGGAGGCGGAAATATTAAAGACAAAA | 160 |
| TRAV41 | GACGTGTGCTCTTCCGATCTGCATGGAAGATTAATTGCCACAATA | 161 |
| TRBV1 | GACGTGTGCTCTTCCGATCTCTGACAGCTCTCGCTTATACCTTCA | 162 |
| TRBV2 | GACGTGTGCTCTTCCGATCTGCCTGATGGATCAAATTTCACTCTG | 163 |
| TRBV3 | GACGTGTGCTCTTCCGATCTAATGAAACAGTTCCAAATCGMTTCT | 164 |
| TRBV4 | GACGTGTGCTCTTCCGATCTCCAAGTCGCTTCTCACCTGAAT | 165 |
| TRBV5-1 | GACGTGTGCTCTTCCGATCTCGCCAGTTCTCTAACTCTCGCTCT | 166 |
| TRBV5-2 | GACGTGTGCTCTTCCGATCTTTACTGAGTCAAACACGGAGCTAGG | 167 |
| TRBV5-3 | GACGTGTGCTCTTCCGATCTCTCTGAGATGAATGTGAGTGCCTTG | 168 |
| TRBV5-4/5/6/7/8 | GACGTGTGCTCTTCCGATCTCTGAGCTGAATGTGAACGCCTTG | 169 |
| TRBV6-1 | GACGTGTGCTCTTCCGATCTTCTCCAGATTAAACAAACGGGAGTT | 170 |
| TRBV6-2/3 | GACGTGTGCTCTTCCGATCTCTGATGGCTACAATGTCTCCAGATT | 171 |
| TRBV6-4 | GACGTGTGCTCTTCCGATCTAGTGTCTCCAGAGCAAACACAGATG | 172 |
| TRBV6-5/6/7 | GACGTGTGCTCTTCCGATCTGTCTCCAGATCAAMCACAGAGGATT | 173 |
| TRBV6-8/9 | GACGTGTGCTCTTCCGATCTAAACACAGAGGATTTCCCRCTCAG | 174 |
| TRBV7-1 | GACGTGTGCTCTTCCGATCTGTCTGAGGGATCCATCTCCACTC | 175 |
| TRBV7-2 | GACGTGTGCTCTTCCGATCTTCGCTTCTCTGCAGAGAGGACTGG | 176 |
| TRBV7-3 | GACGTGTGCTCTTCCGATCTCTGAGGGATCCGTCTCTACTCTGAA | 177 |
| TRBV7-4/8 | GACGTGTGCTCTTCCGATCTCTGAGRGATCCGTCTCCACTCTG | 178 |
| TRBV7-5 | GACGTGTGCTCTTCCGATCTGGTCTGAGGATCTTTCTCCACCT | 179 |
| TRBV7-6/7 | GACGTGTGCTCTTCCGATCTGAGGGATCCATCTCCACTCTGAC | 180 |
| TRBV7-9 | GACGTGTGCTCTTCCGATCTCTGCAGAGAGGCCTAAGGGATCT | 181 |
| TRBV8-1 | GACGTGTGCTCTTCCGATCTAAGCTCAAGCATTTTCCCTCAAC | 182 |
| TRBV8-2 | GACGTGTGCTCTTCCGATCTATGTCACAGAGGGGTACTGTGTTTC | 183 |
| TRBV9 | GACGTGTGCTCTTCCGATCTACAGTTCCCTGACTTGCACTCTG | 184 |
| TRBV10-1/3 | GACGTGTGCTCTTCCGATCTACAAAGGAGAAGTCTCAGATGGCTA | 185 |
| TRBV10-2 | GACGTGTGCTCTTCCGATCTTGTCTCCAGATCCAAGACAGAGAA | 186 |
| TRBV11 | GACGTGTGCTCTTCCGATCTCTGCAGAGAGGCTCAAAGGAGTAG | 187 |
| TRBV12-1/2 | GACGTGTGCTCTTCCGATCTATCATTCTCYACTCTGAGGATCCAR | 188 |
| TRVB12-3/4/5 | GACGTGTGCTCTTCCGATCTACTCTGARGATCCAGCCCTCAGAAC | 189 |
| TRBV13 | GACGTGTGCTCTTCCGATCTCAGCTCAACAGTTCAGTGACTATCAT | 190 |
| TRBV14 | GACGTGTGCTCTTCCGATCTGAAAGGACTGGAGGGACGTATTCTA | 191 |
| TRBV15 | GACGTGTGCTCTTCCGATCTGCCGAACACTTCTTTCTGCTTTCT | 192 |
| TRBV16 | GACGTGTGCTCTTCCGATCTATTTTCAGCTAAGTGCCTCCCAAAT | 193 |
| TRBV17 | GACGTGTGCTCTTCCGATCTCACAGCTGAAAGACCTAACGGAAC | 194 |
| TRBV18 | GACGTGTGCTCTTCCGATCTATTTTCTGCTGAATTTCCCAAAGAG | 195 |
| TRBV19 | GACGTGTGCTCTTCCGATCTGTCTCTCGGGAGAAGAAGGAATC | 196 |
| TRBV20-1 | GACGTGTGCTCTTCCGATCTGACAAGTTTCTCATCAACCATGCAA | 197 |
| TRBV21-1 | GACGTGTGCTCTTCCGATCTCAATGCTCCAAAAACTCATCCTGT | 198 |
| TRBV22-1 | GACGTGTGCTCTTCCGATCTAGGAGAAGGGGCTATTTCTTCTCAG | 199 |
| TRBV23-1 | GACGTGTGCTCTTCCGATCTATTCTCATCTCAATGCCCCAAGAAC | 200 |
| TRBV24-1 | GACGTGTGCTCTTCCGATCTGACAGGCACAGGCTAAATTCTCC | 201 |
| TRBV25-1 | GACGTGTGCTCTTCCGATCTAGTCTCCAGAATAAGGACGGAGCAT | 202 |
| TRBV26 | GACGTGTGCTCTTCCGATCTCTCTGAGGGGTATCATGTTTCTTGA | 203 |
| TRBV27 | GACGTGTGCTCTTCCGATCTCAAAGTCTCTCGAAAAGAGAAGAGGA | 204 |
| TRBV28 | GACGTGTGCTCTTCCGATCTAAGAAGGAGCGCTTCTCCCTGATT | 205 |
| TRBV29-1 | GACGTGTGCTCTTCCGATCTCGCCCAAACCTAACATTCTCAA | 206 |
| TRBV30 | GACGTGTGCTCTTCCGATCTCCAGAATCTCTCAGCCTCCAGAC | 207 |

3rd PCR:

| | | |
|---|---|---|
| 3rd PCR reverse | AATGATACGGCGACCACCGAGATCTACACTCTTTCCCTACACGAC | 208 |
| 3rd PCR forward | CAAGCAGAAGACGGCATACGAGATAA XXXXXX | 209 |
| | GTGACTGGAGTTCAGACGTGTGCTCTTCCGATCT | |

3′seTCR

| | | |
|---|---|---|
| RT: | | |
| RT | AAGCAGTGGTATCAACGCAGAGT XXXXX TTT TTT TTT TTT TTT | 210 |
| | TTT TTT TTT TTT TTT VN | |
| TSO | | 211 |

1st PCR:

| | | |
|---|---|---|
| 1st PCR primer | AAGCAGTGGTATCAACGCAGAGT | 212 |

2nd PCR:

| | | |
|---|---|---|
| 2nd PCR reverse | AAGCAGTGGTATCAACGCAGAGT | 213 |

2nd PCR forward:

| | | |
|---|---|---|
| TRAV1-1/2 | GCACCCACATTTCTKTCTTACAATG | 214 |
| TRAV2 | ATGTGCACCAAGACTCCTTGTTAAA | 215 |
| TRAV3 | GCAGCTATGGCTTTGAAGCTG | 216 |
| TRAV8 | AAVGGYTTTGAGGCTGAATTT | 217 |
| TRAV4 | CAAGACAAAAGTTACAAACGAAGTGG | 218 |
| TRAV5 | TGGACATGAAACAAGACCAAAGACT | 219 |
| TRAV6 | AAAAAGGAAAGAAAGACTGAAGGT | 220 |
| TRAV7 | TCAGCTGGATATGAGAAGCAGAAAG | 221 |
| TRAV9 | AAGGGAAGSAACAAAGGTTTTGAAG | 222 |
| TRAV10 | AGAACACAAAGTCGAACGGAAGATA | 223 |
| TRAV11/15 | TTGTGTCTTTGACCTTAATTCAATC | 224 |
| TRAV12 | TCARTGTTCCAGAGGGAGCCAYT | 225 |
| TRAV13 | CTGAGTGTCCAGGAGGGWGACA | 226 |
| TRAV14 | AGCAGTGGGGAAATGATTTTTCTT | 227 |
| TRAV16 | TCTAGAGAGAGCATCAAAGGCTTCA | 228 |
| TRAV17 | CGTTCAAATGAAAGAGAGAAACACA | 229 |
| TRAV18 | CCTGAAAAGTTCAGAAAACCAGGAG | 230 |
| TRAV19 | CCTTATTCGTCGGAACTCTTTTGAT | 231 |
| TRAV20 | CTGGGGAAGAAAAGGAGAAAGAAAG | 232 |
| TRAV21 | CAGAGAGAGCAAACAAGTGGAAGAC | 233 |
| TRAV22 | CATCAACCTGTTTTACATTCCCTCA | 234 |
| TRAV23 | GCATTATTGATAGCCATACGTCCAG | 235 |
| TRAV24 | TAAATGGGGATGAAAAGAAGAAAGG | 236 |
| TRAV25 | CTGGTGGACATCCCGTTTTT | 237 |
| TRAV26 | ATTGGTATCGACAGMTTCMCTCC | 238 |
| TRAV27 | CCTGTCCTCCTGGTGACAGTAGTTA | 239 |
| TRAV28 | GGACCCCTCATGTCCTTATTTAACA | 240 |
| TRAV29 | TGCTGAAGGTCCTACATTCCTGATA | 241 |
| TRAV30 | CCCGTCTTCCTGATGATATTACTGA | 242 |
| TRAV31 | GAAGATTATTTTCCTCATTTATCAGC | 243 |
| TRAV32 | GGGAAGGCCCTAATATCTTAATGGA | 244 |
| TRAV33 | CCCAGTGAAGAGATGGTTTTCCTTA | 245 |
| TRAV34 | TGAAGGTCTTATCTTCTTGATGATGC | 246 |
| TRAV35 | AGGTCCTGTCCTCTTGATAGCCTTA | 247 |
| TRAV36 | GGAAAAGAAAGCTCCCACATTTCTA | 248 |
| TRAV37 | CCTCATTTCCCTGATACAAATGCTA | 249 |
| TRAV38 | AGCAGGCAGATGATTCTCGTTATTC | 250 |
| TRAV39 | GTCTGGAATCTCTGTTTGTGTTGCT | 251 |
| TRAV40 | TGCAGCTTCTTCAGAGAGAGACAAT | 252 |
| TRAV41 | GCATTGTTTCCTTGTTTATGCTGAG | 253 |
| TRBV1 | AAGAAATCCCTGGAGTTCATGTTTT | 254 |
| TRBV2 | GTACAGACAAATCTTGGGGCAGAAA | 255 |
| TRBV3 | TCTGGGCCATRATRCTATGTATTGG | 256 |
| TRBV4 | AGTGTGCCAAGTCGCTTCTCAC | 257 |
| TRBV5-1/2/3/4/5/6/7 | GGGCCCCAGTTTATCTTTCAGTAT | 258 |
| TRBV5-8 | CAGYTCCTCCTTTGGTATGACGAG | 259 |
| TRBV6-1 | GAGGGTACCACTGACAAAGGAGAAG | 260 |
| TRBV6-2/3 | ACTCAGTTGGTGAGGGTACAACTGC | 261 |
| TRBV6-4 | AGGTACCACTGGCAAAGGAGAAGT | 262 |
| TRBV6-5/6 | TCAGTTGGTGCTGGTATCACTGAY | 263 |
| TRBV6-7 | TGCTCTCACTGACAAAGGAGAAGTT | 264 |
| TRBV6-8 | TGCTGCTGGTACTACTGACAAAGAA | 265 |
| TRBV6-9 | GCTGGTATCACTGACAAAGGAGAAG | 266 |
| TRBV7-1/2/3 | CAGGTCATAMTGCCCTTTAYTGGT | 267 |
| TRBV7-4 | GACTTACTCCCAGAGTGATGCTCAA | 268 |
| TRBV7-5/6/7/9 | AGGGCCMAGAGTTTCTGACTTMCTT | 269 |
| TRBV7-8 | GCCAGAGTTTCTGACTTATTTCCAG | 270 |
| TRBV8-1 | TGCTCAGATTAGGAACCATTATTCA | 271 |
| TRBV8-2 | AACAGTGTTCTGATATCGACAGGA | 107 |
| TRBV9 | GTACTGGTACCAACAGAGCCTGGAC | 272 |
| TRBV10 | GGTATCGACAAGACCYGGGRCAT | 273 |
| TRBV11 | ACAGTTGCCTAAGGATCGATTTTCT | 274 |
| TRBV12-1/2 | CAGGGACTGGAATTGCTGARTTACT | 275 |
| TRVB12-3/4/5 | CAGGGACTGGAATTGCTGARTTACT | 276 |
| TRBV13 | TTCGTTTTATGAAAAGATGCAGAGC | 277 |
| TRBV14 | ATCGATTCTTAGCTGAAAGGACTGG | 278 |
| TRBV15 | AGACACCCCTGATAACTTCCAATCC | 279 |
| TRBV16 | AAACAGGTATGCCCAAGGAAAGATT | 280 |
| TRBV17 | AAACATTGCAGTTGATTCAGGGATG | 281 |
| TRBV18 | CATAGATGAGTCAGGAATGCCAAAG | 282 |
| TRBV19 | TCAGAAAGGAGATATAGCTGAAGGGTA | 283 |
| TRBV20-1 | CAAGGCCACATACGAGCAAGGCGTC | 284 |
| TRBV21-1 | TCAGAAAGCAGAAATAATCAATGAGC | 285 |
| TRBV22 -1 | GAGGAGATCTAACTGAAGGCTACGTG | 286 |
| TRBV23-1 | CAAGAAACGGAGATGCACAAGAAG | 287 |
| TRBV24-1 | CGGTTGATCTATTACTCCTTTGATGTC | 288 |
| TRBV25-1 | AATTCCACAGAGAAGGGAGATCTTT | 289 |
| TRBV26 | ACTGGGAGCACTGAAAAAGGAGATA | 290 |
| TRBV27 | TTCAATGAATGTTGAGGTGACTGAT | 291 |
| TRBV28 | CGGCTGATCTATTTCTCATATGATGTT | 292 |
| TRBV29-1 | GACACTGATCGCAACTGCAAAT | 293 |
| TRBV30 | GCCTCCAGCTGCTCTTCTACTCC | 294 |

3rd PCR:

| | | |
|---|---|---|
| 3rd PCR | AAGCAGTGGTATCAACGCAGAGT | 295 |
| reverse | | |

3rd PCR forward:

| | | |
|---|---|---|
| TRAV1-1/2 | GACGTGTGCTCTTCCGATCTGAMAGGTCGTTTTTCTTCATTCCTT | 296 |
| TRAV2 | GACGTGTGCTCTTCCGATCTAGGGACGATACAACATGACCTATGA | 297 |
| TRAV3/8-2/4/5/6/7 | GACGTGTGCTCTTCCGATCTTCCTTCCACCTGAVGAAACC | 298 |
| TRAV8-1/2/3 | GACGTGTGCTCTTCCGATCTTTYAATCTGAGGAAACCCTCTGTG | 299 |
| TRAV4 | GACGTGTGCTCTTCCGATCTGACAGAAAGTCCAGCACTCTGAGC | 300 |
| TRAV5 | GACGTGTGCTCTTCCGATCTGGATAAACATCTGTCTCTGCGCATT | 301 |
| TRAV6 | GACGTGTGCTCTTCCGATCTCACCTTTGATACCACCCTTAAMCAG | 302 |
| TRAV7 | GACGTGTGCTCTTCCGATCTTTACTGAAGAATGGAAGCAGCTTGT | 303 |
| TRAV9 | GACGTGTGCTCTTCCGATCTCGTAARGAAACCACTTCTTTCCACT | 304 |
| TRAV10 | GACGTGTGCTCTTCCGATCTAAGCAAAGCTCTCTGCACATCAC | 305 |
| TRAV11/15 | GACGTGTGCTCTTCCGATCTGCTTGGAAAAGARAARTTTTATAGTG | 306 |
| TRAV12 | GACGTGTGCTCTTCCGATCTGAAGATGGAAGGTTTACAGCACA | 307 |
| TRAV13 | GACGTGTGCTCTTCCGATCTTYATTATAGACATTCGTTCAAATRTGG | 308 |
| TRAV14 | GACGTGTGCTCTTCCGATCTTTGAATTTCCAGAAGGCAAGAAAAT | 309 |
| TRAV16 | GACGTGTGCTCTTCCGATCTGACCTTAACAAAGGCGAGACATCTT | 310 |
| TRAV17 | GACGTGTGCTCTTCCGATCTCTTGACACTTCCAAGAAAAGCAGTT | 311 |
| TRAV18 | GACGTGTGCTCTTCCGATCTTTTTCAGGCCAGTCCTATCAAGAGT | 312 |
| TRAV19 | GACGTGTGCTCTTCCGATCTTGAAATAAGTGGTCGGTATTCTTGG | 313 |
| TRAV20 | GACGTGTGCTCTTCCGATCTAGCCACATTAACAAAGAAGGAAAGC | 314 |
| TRAV21 | GACGTGTGCTCTTCCGATCTTTAATGCCTCGCTGGATAAATCAT | 315 |
| TRAV22 | GACGTGTGCTCTTCCGATCTGCTACGGAACGCTACAGCTTATTG | 316 |
| TRAV23 | GACGTGTGCTCTTCCGATCTTGAGTGAAAAGAAAGAAGGAAGATTCA | 317 |
| TRAV24 | GACGTGTGCTCTTCCGATCTTACCAAGGAGGGTTACAGCTATTTG | 318 |
| TRAV25 | GACGTGTGCTCTTCCGATCTTGGAGAAGTGAAGAAGCAGAAAAGA | 319 |
| TRAV26 | GACGTGTGCTCTTCCGATCTAAGACAGAAAGTCCAGYACCTTGAT | 320 |
| TRAV27 | GACGTGTGCTCTTCCGATCTTGGAGAAGTGAAGAAGCTGAAGAGA | 321 |
| TRAV28 | GACGTGTGCTCTTCCGATCTGAAGACTAAAATCCGCAGTCAAAGC | 322 |
| TRAV29 | GACGTGTGCTCTTCCGATCTTCCATTAAGGATAAAAATGAAGATGGA | 323 |
| TRAV30 | GACGTGTGCTCTTCCGATCTAAGCRGCAAAGCTCCCTGTACCTTA | 324 |
| TRAV31 | GACGTGTGCTCTTCCGATCTAATGCGACACAGGGTCAATATTCT | 325 |
| TRAV32 | GACGTGTGCTCTTCCGATCTTGTGGATAGAAAACAGGACAGAAGG | 326 |
| TRAV33 | GACGTGTGCTCTTCCGATCTTAAGTCAAATGCAAAGCCTGTGAAC | 327 |
| TRAV34 | GACGTGTGCTCTTCCGATCTGGGGAAGAGAAAAGTCATGAAAAGA | 328 |
| TRAV35 | GACGTGTGCTCTTCCGATCTGGAAGACTGACTGCTCAGTTTGGTA | 329 |
| TRAV36 | GACGTGTGCTCTTCCGATCTTGGAATTGAAAAGAAGTCAGGAAGA | 330 |
| TRAV37 | GACGTGTGCTCTTCCGATCTAGAAGATCAGTGGAAGATTCACAGC | 331 |
| TRAV38 | GACGTGTGCTCTTCCGATCTAGAAAGCAGCCAAATCCTTCAGTCT | 332 |
| TRAV39 | GACGTGTGCTCTTCCGATCTGACGATTAATGGCCTCACTTGATAC | 333 |
| TRAV40 | GACGTGTGCTCTTCCGATCTGGAGGCGGAAATATTAAAGACAAAA | 334 |
| TRAV41 | GACGTGTGCTCTTCCGATCTGCATGGAAGATTAATTGCCACAATA | 335 |
| TRBV1 | GACGTGTGCTCTTCCGATCTCTGACAGCTCTCGCTTATACCTTCA | 336 |
| TRBV2 | GACGTGTGCTCTTCCGATCTGCCTGATGGATCAAATTTCACTCTG | 337 |
| TRBV3 | GACGTGTGCTCTTCCGATCTAATGAAACAGTTCCAAATCGMTTCT | 338 |
| TRBV4 | GACGTGTGCTCTTCCGATCTCCAAGTCGCTTCTCACCTGAAT | 339 |
| TRBV5-1 | GACGTGTGCTCTTCCGATCTCGCCAGTTCTCTAACTCTCGCTCT | 340 |
| TRBV5-2 | GACGTGTGCTCTTCCGATCTTTACTGAGTCAAACACGGAGCTAGG | 341 |
| TRBV5-3 | GACGTGTGCTCTTCCGATCTCTCTGAGATGAATGTGAGTGCCTTG | 342 |
| TRBV5-4/5/6/7/8 | GACGTGTGCTCTTCCGATCTCTGAGCTGAATGTGAACGCCTTG | 343 |
| TRBV6-1 | GACGTGTGCTCTTCCGATCTTCTCCAGATTAAACAAACGGGAGTT | 344 |
| TRBV6-2/3 | GACGTGTGCTCTTCCGATCTCTGATGGCTACAATGTCTCCAGATT | 345 |
| TRBV6-4 | GACGTGTGCTCTTCCGATCTAGTGTCTCCAGAGCAAACACAGATG | 346 |
| TRBV6-5/6/7 | GACGTGTGCTCTTCCGATCTGTCTCCAGATCAAMCACAGAGGATT | 347 |
| TRBV6-8/9 | GACGTGTGCTCTTCCGATCTAAACACAGAGGATTTCCCRCTCAG | 348 |
| TRBV7-1 | GACGTGTGCTCTTCCGATCTGTCTGAGGGATCCATCTCCACTC | 349 |
| TRBV7-2 | GACGTGTGCTCTTCCGATCTTCGCTTCTCTGCAGAGAGGACTGG | 350 |
| TRBV7-3 | GACGTGTGCTCTTCCGATCTCTGAGGGATCCGTCTCTACTCTGAA | 351 |
| TRBV7-4/8 | GACGTGTGCTCTTCCGATCTCTGAGRGATCCGTCTCCACTCTG | 352 |
| TRBV7-5 | GACGTGTGCTCTTCCGATCTGGTCTGAGGATCTTTCTCCACCT | 353 |
| TRBV7-6/7 | GACGTGTGCTCTTCCGATCTGAGGGATCCATCTCCACTCTGAC | 354 |
| TRBV7-9 | GACGTGTGCTCTTCCGATCTCTGCAGAGAGGCCTAAGGGATCT | 355 |
| TRBV8-1 | GACGTGTGCTCTTCCGATCTAAGCTCAAGCATTTTCCCTCAAC | 356 |
| TRBV8-2 | GACGTGTGCTCTTCCGATCTATGTCACAGAGGGGTACTGTGTTTC | 357 |
| TRBV9 | GACGTGTGCTCTTCCGATCTACAGTTCCCTGACTTGCACTCTG | 358 |
| TRBV10-1/3 | GACGTGTGCTCTTCCGATCTACAAAGGAGAAGTCTCAGATGGCTA | 359 |
| TRBV10-2 | GACGTGTGCTCTTCCGATCTTGTCTCCAGATCCAAGACAGAGAA | 360 |
| TRBV11 | GACGTGTGCTCTTCCGATCTCTGCAGAGAGGCTCAAAGGAGTAG | 361 |
| TRBV12-1/2 | GACGTGTGCTCTTCCGATCTATCATTCTCYACTCTGAGGATCCAR | 362 |
| TRVB12-3/4/5 | GACGTGTGCTCTTCCGATCTACTCTGARGATCCAGCCCTCAGAAC | 363 |
| TRBV13 | GACGTGTGCTCTTCCGATCTCAGCTCAACAGTTCAGTGACTATCAT | 364 |
| TRBV14 | GACGTGTGCTCTTCCGATCTGAAAGGACTGGAGGGACGTATTCTA | 365 |
| TRBV15 | GACGTGTGCTCTTCCGATCTGCCGAACACTTCTTTCTGCTTTCT | 366 |
| TRBV16 | GACGTGTGCTCTTCCGATCTATTTTCAGCTAAGTGCCTCCCAAAT | 367 |
| TRBV17 | GACGTGTGCTCTTCCGATCTCACAGCTGAAAGACCTAACGGAAC | 368 |
| TRBV18 | GACGTGTGCTCTTCCGATCTATTTTCTGCTGAATTTCCCAAAGAG | 369 |
| TRBV19 | GACGTGTGCTCTTCCGATCTGTCTCTCGGGAGAAGAAGGAATC | 370 |
| TRBV20-1 | GACGTGTGCTCTTCCGATCTGACAAGTTTCTCATCAACCATGCAA | 371 |
| TRBV21-1 | GACGTGTGCTCTTCCGATCTCAATGCTCCAAAAACTCATCCTGT | 372 |
| TRBV22-1 | GACGTGTGCTCTTCCGATCTAGGAGAAGGGGCTATTTCTTCTCAG | 373 |
| TRBV23 -1 | GACGTGTGCTCTTCCGATCTATTCTCATCTCAATGCCCCAAGAAC | 374 |
| TRBV24-1 | GACGTGTGCTCTTCCGATCTGACAGGCACAGGCTAAATTCTCC | 375 |
| TRBV25-1 | GACGTGTGCTCTTCCGATCTAGTCTCCAGAATAAGGACGGAGCAT | 376 |
| TRBV26 | GACGTGTGCTCTTCCGATCTCTCTGAGGGGTATCATGTTTCTTGA | 377 |
| TRBV27 | GACGTGTGCTCTTCCGATCTCAAAGTCTCTCGAAAAGAGAAGAGGA | 378 |
| TRBV28 | GACGTGTGCTCTTCCGATCTAAGAAGGAGCGCTTCTCCCTGATT | 379 |
| TRBV29-1 | GACGTGTGCTCTTCCGATCTCGCCCAAACCTAACATTCTCAA | 380 |
| TRBV30 | GACGTGTGCTCTTCCGATCTCCAGAATCTCTCAGCCTCCAGAC | 381 |

4th PCR:

| | | |
|---|---|---|
| 4th PCR | CAAGCAGAAGACGGCATACGAGATAA XXXXXX | 382 |
| forward | GTGACTGGAGTTCAGACGTGTGCTCTTCCGATCT | |
| 4th PCR | AATGATACGGCGACCACCGAGATCTACACTCTTTCCCTACACGACGCTC | 383 |
| reverse | TTCCGATCTNHNHNAAGCAGTGGTATCAACGCAGAGT | |

MIDCIRS TCR

| | | |
|---|---|---|
| TCRB RT: | | |
| RT | ACACTCTTTCCCTACACGACGCTCTTCCGATCT NNNNNNNNNNNNGAC | 384 |
| | CTCGGGTGGGAACAC | |

1st PCR:

| | | |
|---|---|---|
| 1st PCR | ACACTCTTTCCCTACACGAC | 385 |
| reverse | | |

1st PCR forward:

| | | |
|---|---|---|
| TRBV1 | GACGTGTGCTCTTCCGATCTCTGACAGCTCTCGCTTATACCTTCA | 386 |
| TRBV2 | GACGTGTGCTCTTCCGATCTGCCTGATGGATCAAATTTCACTCTG | 387 |
| TRBV3 | GACGTGTGCTCTTCCGATCTAATGAAACAGTTCCAAATCGMTTCT | 388 |
| TRBV4 | GACGTGTGCTCTTCCGATCTCCAAGTCGCTTCTCACCTGAAT | 389 |
| TRBV5-1 | GACGTGTGCTCTTCCGATCTCGCCAGTTCTCTAACTCTCGCTCT | 390 |
| TRBV5-2 | GACGTGTGCTCTTCCGATCTTTACTGAGTCAAACACGGAGCTAGG | 391 |
| TRBV5-3 | GACGTGTGCTCTTCCGATCTCTCTGAGATGAATGTGAGTGCCTTG | 392 |
| TRBV5-4/5/6/7/8 | GACGTGTGCTCTTCCGATCTCTGAGCTGAATGTGAACGCCTTG | 393 |
| TRBV6-1 | GACGTGTGCTCTTCCGATCTTCTCCAGATTAAACAAACGGGAGTT | 394 |
| TRBV6-2/3 | GACGTGTGCTCTTCCGATCTCTGATGGCTACAATGTCTCCAGATT | 395 |
| TRBV6-4 | GACGTGTGCTCTTCCGATCTAGTGTCTCCAGAGCAAACACAGATG | 396 |
| TRBV6-5/6/7 | GACGTGTGCTCTTCCGATCTGTCTCCAGATCAAMCACAGAGGATT | 397 |
| TRBV6-8/9 | GACGTGTGCTCTTCCGATCTAAACACAGAGGATTTCCCRCTCAG | 398 |
| TRBV7-1 | GACGTGTGCTCTTCCGATCTGTCTGAGGGATCCATCTCCACTC | 399 |
| TRBV7-2 | GACGTGTGCTCTTCCGATCTTCGCTTCTCTGCAGAGAGGACTGG | 400 |
| TRBV7-3 | GACGTGTGCTCTTCCGATCTCTGAGGGATCCGTCTCTACTCTGAA | 401 |
| TRBV7-4/8 | GACGTGTGCTCTTCCGATCTCTGAGRGATCCGTCTCCACTCTG | 402 |
| TRBV7-5 | GACGTGTGCTCTTCCGATCTGGTCTGAGGATCTTTCTCCACCT | 403 |
| TRBV7-6/7 | GACGTGTGCTCTTCCGATCTGAGGGATCCATCTCCACTCTGAC | 404 |
| TRBV7-9 | GACGTGTGCTCTTCCGATCTCTGCAGAGAGGCCTAAGGGATCT | 405 |
| TRBV8-1 | GACGTGTGCTCTTCCGATCTAAGCTCAAGCATTTTCCCTCAAC | 406 |
| TRBV8-2 | GACGTGTGCTCTTCCGATCTATGTCACAGAGGGGTACTGTGTTTC | 407 |
| TRBV9 | GACGTGTGCTCTTCCGATCTACAGTTCCCTGACTTGCACTCTG | 408 |
| TRBV10-1/3 | GACGTGTGCTCTTCCGATCTACAAAGGAGAAGTCTCAGATGGCTA | 409 |
| TRBV10-2 | GACGTGTGCTCTTCCGATCTTGTCTCCAGATCCAAGACAGAGAA | 410 |
| TRBV11 | GACGTGTGCTCTTCCGATCTCTGCAGAGAGGCTCAAAGGAGTAG | 411 |
| TRBV12-1/2 | GACGTGTGCTCTTCCGATCTATCATTCTCYACTCTGAGGATCCAR | 412 |
| TRVB12-3/4/5 | GACGTGTGCTCTTCCGATCTACTCTGARGATCCAGCCCTCAGAAC | 413 |
| TRBV13 | GACGTGTGCTCTTCCGATCTCAGCTCAACAGTTCAGTGACTATCAT | 414 |
| TRBV14 | GACGTGTGCTCTTCCGATCTGAAAGGACTGGAGGGACGTATTCTA | 415 |
| TRBV15 | GACGTGTGCTCTTCCGATCTGCCGAACACTTCTTTCTGCTTTCT | 416 |
| TRBV16 | GACGTGTGCTCTTCCGATCTATTTTCAGCTAAGTGCCTCCCAAAT | 417 |
| TRBV17 | GACGTGTGCTCTTCCGATCTCACAGCTGAAAGACCTAACGGAAC | 418 |
| TRBV18 | GACGTGTGCTCTTCCGATCTATTTTCTGCTGAATTTCCCAAAGAG | 419 |
| TRBV19 | GACGTGTGCTCTTCCGATCTGTCTCTCGGGAGAAGAAGGAATC | 420 |
| TRBV20-1 | GACGTGTGCTCTTCCGATCTGACAAGTTTCTCATCAACCATGCAA | 421 |
| TRBV21-1 | GACGTGTGCTCTTCCGATCTCAATGCTCCAAAAACTCATCCTGT | 422 |
| TRBV22-1 | GACGTGTGCTCTTCCGATCTAGGAGAAGGGGCTATTTCTTCTCAG | 423 |
| TRBV23 -1 | GACGTGTGCTCTTCCGATCTATTCTCATCTCAATGCCCCAAGAAC | 424 |
| TRBV24-1 | GACGTGTGCTCTTCCGATCTGACAGGCACAGGCTAAATTCTCC | 425 |
| TRBV25-1 | GACGTGTGCTCTTCCGATCTAGTCTCCAGAATAAGGACGGAGCAT | 426 |
| TRBV26 | GACGTGTGCTCTTCCGATCTCTCTGAGGGGTATCATGTTTCTTGA | 427 |
| TRBV27 | GACGTGTGCTCTTCCGATCTCAAAGTCTCTCGAAAAGAGAAGAGGA | 428 |
| TRBV28 | GACGTGTGCTCTTCCGATCTAAGAAGGAGCGCTTCTCCCTGATT | 429 |
| TRBV29-1 | GACGTGTGCTCTTCCGATCTCGCCCAAACCTAACATTCTCAA | 430 |
| TRBV30 | GACGTGTGCTCTTCCGATCTCCAGAATCTCTCAGCCTCCAGAC | 431 |

2nd PCR:

| | | |
|---|---|---|
| 2nd PCR reverse | AATGATACGGCGACCACCGAGATCTACACTCTTTCCCTACACGAC | 432 |
| 2nd PCR forward | CAAGCAGAAGACGGCATACGAGATAA XXXXXX | 433 |
| | GTGACTGGAGTTCAGACGTGTGCTCTTCCGATCT | |

TCRA RT:

| | | |
|---|---|---|
| RT | ACACTCTTTCCCTACAGACGCTCTTCCGATCT NNNNNNNNNNNN | 434 |
| | GGTACACGGCAGGGTCAG | |

1st PCR:

| | | |
|---|---|---|
| 1st PCR reverse | ACACTCTTTCCCTACACGAC | 435 |

1st PCR forward:

| | | |
|---|---|---|
| TRAV1-1/2 | GACGTGTGCTCTTCCGATCTGAMAGGTCGTTTTTCTTCATTCCTT | 436 |
| TRAV2 | GACGTGTGCTCTTCCGATCTAGGGACGATACAACATGACCTATGA | 437 |
| TRAV3/8-2/4/5/6/7 | GACGTGTGCTCTTCCGATCTTCCTTCCACCTGAVGAAACC | 438 |
| TRAV8-1/2/3 | GACGTGTGCTCTTCCGATCTTTYAATCTGAGGAAACCCTCTGTG | 439 |
| TRAV4 | GACGTGTGCTCTTCCGATCTGACAGAAAGTCCAGCACTCTGAGC | 440 |
| TRAV5 | GACGTGTGCTCTTCCGATCTGGATAAACATCTGTCTCTGCGCATT | 441 |
| TRAV6 | GACGTGTGCTCTTCCGATCTCACCTTTGATACCACCCTTAAMCAG | 442 |
| TRAV7 | GACGTGTGCTCTTCCGATCTTTACTGAAGAATGGAAGCAGCTTGT | 443 |
| TRAV9 | GACGTGTGCTCTTCCGATCTCGTAARGAAACCACTTCTTTCCACT | 444 |
| TRAV10 | GACGTGTGCTCTTCCGATCTAAGCAAAGCTCTCTGCACATCAC | 445 |
| TRAV11/15 | GACGTGTGCTCTTCCGATCTGCTTGGAAAAGARAARTTITATAGTG | 446 |
| TRAV12 | GACGTGTGCTCTTCCGATCTGAAGATGGAAGGTTTACAGCACA | 447 |
| TRAV13 | GACGTGTGCTCTTCCGATCTTYATTATAGACATTCGTTCAAATRTGG | 448 |
| TRAV14 | GACGTGTGCTCTTCCGATCTTTGAATTTCCAGAAGGCAAGAAAAT | 449 |
| TRAV16 | GACGTGTGCTCTTCCGATCTGACCTTAACAAAGGCGAGACATCTT | 450 |
| TRAV17 | GACGTGTGCTCTTCCGATCTCTTGACACTTCCAAGAAAAGCAGTT | 451 |
| TRAV18 | GACGTGTGCTCTTCCGATCTTTTTCAGGCCAGTCCTATCAAGAGT | 452 |
| TRAV19 | GACGTGTGCTCTTCCGATCTTGAAATAAGTGGTCGGTATTCTTGG | 453 |
| TRAV20 | GACGTGTGCTCTTCCGATCTAGCCACATTAACAAAGAAGGAAAGC | 454 |
| TRAV21 | GACGTGTGCTCTTCCGATCTTTAATGCCTCGCTGGATAAATCAT | 455 |
| TRAV22 | GACGTGTGCTCTTCCGATCTGCTACGGAACGCTACAGCTTATTG | 456 |
| TRAV23 | GACGTGTGCTCTTCCGATCTTGAGTGAAAAGAAAGAAGGAAGATTCA | 457 |
| TRAV24 | GACGTGTGCTCTTCCGATCTTACCAAGGAGGGTTACAGCTATTTG | 458 |
| TRAV25 | GACGTGTGCTCTTCCGATCTTGGAGAAGTGAAGAAGCAGAAAAGA | 459 |
| TRAV26 | GACGTGTGCTCTTCCGATCTAAGACAGAAAGTCCAGYACCTTGAT | 460 |
| TRAV27 | GACGTGTGCTCTTCCGATCTTGGAGAAGTGAAGAAGCTGAAGAGA | 461 |
| TRAV28 | GACGTGTGCTCTTCCGATCTGAAGACTAAAATCCGCAGTCAAAGC | 462 |
| TRAV29 | GACGTGTGCTCTTCCGATCTTCCATTAAGGATAAAAATGAAGATGGA | 463 |
| TRAV30 | GACGTGTGCTCTTCCGATCTAAGCRGCAAAGCTCCCTGTACCTTA | 464 |
| TRAV31 | GACGTGTGCTCTTCCGATCTAATGCGACACAGGGTCAATATTCT | 465 |
| TRAV32 | GACGTGTGCTCTTCCGATCTTGTGGATAGAAAACAGGACAGAAGG | 466 |
| TRAV33 | GACGTGTGCTCTTCCGATCTTAAGTCAAATGCAAAGCCTGTGAAC | 467 |
| TRAV34 | GACGTGTGCTCTTCCGATCTGGGGAAGAGAAAAGTCATGAAAAGA | 468 |
| TRAV35 | GACGTGTGCTCTTCCGATCTGGAAGACTGACTGCTCAGTTTGGTA | 469 |
| TRAV36 | GACGTGTGCTCTTCCGATCTTGGAATTGAAAAGAAGTCAGGAAGA | 470 |
| TRAV37 | GACGTGTGCTCTTCCGATCTAGAAGATCAGTGGAAGATTCACAGC | 471 |
| TRAV38 | GACGTGTGCTCTTCCGATCTAGAAAGCAGCCAAATCCTTCAGTCT | 472 |
| TRAV39 | GACGTGTGCTCTTCCGATCTGACGATTAATGGCCTCACTTGATAC | 473 |
| TRAV40 | GACGTGTGCTCTTCCGATCTGGAGGCGGAAATATTAAAGACAAAA | 474 |
| TRAV41 | GACGTGTGCTCTTCCGATCTGCATGGAAGATTAATTGCCACAATA | 475 |

2nd PCR:

| | | |
|---|---|---|
| 2nd PCR reverse | AATGATACGGCGACCACCGAGATCTACACTCTTTCCCTACACGAC | 476 |
| 2nd PCR forward | CAAGCAGAAGACGGCATACGAGATAA XXXXXX | 477 |
| | GTGACTGGAGTTCAGACGTGTGCTCTTCCGATCT | |

Mouse TCR MIDCIRS

| | | |
|---|---|---|
| TCRA | | |
| RT: | | |
| TRAC_12N | ACACTCTTTCCCTACACGACGCTCTTCCGATCTNNNNNNNNNNNNAGCA | 478 |
| | GGTTCTGGGTTCTGGAT | |
| 1st PCR: | 2nd PCR: | |
| 1st PCR reverse | 2nd PCR reverse | |

1st PCR forward:

| | | |
|---|---|---|
| TRAV1 | GACGTGTGCTCTTCCGATCTCAGTTACCTGCTTCTGACAGAGC | 479 |
| TRAV10 | GACGTGTGCTCTTCCGATCTAAAGCCAAACGATTCTCCCTGC | 480 |
| TRAV11 | GACGTGTGCTCTTCCGATCTAGATGCTAAGCACAGCACGCT | 481 |
| TRAV12 | GACGTGTGCTCTTCCGATCTTCCATAAGAGCAGCAGCTCCT | 482 |
| TRAV13 -1 | GACGTGTGCTCTTCCGATCTGCTCTTTGCACATTTCCTCCTCC | 483 |
| TRAV13-2 | GACGTGTGCTCTTCCGATCTGCTCTTTGACTATATCCTCCTCC | 484 |
| TRAV14 | GACGTGTGCTCTTCCGATCTTCTCCTTGCACATYRHAGACTCT | 485 |
| TRAV15-1 | GACGTGTGCTCTTCCGATCTTCCATCAGCCTTRTCATTTCARC | 486 |
| TRAV15-2 | GACGTGTGCTCTTCCGATCTGCAKAACTTAGAACATSTTCACAGG | 487 |
| TRAV16 | GACGTGTGCTCTTCCGATCTAGTTCCATCGGACTCATCATCAC | 488 |
| TRAV17 | GACGTGTGCTCTTCCGATCTTCAACCTGAAGAAATCCCCAGC | 489 |
| TRAV18 | GACGTGTGCTCTTCCGATCTGCTCCCTGTTCATCGCCAGA | 490 |
| TRAV19 | GACGTGTGCTCTTCCGATCTAACAAAAGYGGCAAACACTKC | 491 |
| TRAV2 | GACGTGTGCTCTTCCGATCTCGGAAGCTCAGCACTCTGAG | 492 |
| TRAV20 | GACGTGTGCTCTTCCGATCTGCGTCTCCTTACATATAACAGC | 493 |
| TRAV21 | GACGTGTGCTCTTCCGATCTCTGACAGAAAGTCAAGCACCTY | 494 |
| TRAV22 | GACGTGTGCTCTTCCGATCTGCTCTTTTCCCTGCTCACAAAGG | 495 |
| TRAV23 | GACGTGTGCTCTTCCGATCTTGCACTTCTCCCCTGCACTT | 496 |
| TRAV3-1 | GACGTGTGCTCTTCCGATCTTCTCTCTATCTGAACATCACAGCA | 497 |
| TRAV3-2 | GACGTGTGCTCTTCCGATCTACTCTCTCTGAACCTCACAGCT | 498 |
| TRAV4 | GACGTGTGCTCTTCCGATCTDCTACAGCACCCYGCACA | 499 |
| TRAV5-1 | GACGTGTGCTCTTCCGATCTTTCTCCCTGCACAWCACAGACA | 500 |
| TRAV5-2 | GACGTGTGCTCTTCCGATCTACCCTTCTCCCTACACATCATA | 501 |
| TRAV5-3 | GACGTGTGCTCTTCCGATCTACACCTTTCCCTGCACATTACAG | 502 |
| TRAV5-4 | GACGTGTGCTCTTCCGATCTCTGGATAAGAAAGGCAAACACATC | 503 |
| TRAV6-1 | GACGTGTGCTCTTCCGATCTTCCTTCCACTTRCRGAAAGC | 504 |
| TRAV6-2 | GACGTGTGCTCTTCCGATCTTTCCTTCCACTTGCAGAAAACC | 505 |
| TRAV7-1 | GACGTGTGCTCTTCCGATCTGCTACACATCAGAGACTCCCA | 506 |
| TRAV7-2 | GACGTGTGCTCTTCCGATCTCCTGCACATCARAGACTCCCA | 507 |
| TRAV7-3 | GACGTGTGCTCTTCCGATCTCCTACACATCAGAGARCCRCA | 508 |
| TRAV7-4 | GACGTGTGCTCTTCCGATCTCCTGCACATCAGAGAGTCGC | 509 |
| TRAV8-1 | GACGTGTGCTCTTCCGATCTCCTTGACACYTCCAGCCARAG | 510 |
| TRAV9 | GACGTGTGCTCTTCCGATCTCTGAGTTCAGCAAGAGYRACTCT | 511 |

2nd PCR:

| | | |
|---|---|---|
| 2nd PCR reverse | AATGATACGGCGACCACCGAGATCTACACTCTTTCCCTACACGAC | 512 |
| 2nd PCR forward | CAAGCAGAAGACGGCATACGAGATAA XXXXXX | 513 |
| | GTGACTGGAGTTCAGACGTGTGCTCTTCCGATCT | |
| | (X indicates fixed library index) | |

TCRB

| | | |
|---|---|---|
| RT: | | |
| TRBC_12N | ACACTCTTTCCCTACACGACGCTCTTCCGATCTNNNNNNNNNNNNGGGT | 514 |
| | GGAGTCACATTTCTCAGA | |

1st PCR

| | | |
|---|---|---|
| 1st PCR reverse | ACACTCTTTCCCTACACGAC | 515 |

1st PCR forward:

| | | |
|---|---|---|
| TRBV1 | GACGTGTGCTCTTCCGATCTTCACTGATACGGAGCTGAGGC | 516 |
| TRBV10 | GACGTGTGCTCTTCCGATCTGCTTTCCCCTGACATTAGAGTCA | 517 |
| TRBV11 | GACGTGTGCTCTTCCGATCTTCCTACTCTATTCTGAAGACCCAG | 518 |
| TRBV12-1 | GACGTGTGCTCTTCCGATCTCTCTGARATGAACATGAGTGCCT | 519 |
| TRBV12-2 | GACGTGTGCTCTTCCGATCTAATCCAACAGTTCAACGACTTTT | 520 |
| TRBV13-1 | GACGTGTGCTCTTCCGATCTGACTTCTTCCTCCTGCTGGAA | 521 |
| TRBV13-2/3 | GACGTGTGCTCTTCCGATCTTTCTCYCTCATTCTGGAGTTGG | 522 |
| TRBV14 | GACGTGTGCTCTTCCGATCTCTCCACTCTCAAGATCCAGTCTG | 523 |
| TRBV15 | GACGTGTGCTCTTCCGATCTCCTTCTCCACTCTGAAGATTCAAC | 524 |
| TRBV16 | GACGTGTGCTCTTCCGATCTGTCGCACTCAACTCTGAAGATCC | 525 |
| TRBV17 | GACGTGTGCTCTTCCGATCTTCTGCTCTCTCTACATTGGCTCTG | 526 |
| TRBV18 | GACGTGTGCTCTTCCGATCTGGAACCCAACATCCTAAAGTGG | 527 |
| TRBV19 | GACGTGTGCTCTTCCGATCTTCTCTCACTGTGACATCTGCCC | 528 |
| TRBV2 | GACGTGTGCTCTTCCGATCTCCATTTAGACCTTCAGATCACAGC | 529 |
| TRBV20 | GACGTGTGCTCTTCCGATCTCATCAGTCATCCCAACTTATCCTT | 530 |
| TRBV21 | GACGTGTGCTCTTCCGATCTATGTACCATAGAGATCCAGTCCAG | 531 |
| TRBV22 | GACGTGTGCTCTTCCGATCTGCAGCTTGGAAATCAGTTCCTC | 532 |
| TRBV23 | GACGTGTGCTCTTCCGATCTCTGGGAATCAGAACGTGCGAA | 533 |
| TRBV24 | GACGTGTGCTCTTCCGATCTGCATCCTGGAAATCCTATCCTCT | 534 |
| TRBV25 | GACGTGTGCTCTTCCGATCTCTCATCCTTCATCTTGGAAATGC | 535 |
| TRBV26 | GACGTGTGCTCTTCCGATCTCAGCCTAGAAATTCAGTCCTCTG | 536 |
| TRBV27 | GACGTGTGCTCTTCCGATCTGAATCCTACCTCATGTTAAGCACA | 537 |
| TRBV28 | GACGTGTGCTCTTCCGATCTAAATCTTCCAGCATCGACCAGG | 538 |
| TRBV29 | GACGTGTGCTCTTCCGATCTAGCATTTCTCCCTGATTCTGGA | 539 |
| TRBV3 | GACGTGTGCTCTTCCGATCTCTCTGAAAATCCAACCCACAGC | 540 |
| TRBV30 | GACGTGTGCTCTTCCGATCTCGTTGACAGTGAACAATGCAAGG | 541 |
| TRBV31 | GACGTGTGCTCTTCCGATCTTTCATCCTAAGCACGGAGAAGC | 542 |
| TRBV4 | GACGTGTGCTCTTCCGATCTTCAGATAAAGCTCATTTGAATCTTCG | 543 |
| TRBV5 | GACGTGTGCTCTTCCGATCTAGACAGCTCCAAGCTACTTTTACA | 544 |
| TRBV6 | GACGTGTGCTCTTCCGATCTGGATTGTTCTCCACTCTGAAGATT | 546 |
| TRBV7 | GACGTGTGCTCTTCCGATCTCAATTTGGTGACTAGCATCCTGAA | 547 |
| TRBV8 | GACGTGTGCTCTTCCGATCTCACAGAGGACTTCACCTTCACTG | 548 |
| TRBV9 | GACGTGTGCTCTTCCGATCTCTCCTTCTCCATGTTGAAGAGCC | 549 |

2nd PCR:

| | | |
|---|---|---|
| 2nd PCR reverse | AATGATACGGCGACCACCGAGATCTACACTCTTTCCCTACACGAC | 550 |
| 2nd PCR forward | CAAGCAGAAGACGGCATACGAGATAA XXXXXX | 551 |
| | GTGACTGGAGTTCAGACGTGTGCTCTTCCGATCT (X indicates | |
| | fixed library index) | |

Mouse Ab MIDCIRS

| | | |
|---|---|---|
| RT primer | | |
| mIgM_RT_12N_ | ACACTCTTTCCCTACACGACGCTCTTCCGATCTNNNNNNNNNNNNGATG | 552 |
| partialPE1 | ACTTCAGTGTTGTTCTGG | |
| mIgG_RT_12N_partialPE1 | ACACTCTTTCCCTACACGACGCTCTTCCGATCTNNNNNNNNNNNNCAGG | 553 |
| | GATCCAGAGTTCC | |
| mIgA_RT_12N_partialPE1 | ACACTCTTTCCCTACACGACGCTCTTCCGATCTNNNNNNNNNNNNCAGG | 554 |
| | TCACATTCATCGTG | |
| mIgD_RT_12N_partialPE1 | ACACTCTTTCCCTACACGACGCTCTTCCGATCTNNNNNNNNNNNNAGTG | 555 |
| | GCTGACTTCCAA | |
| mIgE_RT_12N_partialPE1 | ACACTCTTTCCCTACACGACGCTCTTCCGATCTNNNNNNNNNNNNCACA | 556 |
| | GTGCTCATGTTCAGG | |

1st PCR forward primer-1

| | | |
|---|---|---|
| mVH1.1_partialPE2 | GACGTGTGCTCTTCCGATCTAGRTYCAGCTGCARCAGTCT | 557 |
| mVH1.2_partialPE2 | GACGTGTGCTCTTCCGATCTAGGTCCAACTGCAGCAGCC | 558 |
| mVH2_partialPE2 | GACGTGTGCTCTTCCGATCTTCTGCCTGGTGACWTTCCCA | 559 |
| mVH3_partialPE2 | GACGTGTGCTCTTCCGATCTGTGCAGCTTCAGGAGTCAG | 560 |
| mVH4_partialPE2 | GACGTGTGCTCTTCCGATCTGAGGTGAAGCTTCTCGAGTC | 561 |
| mVH5_partialPE2 | GACGTGTGCTCTTCCGATCTGAAGTGAAGCTGGTGGAGTC | 562 |
| mVH6_partialPE2 | GACGTGTGCTCTTCCGATCTATGKACTTGGGACTGARCTGT | 563 |
| mVH7_partialPE2 | GACGTGTGCTCTTCCGATCTCAGTGTGAGGTGAAGCTGGT | 564 |
| mVH8_partialPE2 | GACGTGTGCTCTTCCGATCTCCAGGTTACTCTGAAAGAGTC | 565 |
| mVH9_partialPE2 | GACGTGTGCTCTTCCGATCTTGTGGACCTTGCTATTCCTGA | 566 |
| mVH10_partialPE2 | GACGTGTGCTCTTCCGATCTTGTTGGGGCTGAAGTGGGTTT | 567 |
| mVH11_partialPE2 | GACGTGTGCTCTTCCGATCTATGGAGTGGGAACTGAGCTTA | 568 |
| mVH12_partialPE2 | GACGTGTGCTCTTCCGATCTAGCTTCAGGAGTCAGGACC | 569 |
| mVH13_partialPE2 | GACGTGTGCTCTTCCGATCT CAGGTGCAGCTTGTAGAGAC | 570 |
| mVH14_partialPE2 | GACGTGTGCTCTTCCGATCT ATGCAGCTGGGTCATCTTCTT | 571 |
| mVH15_partialPE2 | GACGTGTGCTCTTCCGATCTGACTGGATTTGGATCACKCTC | 572 |
| mVH16_partialPE2 | GACGTGTGCTCTTCCGATCTTGGAGTTTGGACTTAGTTGGG | 573 |

1st PCR reverse primer

| | | |
|---|---|---|
| ILLUPE1adaptor_short | ACACTCTTTCCCTACACGAC | 574 |

2nd PCR:

| | | |
|---|---|---|
| 2nd PCR reverse | AATGATACGGCGACCACCGAGATCTACACTCTTTCCCTACACGAC | 575 |
| 2nd PCR forward | CAAGCAGAAGACGGCATACGAGATAA XXXXXX | 576 |
| | GTGACTGGAGTTCAGACGTGTGCTCTTCCGATCT (X indicates | |
| | fixed library index) | |

Human Ab MIDCIRS RT primer

| | | |
|---|---|---|
| IgHG1/2/3/4 | ACACTCTTTCCCTACACGACGCTCTTCCGATCTN1ThNNNNNNNNNAGT | 577 |
| | CCTTGACCAGGCAGC | |
| IgHA1/2 | ACACTCTTTCCCTACACGACGCTCTTCCGATCTN NNNNNN | 578 |
| | GAYGACCACGTTCCCATCT | |
| IgM | ACACTCTTTCCCTACACGACGCTCTTCCGATCTN1ThNNNNNNNNN | 579 |
| | GGGAATTCTCACAGGAGACG | |
| IgE | ACACTCTTTCCCTACACGACGCTCTTCCGATCTN1ThNNNNNNNNN | 580 |
| | GAAGACGGATGGGCTCTGT | |
| IgD | ACACTCTTTCCCTACACGACGCTCTTCCGATCTN1ThNNNNNNNNN | 581 |
| | GGGTGTCTGCACCCTGATA | |

1st PCR forward pnmers

| | | |
|---|---|---|
| ILLUPE2LR1 | GACGTGTGCTCTTCCGATCTCGCAGACCCTCTCACTCAC | 582 |
| ILLUPE2LR2 | GACGTGTGCTCTTCCGATCTTGGAGCTGAGGTGAAGAAGC | 583 |
| ILLUPE2LR3 | GACGTGTGCTCTTCCGATCTTGCAATCTGGGTCTGAGTTG | 584 |
| ILLUPE2LR4 | GACGTGTGCTCTTCCGATCTGGCTCAGGACTGGTGAAGC | 585 |
| ILLUPE2LR5 | GACGTGTGCTCTTCCGATCTTGGAGCAGAGGTGAAAAAGC | 586 |
| ILLUPE2LR6 | GACGTGTGCTCTTCCGATCTGGTGCAGCTGTTGGAGTCT | 587 |
| ILLUPE2LR7 | GACGTGTGCTCTTCCGATCTACTGTTGAAGCCTTCGGAGA | 588 |
| ILLUPE2LR8 | GACGTGTGCTCTTCCGATCTAAACCCACACAGACCCTCAC | 589 |
| ILLUPE2LR9 | GACGTGTGCTCTTCCGATCTAGTCTGGGGCTGAGGTGAAG | 590 |
| ILLUPE2LR10 | GACGTGTGCTCTTCCGATCTGGCCCAGGACTGGTGAAG | 591 |
| ILLUPE2LR11 | GACGTGTGCTCTTCCGATCTGGTGCAGCTGGTGGAGTC | 592 |

1^{st} PCR reverse primer

| | | |
|---|---|---|
| ILLUPE1adaptor_short | ACACTCTTTCCCTACACGAC | 593 |

2nd PCR:

| | | |
|---|---|---|
| 2nd PCR reverse | AATGATACGGCGACCACCGAGATCTACACTCTTTCCCTACACGACCAAG | 594 |
| 2nd PCR forward | CAGAAGACGGCATACGAGATAA XXXXXX | 595 |
| | GTGACTGGAGTTCAGACGTGTGCTCTTCCGATCT (X indicates | |
| | fixed library index) | |

C. Amplification of Variable Immune Sequences

Polymerase chain reaction (PCR) can be used to amplify the relevant variable immune regions after reverse transcription has attached the MID to each cDNA. In some embodiments, the region to be amplified includes the full clonal sequence or a subset of the clonal sequence, including the V-D junction, D-J junction of an immunoglobulin or T-cell receptor gene, the full variable region of an immunoglobulin or T-cell receptor gene, the antigen recognition region, or a CDR, e.g., complementarity determining region 3 (CDR3).

In some embodiments, the variable immune sequence is amplified using a primary and a secondary amplification step. Each of the different amplification steps can comprise different primers. The different primers can introduce sequence not originally present in the immune gene sequence. For example, the amplification procedure can add one or more tags to the 5′ and/or 3′ end of amplified immunoglobulin sequence. The tag can be a sequence that facilitates subsequent sequencing of the amplified DNA. The tag can be a sequence that facilitates binding the amplified sequence to a solid support. The tag can be a barcode or label to facilitate identification of the amplified immunoglobulin sequence.

Other methods for amplification may not employ any primers in the V region. Instead, a specific primer can be used from the C segment and a generic primer can be put in the other side (5′). The generic primer can be appended in the cDNA synthesis through different methods including the well described methods of strand switching. Similarly, the generic primer can be appended after cDNA synthesis through different methods including ligation.

Other means of amplifying nucleic acid that can be used in the methods of the invention include, for example, reverse transcription-PCR, real-time PCR, quantitative real-time PCR, digital PCR (dPCR), digital emulsion PCR (dePCR), clonal PCR, amplified fragment length polymorphism PCR (AFLP PCR), allele specific PCR, assembly PCR, asymmetric PCR (in which a great excess of primers for a chosen strand is used), colony PCR, helicase-dependent amplification (HDA), Hot Start PCR, inverse PCR (IPCR), in situ PCR, long PCR (extension of DNA greater than about 5 kilobases), multiplex PCR, nested PCR (uses more than one pair of primers), single-cell PCR, touchdown PCR, loop-mediated isothermal PCR (LAMP), and nucleic acid sequence based amplification (NASBA). Other amplification schemes include: Ligase Chain Reaction, Branch DNA Amplification, Rolling Circle Amplification, Circle to Circle Amplification, SPIA amplification, Target Amplification by Capture and Ligation (TACL) amplification, and RACE amplification.

In particular aspects, RACE amplification is used in the current methods. The SMART (Switching Mechanism at the 5′ end of RNA template) system (CLONTECH) is based on the non-templated addition of polyC to nascent cDNA by reverse transcriptase. The double-stranded cDNA sequences that are produced contain a common, specific anchor sequence at their 5′ ends. Using the SMART system, a 5′-RACE PCR reaction is performed in which the specific (SMART) anchor sequence also serves as the 5′ primer-binding site and is coupled with a 3′ degenerate antisense primer that complements a short region of predicted amino acid sequence identity.

The SMART technology can be combined with semi-nested PCR to fully capture and amplify variable immune regions and prepare libraries for sequencing, such as on Illumina® platforms. Briefly, first-strand cDNA synthesis is dT-primed (TCR dT Primer) and performed by the MMLV-derived SMARTScribe Reverse Transcriptase (RT), which adds non-templated nucleotides upon reaching the 5′ end of each mRNA template. The SMART-Seq Oligonucleotide—enhanced with Locked Nucleic Acid (LNA) technology for increased sensitivity and specificity—then anneals to the non-templated nucleotides, and serves as a template for the incorporation of an additional sequence of nucleotides to the first-strand cDNA by the RT (i.e., the template-switching step). This additional sequence—referred to as the “SMART sequence”—serves as a primer-annealing site for subsequent rounds of PCR, ensuring that only sequences from full-length cDNAs undergo amplification. Following reverse transcription and extension, two rounds of PCR are performed in succession to amplify cDNA sequences corresponding to variable regions. The first PCR uses the first-strand cDNA as a template and includes a forward primer with complementarity to the SMART sequence (SMART Primer 1), and a reverse primer that is complementary to the constant (i.e. non-variable) region (e.g., of either TCR-α or TCR-β); both reverse primers may be included in a single reaction if analysis of both TCR subunit chains is desired. By priming from the SMART sequence and constant region, the first PCR specifically amplifies the entire variable region and a considerable portion of the constant region. The second PCR takes the product from the first PCR as a template, and uses semi-nested primers to amplify the entire variable region and a portion of the constant region. Included in the forward and reverse primers are adapter and index sequences which are compatible with the Illumina sequencing platform (read 2+i7+P7 and read 1+i5+P5, respectively). Following post-PCR purification, size selection, and quality analysis, the library is ready for Illumina sequencing.

D. Sequencing

Any technique for sequencing nucleic acids known to those skilled in the art can be used in the methods of the present disclosure. DNA sequencing techniques include classic dideoxy sequencing reactions (Sanger method) using labeled terminators or primers and gel separation in slab or capillary, sequencing-by-synthesis using reversibly terminated labeled nucleotides, pyrosequencing, 454 sequencing, allele specific hybridization to a library of labeled oligonucleotide probes, sequencing-by-synthesis using allele specific hybridization to a library of labeled clones that is followed by ligation, real time monitoring of the incorporation of labeled nucleotides during a polymerization step, and SOLiD sequencing. The input RNA may be 10%, 15%, 30%, or higher.

In certain embodiments, the sequencing technique used in the methods of the provided invention generates at least 100 reads per run, at least 200 reads per run, at least 300 reads per run, at least 400 reads per run, at least 500 reads per run, at least 600 reads per run, at least 700 reads per run, at least 800 reads per run, at least 900 reads per run, at least 1000 reads per run, at least 5,000 reads per run, at least 10,000 reads per run, at least 50,000 reads per run, at least 100,000 reads per run, at least 500,000 reads per run, at least 1,000,000 reads per run, at least 2,000,000 reads per run, at least 3,000,000 reads per run, at least 4,000,000 reads per run at least 5000,000 reads per runs at least 6,000,000 reads per run at least 7,000,000 reads per run at least 8,000,000 reads per runs at least 9,000,000 reads per run, or at least 10,000,000 reads per run.

In some embodiments the number of sequencing reads per B cell sampled should be at least 2 times the number of B cells sampled, at least 3 times the number of B cells sampled, at least 5 times the number of B cells sampled, at least 6 times the number of B cells sampled, at least 7 times the number of B cells sampled, at least 8 times the number of B cells sampled, at least 9 times the number of B cells sampled, or at least at least 10 times the number of B cells The read depth allows for accurate coverage of B cells sampled, facilitates error correction, and ensures that the sequencing of the library has been saturated.

In some embodiments the number of sequencing reads per T-cell sampled should be at least 2 times the number of T-cells sampled, at least 3 times the number of T-cells sampled, at least 5 times the number of T-cells sampled, at least 6 times the number of T-cells sampled, at least 7 times the number of T-cells sampled, at least 8 times the number of T-cells sampled, at least 9 times the number of T-cells sampled, or at least at least 10 times the number of T-cells The read depth allows for accurate coverage of T-cells sampled, facilitates error correction, and ensures that the sequencing of the library has been saturated.

In certain embodiments, the sequencing technique used in the methods of the provided invention can generate about 30 bp, about 40 bp, about 50 bp, about 60 bp, about 70 bp, about 80 bp, about 90 bp, about 100 bp, about 110, about 120 by per read, about 150 bp, about 200 bp, about 250 bp, about 300 bp, about 350 bp, about 400 bp, about 450 bp, about 500 bp, about 550 bp, about 600 bp, about 700 bp, about 800 bp, about 900 bp, or about 1,000 by per read. For example, the sequencing technique used in the methods of the provided invention can generate at least 30, 40, 50, 60, 70, 80, 90, 100, 110, 120, 150, 200, 250, 300, 350, 400, 450, 500, 550, 600, 650, 700, 750, 800, 850, 900, 950, or 1,000 by per read.

1. HiSeg™ and MiSeg™ Sequencing

In particular aspects, the sequencing technologies used in the methods of the present disclosure include the HiSEQ™ system (e.g., HiSEQ2000™ and HiSEQIOOO™) and the MiSEQ™ system from Illumina, Inc. The HiSEQ™ system is based on massively parallel sequencing of millions of fragments using attachment of randomly fragmented genomic DNA to a planar, optically transparent surface and solid phase amplification to create a high density sequencing flow cell with millions of clusters, each containing about 1,000 copies of template per sq. cm. These templates are sequenced using four-color DNA sequencing-by-synthesis technology. The MiSEQ™ system uses TruSeq, Illumina's reversible terminator-based sequencing-by-synthesis.

2. True Single Molecule Sequencing

A sequencing technique that can be used in the methods of the resent disclosure includes, for example, Helicos True Single Molecule Sequencing (tSMS) (Harris T. D. et al. (2008) Science 320: 106-109). In the tSMS technique, a DNA sample is cleaved into strands of approximately 100 to 200 nucleotides, and a polyA sequence is added to the 3′ end of each DNA strand. Each strand is labeled by the addition of a fluorescently labeled adenosine nucleotide. The DNA strands are then hybridized to a flow cell, which contains millions of oligo-T capture sites that are immobilized to the flow cell surface. The templates can be at a density of about 100 million templates/cm². The flow cell is then loaded into an instrument, e.g., HeliScope™. sequencer, and a laser illuminates the surface of the flow cell, revealing the position of each template. A CCD camera can map the position of the templates on the flow cell surface. The template fluorescent label is then cleaved and washed away. The sequencing reaction begins by introducing a DNA polymerase and a fluorescently labeled nucleotide. The oligo-T nucleic acid serves as a primer. The polymerase incorporates the labeled nucleotides to the primer in a template directed manner. The polymerase and unincorporated nucleotides are removed. The templates that have directed incorporation of the fluorescently labeled nucleotide are detected by imaging the flow cell surface. After imaging, a cleavage step removes the fluorescent label, and the process is repeated with other fluorescently labeled nucleotides until the desired read length is achieved. Sequence information is collected with each nucleotide addition step.

3. 454 Sequencing

Another example of a DNA sequencing technique that can be used in the methods of the present disclosure is 454 sequencing (Roche) (Margulies, M et al. 2005, Nature, 437, 376-380). 454 sequencing involves two steps. In the first step, DNA is sheared into fragments of approximately 300-800 base pairs, and the fragments are blunt ended. Oligonucleotide adaptors are then ligated to the ends of the fragments. The adaptors serve as primers for amplification and sequencing of the fragments. The fragments can be attached to DNA capture beads, e.g., streptavidin-coated beads using, e.g., Adaptor B, which contains 5′-biotin tag. The fragments attached to the beads are PCR amplified within droplets of an oil-water emulsion. The result is multiple copies of clonally amplified DNA fragments on each bead. In the second step, the beads are captured in wells (pico-liter sized). Pyrosequencing is performed on each DNA fragment in parallel. Addition of one or more nucleotides generates a light signal that is recorded by a CCD camera in a sequencing instrument. The signal strength is proportional to the number of nucleotides incorporated.

Pyrosequencing makes use of pyrophosphate (PPi) which is released upon nucleotide addition. PPi is converted to ATP by ATP sulfurylase in the presence of adenosine 5′ phosphosulfate. Luciferase uses ATP to convert luciferin to oxyluciferin, and this reaction generates light that is detected and analyzed.

4. Genome Sequencer FLX™

Another example of a DNA sequencing technique that can be used in the present methods is the Genome Sequencer FLX systems (Roche/454). The Genome Sequences FLX systems (e.g., GS FLX/FLX+, GS Junior) offer more than 1 million high-quality reads per run and read lengths of 400 bases. These systems are ideally suited for de novo sequencing of whole genomes and transcriptomes of any size, metagenomic characterization of complex samples, or resequencing studies.

5. SOLiD™ Sequencing

Another example of a DNA sequencing technique that can be used in the methods of the present disclosure is SOLiD technology (Life Technologies, Inc.). In SOLiD sequencing, genomic DNA is sheared into fragments, and adaptors are attached to the 5′ and 3′ ends of the fragments to generate a fragment library. Alternatively, internal adaptors can be introduced by ligating adaptors to the 5′ and 3′ ends of the fragments, circularizing the fragments, digesting the circularized fragment to generate an internal adaptor, and attaching adaptors to the 5′ and 3′ ends of the resulting fragments to generate a mate-paired library. Next, clonal bead populations are prepared in microreactors containing beads, primers, template, and PCR components. Following PCR, the templates are denatured and beads are enriched to separate the beads with extended templates. Templates on the selected beads are subjected to a 3′ modification that permits bonding to a glass slide.

The sequence can be determined by sequential hybridization and ligation of partially random oligonucleotides with a central determined base (or pair of bases) that is identified by a specific fluorophore. After a color is recorded, the ligated oligonucleotide is cleaved and removed and the process is then repeated.

6. Ion Torrent™ Sequencing

Another example of a DNA sequencing technique that can be used in the methods of the present disclosure is the IonTorrent system (Life Technologies, Inc.). Ion Torrent uses a high-density array of micro-machined wells to perform this biochemical process in a massively parallel way. Each well holds a different DNA template. Beneath the wells is an ion-sensitive layer and beneath that a proprietary Ion sensor. If a nucleotide, for example a C, is added to a DNA template and is then incorporated into a strand of DNA, a hydrogen ion will be released. The charge from that ion will change the pH of the solution, which can be detected by the proprietary ion sensor. The sequencer will call the base, going directly from chemical information to digital information. The Ion Personal Genome Machine (PGM™) sequencer then sequentially floods the chip with one nucleotide after another. If the next nucleotide that floods the chip is not a match, no voltage change will be recorded and no base will be called. If there are two identical bases on the DNA strand, the voltage will be double, and the chip will record two identical bases called. Because this is direct detection—no scanning, no cameras, no light—each nucleotide incorporation is recorded in seconds.

7. SOLEXA™ Sequencing

Another example of a sequencing technology that can be used in the methods of the present disclosure is SOLEXA sequencing (Illumina). SOLEXA sequencing is based on the amplification of DNA on a solid surface using fold-back PCR and anchored primers. Genomic DNA is fragmented, and adapters are added to the 5′ and 3′ ends of the fragments. DNA fragments that are attached to the surface of flow cell channels are extended and bridge amplified. The fragments become double stranded, and the double stranded molecules are denatured. Multiple cycles of the solid-phase amplification followed by denaturation can create several million clusters of approximately 1,000 copies of single-stranded DNA molecules of the same template in each channel of the flow cell. Primers, DNA polymerase and four fluorophore-labeled, reversibly terminating nucleotides are used to perform sequential sequencing. After nucleotide incorporation, a laser is used to excite the fluorophores, and an image is captured and the identity of the first base is recorded. The 3′ terminators and fluorophores from each incorporated base are removed and the incorporation, detection and identification steps are repeated.

8. SMRT™ Sequencing

Another example of a sequencing technology that can be used in the methods of the present disclosure includes the single molecule, real-time (SMRT™) technology of Pacific Biosciences. In SMRT™, each of the four DNA bases is attached to one of four different fluorescent dyes. These dyes are phospholinked. A single DNA polymerase is immobilized with a single molecule of template single stranded DNA at the bottom of a zero-mode waveguide (ZMW). A ZMW is a confinement structure which enables observation of incorporation of a single nucleotide by DNA polymerase against the background of fluorescent nucleotides that rapidly diffuse in and out of the ZMW (in microseconds). It takes several milliseconds to incorporate a nucleotide into a growing strand. During this time, the fluorescent label is excited and produces a fluorescent signal, and the fluorescent tag is cleaved off. Detection of the corresponding fluorescence of the dye indicates which base was incorporated. The process is repeated.

9. Nanopore Sequencing

Another example of a sequencing technique that can be used is nanopore sequencing (Soni G V and Meller A. (2007) Clin Chem 53: 1996-2001). A nanopore is a small hole, of the order of 1 nanometer in diameter. Immersion of a nanopore in a conducting fluid and application of a potential across it results in a slight electrical current due to conduction of ions through the nanopore. The amount of current which flows is sensitive to the size of the nanopore. As a DNA molecule passes through a nanopore, each nucleotide on the DNA molecule obstructs the nanopore to a different degree. Thus, the change in the current passing through the nanopore as the DNA molecule passes through the nanopore represents a reading of the DNA sequence.

E. Clustering-Based Analysis

Sequencing allows for the presence of multiple variable immune sequences to be detected and quantified in a heterogeneous biological sample. The high throughput sequencing provides a very large dataset, which is then analyzed in order to establish the immune repertoire.

High-throughput analysis can be achieved using one or more bioinformatics tools, such as ALLPATHS (a whole genome shotgun assembler that can generate high quality assemblies from short reads), Arachne (a tool for assembling genome sequences from whole genome shotgun reads, mostly in forward and reverse pairs obtained by sequencing cloned ends, BACCardl (a graphical tool for the validation of genomic assemblies, assisting genome finishing and intergenome comparison), CCRaVAT & QuTie (enables analysis of rare variants in large-scale case control and quantitative trait association studies), CNV-seq (a method to detect copy number variation using high throughput sequencing), Elvira (a set of tools/procedures for high throughput assembly of small genomes (e.g., viruses)), Glimmer (a system for finding genes in microbial DNA, especially the genomes of bacteria, archaea and viruses), gnumap (a program designed to accurately map sequence data obtained from next-generation sequencing machines), Goseq (an R library for performing Gene Ontology and other category based tests on RNA-seq data which corrects for selection bias), ICAtools (a set of programs useful for medium to large scale sequencing projects), LOCAS, a program for assembling short reads of second generation sequencing technology, Maq (builds assembly by mapping short reads to reference sequences, MEME (motif-based sequence analysis tools, NGSView (allows for visualization and manipulation of millions of sequences simultaneously on a desktop computer, through a graphical interface, OSLay (Optimal Syntenic Layout of Unfinished Assemblies), Perm (efficient mapping for short sequencing reads with periodic full sensitive spaced seeds, Projector (automatic contig mapping for gap closure purposes), Qpalma (an alignment tool targeted to align spliced reads produced by sequencing platforms such as Illumina, Solexa, or 454), RazerS (fast read mapping with sensitivity control), SHARCGS (SHort read Assembler based on Robust Contig extension for Genome Sequencing; a DNA assembly program designed for de novo assembly of 25-40mer input fragments and deep sequence coverage), Tablet (next generation sequence assembly visualization), and Velvet (sequence assembler for very short reads).

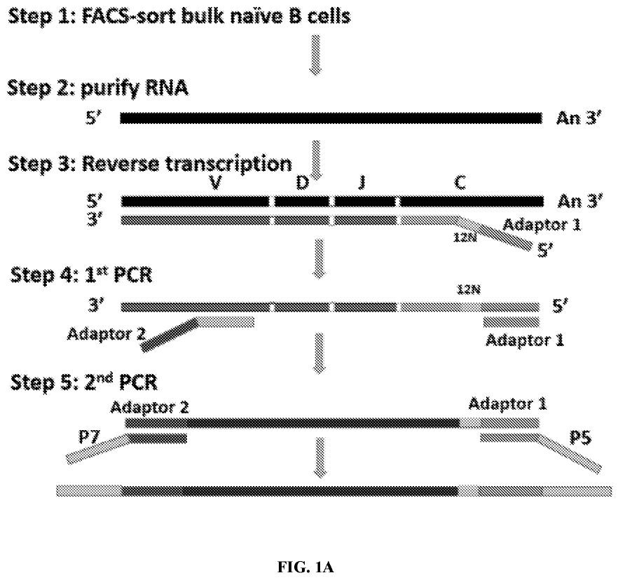

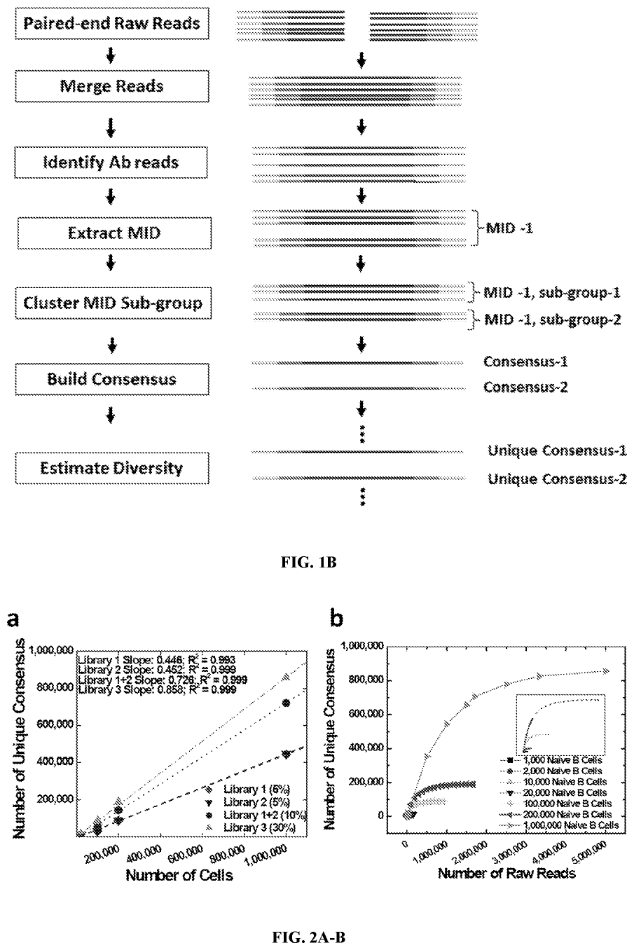

An exemplary method of data analysis steps are summarized in the flow chart of FIG. 1B. The paired-end sequencing reads are first merged and immunological receptor reads are identified. Then reads are grouped according to the MID. Next, a clustering method is used to further separate different types of RNA molecules that are tagged with the same MID into sub-groups. Bias and error in amplification and/or sequencing may be reduced by identification of consensus sequences. In certain aspects, RNA molecules sharing a unique identification nucleotide sequence (UID) may be identified (e.g. classified) as belonging to the same consensus sequence. Consensus sequences may be used to average out error from the amplification and/or sequencing steps. Clustering threshold is an important parameter to consider. This threshold needs to be optimized to group reads that are different due to sequencing and PCR errors into the same MID sub-group but exclude reads that are derived from different antibody sequences. RNA controls with known sequences are used to set the threshold (Levenshtein distance) to be 15% of the read length. Next, a consensus sequence is generated from each sub-group within a MID group by considering the number of reads in each sub-group and their quality scores. Each MID sub-group is equivalent to an RNA molecule.

Raw reads may be split into MID groups according to their barcodes. For each MID group, quality threshold clustering was used to cluster similar reads. This process groups reads derived from a common template RNA molecule together while separating reads derived from distinct RNA molecules. A Levenshtein distance this is calibrated using RNA controls with known sequences and may be set as 15% of the read length as the threshold. For each sub-group, a consensus sequence is built based on the average nucleotide at each position, weighted by the quality score. In the case that there are only two reads in an MID sub-group, they are only considered useful reads if both were identical. Each MID sub-group is equivalent to an RNA molecule. Next, all of the identical consensus are merged to form unique consensus sequences, or unique RNA molecules, which are used to estimate the diversity and assess the sequencing depth in rarefaction analysis.

To calculate the total diversity, multiple consensus with the exact same sequences (RNA molecules that originated from the same cell) are combined and the number of unique consensus sequences are counted. The approach described here that further clusters reads under the same MID is useful when the total number of receptor transcript information for a given sample is unknown or when shorter MIDs are preferred to maintain reverse transcription efficiency. The estimation of diversity is affected by the initial RNA sampling depth (percentage of initial RNA used to construct the sequencing library). A statistical model was used to estimate the diversity coverage for the naïve B cells that were sorted based on RNA sampling depth. For N RNA molecules, there are K different RNA clones. The copy number of each RNA clone is m. When n RNA molecules are sampled from this population, the possible detected diversity T can be described by the following formula:

$$E ( T ) = K - \frac{{\sum }_{i = 1}^{K} ( \begin{matrix}N - {m}_{i} \\ n\end{matrix} )}{( \begin{matrix}N \\ n\end{matrix} )} \qquad ( 1 )$$

It can be assumed that all RNA clones have the same number of RNA copies:

mm1=mm2= . . . =mmKK=mm

This is reasonable because naïve B cells bears minimum clonal expansion. Then the percentage of the RNA diversity coverage can be estimated as:

$$P ( T ) = \frac{E ( T )}{K} = 1 - \frac{( \begin{matrix}N - m \\ n\end{matrix} )}{( \begin{matrix}N \\ n\end{matrix} )} \qquad ( 2 )$$

After clustering MID sub-groups, the error rate can be calculated for raw reads. For each MID subgroup, there is a consensus sequence. The difference between the consensus sequence and reads can be considered as the error generated in either PCR or sequencing.

So the error-rate can be calculated using the following formula:

$$ErrorRate ( Raw ) = \frac{{\sum }_{i = 1}^{N} Diff ( i , I )}{N \times L}$$

where Diff(i,I) is the Hamming distance between the reads i and the consensus sequence in MID Sub-group I; N is the number of reads in MID Sub-group I; L is the length of reads.

In order to estimate the improved error rate for using MID sub-groups, the raw reads from one library were divided into two datasets equally. The same MID sub-group generating process was done on both datasets. By comparing the differences of consensus sequences with identical MID between these two datasets, the improved error rate for using MID sub-groups was calculated as:

$$ErrorRate ( MID ) = \frac{{\sum }_{I , J} Diff ( I , J ) \times Ni}{{\sum }_{I} Ni \times L}$$

where Diff(I,J) is the Hamming distance between the consensus I and consensus J, which have the identical MID. Ni is the number of reads in MID sub-group I, L is the length of reads.

The results of the analysis may be referred to herein as an immune repertoire analysis result, which may be represented as a dataset that includes sequence information, representation of V, D, J, C, VJ, VDJ, VJC, VDJC, antibody heavy chain, antibody light chain, CDR3, or T-cell receptor usage, representation for abundance of V, D, J, C, VJ, VDJ, VJC, VDJC, antibody heavy chain, antibody light chain, CDR3, or T-cell receptor and unique sequences; representation of mutation frequency, correlative measures of VJ V, D, J, C, VJ, VDJ, VJC, VDJC, antibody heavy chain, antibody light chain, CDR3, or T-cell receptor usage. Such results may then be output or stored, e.g. in a database of repertoire analyses, and may be used in comparisons with test results, and reference results.

After obtaining an immune repertoire analysis result from the sample being assayed, the repertoire can be compared with a reference or control repertoire to make a diagnosis, prognosis, analysis of drug effectiveness, or other desired analysis. A reference or control repertoire may be obtained by the methods of the invention, and will be selected to be relevant for the sample of interest. A test repertoire result can be compared to a single reference/control repertoire result to obtain information regarding the immune capability and/or history of the individual from which the sample was obtained.

Alternately, the obtained repertoire result can be compared to two or more different reference/control repertoire results to obtain more in-depth information regarding the characteristics of the test sample. For example, the obtained repertoire result may be compared to a positive and negative reference repertoire result to obtain confirmed information regarding whether the phenotype of interest. In another example, two “test” repertoires can also be compared with each other. In some cases, a test repertoire is compared to a reference sample and the result is then compared with a result derived from a comparison between a second test repertoire and the same reference sample.

Determination or analysis of the difference values, i.e., the difference between two repertoires can be performed using any conventional methodology, where a variety of methodologies are known to those of skill in the array art, e.g., by comparing digital images of the repertoire output, or by comparing databases of usage data.

A statistical analysis step can then be performed to obtain the weighted contribution of the sequence prevalence, e.g. V, D, J, C, VJ, VDJ, VJC, VDJC, antibody heavy chain, antibody light chain, CDR3, T-cell receptor usage, or mutation analysis. For example, nearest shrunken centroids analysis may be applied as described in Tibshirani et al., 2002 to compute the centroid for each class, then compute the average squared distance between a given repertoire and each centroid, normalized by the within-class standard deviation.

A statistical analysis may comprise use of a statistical metric (e.g., an entropy metric, an ecology metric, a variation of abundance metric, a species richness metric, or a species heterogeneity metric) in order to characterize diversity of a set of immunological receptors. Methods used to characterize ecological species diversity can also be used in the present disclosure. See, e.g., Peet, 1974. A statistical metric may also be used to characterize variation of abundance or heterogeneity. An example of an approach to characterize heterogeneity is based on information theory, specifically the Shannon-Weaver entropy, which summarizes the frequency distribution in a single number.

The classification can be probabilistically defined, where the cut-off may be empirically derived. In one embodiment of the invention, a probability of about 0.4 can be used to distinguish between individuals exposed and not-exposed to an antigen of interest, more usually a probability of about 0.5, and can utilize a probability of about 0.6 or higher. A “high” probability can be at least about 0.75, at least about 0.7, at least about 0.6, or at least about 0.5. A “low” probability may be not more than about 0.25, not more than 0.3, or not more than 0.4. In many embodiments, the above-obtained information is employed to predict whether a host, subject or patient should be treated with a therapy of interest and to optimize the dose therein.

### III. Methods of Use

Embodiments of the present disclosure provide methods for monitoring the immune repertoire including antibody repertoire as well as T cells and B cells. B cells divide rapidly after contact with an antigen giving rise to a population of B cells that all have very similar antibody sequences, differing only due to somatic hypermutation. By clustering these cells, clonal lineages or families of B cells are identified.

The present disclosure further provides methods for the prevention, treatment, detection, diagnosis, prognosis, or research into any condition or symptom of any condition, including cancer, inflammatory diseases, autoimmune diseases, allergies and infections of an organism. The organism is preferably a human subject but can also be derived from non-human subjects, e.g., non-human mammals. Examples of non-human mammals include, but are not limited to, non-human primates (e.g., apes, monkeys, gorillas), rodents (e.g., mice, rats), cows, pigs, sheep, horses, dogs, cats, or rabbits.

Examples of cancers include prostrate, pancreas, colon, brain, lung, breast, bone, and skin cancers. Examples of inflammatory conditions include irritable bowel syndrome, ulcerative colitis, appendicitis, tonsilitis, dermatitis. Examples of atopic conditions include allergies, and asthma. Examples of autoimmune diseases include IDDM, RA, MS, SLE, Crohn's disease, and Graves' disease. Autoimmune diseases also include Celiac disease, and dermatitis herpetiformis. For example, determination of an immune response to cancer antigens, autoantigens, pathogenic antigens, or vaccine antigens is of interest.

In some aspects, nucleic acids (e.g., genomic DNA, mRNA, etc.) are obtained from an organism after the organism has been challenged with an antigen (e.g., vaccinated). In other cases, the nucleic acids are obtained from an organism before the organism has been challenged with an antigen (e.g., vaccinated). Comparing the diversity of the immunological receptors present before and after challenge, may assist the analysis of the organism's response to the challenge.

Methods are also provided for optimizing therapy, by analyzing the immune repertoire in a sample, and based on that information, selecting the appropriate therapy, dose, and treatment modality that is optimal for stimulating or suppressing a targeted immune response, while minimizing undesirable toxicity. The treatment is optimized by selection for a treatment that minimizes undesirable toxicity, while providing for effective activity. For example, a patient may be assessed for the immune repertoire relevant to an autoimmune disease, and a systemic or targeted immunosuppressive regimen may be selected based on that information.

A signature repertoire for a condition can refer to an immune repertoire result that indicates the presence of a condition of interest. For example a history of cancer (or a specific type of allergy) may be reflected in the presence of immune receptor sequences that bind to one or more cancer antigens. The presence of autoimmune disease may be reflected in the presence of immune receptor sequences that bind to autoantigens. A signature can be obtained from all or a part of a dataset, usually a signature will comprise repertoire information from at least about 100 different immune receptor sequences, at least about 10² different immune receptor sequences, at least about 10³ different immune receptor sequences, at least about 10⁴ different immune receptor sequences, at least about 10⁵ different immune receptor sequences, or more. Where a subset of the dataset is used, the subset may comprise, for example, alpha TCR, beta TCR, MHC, IgH, IgL, or combinations thereof.

The classification methods described herein are of interest as a means of detecting the earliest changes along a disease pathway (e.g., a carcinogenesis pathway, or inflammatory pathway), and/or to monitor the efficacy of various therapies and preventive interventions.

The methods disclosed herein can also be utilized to analyze the effects of agents on cells of the immune system. For example, analysis of changes in immune repertoire following exposure to one or more test compounds can performed to analyze the effect(s) of the test compounds on an individual. Such analyses can be useful for multiple purposes, for example in the development of immunosuppressive or immune enhancing therapies.

Agents to be analyzed for potential therapeutic value can be any compound, small molecule, protein, lipid, carbohydrate, nucleic acid or other agent appropriate for therapeutic use. Preferably tests are performed in vivo, e.g. using an animal model, to determine effects on the immune repertoire.

Agents of interest for screening include known and unknown compounds that encompass numerous chemical classes, primarily organic molecules, which may include organometallic molecules, and genetic sequences. An important aspect of the invention is to evaluate candidate drugs, including toxicity testing.

In addition to complex biological agents candidate agents include organic molecules comprising functional groups necessary for structural interactions, particularly hydrogen bonding, and typically include at least an amine, carbonyl, hydroxyl or carboxyl group, frequently at least two of the functional chemical groups. The candidate agents can comprise cyclical carbon or heterocyclic structures and/or aromatic or polyaromatic structures substituted with one or more of the above functional groups. Candidate agents can also be found among biomolecules, including peptides, polynucleotides, saccharides, fatty acids, steroids, purines, pyrimidines, derivatives, structural analogs or combinations thereof. In some instances, test compounds may have known functions (e.g., relief of oxidative stress), but may act through an unknown mechanism or act on an unknown target. Included are pharmacologically active drugs, and genetically active molecules. Compounds of interest include chemotherapeutic agents, and hormones or hormone antagonists. Exemplary of pharmaceutical agents suitable for this invention are those described in, “The Pharmacological Basis of Therapeutics,” Goodman and Oilman, McGraw-Hill, New York, N.Y., (1996), Ninth edition, under the sections: Water, Salts and Ions; Drugs Affecting Renal Function and Electrolyte Metabolism; Drugs Affecting Gastrointestinal Function; Chemotherapy of Microbial Diseases; Chemotherapy of Neoplastic Diseases; Drugs Acting on Blood-Forming organs; Hormones and Hormone Antagonists; Vitamins, Dermatology; and Toxicology, all incorporated herein by reference.

### IV. Kits

Also provided herein are reagents and kits thereof for practicing one or more of the above-described methods. Reagents of interest include reagents specifically designed for use in production of the above described immune repertoire analysis. For example, reagents can include primer sets for cDNA synthesis, for PCR amplification and/or for high throughput sequencing of a class or subtype of immunological receptors. Gene specific primers and methods for using the same are described in U.S. Pat. No. 5,994,076, the disclosure of which is herein incorporated by reference. The gene specific primer collections can include only primers for immunological receptors, or they may include primers for additional genes, e.g., housekeeping genes, controls, etc.

The kits of the present disclosure can include the above described gene specific primer collections. The kits can further include a software package for statistical analysis, and may include a reference database for calculating the probability of a match between two repertoires. The kit may include reagents employed in the various methods, such as primers for generating target nucleic acids, dNTPs and/or rNTPs, which may be either premixed or separate, one or more uniquely labeled dNTPs and/or rNTPs, such as biotinylated or Cy3 or Cy5 tagged dNTPs, gold or silver particles with different scattering spectra, or other post synthesis labeling reagent, such as chemically active derivatives of fluorescent dyes, enzymes, such as reverse transcriptases, DNA polymerases, RNA polymerases, and the like, various buffer mediums, e.g. hybridization and washing buffers, prefabricated probe arrays, labeled probe purification reagents and components, like spin columns, etc., signal generation and detection reagents, e.g. streptavidin-alkaline phosphatase conjugate, chemifluorescent or chemiluminescent substrate, and the like.

In addition to the above components, the kits may further include instructions for practicing the present methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, or in a package insert. Yet another means would be a computer readable medium, e.g., diskette, CD, etc., on which the information has been recorded. Yet another means that may be present is a website address which may be used via the internet to access the information at a removed, site. Any convenient means may be present in the kits.

The above-described analytical methods may be embodied as a program of instructions executable by computer to perform the different aspects of the invention. Any of the techniques described above may be performed by means of software components loaded into a computer or other information appliance or digital device. When so enabled, the computer, appliance or device may then perform the above-described techniques to assist the analysis of sets of values associated with a plurality of genes in the manner described above, or for comparing such associated values. The software component may be loaded from a fixed media or accessed through a communication medium such as the internet or other type of computer network. The above features are embodied in one or more computer programs may be performed by one or more computers running such programs.

Software products (or components) may be tangibly embodied in a machine-readable medium, and comprise instructions operable to cause one or more data processing apparatus to perform operations comprising: a) clustering sequence data from a plurality of immunological receptors or fragments thereof; and b) providing a statistical analysis output on said sequence data. Also provided herein are software products (or components) tangibly embodied in a machine-readable medium, and that comprise instructions operable to cause one or more data processing apparatus to perform operations comprising: storing sequence data for more than 10², 10³, 10⁴, 10⁵, 10⁶, 10⁷, 10⁸, 10⁹, 10¹⁰, 10¹¹, or 10¹² immunological receptors or more than 10², 10³, 10⁴, 10⁵, 10⁶, 10⁷, 10⁸, 10⁹, 10¹⁰, 10¹¹, or 10¹² sequence reads.

In some examples, a software product (or component) includes instructions for assigning the sequence data into V, D, J, C, VJ, VDJ, VJC, VDJC, or VJ/VDJ lineage usage classes or instructions for displaying an analysis output in a multi-dimensional plot.

In some cases, a multidimensional plot enumerates all possible values for one of the following: V, D, J, or C. (e.g., a three-dimensional plot that includes one axis that enumerates all possible V values, a second axis that enumerates all possible D values, and a third axis that enumerates all possible J values). In some cases, a software product (or component) includes instructions for identifying one or more unique patterns from a single sample correlated to a condition. The software product (or component) may also include instructions for normalizing for amplification bias. In some examples, the software product (or component) may include instructions for using control data to normalize for sequencing errors or for using a clustering process to reduce sequencing errors. A software product (or component) may also include instructions for using two separate primer sets or a PCR filter to reduce sequencing errors.

### V. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Immune Repertoire Sequencing Method

In IR-seq, the first consideration of using MIDs is its optimum length and resultant barcode diversity. This is related to the overall number of antigen receptor transcripts in the sample. In order to tag each RNA molecule with a unique MID, MIDs must be designed with sufficient length (diversity) to cover each individual molecule. However, this requires knowledge of the total RNA molecules in the sample, which is often hard to obtain for samples containing highly expanded cells with increased antigen receptor transcripts, such as plasmablasts. In addition, longer MIDs decrease the reverse transcription efficiency.

Thus, a reduced MID length was used to develop a more generalized approach to identify each individual transcript using a sequence-similarity based clustering method, also referred to herein as molecular identification clustering-based immune repertoire sequencing (MIDCIRS), to separate sequencing reads into subgroups within a group of sequencing reads that have the same MID (FIG. 1). MIDs were tagged to cDNA during the reverse transcription step by fusing gene-specific primers specific to the constant region of the antibody heavy chain with 12 nucleotide MIDs and a sequencer-specific adaptor (FIG. 1A, and Table 1). Resulted paired-end sequencing reads were first merged and antibody reads were identified. Then reads were grouped according to the MID. Next, a clustering method was used to further separate different types of RNA molecules that were tagged with the same MID into sub-groups.

Clustering threshold is an important parameter to consider. This threshold needs to be optimized to group reads that are different due to sequencing and PCR errors into the same MID sub-group but exclude reads that are derived from different antibody sequences. RNA controls with known sequences were used to set the threshold (Levenshtein distance) to be 5% of the read length. Next, a consensus sequence was generated from each sub-group within a MID group by considering the number of reads in each sub-group and their quality scores. Each MID sub-group is equivalent to an RNA molecule. To calculate the total diversity, multiple consensus with the exact same sequences (RNA molecules that originated from the same cell) were combined and the number of unique consensus sequences were counted (FIG. 2). The approach described here that further clusters reads under the same MID is useful when the total number of receptor transcript information for a given sample is unknown or when shorter MIDs are preferred to maintain reverse transcription efficiency.

MID Clustering-Based IR-Seq has a Good Dynamic Range that Works on as Few as 1,000 Naïve B Cells:

To validate the method and test its dynamic range of amplification efficiency on samples with a large range of cell numbers, human naïve B cells were sorted into different amounts, from as few as 1,000 to as many as 1,000,000 cells, and libraries were prepared and analyzed as described above. 95% of the paired-end sequencing reads could be merged to form the full length heavy chain sequences (Table 2). Among them, an average of 78% of the sequencing reads were antibody heavy chain sequences. These numbers increased to 97% with increased cell input (Table 2).

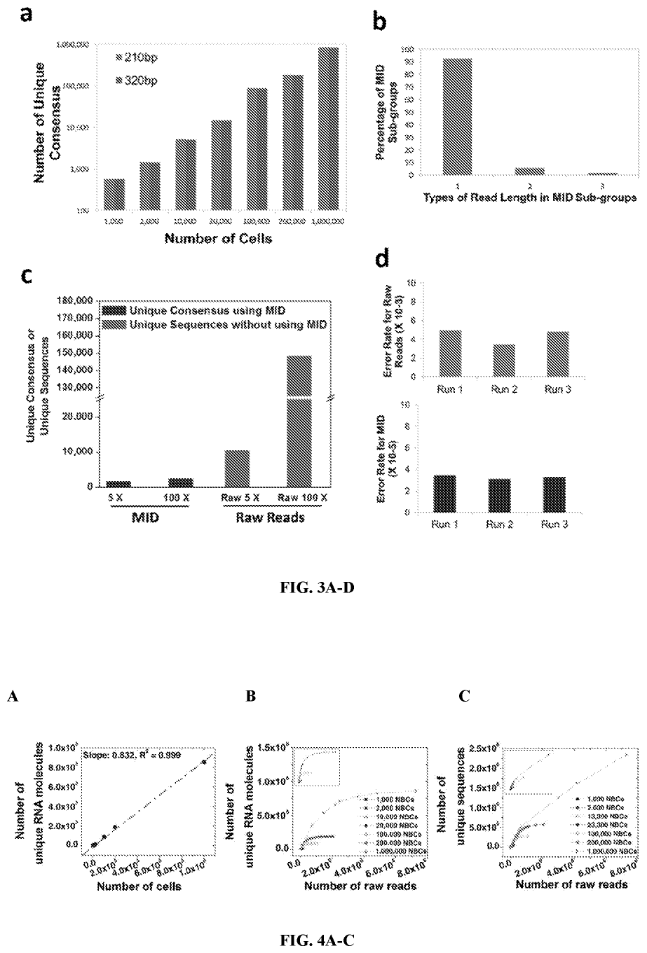

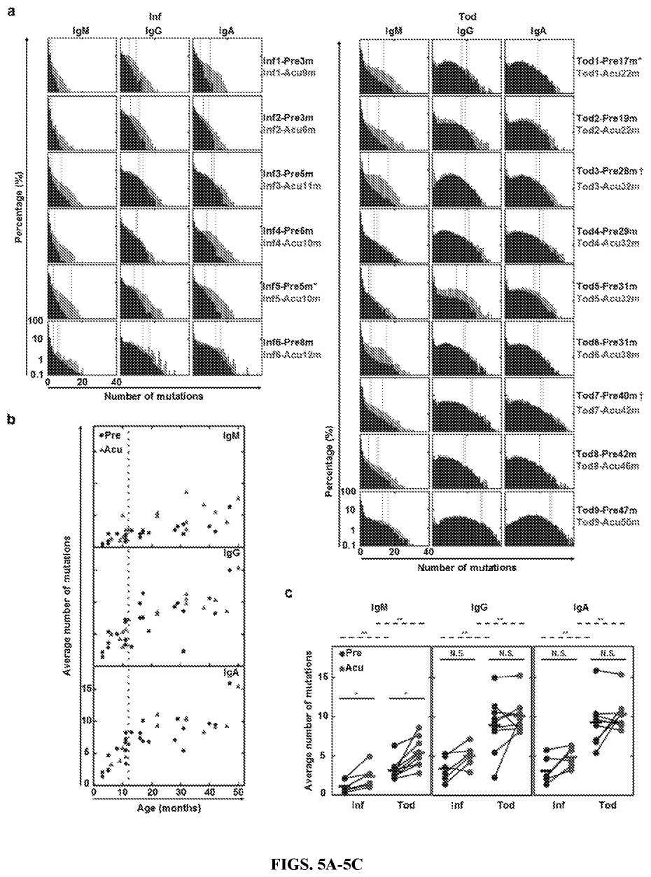

To test the sample input needed to cover the diversity, three independent libraries were prepared using either 5% of total RNA twice (technical replicate, library 1 and 2) or 30% of total RNA (library 3). The sequencing reads of the two 5% RNA were combined and referred to as library 1+2. After going through clustering, consensus generation, and combining unique consensus sequences, the resulted diversity estimates for different cell populations displayed a strong correlation with cell numbers. The observed diversity was also proportional to the RNA input, with a slope from 0.45 for 5% RNA input to 0.73 for 10% RNA input, and to 0.86 for 30% RNA input (FIG. 2A). These observed diversities and slopes are consistent with the model prediction (FIGS. 5 and 6), which demonstrated the efficiency of the protocol in amplifying a low copy number transcript, such as antibody sequences from naïve cells and low cell numbers. It also demonstrated the large dynamic range that the method provided. The two 5% RNA input technical replicates demonstrated good repeatability (FIG. 3A).

Sequencing depth is another important factor to consider when designing an IR-seq experiment. To take advantage of using MIDs to mitigate errors, an optimal sequencing depth is needed where there are multiple sequencing reads in each sub-group and MIDs that appear only once with one sequencing read are a minor population. For each library, sequencing was performed at five times the cell number and it was observed that about 92% of the reads belong to MIDs with two or more reads (Table 2). In addition, there must be sufficient reads to discover all possible diversity in a sample, which is important in estimating the repertoire diversity. A rarefaction analysis was performed by subsampling reads to different amounts. For all cell numbers, the rarefaction curves reached a plateau at the current sequencing depth, which is five times the cell number, suggesting that even if more sequencing was performed, it is not likely that new diversities would appear. For all libraries, sequencing two times the cell number seemed to cover most of the diversity in these samples (FIG. 2B). Although, the optimum sequencing depth is likely to change depending on sample format, e.g. peripheral blood mononuclear cells collected after immunization. The rarefaction curve provides a robust check for the sequencing depth when analyzing more complex samples.

MID Clustering-Based IR-Seq is Robust in Repertoire Diversity Estimation:

Having understood the sample input amount and sequencing depth required for repertoire sequencing, the robustness of this method was tested by designing a set of metrics to check its performance. Since naïve B cells were used and the somatic hypermutation rate is extremely low in these cells, including extra sequences on the variable region of the antibody heavy chain in the analysis would not increase overall diversity discovered if the sequencing reads were properly clustered. As expected, the diversity did not change significantly when considering either 210 bp or 320 bp in merged read length (FIG. 3A) with 98% unique consensus shared between two lengths. Using antibody sequences generated from single naïve B cells, it was verified that naïve B cells rarely have somatic mutations, each naïve B cell expresses a distinct heavy chain sequence, and less than 4.2% of the naïve B cells have a non-productive heavy chain, which are consistent with B cell development (Brezinschek et al., 1995).

Another parameter that was used to check the robustness of MID clustering-based IR-seq in estimating the diversity was to check the read length in each MID sub-group. If the clustering threshold is optimum, then the read length should be the same in each sub-group. More than 95% of sub-groups harbor reads with the same length (FIG. 3B). In addition, a probability model was applied to predict the antibody transcript copy number based on observed diversity depending on amount of RNA input. The results showed that a copy number of 12 is consistent with the total diversity and unique consensus size that was observed, which is equivalent to the number of RNA molecules in a cell. This number is also consistent with previously published antibody copy numbers for naïve B cells (Jack and Wabl 1988). These comparisons demonstrated the robustness of the chosen clustering threshold.

MID Clustering-Based IR-Seq Significantly Reduces Error Rate:

Next, the error rate was examined with or without using MID clustering-based IR-seq. Because the diversity among hundreds of millions of antigen receptors lies in a short stretch of DNA about 60 nucleotides, often two distinct sequences are different by only a few nucleotides. In addition, somatic hypermuation, a process that further diversifies the antibody gene sequences, has a mutation rate that is comparable to the error rate of the next-generation sequencers. This makes estimating the total antigen receptor diversity and tracing the mutational evolution of antibody gene sequences difficult. Using MIDs can reduce the error rate by several orders magnitude and enable an accurate sequencing and diversity comparison. By comparing individual reads within a sub-group to the consensus read, the observed error rate was similar to Illumina, which is about 0.5% (Loman et al., 2012; Vollmers et al., 2013). To calculate the improved error rate using the MID clustering-based IR-seq, the total reads were split into two groups, clustering was performed separately, and the consensus of overlapping sub-groups from these two sub-samples was compared. The resulted error rate was 130-fold smaller than the current error rate, which reached a quality score of Q45. In addition, while the raw error rate fluctuated between runs as demonstrated by the error rate from three runs (FIG. 3D, top panel), the improved error rate after using MIDs for these three runs almost did not fluctuate (FIG. 3D, bottom panel). This comparison can also be used to guide the cluster generation on the sequencer to maximize the sequence yield without comprising the sequence quality. Without MIDs, the diversity estimate is massively inflated with errors due to PCR and sequencing as demonstrated in one experiment where 1.3 million reads were obtained for one library made from 10,000 cells. It generated 258,320 unique raw reads and, even after removal of unique sequences represented by only one read, there were still 148,680 unique sequences, which is impossible for a total of 10,000 cells (FIG. 3C). This demonstrates the necessity of using MID clustering-based IR-seq in immune repertoire sequencing.

### Example 2—Methods and Materials

Cell Sorting:

Human PBMCs were purified from blood bank donor samples. Naïve B cells were sorted based on the phenotype of CD3⁻CD19⁺CD20⁺CD27⁻CD38⁻ (antibodies from BioLegend). Cells were lysed in RLT Plus buffer (Qiagen) supplemented with 1% β-mercaptoethanol (Sigma).

Bulk Antibody Sequencing Library Generation:

MIDs were added during the reverse transcription step through the use of fusion primers, which contain the partial illumina P5 sequencing adaptor followed by twelve random nucleotides and primers to the constant region of five antibody isotypes. Eleven leader region primers that were previously designed (Jiang et al., 2013) were fused to a partial Illumina P7 adaptor. Full Illumina adaptors were added during the second PCR step along with library indexes. Total RNA was purified using All Prep DNA/RNA kit (Qiagen). Different amount of input materials were used for reverse transcription as indicated in figures. Superscript III (Life Technologies) was used for the reverse transcription step with manufacturer's suggested concentrations followed by an Exonuclease I (New England Biolabs) treatment step. Takara Ex Taq HS polymerase (clone Tech) was used for the PCR with initial denature at 95° C. for 3 mins, followed by 20 cycles of 95° C. for 30s, 57° C. for 30s, and 72° C. for 2 mins. The second PCR was performed with following programs: initial denature at 95° C. for 3 mins, followed by 10 cycles of 95° C. for 30s, 57° C. for 30s, and 72° C. for 2 mins. Libraries were gel purified and quantified by qPCR Library Quantification Kit (KAPA biosystems) and sequenced on Illumina Mi-seq with paired-end 250 bp read.

Preliminary Read Processing:

Raw reads from Illumina MiSeq PE250 were first cleaned up following steps outlines in FIG. 1B. Only those reads that matched exactly to the corresponding sample's molecular index were included for further process. The end of each raw read was trimmed to maintain all bases having a quality score of 25 or higher. Reads 1 and Reads 2 were merged by SeqPrep tool (https://github.comjstjohn/SeqPrep). The merged reads were filtered with specific V-gene and constant region primers to determine immunoglobulin (Ig) sequencing reads. The retained reads were truncated to 210 bp or 320 bp, two kinds of lengths for the following analysis. Read numbers after various filters are listed in Table 2.

MID Sub-Group Generating:

Raw reads were split into MID groups according to the 12nt barcodes. For each MID group, a quality threshold (QT) clustering was used to cluster similar reads. This process is primarily used to group reads derived from a common ancestor RNA molecule and separate reads derived from distinct RNAs. The Levenshtein distance of 5% was used to set the threshold. This was calibrated using RNA controls with known sequences (FIG. 1). For each subgroup, a consensus sequence was built based on the majority nucleotide weighted by quality score at each position. In the case that there were only two reads in a MID sub-group, they were only considered useful reads if they were identical. Each MID sub-group is equivalent to an RNA molecule. Next, all of the identical consensus were merged to form a unique consensus, which was used to estimate the diversity and assess the sequencing depth in rarefaction analysis.

TABLE 2
Sequencing read statistics.

| | | | | | | | | Number of |
|---|---|---|---|---|---|---|---|---|
| | | | | | Number | Number | | useful MIDs |
| | | Number | Number | Number | of reads | of reads | Number | containing |
| | Number | of raw | of merged | of Ig | truncated | truncated | of useful | more than one |
| Library | of cells | reads | reads | reads | to 210bp | to 320bp | MIDs^{a} | sub-group^{b} |
| Library 1 | 1,000 | 18,811 | 15,753 | 3,430 | 3,430 | 3,422 | 180 | 0 |
| (5% RNA) | 2,000 | 15,625 | 15,098 | 8,583 | 8,583 | 8,494 | 518 | 1 |
| | 10,000 | 1,374,000 | 1,273,869 | 1,166,493 | 1,166,467 | 1,162,390 | 1,102 | 2 |
| | 20,000 | 509,519 | 491,782 | 456,993 | 456,990 | 456,089 | 2,463 | 51 |
| | 100,000 | 949,284 | 928,711 | 876,730 | 876,721 | 875,089 | 5,092 | 41 |
| | 200,000 | 1,885,402 | 1,845,918 | 1,748,669 | 1,748,655 | 1,745,054 | 32,414 | 265 |
| | 1,000,000 | 5,411,037 | 5,287,615 | 5,118,134 | 5,118,129 | 5,073,895 | 603,354 | 15,247 |
| Library 2 | 1,000 | 6,236 | 6,104 | 4,432 | 4,432 | 4,408 | 151 | 1 |
| (5% RNA) | 2,000 | 42,457 | 41,501 | 15,000 | 15,000 | 10,380 | 501 | 1 |
| | 10,000 | 60,109 | 55,773 | 53,174 | 53,174 | 52,401 | 1,882 | 11 |
| | 20,000 | 153,007 | 148,420 | 91,638 | 91,637 | 90,424 | 5,756 | 19 |
| | 100,000 | 466,492 | 455,501 | 441,012 | 441,007 | 437,148 | 42,752 | 124 |
| | 200,000 | 1,218,051 | 1,191,089 | 1,154,955 | 1,154,942 | 1,144,292 | 125,430 | 747 |
| | 1,000,000 | 4,847,676 | 4,739,171 | 4,654,316 | 4,654,287 | 4,615,423 | 594,353 | 14,100 |
| Library 3 | 1,000 | 46,320 | 22,742 | 9,201 | 9,201 | 9,149 | 797 | 1 |
| (30% RNA) | 2,000 | 44,846 | 18,602 | 17,421 | 17,421 | 17,267 | 2,176 | 2 |
| | 10,000 | 228,711 | 99,370 | 62,242 | 62,242 | 61,121 | 7,102 | 9 |
| | 20,000 | 293,279 | 196,570 | 184,754 | 184,746 | 182,818 | 23,991 | 49 |
| | 100,000 | 1,153,763 | 1,074,771 | 1,048,523 | 1,048,513 | 1,041,048 | 165,663 | 1,137 |
| | 200,000 | 2,191,738 | 2,107,762 | 2,059,944 | 2,059,917 | 2,045,047 | 404,225 | 7,239 |
| | 1,000,000 | 7,494,809 | 7,342,163 | 7,258,253 | 7,258,195 | 7,207,962 | 1,516,098 | 108,172 |

^{a}A useful MID should have more than two reads. If there are only two reads in a MID, they should be identical, otherwise, this MIG group is discarded.
^{b}The number of MIDs containing more than one type of antibody heavy chain transcripts.

Diversity Coverage and RNA Copy Number Simulation:

The estimation of diversity will be affected by the initial RNA sampling depth (percentage of initial RNA used to construct the sequencing library). A statistical model was used to estimate the diversity coverage for the naïve B cells that were sorted based on RNA sampling depth. The possible RNA diversity coverage was estimated for RNA copy numbers in range of 1 to 20, with the initial sampling amount 5%, 10% and 30% of total RNA molecules. The predicted values matched experimental results well. The copy number estimate was also verified by examining the MID sub-group size distribution of the unique consensus. Only less than 10 unique consensus out of 562,681 were represented by more than 15 MID sub-groups while plasmablasts can have 100 to 1000 times more Ig transcripts compared to naïve B cells.

### Example 3—Application of Immune Repertoire Sequencing in Malaria

As a proof of principle, the MID clustering-based immune repertoire sequencing was used to examine the antibody repertoire diversification in infants (<12 months old) and toddlers (12-42 months old) from a malaria endemic region in Mali before and during acute Plasmodium falciparum infection. Although the antibody repertoire in fetuses, cord blood, young adults, and the elderly, have been studied, infants and toddlers are among the most vulnerable age groups to many pathogenic challenges, yet their immune repertoires are not well understood. It is commonly believed that infants have poorer responses to vaccines than toddlers because of their developing immune system. Thus, understanding how the antibody repertoire develops and diversifies during a natural infection, such as malaria, not only provides valuable insight into B cell ontology in humans, but also provides critical information for vaccine development for these two vulnerable age groups. Using peripheral blood mononuclear cells (PBMCs), MBCs, and PBs from 12 children aged 3 to 42 months old, it was discovered that infants and toddlers used the same V, D, and J combination frequencies and had similar complementarity determining region 3 (CDR3) length distributions.

The 12 random nucleotide MIDs were used identify each individual transcript using a sequence-similarity-based clustering method to separate a group of sequencing reads with the same MID into sub-groups as described in Example 1. Consensus sequences were then built by taking the average nucleotide at each position within a sub-group, weighted by the quality score. Each consensus sequence represents an RNA molecule, and identical consensus sequences can be merged into unique consensus sequences, or unique RNA molecules (FIG. 1).

MIDCIRS Yields High Accuracy and Coverage Down to 1000 Cells:

Sorted naïve B cells with varying numbers (10³ to 10⁶) were used to test the dynamic range of MIDCIRS. The resulting diversity estimates, or different types of antibody sequences, display a strong correlation with cell numbers at 83% coverage (FIG. 4C, slope). Previous studies have shown that about 80% of naïve B cells express distinct heavy chain genes (DeKosky et al., 2013), thus the present method achieves a comprehensive diversity coverage that is much higher than other MID-based antibody repertoire sequencing techniques.

Rarefaction analysis was performed by subsampling sequencing reads to different amounts and then computing the diversity to test the effect of sequencing depth and error rate on MIDCIRS. On average, the rarefaction curves reach a plateau at a sequencing depth of around three times the cell number using MIDCIRS, suggesting that sequencing more will not discover further diversity (FIG. 4D). In contrast, without using MIDCIRS, the number of unique sequences continues to increase well beyond the number of cells for all samples (FIG. 4E). Optimum sequencing depth is likely to change depending on sample composition (e.g. PBMCs after immunization). Consistent with previous MID-based IR-seq experiments (Vollmers et al., 2013), MIDCIRS reduces the error rate to 1/130^{th} of the Illumina error rate, providing the accuracy necessary to distinguish genuine SHMs (1 in 1,000 nucleotides) from PCR and sequencing errors (1 in 200 nucleotides) (FIG. 11).

Infants and Toddlers have Similar VDJ Usage and CDR3 Lengths:

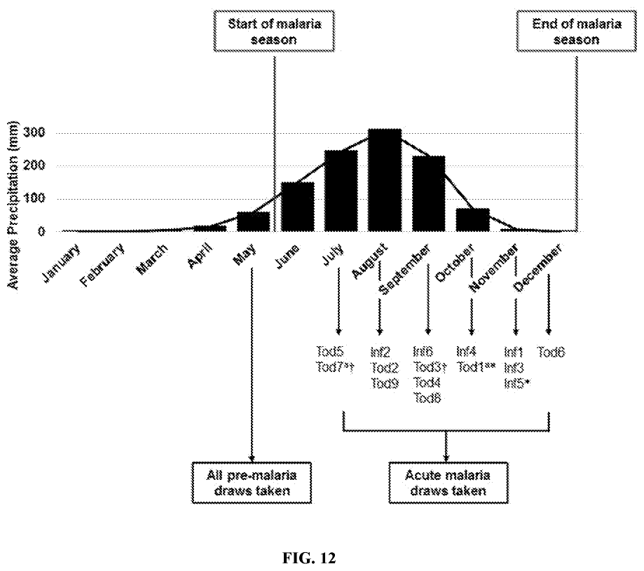

Equipped with this ultra-accurate and high-coverage antibody repertoire sequencing tool, it was used to study the antibody repertoire of infants and toddlers residing in a malaria endemic region of Mali. From an ongoing malaria cohort study, paired PBMC samples were collected before and during acute febrile malaria from 13 children aged 3 to 47 months old (FIG. 12 and Table 4). Two of the children were followed for an additional year, giving 15 total paired PBMC samples. An average of 3.8 million PBMCs per sample were directly lysed for RNA purification. All PBMCs were subjected to MIDCIRS analysis. An average of 3.75 million sequencing reads were obtained for each PBMC sample (Table 5).

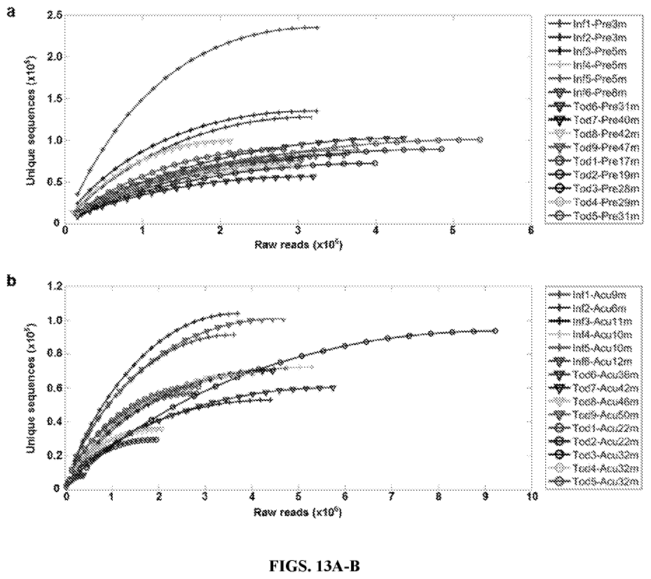

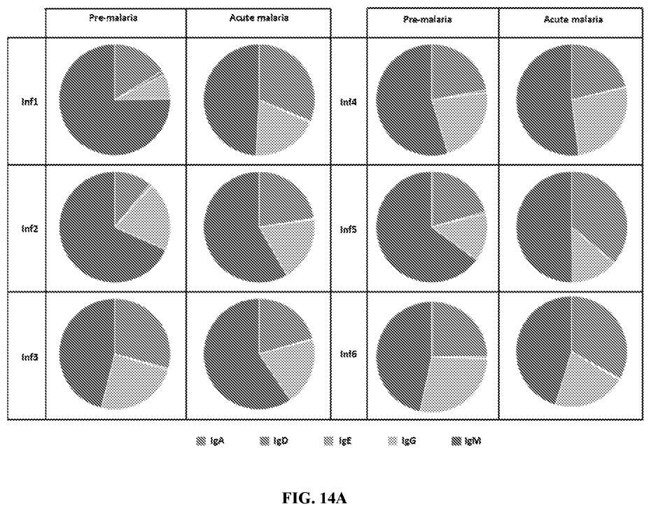

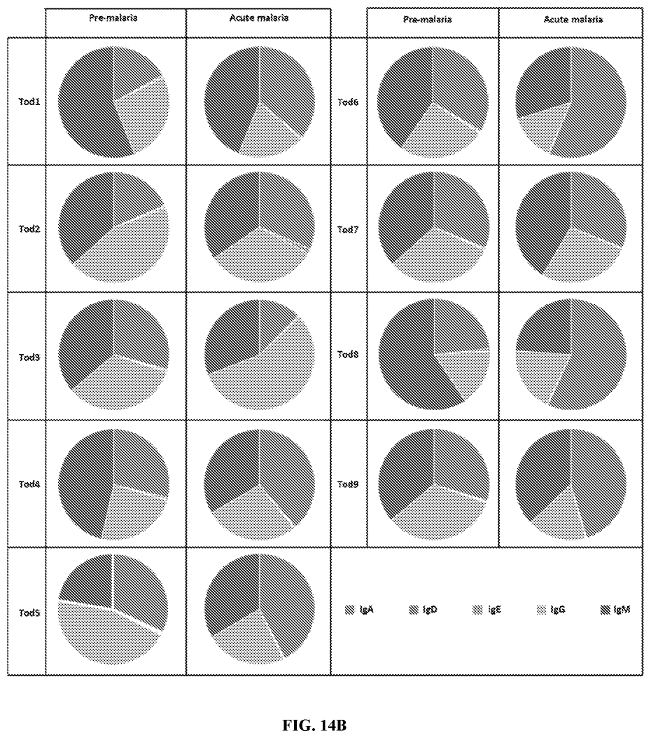

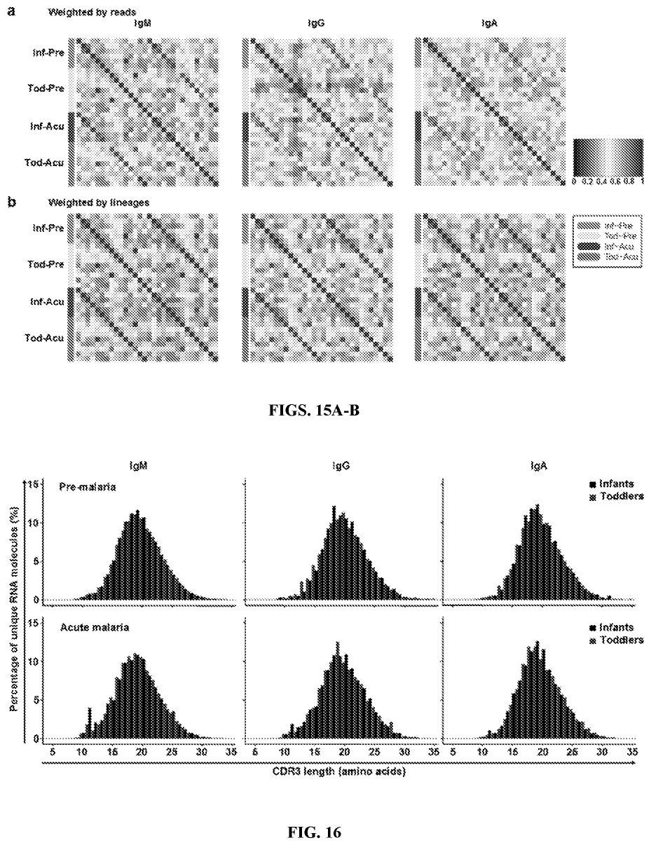

For all PBMC samples, sequencing approximately the same number of reads as the cell numbers saturates the rarefaction curve (FIG. 13). VDJ gene usage is highly correlated for IgM between infants and toddlers regardless of weighting the correlation coefficient by the number of sequencing reads or clonal lineages (FIG. 15), demonstrating that the same mechanism of VDJ recombination is used to generate the primary antibody repertoire in infants and toddlers. Weighting on the number of clonal lineages in each VDJ class increases the correlation for IgG and IgA compared with weighting on the number of reads in each VDJ class (FIG. 15). The diagonal lines in each panel indicate same sample self-correlation, and the two shorter off-diagonal lines indicate correlations from two timepoints of the same individual. These data recapitulate previous observations from our study in zebrafish that clonal expansion-induced differences on the number of reads in each VDJ class can confound the highly similar VDJ usage during B cell ontology. In addition, infants and toddlers have similar CDR3 length distributions across the three isotypes and both timepoints (FIG. 16), consistent with recent studies of PBMCs from 9 month olds infants and adults and confirming the previous results that an adult-like distribution of CDR3 length is achieved around two months of age (Schroeder et al., 2001).

Both Infants and Toddlers have Unexpectedly High SHM:

SHM is an important characteristic of antibody repertoire secondary diversification due to antigen stimulation. Although it has been demonstrated before that infants have fewer mutations in their antibody sequences than toddlers and adults, the limited number of sequences for only a few V genes does not provide convincing evidence of the levels of SHM in infants. A recent study using the first generation of IR-seq showed that two 9-month-old infants averaged at least 6 SHMs in IgM of an average length of 500 nucleotides. These numbers are equivalent to, if not higher than, reported SHM rates in IgM sequences from healthy adults day 7 post influenza vaccination and are much higher than a low-throughput infant study using a few V genes and limited antibody sequences. Due to inherent errors associated with the first generation of IR-seq as discussed above, it is possible that PCR and sequencing errors played a role. In addition, it remains unclear if infants (<12 months old) are able to generate a significant number of mutations in response to infection, which would demonstrate their capacity to diversify the antibody repertoire.

Here, it was shown that infants (<12 months old) and toddlers (12-47 months old) reach an unexpectedly high level of SHMs in all 3 major isotypes, particularly IgG and IgA (FIG. 5A). While the mutation distributions remain in the low end of the spectrum for IgM, the number of mutations is significantly higher in IgG and IgA for both age groups. The threshold for the 10% most highly mutated unique RNA molecules is around 10 in infant IgG and IgA sequences (FIG. 5A, Infants, right of the long vertical lines) and around 20 in toddler IgG and IgA sequences (FIG. 5A, Toddlers, right of the long vertical lines). To minimize any possible inflation of SHMs, all sequences that were mapped to novel alleles were excluded, which were identified by both TIgGER and inspecting IgM sequences. These putative novel alleles account for 8% of all unique sequences on average (Table 6). Naïve B cells from these same patients, sorted as a control, harbor only 0.55 mutations on average, as expected (Table 7). Upon acute malaria infection, the SHM histogram shifts rightward for almost all isotypes in almost all individuals (FIG. 5A, the right shift of light long vertical line compared to dark long vertical line), including infants. These results demonstrate high levels of SHM that exceed what have been documented previously (Ridings et al., 1997).

SHM Load is Distinct Between Infants and Toddlers:

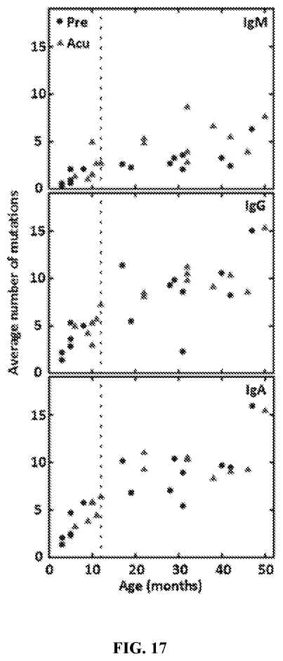

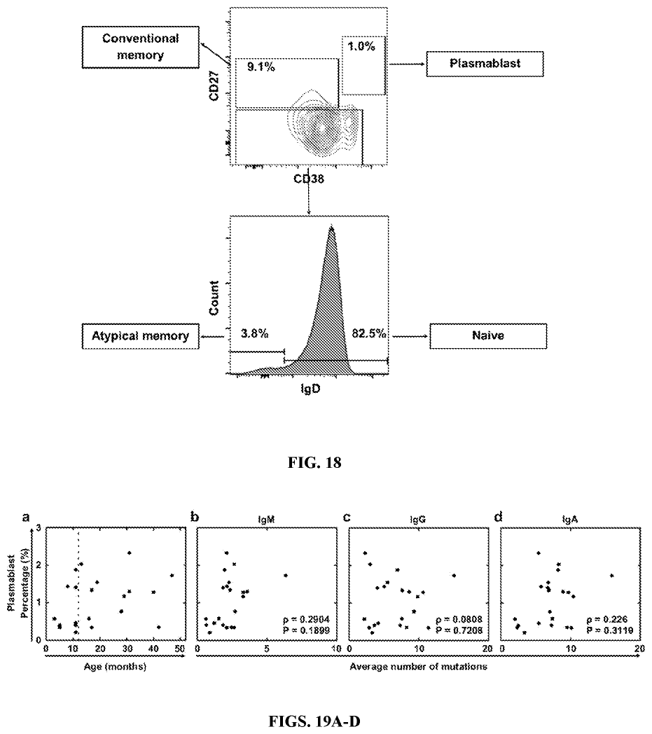

The differences in the shapes of SHM distributions of infants and toddlers, steadily decreasing from unmutated for infants in all three isotypes while peaking around 10 for toddlers in IgG and IgA (FIG. 5A), suggest that the total SHM load might reflect the history of interactions between the antibody repertoire and the environment, including malaria exposure. Since the malaria season is synchronized with the 6-month rainy season (FIG. 12), and >90% of the individuals in this cohort are infected with P. falciparum during the annual malaria season, it was hypothesized that the SHM load would increase with age. However, it was found that the SHM load rapidly increases with age in infancy and then appears to plateau around 12 months of age in an initial smaller set of children with paired pre-malaria and acute malaria PBMC samples (FIG. 17). 9 pre-malaria samples around the infant and toddler transition (5 of 11 months old and 4 of 13 to 17 months old) were added. The two-staged trend of SHM load remains for all three isotypes (FIG. 5B), with samples around the transition having the largest variation. Detailed comparisons show that, consistent with the two-stage trend, toddlers have a higher SHM load compared with infants for all three isotypes at both pre-malaria and acute malaria timepoints (FIG. 5C, comparison between age groups). Although there is a significant increase on SHM load upon acute malaria infection in IgM for both infants and toddler, bulk PBMC analysis does not show a significant increase in IgG or IgA, possibly because of the already elevated SHM base level. This, along with the two-stage trend (FIG. 5B), suggests that 12 months is an important developmental threshold for secondary antibody repertoire diversification: before this threshold, the global repertoire is quite naïve but can quickly diversify upon a natural infection.

Higher Memory B Cell Percentage Results in Higher SHM Load:

This unexpected developmental threshold of secondary antibody repertoire diversification prompted focus on B cell subset composition changes and ask whether they correlate with this two-staged SHM load. Flow cytometry analysis reveals that naïve B cells decrease from about 95% in 3-month-old infants to about 80% in toddlers (FIG. 6A). Conversely, memory B cells increase from about 4% in 3-month-old infants to about 15% in toddlers (FIG. 6F). As the two-stage SHM load analysis suggests, 12 months appears to divide the samples into two age groups, with a large variation at the infant to toddler transition and in the toddler group. Infants have a significantly more naive B cells and fewer memory B cells than toddlers (FIG. 6B, G). Plasmablast percentages fluctuated in a much smaller range (FIG. 19). With a similar two-staged trend observed for B cell subset percentages, it was hypothesized that the B cell subset percentage would correlate with SHM load. Indeed, further analysis showed that the decrease in naive B cell percentage and the increase in memory B cell percentage correlate well with SHM load across IgM, IgG, and IgA isotypes (FIGS. 6C-E and H-J), which supports the initial hypothesis that 12 months separates infants from toddlers in both SHM load and B cell composition changes. These data suggest that memory B cells contribute significantly to the developing antibody repertoire, and their composition is essential in secondary antibody repertoire diversification.

SHMs are Similarly Selected in Infants and Toddlers:

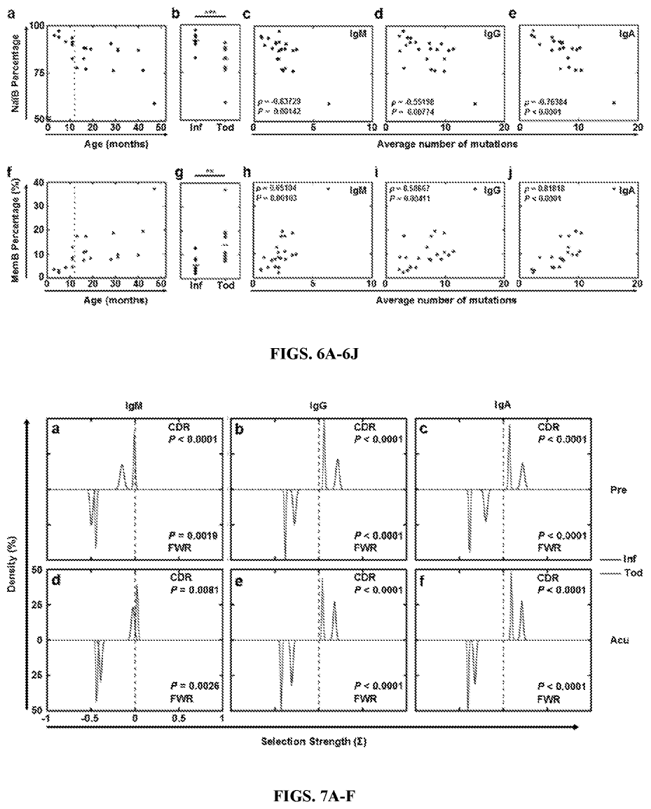

One of the key features of antibody affinity maturation is antigen selection pressure imposed on an antibody, which is reflected in the enrichment of replacement mutations in the CDRs, the parts of the antibody that interact with antigens, and the depletion of replacement mutations in the framework regions (FWRs), the parts of the antibody responsible for proper folding. The unexpectedly high level of SHMs observed in infants prompted us to ask whether those SHMs have characteristics of antigen selection, as seen in older children and adults. As previous studies have shown that infants have limited CD4 T cell responses and neonatal mice exhibit poor germinal center formation (PrabhuDas et al., 2011), it was hypothesized that infant antibody sequences would display weaker signs of antigen selection. Here, BASELINe (Yaari et al., 2012) was used to compare the selection strength. BASELINe quantifies the likelihood that the observed frequency of replacement mutations differs from the expected frequency under no selection; a higher frequency implies positive selection and a lower frequency implies negative selection, and the degree of divergence from no selection relates to the selection strength. Surprisingly, despite infants harboring fewer overall mutations, these mutations are positively selected in the CDRs and negatively selected in the FWRs in both IgG and IgA (FIG. 7B, C, E, F). Contrary to the hypothesis that infants would have a lower selection strength than toddlers, for both IgG and IgA, infants actually have a higher selection strength at both pre-malaria and acute malaria timepoints (FIG. 7). The lower selection strength in infant IgM sequences at the pre-malaria timepoint is significantly higher during acute malaria infection (FIG. 7A, D, CDR black curves between two timepoints, P<0.0001 [numerical integration, as previously described (Yaari et al., 2012)]), suggesting that the significant increase in SHM is antigen-driven and selected upon. In order to compare with a large amount of historical adult data, replacement to silent mutation ratios (R/S ratios) were calculated, which are about 2-3:1 in FWRs and 5:1 in CDRs for both infants and toddlers (Table 8). These results are similar to adults and much higher than what has been reported for children previously using a very limited number of sequences. It was also noticed that R/S ratio in the FWRs of IgM was much higher in infants, contrary to the BASELINe results, which highlights the importance of incorporating the expected replacement frequency when considering selection pressure. These results suggest that as an end result of interactions between antigen selection and SHM, the degree of antibody amino acid changes is comparable in infants, toddlers, and adults. It also suggests that cellular and molecular machineries for antigen selection are already in place in infants.

Clonal Lineages Diversify Upon Acute Febrile Malaria:

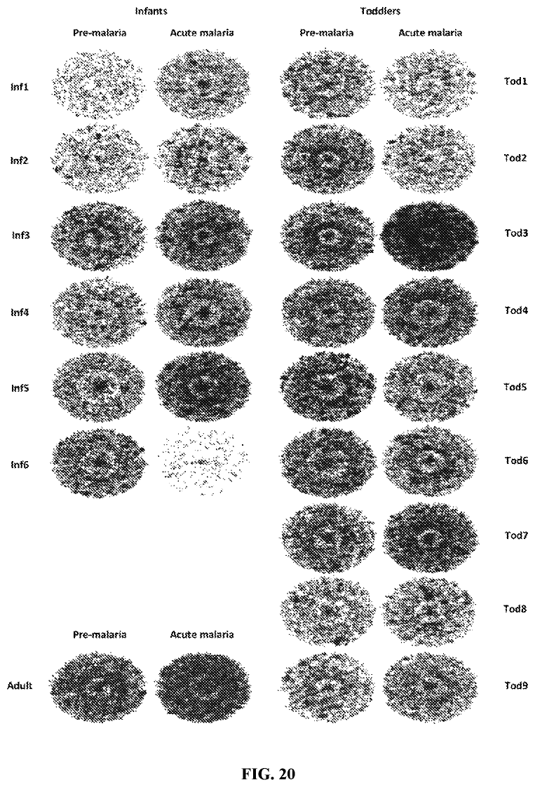

The exhaustive sequencing data obtained by MIDCIRS offers the possibility to reconstruct clonal lineages that trace B cell development. Clonal lineages contain different species of unique antibody sequences that could be progenies derived from the same ancestral B cell. B cell clonal lineage analysis has been used to track affinity maturation and sequence evolution of HIV broadly neutralizing antibodies. Using a clustering method with a pre-determined threshold (90% similarity on nucleotide sequence at CDR3), it was previously demonstrated that B cell clonal lineages could be informatically defined and contain pathogen-specific antibody sequences. In addition, the clonal lineage analysis also highlighted the lack of antibody diversification in the elderly after influenza vaccination. Using the same approach and a similar threshold, it was aimed to answer whether infants and toddlers are able to diversify antibody clonal lineages in response to infection and, if so, whether they have a similar ability to do so, which was previously impossible to answer due to technical limitations. To do this, structures of informatically defined clonal lineages were visualized for the entire antibody repertoire (FIG. 20). Each oval lineage map represents an individual PBMC sample at one timepoint. Densely packed individual lineages are not easily identified visually in FIG. 20; however, dark areas indicate that clonal lineages are already complex in this cohort of infants as young as 3 months old and can be further diversified upon acute febrile malaria.

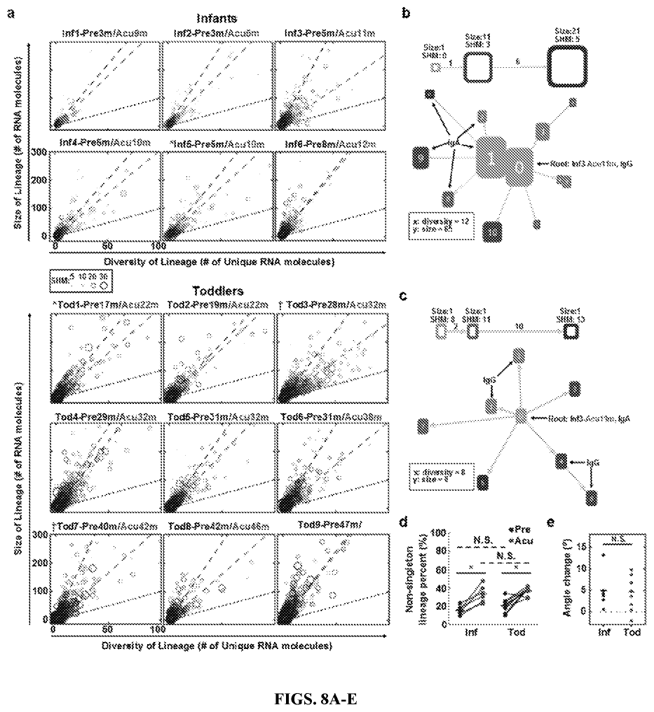

The densely packed lineages could result from large lineage sizes (one unique RNA molecule with many copies), large lineage diversities (many unique RNA molecules), or a combination of the two. To closely examine the possible differences in the degree of this intra-clonal lineage expansion and diversification between infants and toddlers, especially upon acute febrile malaria, the global lineage structure was projected (FIG. 20) onto diversity and size of lineage axes (FIG. 8A). Each circle represents an individual lineage, with the area of the circle proportional to the SHM load (average mutations of the lineage). This analysis effectively captures five parameters that quantify lineage complexity in a sample: number of total clonal lineages (number of circles), diversity of each lineage (x-axis position, number of unique RNA molecules in a lineage), size of each lineage (y-axis position, number of total RNA molecules in a lineage), SHM load of each lineage (area of circle, key is located in between the infant and toddler panels in FIG. 8A), and the extent of clonal expansion of each lineage (distance from y=x parity line; no clonally expanded RNA molecules within a lineage if it is on parity line or pure clonal expanded RNA molecules if it is in the top left quadrant of each panel).

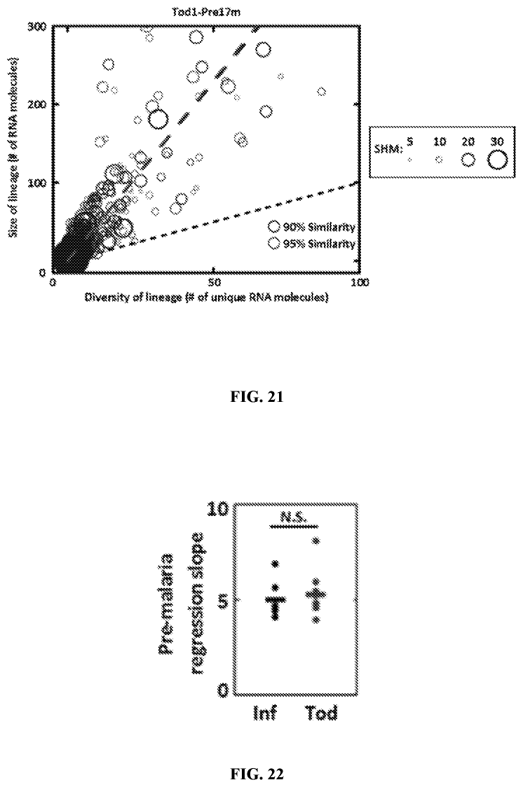

FIG. 8A, C are two example lineages selected to display the full lineage structures to demonstrate a lineage with diversification and clonal expansion (FIG. 8B refers to letter “b” indicated in FIG. 8Aa, Inf3) and another one with diversification but without clonal expansion (FIG. 8C refers to letter “c” indicated in FIG. 8A, Inf3). Both are represented by a single circle in FIG. 8A, but their locations in FIG. 8A depend on the numbers of RNA molecules (y-axis) and numbers of unique RNA molecules (x-axis). Lineage “c” (c in FIG. 8A, Inf3, zoomed in view in FIG. 8C) that lies away from the origin and near the black y=x parity line consists of 8 unique sequences, each represented by only one RNA molecule, indicating extensive lineage diversification but no clonal expansion. Lineage “b” (b in FIG. 8A, Inf3, zoomed in view in FIG. 8B) that lies far from the parity line is dominated by two unique RNA molecules each with about 20 copies (FIG. 8B, height of nodes), indicating extensive clonal expansion of particular sequences in addition to diversification. Changing lineage forming threshold from 90% to 95% does not change the overall structure of the lineages (FIG. 21).

This five-dimension lineage analysis reveals that infants as young as 3 months old can generate extensive lineage structures, with many lineages containing more than 20 different types of antibody sequences and 50 RNA molecules (FIG. 8A). Toddlers have many more lineages with higher levels of both size and diversity. However, in both infants and toddlers, the majority of clonal lineages are singleton lineages consisting of only one RNA molecule (FIG. 8D), consistent with the flow cytometry analysis that the bulk of the B cell repertoire is naive in these young children (FIG. 6). Upon acute malaria infection, the fraction of non-singleton lineages increases in both infants and toddlers (FIG. 8D).

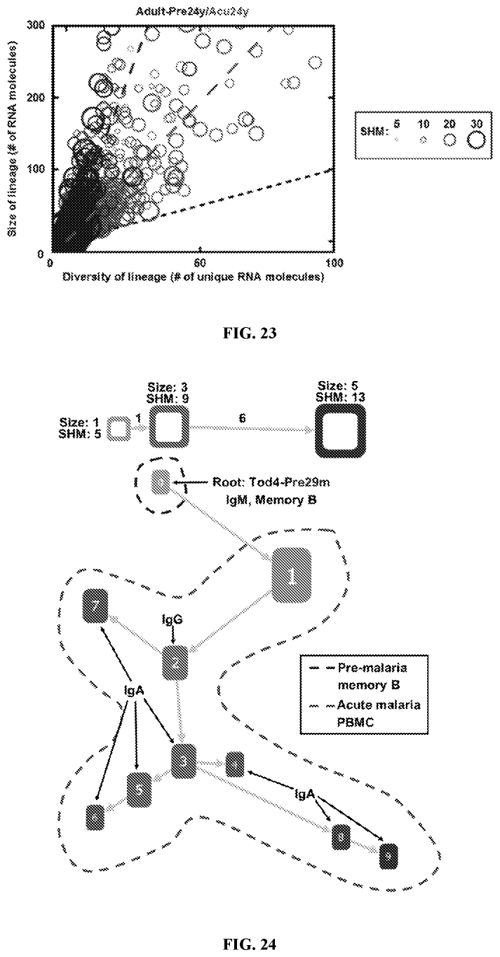

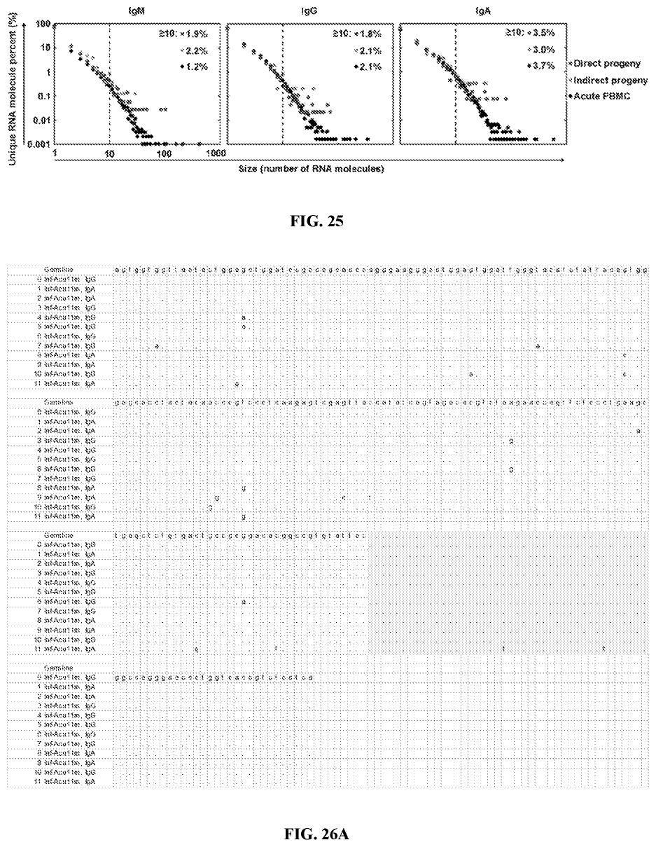

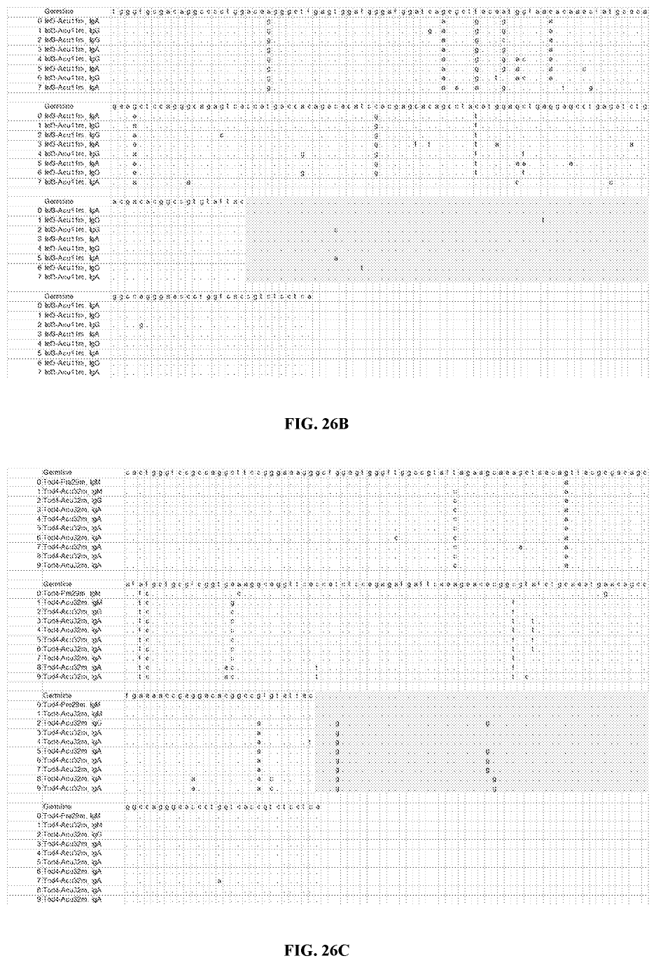

In order to tease out whether these non-singleton lineages diversify or clonally expand upon acute infection, linear regressions were fit to the lineage diversity-size plots. An immune response against an infection can have a two-fold effect on the lineage landscape: antigen stimulation can cause clonal expansion, which would shift the lineage up on the y-axis, and SHM and affinity maturation, which would shift the lineage to the right on the x-axis. This balance between clonal expansion and diversification is depicted by the slope of the linear regression (FIG. 8A, dashed dark lines for pre-malaria samples and dashed light lines for acute malaria samples). It was hypothesized that the lower absolute SHM load of infants would imply a defect in the ability to diversify clonal lineages in response to infection, leading the slope change from pre-malaria to acute malaria to be low (a small angle between blue and pink dashed lines) or even negative (pink dashed line is closer to y-axis than blue dashed line). Surprisingly, the analysis shows that infants diversify their clonal lineages in a similar manner as toddlers in response to acute malaria (FIG. 8E). As singleton lineages do not bear any weight on the linear regression, the analysis shows that the increasing fraction of non-singleton lineages upon malaria infection is similarly diversified between infants and toddlers, which is also similar to a young adult at pre-malaria and acute malaria (FIG. 23). However, this sharply contrasts with what had previously been observed in the elderly following influenza vaccination, where clonal expansion dominated. Among clonally expanding and diversifying B cell clones during an infection, only a subset of the cells comprising the clonal burst remain once the infection has been cleared. Thus, the characteristic change in the lineage size/diversity linear regression slope upon infection is expected to subside as time passes since the acute infection. Indeed, comparing the pre-malaria lineage size/diversity linear regression slopes reveals no difference between infants (who have not experienced malaria before) and toddlers (who have experienced malarias in previous years) (FIG. 22). These results highlight the unexpected capability of young children's antibody repertoire in response to a natural infection.

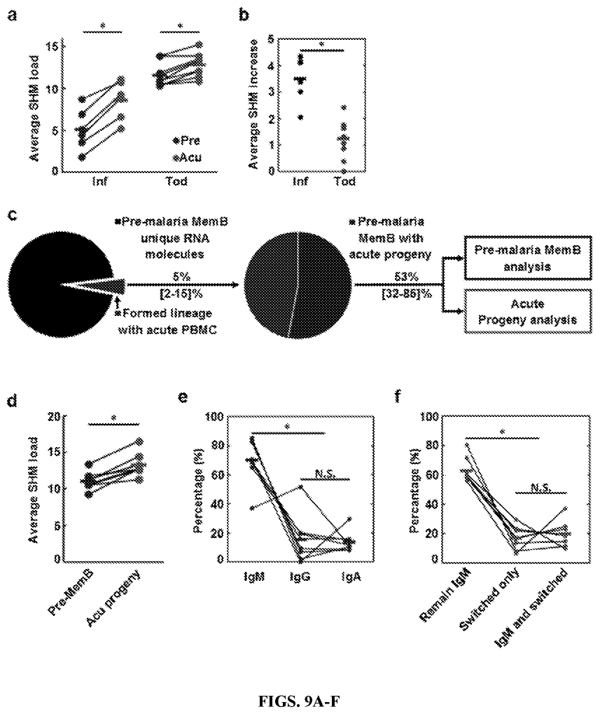

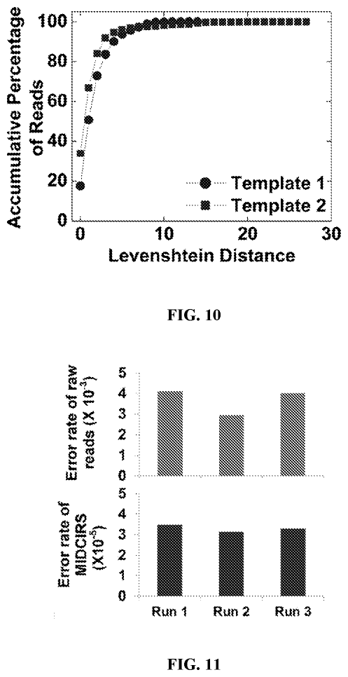

SHM load increases upon an acute febrile malaria infection: The plateau observed on SHM load in toddlers at both pre- and acute malaria (FIG. 5B) and the lack of a SHM difference in IgG and IgA between pre- and acute malaria (FIG. 5C) seems to suggest that the experienced part of the repertoire does not respond to malaria infection by inducing SHM. However, it could be that only a portion of the bulk antibody repertoire responds to the infection and there is already a high level of baseline SHMs as revealed by the histogram analysis (FIG. 5A). Since the lineage diversification was seen upon malaria infection in FIG. 5, it was hypothesized that examining the SHMs from sequences in two-timepoint-shared lineages (lineages containing both pre-malaria and acute malaria sequences) would enable us to quantify the infection-induced SHM increase from the highly mutated background. To test this, all sequences were pooled from both timepoints, including sorted memory B cells at pre-malaria, and generated lineages again using the 90% similarity threshold at CDR. Two-timepoint-shared lineages were found in all individuals analyzed (Table 9). Consistent with the observation that toddlers already have a diverse and expanded antibody repertoire compared to infants, there are more shared lineages in toddlers than infants (Table 9). SHMs were tallied for sequences from pre-malaria and acute malaria in the two-timepoint-shared lineages separately. Consistent with the hypothesis, both infants and toddlers significantly increase SHM upon infection (FIG. 9A). Indeed, toddlers had a higher pre-malaria SHM level compared to infants (FIG. 9A). Surprisingly, infants were able to induce more SHMs compared to toddlers (FIG. 9B). These data suggested that indeed both infants and toddlers induce SHMs upon malaria infection.

Memory B Cells Further Diversify Upon Malaria Rechallenge:

The importance of IgM-expressing memory B cells has been reported in mice in several studies (Kaji et al., 2012), including a mouse model of malaria infection. However, fewer studies have examined these cells in humans, and their composition and role in repertoire diversification upon rechallenge remains elusive. It is widely believed that they may retain the capacity to introduce further mutations and class switch. However, sequence-based clonal lineage evidence is lacking. The paired samples before and during acute malaria from toddlers who experienced malaria in previous years provided an opportunity to investigate the role of memory B cells in repertoire diversification upon rechallenge in children.

Here, two-timepoint-shared lineages were focused on that harbor sequences from pre-malaria memory B cells. Given the significant increase of SHM we identified at acute malaria sequences over pre-malaria sequences in two-timepoint-shared lineages (FIG. 9A), it was reasoned that the high repertoire coverage of MIDCIRS should enable us to identify a large number of two-timepoint-shared lineages that contain these memory B cells, and these memory B cells should have mutated progenies at the acute malaria timepoint. To ensure that sequence progenies of these pre-malaria memory B cells were identified, an antibody lineage structure construction algorithm was employed, COLT (Chen et al., 2016). COLT considers isotype, sampling time, and SHM pattern when constructing an antibody lineage, which allows tracing, at the sequence level, the acute progeny of these memory B cells. As illustrated by FIG. 24, this COLT-generated lineage tree depicts a pre-malaria memory B cell sequence serving as a parent node to sequences derived from the acute malaria timepoint. This analysis is much more stringent in identifying sequence progenies than simply judging if a pre-malaria memory B cell sequence is grouped with acute malaria PBMC sequences.

On average, 5% of unique sequences from 10,000 sorted memory B cells form lineages with acute malaria PBMC sequences (FIG. 9C, dark slice of the first pie). COLT analysis on these pre-malaria memory B cell-containing lineages shows that 53% contain traceable progeny sequences from the acute malaria PBMCs (FIG. 9C, lighter slice of the second pie). Overall, there is a significant increase of SHM in these acute malaria progenies compared with their ancestor pre-malaria memory B cells (FIG. 9D). These progeny-bearing pre-malaria memory B cells express all three major isotypes, with IgM being the dominant species (FIG. 9E). Investigating their isotype switching capacity reveals that about 60% of the IgM pre-malaria memory B cells maintain IgM as progenies; however, about 20% only have isotype-switched progenies detected while the remaining 20% have both IgM and isotype switched progenies (FIG. 9F). These pre-malaria IgM memory B cells largely retain IgM expression while further introducing SHM upon rechallenge. Thus, these analyses show multi-facet diversification potential of young children's memory B cells in a natural infection rechallenge.

### Example 4—Materials and Methods

Cohort: Human PBMCs for method validation were purified from de-identified blood bank donor samples. This protocol was approved by the Institutional Review Board of the University of Texas at Austin as non-human subject research.

Infant and toddler PMBC samples from 19 residents of Kalifabougou, Mali, ranging from 3 months old to 42 months old, were collected from a much bigger ongoing malaria cohort study₁ and analyzed as summarized in Table 4. Enrollment exclusion criteria were hemoglobin level <7 g/dL, axillary temperature ≥37.5° C., acute systemic illness, use of antimalarial or immunosuppressive medications in the past 30 days, and pregnancy. The research definition of malaria was an axillary temperature of ≥37.5° C., ≥2500 asexual parasites/μL of blood, and no other cause of fever discernible by physical exam. The Ethics Committee of the Faculty of Medicine, Pharmacy, and Dentistry at the University of Sciences, Technique, and Technology of Bamako, and the Institutional Review Board of the National Institute of Allergy and Infectious Diseases, National Institutes of Health, approved the malaria study, from which we obtained frozen PBMCs. Written informed consent was obtained from adult participants and from the parents or guardians of participating children. The study is registered in the ClinicalTrials.gov database (NCT01322581).

For this study, subjects were chosen based on the availability of frozen PBMCs in the age range specified. Blood draws were taken before the rainy season, when mosquitos are not rampant and the cases of malaria are low, and during acute febrile malaria. Patients were labeled for analysis by the age, in months, at the time of the preseason blood draw. Multiple patients of the same age were distinguished by the suffixes “A”, “B”, “C”, and “D,” when applicable. Samples collected before the beginning of the rainy season that tested PCR negative for Plasmodium falciparum and Plasmodium malariae were designated “pre-malaria”. Samples collected 7 days into acute febrile malaria infection were designated “acute malaria”. Among them, 2 subjects were tracked for 2 consecutive years, 5 subjects did not have acute febrile malaria for the first year, 1 subject withdrew from the study, and 1 subject's acute malaria sample was committed to alternate projects and thus were not available for this study as indicated by the different footnotes in Table 3. Some samples had insufficient cells for FACS sorting, as indicated by I.S. in Table 3. Authors were not blinded to neither the age group allocation nor the sample collection time.

TABLE 3
Sequencing read statistics for control libraries.

| | | | | | | | | Number of |
|---|---|---|---|---|---|---|---|---|
| | | | | | Number | | Percentage | useful MIDs |
| | | Number | Number | Number | of reads | Number | of Reads | containing |
| | Number | of raw | of merged | of Ig | truncated | of useful | in useful | more than one |
| Library | of cells | reads | reads | reads | to 320bp | MIDs^{a} | MIDs | sub-group^{b} |
| Libraries | 1,000 | 46,320 | 22,742 | 9,201 | 9,149 | 797 | 94.30 | 1 |
| for naive B | 2,000 | 44,846 | 18,602 | 17,421 | 17,267 | 2,176 | 93.29 | 2 |
| cells from | 10,000 | 228,711 | 99,370 | 62,242 | 61,121 | 7,102 | 94.73 | 9 |
| healthy | 20,000 | 293,279 | 196,570 | 184,754 | 182,818 | 23,991 | 93.27 | 49 |
| controls | 100,000 | 1,153,763 | 1,074,771 | 1,048,523 | 1,041,048 | 165,663 | 92.63 | 1,137 |
| | 200,000 | 2,191,738 | 2,107,762 | 2,059,944 | 2,045,047 | 404,225 | 91.41 | 7,239 |
| | 1,000,000 | 7,494,809 | 7,342,163 | 7,258,253 | 7,207,962 | 1,516,098 | 86.44 | 108,172 |

^{a}A useful MID has more than two reads. If there are only two reads in a MID, they are discarded unless they are identical.
^{b}The number of MIDs containing more than one type of antibody heavy chain transcripts.

TABLE 5
Cohort and Cell Type Availability

| | Pre-malaria | | | | | Acute malaria | | | |
|---|---|---|---|---|---|---|---|---|---|
| Patient | Pre-Index | Pre-Age | | PBMC | Memory B | Acute-Index | Acute Age | | PBMC |
| Inf1 | Inf1-Pre3 m | 3 | m | Yes | I.S. | Inf1-Acu9 m | 9 | m | Yes |
| Inf2 | Inf2-Pre3 m | 3 | m | Yes | J.F. | Inf2-Acu6 m | 6 | m | Yes |
| Inf3 | Inf3-Pre5 m | 3 | m | Yes | I.S. | Inf3-Acu11 m | 11 | m | Yes |
| Inf4 | Inf4-Pre5 m | 5 | m | Yes | J.F. | Inf4-Acu10 m | 10 | m | Yes |
| Inf5* | Inf5-Pre5 m | 5 | m | Yes | J.F. | Inf5-Acu10 m | 10 | m | Yes |
| Inf6 | Inf6-Pre8 m | 8 | m | Yes | J.F. | Inf6-Acu12 m | 12 | m | Yes |
| Inf7 | Inf7-Pre11 m | 11 | m | Yes | Yes | N.A. | N.A. | | N.A. |
| Inf8 | Inf8-Pre11 m | 11 | m | Yes | Yes | N.A. | N.A. | | N.A. |
| Inf9 | Inf9-Pre11 m | 11 | m | Yes | Yes | N.A. | N.A. | | N.A. |
| Inf10 | Inf10-Pre11 m | 11 | m | Yes | Yes | N.A. | N.A. | | N.A. |
| Inf11 | Inf11-Pre11 m | 11 | m | Yes | Yes | N.A. | N.A. | | N.A. |
| Tod1* | Tod1-Pre17 m | 17 | m | Yes | Yes | Tod1-Acu22 m | 22 | m | Yes |
| Tod2 | Tod2-Pre19 m | 19 | m | Yes | Yes | Tod2-Acu22 m | 22 | m | Yes |
| Tod3† | Tod3-Pre28 m | 28 | m | Yes | Yes | Tod3-Acu32 m | 32 | m | Yes |
| Tod4 | Tod4-Pre29 m | 29 | m | Yes | Yes | Tod4-Acu32 m | 32 | m | Yes |
| Tod5 | Tod5-Pre31 m | 31 | m | Yes | J.F. | Tod5-Acu32 m | 32 | m | Yes |
| Tod6 | Tod6-Pre31 m | 31 | m | Yes | Yes | Tod6-Acu38 m | 38 | m | Yes |
| Tod7† | Tod7-Pre40 m | 40 | m | Yes | Yes | Tod7-Acu42 m | 42 | m | Yes |
| Tod8 | Tod8-Pre42 m | 42 | m | Yes | Yes | Tod8-Acu46 m | 46 | m | Yes |
| Tod9 | Tod9-Pre47 m | 47 | m | Yes | Yes | Tod9-Acu50 m | 50 | m | Yes |

| | | | | | | | | |
|---|---|---|---|---|---|---|---|---|
| Tod10 | Tod10-Pre13 m | 13 | m | Yes | Yes | N.A. | N.A. | N.A. |
| Tod11 | Tod11-Pre16 m | 16 | m | Yes | Yes | N.A. | N.A. | N.A. |
| Tod12 | Tod12-Pre17 m | 17 | m | Yes | Yes | N.A. | N.A. | N.A. |
| Tod13 | Tod13-Pre17 m | 17 | m | Yes | Yes | N.A. | N.A. | N.A. |

I.S. indicates insufficient cells for FACS sorting.
W.D. indicates withdraw from the study
N.F.M indicates no incidence of febrile malaria in that year N.A indicates samples were not available.
*same individual
†same individual

Cell Sorting:

Naïve B cells (NBCs) were FACS sorted based on the phenotype of CD3−CD19+CD20+CD27−CD38−. For malaria samples, up to 5,000,000 PBMCs were lysed directly. From the remaining PBMCs, up to 2,000 plasmablasts (PBs) were FACS sorted based on the phenotype of CD4−CD8−CD14−CD56−CD19+CD27_{bright}CD38_{bright}, and up to 10,000 memory B cells (MBCs) were sorted based on the phenotype of CD4−CD8−CD14−CD56−CD19+CD27+CD38lo. Cells were lysed in RLT Plus buffer (Qiagen) supplemented with 1% β-mercaptoethanol (Sigma). The following antibody clones were obtained from Biolegend: OKT3 (CD3), RPA-T4 (CD4), HCD14 (CD14), 2H7 (CD20), O323 (CD27), HIT2 (CD38), MEM-188 (CD56). The following antibody clones were obtained from BD Biosciences: RPA-T8 (CD8) and SJ25C1 (CD19).

Bulk Antibody Sequencing Library Generation and Sequencing:

MIDs were added during the reverse transcription step through the use of fusion primers, which contain the partial Illumina P5 sequencing adaptor followed by twelve random nucleotides and primers to the constant region of five antibody isotypes. Eleven leader region primers were fused to partial Illumina P7 adaptor. Full Illumina adaptors were added during the second PCR step along with library indexes. Total RNA was purified using All Prep DNA/RNA kit (Qiagen) following the manufacturer's protocol. cDNA synthesis was done using Superscript III (Life Technologies). After free primer removal, Takara Ex Taq HS polymerase (clone Tech) was used for both PCR reactions. The first PCR was performed with the following program: initial denature at 95° C. for 3 minutes, followed by 20 cycles of 95° C. for 30 seconds, 57° C. for 30 seconds, and finally 72° C. for 2 minutes with a 4° C. hold. The second PCR was performed with the following program: initial denature at 95° C. for 3 minutes, followed by 10 cycles of 95° C. for 30 seconds, 57° C. for 30 seconds, and finally 72° C. for 2 minutes with a 4° C. hold. Libraries were gel purified and quantified by qPCR Library Quantification Kit (KAPA biosystems) and sequenced on Illumina Mi-seq with paired-end 250 bp read. The list of primers for RT and PCR can be found in Table 1. All sequencing reads were generated on Illumina Mi-seq using 2×250 bp mode. Libraries were sequenced multiple times until saturated based on rarefaction analysis in FIG. 11. Reads from all runs were combined and analyzed.

Preliminary Read Processing:

Raw reads from Illumina MiSeq PE250 were first cleaned up following steps outlines in FIG. 1. Only reads that exactly matched the corresponding library indices were included for further processing. The end of each raw read was trimmed such that all bases had a quality score of 25 or higher. Reads 1 and 2 were merged using the SeqPrep tool. The merged reads were filtered with specific V-gene and constant region primers to determine immunoglobulin (Ig) sequencing reads. The primers were then truncated from the reads. The retained reads were further truncated to 320 bp for the NBCs in method verification experiments and 330 bp for samples from malaria cohort. Read numbers after each filter are listed in Table 2 and 4.

TABLE 5
Sequencing read statistics of PBMCs from malaria cohort.

| | | | | | Unique |
|---|---|---|---|---|---|
| | | | Mapped | Percent | RNA |
| Sample | PBMCs^{a} | Raw reads | reads | Mapped | molecules |
| Inf1-Pre3m | 3,000,000 | 3,246,180 | 2,989,252 | 92.1% | 41,842 |
| Inf1-Acu9m | 3,000,000 | 3,608,436 | 3,348,589 | 92.8% | 32,800 |
| Inf2-Pre3m | 3,000,000 | 3,176,623 | 2,987,587 | 94.0% | 35,379 |
| Inf2-Acu6m | 3,000,000 | 3,689,115 | 3,481,675 | 94.4% | 29,523 |
| Inf3-Pre5m | 4,150,000 | 3,242,619 | 3,070,458 | 94.7% | 37,234 |
| Inf3-Acu11m | 5,000,000 | 4,396,739 | 4,153,830 | 94.5% | 42,634 |
| Inf4-Pre5m | 5,000,000 | 3,048,762 | 2,810,018 | 92.2% | 45,445 |
| Inf4-Acu10m | 3,700,000 | 5,287,767 | 4,864,629 | 92.0% | 29,694 |
| Inf5-Pre5m* | 5,000,000 | 3,764,663 | 3,425,015 | 91.0% | 54,516 |
| Inf5-Acu10m* | 50,00,000 | 4,712,120 | 4,374,600 | 92.8% | 41,774 |
| Inf6-Pre8m | 5,000,000 | 3,588,177 | 3,456,165 | 96.3% | 47,254 |
| Inf6-Acu12m | 400,000 | 395,765 | 378,182 | 95.6% | 03,447 |
| Tod1-Pre17m* | 5,000,000 | 2,816,309 | 2,576,372 | 91.5% | 53,551 |
| Todl-Acu22m* | 1,380,000 | 2,811,617 | 2,593,849 | 92.3% | 12,514 |
| Tod2-Pre19m | 5,000,000 | 4,842,338 | 4,673,875 | 96.5% | 40,600 |
| Tod2-Acu22m | 1,920,000 | 1,956,906 | 1,886,521 | 96.4% | 15,285 |
| Tod3-Pre28m† | 5,000,000 | 3,988,677 | 3,687,883 | 92.5% | 35,567 |
| Tod3-Acu32m† | 5,000,000 | 9,218,255 | 8,565,149 | 92.9% | 47,144 |
| Tod4-Pre29m | 5,000,000 | 2,924,629 | 2,851,964 | 97.5% | 48,950 |
| Tod4-Acu32m | 5,000,000 | 4,004,416 | 3,846,197 | 96.0% | 40,628 |
| Tod5-Pre31m | 5,000,000 | 5,338,867 | 5,126,888 | 96.0% | 31,531 |
| Tod5-Acu32m | 3,000,000 | 2,853,984 | 2,736,902 | 95.9% | 26,955 |
| Tod6-Pre31m | 5,000,000 | 4,356,975 | 4,198,929 | 96.4% | 44,665 |
| Tod6-Acu38m | 2,170,000 | 5,738,001 | 5,460,964 | 95.2% | 22,270 |
| Tod7-Pre40m† | 5,000,000 | 3,192,503 | 2,893,482 | 90.6% | 34,901 |
| Tod7-Acu42m† | 4,740,000 | 4,448,008 | 4,079,432 | 91.7% | 34,185 |
| Tod8-Pre42m | 5,000,000 | 2,120,127 | 2,058,164 | 97.1% | 48,939 |
| Tod8-Acu46m | 2,100,000 | 2,060,234 | 1,986,239 | 96.4% | 17,039 |
| Tod9-Pre47m | 3,000,000 | 3,035,618 | 2,682,991 | 88.4% | 20,094 |
| Tod9-Acu50m | 3,000,000 | 4,678,879 | 3,912,981 | 83.6% | 18,447 |

^{a}Number of PBMCs differs because of the age dependent blood draw volume and cell recovery.
*Same individual
†Same individual

MID Sub-Group Generating:

Raw reads were split into MID groups according to their 12 nucleotide barcodes. For each MID group, quality threshold clustering was used to cluster similar reads. This process groups reads derived from a common template RNA molecule together while separating reads derived from distinct RNA molecules. A Levenshtein distance of 15% of the read length was used as the threshold. This was calibrated using RNA controls with known sequences (FIG. 9). For each sub-group, a consensus sequence was built based on the average nucleotide at each position, weighted by the quality score. In the case that there were only two reads in an MID sub-group, reads were only considered useful if both were identical. Each MID sub-group is equivalent to an RNA molecule. Next, all of the identical consensus were merged to form unique consensus sequences, or unique RNA molecules, which were used to estimate the diversity and assess the sequencing depth in rarefaction analysis (FIG. 4C, D and 11).

VDJ Definition and Mutation Counts:

As described in previous work, similar methods were used to define the V, D, and J gene segments for all sequences. From the International ImMunoGeneTics information system database (IMGT), human heavy chain variable gene segment sequences (249 V-exon, 37 D-exon and 13 J-exon) were downloaded. Each unique sequence was first aligned to all 249 V gene allele. The specific V-allele with a maximum Smith-Waterman score was then assigned. In some cases, newly identified germline alleles, defined either by TIgGER, our method (below), or the combination of the two, were added to the template sequences. J-segments and D-segments were then similarly assigned. The number of mutations from germline sequence was counted as the number of substitutions from the best aligned V and J templates. The CDR3 was omitted due to the difficulty in determining the germline sequence. The germline sequences of V, D, and J gene segments were grouped by combining similar alleles into families using IMGT designation in VDJ correlation plots. In total, 58 V, 27 D, and 6 J families were obtained.

Novel Allele Detection:

To address the possibility of novel germline alleles inflating the observed number of mutations, new germline alleles were assembled. In short, IgM sequences for each subject were aligned and assigned to the traditional V-gene alleles in the IMGT database. If novel alleles exist in subjects, parts of unique RNA sequences will be assigned as mutations when they are actually derived from differences between novel and traditional alleles. The ratios of unmutated unique RNA molecules to those with one, two, three and four mutations compared to the IMGT germline were determined, and if any were found to be less than 2 to 1, the alleles were flagged for further inspection. Unique RNA molecules were used to minimize the contributions of clonal expansion, and IgM sequences were used to minimize the contributions of somatic hypermutation. Sequences within flagged alleles were then aligned to the closest IMGT germline to determine if the mutations are truly polymorphisms. When identical mutation patterns were observed in a minimum of 80% of all sequences in a flagged allele family, it was deemed a novel germline allele. For subjects with sorted NBCs, novel alleles were generated from the NBC BCR sequences to complement those found in the bulk IgM sequences.

TIgGER was used as previously reported as another method to discover novel alleles₅. TIgGER compares the mutation rate at a specific position to the overall number of mutations for sequences within the same assigned V-gene allele. Outliers within the low mutation region suggests the existence of a novel allele, and the shape of the curve can effectively distinguish between individuals homozygous and heterozygous for the novel allele.

The MIDCRS method and TIgGER have an 89% percent overlap in newly identified alleles. Discrepancies between the two methods were treated with a conservative estimation on the number of SHM, meaning novel alleles were liberally included. Non-overlapping novel alleles were manually inspected, and the union of novel alleles detected by TIgGER and the current method was included in mutation analysis shown in the main figures, whereas results using novel alleles detected only by TIgGER were shown in the supplementary information.

Translation from Nucleotide to Amino Acid Sequences:

Nucleotide sequences were translated into amino acid sequences based on codon translation. The unique RNA sequences were inputted to IMGT High V quest to translate into amino acid sequences. The boundary of the CDR3 is defined by IMGT numbering for Ig and two conserved sequence markers of ‘Tyr-(Tyr/Phe)-Cys’ to ‘Trp-Gly.’ CDR3 length was determined according to these anchor residues.

TABLE 6
The percentage of unique RNA sequences assigned to
the novel alleles for each sample. Novel alleles
detected by TIgGER and our method were combined.

| | Percentage of Unique RNA sequences |
|---|---|
| Sample | assigned to novel germline alleles |
| Inf1-Pre3m | 4.81% |
| Inf1-Acu9m | 6.21% |
| Inf2-Pre3m | 8.44% |
| Inf2-Acu6m | 9.11% |
| Inf3-Pre5m | 1.78% |
| Inf3-Acu11m | 4.91% |
| Inf4-Pre5m | 11.83% |
| Inf4-Acu10m | 9.63% |
| Inf5-Pre5m* | 8.19% |
| Inf5-Acu10m* | 7.72% |
| Inf6-Pre8m | 6.02% |
| Inf6-Acu12m | 6.79% |
| Tod1-Pre17m* | 9.82% |
| Tod1-Acu22m* | 7.51% |
| Tod2-Pre19m | 2.54% |
| Tod2-Acu22m | 2.34% |
| Tod3-Pre28m† | 16.91% |
| Tod3-Acu32m† | 15.05% |
| Tod4-Pre29m | 3.61% |
| Tod4-Acu32m | 4.80% |
| Tod5-Pre31m | 6.98% |
| Tod5-Acu32m | 6.79% |
| Tod6-Pre31m | 5.89% |
| Tod6-Acu38m | 4.15% |
| Tod7-Pre40m† | 18.30% |
| Tod7-Acu42m† | 13.84% |
| Tod8-Pre42m | 7.40% |
| Tod8-Acu46m | 5.71% |
| Tod9-Pre47m | 13.10% |
| Tod9-Acu50m | 13.15% |

*Same individual
†Same individual

TABLE 7
Average mutation number of NBCs.

| | | Average number |
|---|---|---|
| Subject | Number of NaiBs | of mutations |
| Inf1-Acu9m | 10000 | 0.31 |
| Inf2-Pre3m | 10000 | 0.20 |
| Inf4-Pre5m | 10000 | 0.29 |
| Inf5-Pre5m | 10000 | 0.27 |
| Inf6-Pre5m* | 10000 | 0.40 |
| Inf6-Acu10m* | 100000 | 1.03 |
| Inf9-Pre11m | 10000 | 0.36 |
| Inf10-Pre11m | 10000 | 0.31 |
| Inf11-Pre11m | 10000 | 0.33 |
| Inf12-Pre11m | 10000 | 0.94 |
| Tod2-Pre16m | 10000 | 0.43 |
| Tod3-Pre17m* | 10000 | 0.79 |
| Tod3-Acu22m* | 10000 | 1.41 |
| Tod4-Pre17m | 10000 | 0.85 |
| Tod6-Pre19m | 10000 | 0.57 |
| Tod7-Pre28m† | 10000 | 0.53 |
| Tod7-Acu32m† | 100000 | 1.05 |
| Tod8-Pre29m | 100000 | 1.07 |
| Tod11-Pre40m† | 10000 | 0.45 |
| Tod11-Acu42m† | 100000 | 1.17 |
| Tod13-Pre42m | 100000 | 1.20 |

*Same individual
†Same individual

TABLE 8
Nucleotide mutations resulting in amino acid substitutions (Replacement, R) or no amino acid
substitutions (silent, S) in the framework region (FWR2 and 3) and complementary determining
regions (CDR1 and 2) of infants (N = 6) and toddlers (N = 9), weighted by unique
RNA molecules. CDR3 and FWR4 were not included in this analysis due to the difficulty determining
the germline sequence. FWR1 for all sequences was also omitted because it was not covered
entirely by some of the primers. Average displayed as mean ± standard deviation.

| | | | FWR | | | CDR | | | Average R/S Ratio | |
|---|---|---|---|---|---|---|---|---|---|---|
| | | | R | S | R/S Ratio | R | S | R/S Ratio | FWR | CDR |
| Infant | Pre | IgM | 0.54 | 0.11 | 4.98 | 0.18 | 0.04 | 5.15 | 3.00 ± 1.12 | 5.54 ± 0.25 |
| | | IgG | 1.54 | 0.70 | 2.21 | 1.36 | 0.24 | 5.67 | | |
| | | IgA | 1.48 | 0.65 | 2.28 | 1.29 | 0.22 | 5.75 | | |
| | Acute | IgM | 1.36 | 0.34 | 4.05 | 0.58 | 0.11 | 5.52 | | |
| | | IgG | 1.88 | 0.85 | 2.22 | 1.62 | 0.30 | 5.35 | | |
| | | IgA | 2.03 | 0.90 | 2.25 | 1.75 | 0.30 | 5.79 | | |
| Toddler | Pre | IgM | 1.12 | 0.35 | 3.20 | 0.58 | 0.11 | 5.54 | 2.41 ± 0.45 | 5.34 ± 0.25 |
| | | IgG | 3.42 | 1.57 | 2.17 | 2.73 | 0.54 | 5.05 | | |
| | | IgA | 3.88 | 1.82 | 2.14 | 3.15 | 0.58 | 5.41 | | |
| | Acute | IgM | 2.16 | 0.79 | 2.73 | 1.33 | 0.24 | 5.44 | | |
| | | IgG | 4.28 | 2.02 | 2.11 | 3.39 | 0.68 | 5.02 | | |
| | | IgA | 4.33 | 2.04 | 2.12 | 3.55 | 0.64 | 5.59 | | |

N.D. indicates not detected
* Same individual
† Same individual

TABLE 9
Pre-malaria and acute malaria shared lineage count.

| | Shared | Unique memory | Containing pre-malaria |
|---|---|---|---|
| Patient | lineages | B cell Sequences | memory B cells |
| Inf1 | 29 | N.A. | N.A. |
| Inf2 | 131 | N.A. | N.A. |
| Inf3 | 215 | N.A. | N.A. |
| Inf4 | 142 | N.A. | N.A. |
| Inf5 | 214 | N.A. | N.A. |
| Inf6 | 83 | N.A. | N.A. |
| Tod1 | 308 | 3,423 | 149 |
| Tod2 | 385 | 7,856 | 145 |
| Tod3† | 1230 | 6,023 | 926 |
| Tod4 | 1194 | 5,073 | 209 |
| Tod5 | 260 | N.A. | N.A. |
| Tod6 | 346 | 6,363 | 111 |
| Tod7† | 472 | 4,771 | 161 |
| Tod8 | 581 | 2,399 | 98 |
| Tod9 | 414 | 2,534 | 135 |

The number of lineages containing sequences from both the pre-malaria and acute malaria timepoints. For malaria-experienced individuals with 10,000 FACS sorted pre-malaria memory B cells available, the number of unique memory B cell sequences and two-timepoint-shared lineages that contain sequences from the sorted memory B cells from the pre-malaria timepoint.
N.A. indicates not applicable
†Same individual

Selection Pressure:

The selection pressure was evaluated via BASELINe. The unique RNA molecules of PBMC, MBC and PB populations were inputted to BASELINe and compared with the closest IMGT germline alleles. The observed number of replacement and silent mutations were compared with the expected number of mutations for the assigned germline sequence. A selection strength value (Σ) and associated P value were generated by BASELINe to indicate the direction, degree, and confidence of selection pressure for CDR (CDR1 and 2) and FR (FR1, 2, and 3) regions for each unique RNA molecule. Selection strength on CDR and FR for unique RNA molecules were binned as a bin-size of 0.05, and percentage of unique RNA molecules falling into each bin was plotted as a selection strength distribution. This distribution was plotted and compared between infants and toddlers and IgM vs IgG+IgA for MBCs and PBs (FIG. 24).

Replacement/Silent Mutation:

According to the amino acid sequence translation results and V/D/J gene templates alignment results, the number of nucleotide mutations resulting in amino acid substitutions (replacement, R) or no amino acid substitutions (silent, S) in FR region (FR1, FR2, and FR3) and CDR region (CDR1 and CDR2) were counted. The number of silent and replacement mutations was averaged in each age-group (Infant and Toddler) and the ratio for silent vs. replacement mutation was calculated. The CDR3 and FR4 were omitted due to the difficulty in determining the germline sequence.

VDJ Usage Correlation:

The correlation of VDJ usage between infants and toddlers were calculated with Pearson Correlation Coefficient as the following formula:

$$corr = \frac{{\sum }_{v = \{ V \} , d = \{ D \} , j = \{ J \}} ( {X}_{vdj} - 〈 X 〉 ) ( {Y}_{vdj} - 〈 Y 〉 )}{\sqrt{{\sum }_{v = \{ V \} , d = \{ D \} , j = \{ J \}} {( {X}_{vdj} - 〈 X 〉 )}^{2} * {\sum }_{v = \{ V \} , d = \{ D \} , j = \{ J \}} {( {Y}_{vdj} - 〈 Y 〉 )}^{2}}}$$

vdj refers to the combination of one v allele family from 58 V gene allele families ({V}), one d allele family from 27 D gene allele families ({D}), and one j allele family from 6 J gene allele families ({J}). For the reads weighted correlation, X_{vdj} and Y_{vdj} refer to the fraction of reads assigned to the respective vdj combination for subjects X and Y, respectively. <X> and <Y> are the average reads across all vdj combinations, i.e. 1/9396, where 9396 is the total possible number of vdj allele family combinations. For the lineage weighted correlation, these parameters refer to the fraction of lineages for each vdj allele family combination.

Clustering Sequences into Clonal Lineages:

Sequences with similar CDR3 are possibly progenies from the same NBC and can be grouped into a clonal lineage. To detect the lineage structure for the antibody repertoire, single linkage clustering was performed, using a re-parameterization of the method described in Jiang et al., 2011, accounting for the larger size of the CDR3 and junction in humans as compared to zebrafish. RNA sequences with the same V and J allele assignments, the same CDR3 length, and whose CDR3 regions differed by no more than 20% on the nucleotide level were grouped together into a lineage. This is equivalent to a biological clone that underwent clonal expansion. In order to test the robustness of this threshold, we also tried the threshold of 90% similarity for CDR3 region, and it did not change the overall position of each lineage in the diversity-size plot (FIG. 22). Lineage diversity is the number of unique RNA molecules within the lineage, and lineage size is the total number of RNA molecules within the lineage.

Clonal Lineage Diversification:

In order to discuss the clonal lineage diversification, the size and diversity, as described above, were plotted against each other for pre- and acute malaria time points for each patient. The linear regression visualizes the average degree of diversification relative to clonal expansion. A characteristic shift towards further diversification of clonal lineages upon acute malaria infection was evaluated by the decrease in the slope of the linear regression for each infant and toddler. The shift was calculated by the difference between the arctangents of the slopes of the linear regressions. There was no significance difference in the angular shift towards diversification between the infants and toddlers, as determined by two-tailed t-test.

Lineage Structure Visualization:

Representative lineages were selected to visualize the lineage structures and the evolution of antibody sequences. The phylogenic tree was generated by MEGA software with Minimum-Evolution method using 330 bp truncated sequences first, then validated using the full length sequences in each lineage and verified manually. According to the phylogenic information, tree-style lineage structures were generated and visualized by Python Package NetworkX. Each node in the tree indicates one unique RNA molecule in the lineage. The distance between two nodes is correlated to the difference between two unique RNA sequences.

Two-Timepoint-Shared Lineage Analysis:

To test the effects of acute malaria infection on the structure of clonal lineages, RNA molecules from both the pre- and acute malaria timepoints were grouped together and subjected to clustering into clonal lineages as described above. Resulting lineages that contained sequences from both the pre-malaria and acute malaria timepoints were isolated for mutational analysis. Within these shared lineages, the average number of mutations for the pre-malaria sequences was calculated alongside the average number of mutations for the acute malaria sequences (FIG. 9A).

Lineage Structure Visualization:

Representative lineages were selected to visualize the lineage structures and the evolution of antibody sequences. Lineage structures were generated using COLT and validated manually. A lineage visualization tool, COLT-Viz, was implemented. In short, COLT considers constraints (e.g., isotype and timepoint) along with mutational patterns to build lineage trees. The height of each node is proportional to the number of RNA molecules associated with the unique sequence (size), the color of each node relates to the number of SHMs, and the distance between nodes is proportional to the Levenshtein distance between the node sequences.

Pre-Malaria Memory B Cells with Acute Progeny Lineage Analysis:

To determine the fate of the pre-malaria memory B cells upon acute malaria infection, two-timepoint-shared lineages were formed as described above, and lineages containing sequences from both FACS-sorted pre-malaria memory B cells and acute malaria PBMCs were isolated for further analysis. COLT was used to generate lineage tree structures. Pre-malaria memory B cells that served as parent nodes to acute malaria sequences, as exemplified (FIG. 24), were considered “pre-malaria memory B cells with acute progeny” (FIG. 9C-F).

### Example 5—MIDCIRS for Clonality Diversity and Clone Size Quantification

MIDCIRS Sub-Clustering Improves Repertoire Diversity Estimation Accuracy:

Metrics were developed to validate the accuracy of the MIDCIRS sub-clustering method. In addition, the present studies demonstrate the robust ability of MIDCIRS to faithfully represent the diversity and abundance of the TCR repertoire using a large range of RNA inputs.

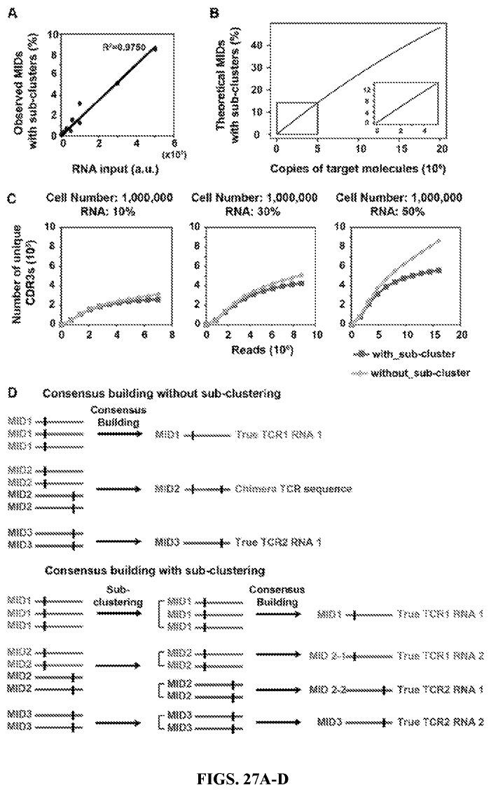

It was reasoned that in order to comprehensively quantify the overall diversity, a large portion of its RNA must be sampled. However, this will inevitably increase the number of TCR transcripts that need to be tagged with MIDs, which increases the portion of MIDs tagging multiple TCR transcripts. It was sought to closely examine the relationship between RNA input and multiple TCR RNA tagging by the same MID. The process of MID labeling can be modeled as a Poisson distribution. The percentage of MIDs with sub-clusters follows an approximate linear trend when the copies of target RNA molecules are less than 5,000,000 (FIG. 27B). To experimentally validate this, MIDCIRS TCR-seq was applied on a range of sorted naïve CD8⁺ T cells (from 20,000 to 1 million) with three different RNA inputs (10%, 30% and 50%) (Table 10). As expected, it was found that the observed percentage of MIDs that need sub-clustering is approximately linear with respect to copies of target RNA molecules used in this study (FIG. 27A). With the highest amount of RNA molecules used in this study, approximately 8.5% of MIDs require further clustering. Thus, MIDCIRS sub-clustering significantly improves repertoire diversity coverage.

TABLE 10
Spike-in Jurkat TCR RNA detection in naïve CD8⁺ T cells.
10 TCR-copy worth of Jurkat RNA was added to each sample during
the reverse transcription step. Number of MIDs for RNA molecules
that are tagged with Jurkat TCR sequences were counted.

| Sample | Jurkat TCR copies detected |
|---|---|
| 20,000Tn_10% RNA | 7 |
| 20,000Tn_30% RNA | 0 |
| 20,000Tn_50% RNA | 1 |
| 100,000Tn_10% RNA | 5 |
| 100,000Tn_30% RNA | 4 |
| 100,000Tn_50% RNA | 1 |
| 200,000Tn_10% RNA | 7 |
| 200,000Tn_30% RNA | 3 |
| 200,000Tn_50% RNA | 3 |
| 1,000,000Tn_10% RNA | 4 |
| 1,000,000Tn_30% RNA | 8 |
| 1,000,000Tn_50% RNA | 17 |

To evaluate the accuracy of the sub-clustering step by an alternative means, the TCR sequence lengths were examined within MIDs that contain sub-clusters. It was reasoned that if indeed each TCR RNA molecule was tagged with a unique MID, then the lengths of complementarity-determining region 3 (CDR3) for all reads would be identical under each MID. However, it was shown that of the 8.5% of MIDs that contain sub-clusters, about 87% of MIDs contain TCR sequencing reads of different CDR3 lengths while only 13% have the same length for one million naïve CD8⁺ T cells (50% RNA input). After performing sub-clustering, over 97% of sub-clusters have a uniform length (FIG. 31), demonstrating the accuracy of sub-clustering step in MIDCIRS.

TABLE 11
Metrics of sequencing results of first naïve CD8⁺ T cell experiment.

| | | | | | | Percentage | | | Top |
|---|---|---|---|---|---|---|---|---|---|
| | | | | | | of MIDs | Percentage | | CDR3 |
| | | | Map | Total | Unique | with sub- | of chimera | Top | molecule |
| | Raw | Mappable | percentage | RNA | productive | clusters | sequences | CDR3 | fraction |
| Sample | reads | reads | (%) | molecules | CDR3 | (%) | (%) | molecules * | (%) |
| 20,000 Tn | 402975 | 254228 | 63.09 | 10171 | 4579 | 0.11 | 0.32 | 24 | 0.24 |
| 10% RNA | | | | | | | | | |
| 20,000 Tn | 877556 | 698961 | 79.65 | 18670 | 7253 | 0.34 | 0.42 | 39 | 0.21 |
| 30% RNA | | | | | | | | | |
| 20,000 Tn | 1188083 | 984951 | 82.90 | 18367 | 7495 | 0.32 | 0.70 | 30 | 0.16 |
| 50% RNA | | | | | | | | | |
| 100,000 Tn | 922615 | 766441 | 83.07 | 36949 | 17632 | 0.28 | 0.33 | 89 | 0.24 |
| 10% RNA | | | | | | | | | |
| 100,000 Tn | 2409732 | 2173270 | 90.19 | 72257 | 30428 | 0.70 | 1.58 | 245 | 0.34 |
| 30% RNA | | | | | | | | | |
| 100,000 Tn | 1744861 | 1566048 | 89.75 | 55058 | 27280 | 0.52 | 0.99 | 171 | 0.31 |
| 50% RNA | | | | | | | | | |
| 200,000 Tn | 1000937 | 788947 | 78.82 | 61525 | 34097 | 0.41 | 0.86 | 166 | 0.27 |
| 10% RNA | | | | | | | | | |
| 200,000 Tn | 4224183 | 3902130 | 92.38 | 173224 | 66990 | 1.57 | 5.44 | 498 | 0.29 |
| 30% RNA | | | | | | | | | |
| 200,000 Tn | 3147293 | 2889513 | 91.81 | 154666 | 67607 | 1.28 | 2.64 | 628 | 0.41 |
| 50% RNA | | | | | | | | | |
| 1,000,000 Tn | 7695858 | 6975703 | 90.64 | 514916 | 237331 | 3.19 | 16.14 | 1430 | 0.28 |
| 10% RNA | | | | | | | | | |
| 1,000,000 Tn | 9439612 | 8719649 | 92.37 | 942010 | 382743 | 5.18 | 17.02 | 2387 | 0.25 |
| 30% RNA | | | | | | | | | |
| 1,000,000 Tn | 17021339 | 15979187 | 93.88 | 1606258 | 487295 | 8.52 | 47.45 | 4468 | 0.28 |
| 50% RNA | | | | | | | | | |

TABLE 12
Metrics of sequencing results of second
naïve CD8⁺ T cell experiment.

| | | | | Total | |
|---|---|---|---|---|---|
| | | | Map | RNA | Unique |
| | Raw | Mappable | percent- | mole- | produc- |
| Sample | reads | reads | age (%) | cules | tiveCDR3 |
| 20,000Tn_20% | 334713 | 293943 | 87.82 | 13411 | 7466 |
| 20,000Tn_20% | 310547 | 262774 | 84.62 | 13329 | 7464 |
| 20,000Tn_20% | 526435 | 434432 | 82.52 | 16873 | 8888 |
| 20,000Tn_20% | 447301 | 360520 | 80.60 | 18573 | 8750 |
| 100,000Tn_20% | 1962817 | 1853561 | 94.43 | 94536 | 46272 |
| 100,000Tn_20% | 1575993 | 1481210 | 93.99 | 87887 | 44296 |
| 100,000Tn_20% | 1911879 | 1776146 | 92.90 | 95167 | 46087 |
| 100,000Tn_20% | 1858400 | 1721522 | 92.63 | 114885 | 48601 |

TABLE 13
Metrics of sequencing results of naïve CD8⁺ T cell with MIDICRS and 5′RACE.

| | | | | | | Ratio on |
|---|---|---|---|---|---|---|
| | | | | Map | Unique | unique CDR3 |
| | | Raw | Mappable | percentage | productive | discovered |
| Sample | Protocol | reads | reads | (%) | CDR3 | (MIDCIRS/5′RACE) |
| 20,000Tn_20% RNA_1 | MIDCIRS | 56780 | 46809 | 82.44 | 4202 | 2.77 |
| | 5′RACE | 74603 | 55268 | 74.08 | 1516 | |
| 20,000Tn_20% RNA_2 | MIDCIRS | 53322 | 42036 | 78.83 | 4284 | 2.42 |
| | 5′RACE | 77696 | 61074 | 78.61 | 1767 | |
| 100,000Tn_20% RNA | MIDCIRS | 432015 | 396472 | 91.77 | 28975 | 2.15 |
| | 5′RACE | 406533 | 336487 | 82.77 | 13497 | |
| 200,000Tn_20% RNA_l | MIDCIRS | 815238 | 758556 | 93.05 | 55052 | 1.92 |
| | 5′RACE | 885269 | 734108 | 82.92 | 28705 | |
| 200,000Tn_20% RNA_2 | MIDCIRS | 812503 | 649791 | 79.97 | 51870 | 2.03 |
| | 5′RACE | 813019 | 674146 | 82.92 | 25548 | |

TABLE 14
Metrics of sequencing results of CMV-specific effector CD8⁺ T cell experiments.

| | | | Unique | | |
|---|---|---|---|---|---|
| | Mappable | Total RNA | productive | Top CDR3 | Top T cell |
| Sample | reads | molecules | CDR3 | molecules | clone size (*) |
| 200000 | 2655814 | 324238 | 423 | 216348 | 72116 |
| Teffector_30% RNA | | | | | |
| 20000 | 293931 | 40815 | 88 | 40532 | 13510 |
| Teffector_30% RNA | | | | | |

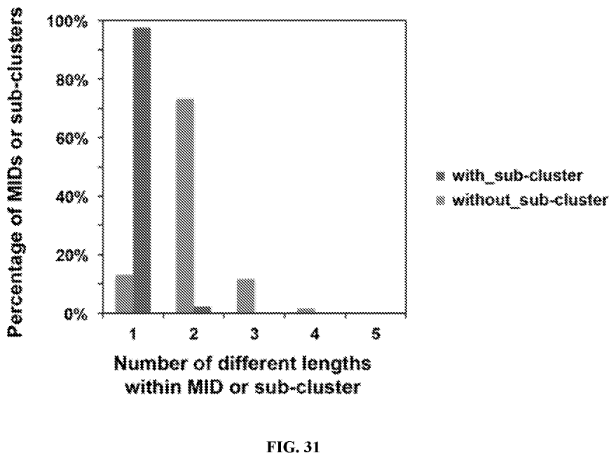

(*): Assuming 3 copies of RNA are recovered per cell according to FIG. 30.

TABLE 15
Digital PCR primers.
Digital PCR primers:

| | |
|---|---|
| RT | TTTTTTTTTTTTTTTTTTTTTTTTVN |
| | (SEQ ID NO: 596) |
| TRBC_F | GAGCCATCAGAAGCAGAGATC |
| | (SEQ ID NO: 597) |
| TRBC_R | CTCCTTCCCATTCACCCAC |
| | (SEQ ID NO: 598) |
| TRBC_Probe | CCACACCCAAAAGGCCACACTG |
| | (SEQ ID NO: 599) |

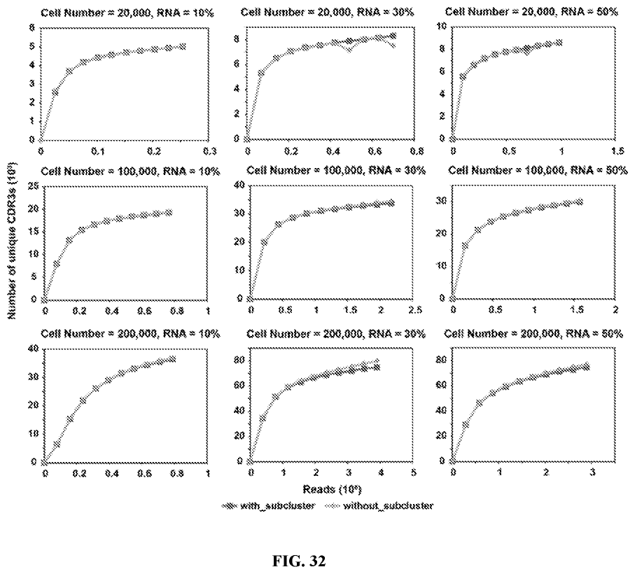

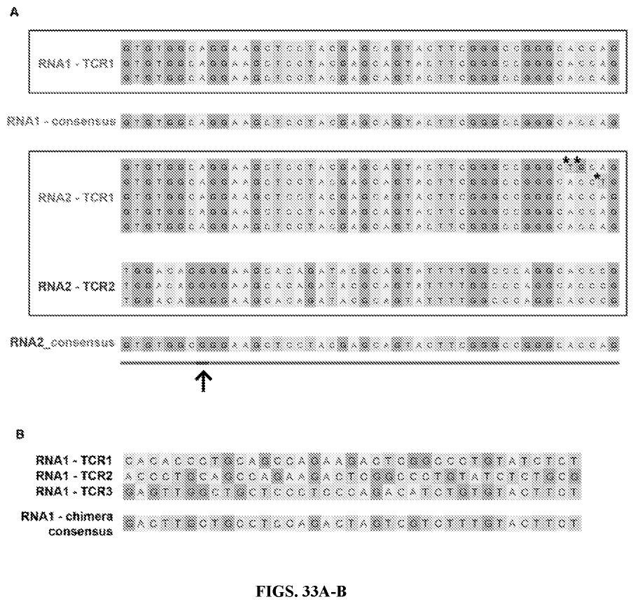

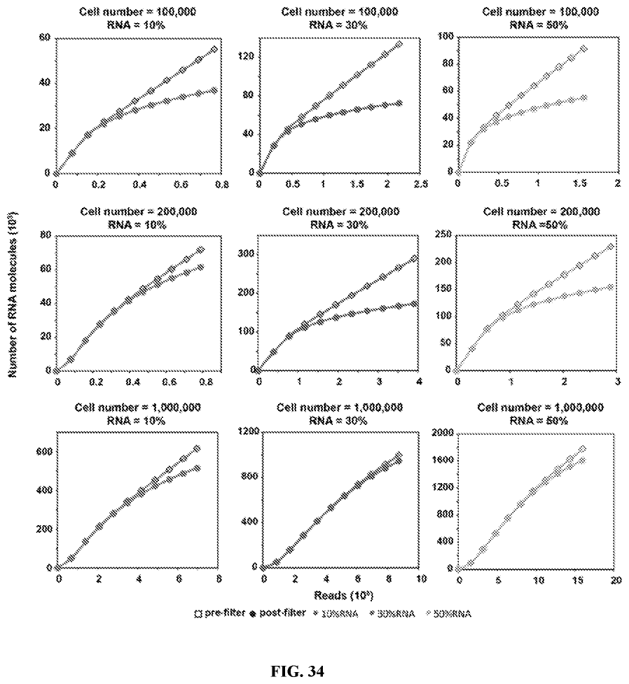

More importantly, it was found that, without performing sub-clustering, the number of unique consensus sequences (unique CDR3 sequences) was overestimated, especially in samples with one million cells (FIGS. 27C, 32). This is because chimera sequences were generated in the consensus building step for two scenarios. In one scenario, multiple true TCR sequences could be tagged with the same MID and quality score weighted consensus building will generate chimera sequences (FIGS. 27D, 33A). In the second scenario, PCR or sequencing errors on MIDs group multiple singletons (MIDs that contain only one read) under the new MID. If sub-clustering is applied, then these singletons will be separated and discarded under the singleton category. However, without sub-clustering, these singletons will be forced to generate a chimera sequence (FIG. 33B). Taken together, these chimera sequences cause over-estimation of the total TCR diversity. The percentage of chimera sequences can be as high as 47% (Table 10). Thus, MIDCIRS not only can increase diversity coverage of CDR3 but improve the accuracy of diversity estimation.

MID Read-Distribution-Based Barcode Correction Improves Accuracy and Sensitivity of Counting TCR Transcripts:

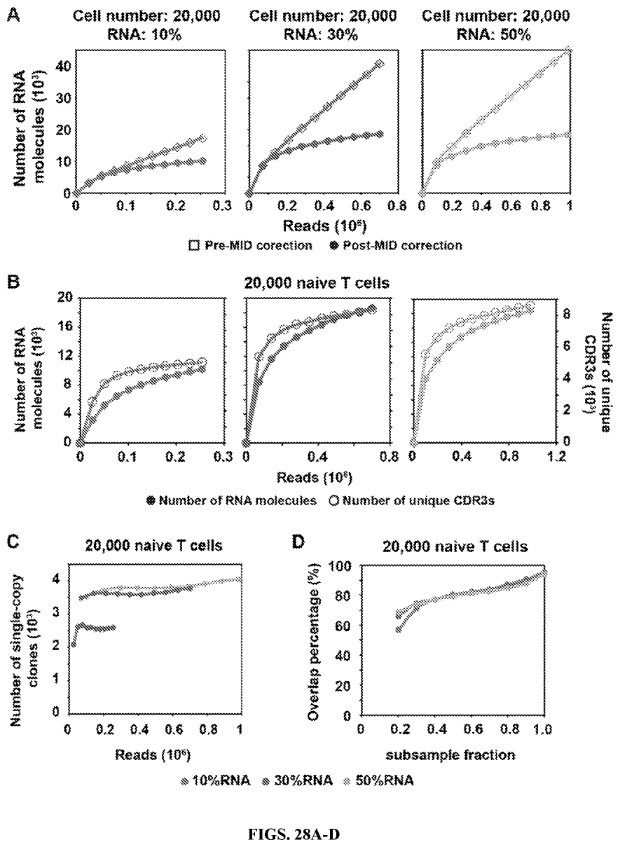

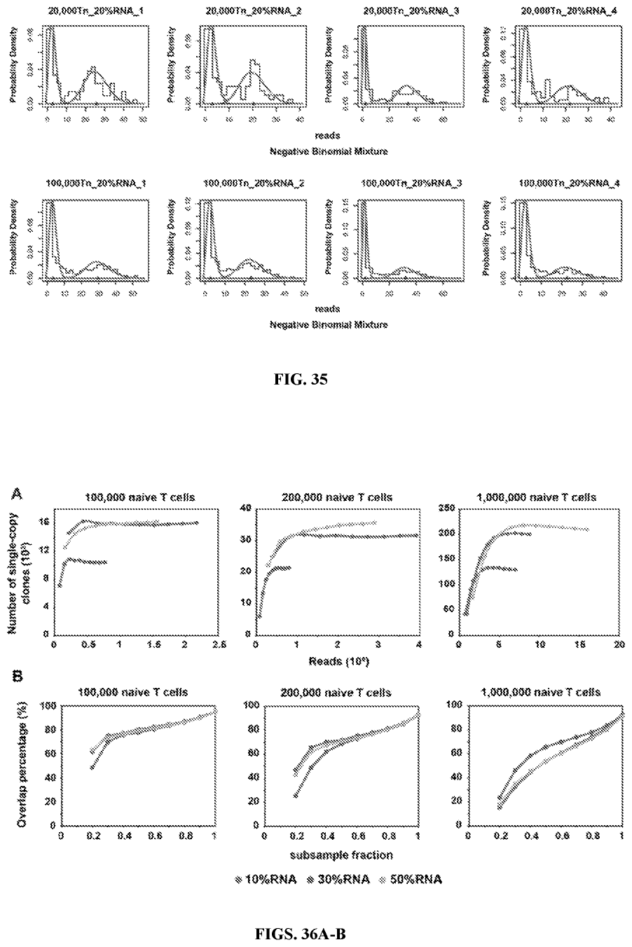

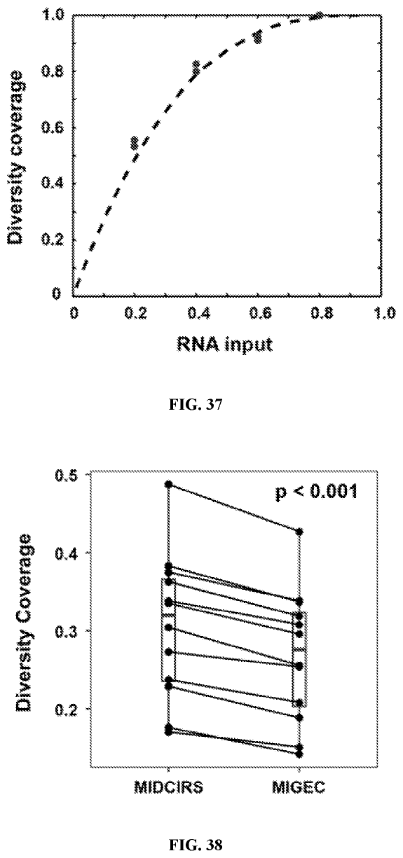

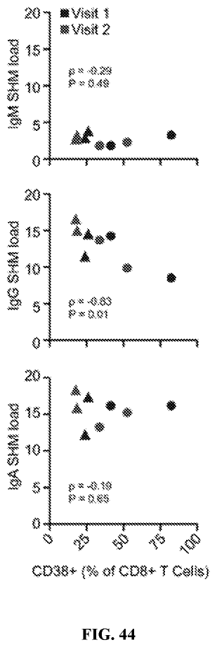

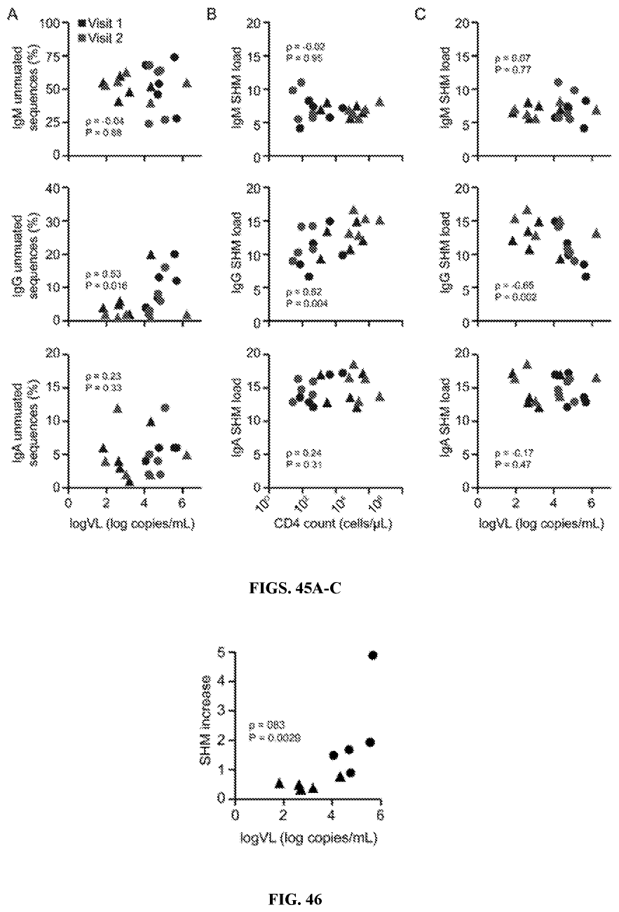

Besides correcting PCR and sequencing errors, MIDs have also been used for absolute quantification of RNA molecule copy number in single cell studies to improve precision. Here, it was demonstrated how to use MIDCIRS TCR-seq to digitally count TCR transcripts. The absolute quantification of TCR transcripts is fundamental for accurate clonal size estimation. It was noticed that PCR and sequencing errors also affected MIDs, as seen in single cell RNA sequencing studies, leading to an inflated number of RNA molecules when libraries were sequenced exhaustively with respective to the total TCR transcripts in the sample (FIGS. 28A and 44). To correct MID errors, singleton reads were removed, which cannot be confidently used in generating MID groups due to sequencing errors. Then, a similar approach was applied in single cell RNA-seq by fitting the distribution of reads under each MID sub-group into two negative binomial distributions (FIG. 35). Erroneous MIDs generated due to PCR errors generally have distinctively lower read counts compared with true MIDs. These two negative binomial distributions distinctly separated true MIDs from erroneous MIDs. MIDs with low read counts were removed accordingly. After MID correction, number of RNA molecules saturated across libraries (FIGS. 28A and 44).

It was found that a shallower sequencing depth is required to saturate unique CDR3s than RNA molecules (FIG. 28B). In addition, the amount of diversity covered increased with increasing RNA input. Thus, to exhaustively measure the TCR repertoire diversity, with 30-50% of RNA input, a sequencing depth equivalent to 10 times the cell number covers most of the CDR3 diversity (FIGS. 27C and 32), while a sequencing depth equivalent to about 100 times the relative RNA input (defined as cell number multiplied by percentage of RNA input) is required to saturate the RNA molecules (FIGS. 28A and 44). For example, 30% RNA of 20,000 cells is equivalent to 6,000 RNA input. Thus, it takes about 600,000 reads to saturate the RNA molecules but only 200,000 reads to saturate the unique CDR3s (FIG. 28A, middle panel).

After MID correction, with optimal sequencing depth, TCR clones were stably detected with a single TCR RNA molecule (single-copy clones with at least two identical sequencing reads). The number of single-copy clones saturates with adequate sequencing depth (FIGS. 28C and 36A). Meanwhile, the degree of overlapping clones was compared within these single-copy clones at different sequencing depths. To do this, each library was sub-sampled to different fractions of the total reads. The overlapping clones were compared between two adjacent sub-samples, and the overlap percentage was calculated by dividing the number of overlapping clones by the total number of clones observed in the deeper sub-sample. Thus, for total of 10 sub-samples, 9 clonal overlap percentages were calculated and plotted with respect to sequencing depth (FIGS. 28D and 36B). More than 90% of single-copy clones were repeatedly detected between the full sequencing reads and the 0.9 sub-sample fraction. The overlap percentage was above 80% for the latter part of curve (FIGS. 28D and 36B), which suggested that optimal sequencing depth was reached to detect single-copy TCR clones.

Estimating TCR RNA Molecule Copy Number and Validation with Digital PCR:

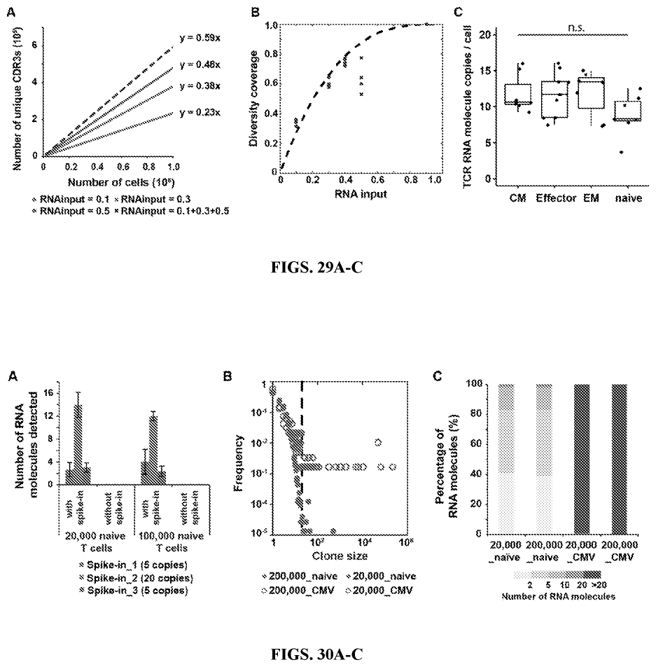

From early analysis, it was known that the diversity coverage of unique CDR3s increased as RNA input increased. Here, an in-depth analysis was performed on the relationship between these two parameters and it was found that the diversity coverage of unique CDR3s increased significantly as the RNA input increased initially, then reached a plateau, which resulted in a nonlinear increasing of the diversity coverage of unique CDR3s (FIGS. 29A and B). It was assumed that total diversity for a sample is the diversity discovered when combining all sequencing reads from 10%, 30%, and 50% RNA input libraries into a pseudo-90% RNA input. With 50% RNA, about 60% of total diversity could be recovered (FIG. 29B).

Since the observed diversity is dependent on total TCR RNA molecules in a sample, which is a function of TCR RNA molecule copy number per cell and RNA input percentage, it was next sought to use a probability model to predict TCR RNA molecule copy number per cell using the observed diversity coverage of unique CDR3s as a function of RNA input percentage. The estimated diversity coverage of different RNA inputs, including 10%, 30% and 50% RNA, was used as well as the computationally combined pseudo-40% (10%+30%) and pseudo-90% RNA inputs as data points to fit the probability model. The best fit resulted in 3 copies of TCR RNA molecule per cell (FIG. 29B). In another independent experiment, RNA from 20,000 and 100,000 naïve CD8⁺ T cells were evenly separated into five aliquots respectively. Four of five aliquots were sequenced (Table 12). Results showed that CDR3 diversity detected by MIDCIRS was very reproducible among the 4 aliquots and was also proportional to the cell input numbers. In addition, the aliquots were bioinformatically combined into pseudo-40%, 60% and 80% of RNA inputs and the diversity coverage was fitted using the probability model described in Example 6. As with previously, the best fit resulted in 3 copies of TCR RNA molecule per cell (FIG. 37).

However, in order to apply this TCR RNA molecule copy number in estimating T cell clone size, the method needed to be validated using a different method and also tested to see if different phenotypes of T cells might have different TCR RNA molecule copy numbers, which would be similar to the differences seeing in naïve B cells and plasmablasts. Next, TCR RNA molecule copy number was validated using digital PCR (dPCR) and it was found that various types of T cells have similar TCR RNA copies (8-12 copies per cell) (FIG. 29C). Thus, with MIDCIRS TCR-seq, about 30% efficiency could be achieved in recovering the target TCR RNA molecules, which is expected given dPCR in a nanoliter volume is more efficient than bulk PCR in tubes. This ratio also established a reference point for rare T cell clone frequency estimate using MIDCIRS method.

Detecting Single Cell Worth of TCR RNA Using MIDCIRS:

The lack of accurate and absolute quantitation of TCR clones limited the evaluation of the sensitivity of various IR-seq methods, which slowed the application of detecting rare TCR clones in both basic research and clinical practice. To address the detection sensitivity using MIDCIRS, control TCR RNA was spiked with varying copy numbers into naïve T cells and validated the robustness of detecting spiked-in TCRs. 5, 20, and 5 copies of three spike-in cell lines with known TCR sequences were added into 20,000 and 100,000 naïve CD8⁺ T cells. 3, 13, and 3 copies of three spike-ins were reliably detected respectively (FIG. 30A).

The ability to detect a single T cell's worth of control RNA was evaluated in a larger number of other T cells. The concentration of TCR RNA molecule from the Jurkat cell line and spiked in 10 copies of TCR RNA into 20,000-1,000,000 naïve CD8⁺ T cells was digitally counted (Table 11). In all 1,000,000 cells that were sequenced, Jurkat TCR sequences were detected (Table 10). This sensitivity was a significant improvement compared with the previous method, which was demonstrated to be 1 in 10,000 (Ruggiero et al., 2015). These results demonstrated that MIDCIRS is highly sensitive, capable of detecting a single cell's amount of TCR transcripts, and rare clones could be readily and robustly detected. Those single-copy clones (minimum two identical reads) we discovered are thus likely to come from single cells (FIGS. 28C and 36A).

Meanwhile, the sensitivity of MIDCIRS and 5′RACE protocol was compared using the diversity coverage as the parameter. Briefly, the 5′RACE protocol that was used in Smart-seq2 protocol was used for TCR repertoire sequencing, which has been demonstrated to significantly improve RNA capture efficiency (Picelli et al., 2013). Equal amounts of RNA (20%) from the same purification was used for both the MIDCIRS and the 5′RACE protocol. Sequencing results were then processed with the MIDCIRS-TCR pipeline and it was found that 5′RACE protocol only recovered about 44% of diversity compared to what MIDCIRS protocol obtained (Table 13). With improved accuracy and sensitivity to detect rare clones, MIDCIRS is promising in being applied to detect MRD after treatment.

Quantifying T Cell Clonal Expansion in Infection Using MIDCIRS:

Accurate quantification of diversity and abundance of T cell clones is important for application of TCR-seq in clinical settings, ranging from prognosis to treatment decision-making. However, there lacks an accurate approach to evaluating the degree of T cell clonal expansion in humans. Therefore, the MIDCIRS TCR-seq was used to examine T cell clonal expansion in infection. 20,000 and 200,000 CMVpp65-specific effector CD8⁺ T cells were sorted from CMV infected patients and 30% of RNA input was used to perform TCR-seq (Table 14). CMV pp65 peptide has been shown to be the immunodominant target of CD8⁺ T cell response (Wills et al., 1996). TCR RNA molecules were digitally counted through the MIDCIRS pipeline. TCR sequences with over 20 copies of RNA molecules were defined as expanded clones according to TCR abundance distribution comparing between naïve CD8⁺ T cells and CMV tetramer positive effector CD8⁺ T cells (FIG. 30B). Over 99% unique RNA molecules were from these expanded clones in CMVpp65-specific effector CD8⁺ T cells. On the other hand, although uneven clonal distribution was observed in naïve CD8⁺ T cells, these expanded clones only account for less than 1% unique RNA molecules (FIG. 30C). The data showed that in CMV infection, single CMV-specific TCR clone can have about 70,000 T cell progenies in 200,000 polyclonal CMV-specific effector CD8⁺ T cells (Table 14). These polyclonal CMV-specific effector CD8⁺ T cells represent about 2.6% of total CD8⁺ T cells. In addition, the previous study showed that tetramer positive polyclonal CMV precursor cells existed at a frequency of 1 in 100,000 CD8⁺ T cells in CMV seronegative individuals. Taken together, these results suggest that single T cell clone can have about 900-fold proliferation in infection in humans. Thus, MIDCIRS can be applied to evaluate clone size and degree of clonal expansion in viral infection.

In this study, MIDCIRS was applied in T cells to demonstrate (1) the necessity of MID sub-clustering to improve accuracy of repertoire diversity estimation; (2) the accuracy of counting TCR RNA molecules via MID read-distribution based barcode correction; (3) the sensitivity of detecting a single cell in as many as one million naïve T cells; and (4) the ability to quantify T cell clonal expansion due to infection in CMV-seropositive patients.

### Example 6—Material and Methods

Naïve CD8⁺ T Cell Sorting:

Human leukocyte reduction system chambers were obtained from deidentified donors at We Are Blood (Austin, Tex.) with strict adherence to guidelines from the Institutional Review Board of the University of Texas at Austin. CD8⁺ T cell enrichment was done following the protocol described previously (Yu et al., 2015) using RosetteSep CD8⁺ T Cell Enrichment Cocktail (STEMCELL) together with Ficoll-Paque (GE Healthcare). Then, RBCs were lysed using ACK Lysing Buffer (Lonza). After washing in phosphate-buffered saline with fetal bovine serum, the cell mixture was passed through a cell strainer (Corning) and ready for use. Naïve CD8⁺ T cells were FACS sorted into RLT Plus buffer (Qiagen) supplemented with 1% β-mercaptoethanol (Sigma) based on the phenotype of CD8⁺CD4⁻CCR7⁺CD45RA⁺ using BD FACSAria II cell sorter.

CMV CD8⁺ T Cell Enrichment and Sorting:

CMVpp65:482-490 (NLVPMVATV) was used to prepare streptamers as previously described (Zhang et al., 2016). Miltenyi anti-phycoerythrin (PE) microbeads and magnetic column were used to bind and enrich CMVpp65-specific T cells (Yu et al., 2015). The flow-through was collected for background staining. The enriched fraction was eluted off the column and washed into cell buffer. The following antibody panel was used to stain both the enriched and flow-through fractions: CD4, CD14, CD16, CD19, CD32, and CD56 (BioLegend) as a dump channel to stain residual non-CD8 T cells, and CD45RA, CCR7, CD27 and IL7R (BioLegend). 7-Aminoactinomycin D was used as a viability marker. Dump⁻Streptmer⁺CD45RA⁺CCR7⁻CD27⁻IL7R^{lo} live T cells were sorted into RLT Plus buffer supplemented with 1% β-mercaptoethanol using BD FACSAria II cell sorter.

Bulk TCR Library Generation and Sequencing:

Total RNA was purified using All Prep DNA/RNA kit (Qiagen) following the manufacturer's protocol. Library preparation and QC were similar to protocols described in Example 4 using TCR primers (Table 15). Reads of the same library from all runs were combined and analyzed.

Digital PCR of TCR:

Total RNA purified from sorted CD8⁺ T cells and cultured CMV-specific CD8⁺ T cell lines were reverse transcribed with polyT primers (Supplementary Table S5) using Superscript III in 20 ul reaction following the manufacturer's protocol. 2 ul of cDNA was subsequently used on QuantStudio 3D digital PCR system following manufacturer's protocol.

Preliminary Read Processing:

A similar procedure as described in Example 4 was used to generate consensus sequences. First, only reads that have exact TCR constant sequences were kept for further analysis. These reads were then cut to 150nt starting from constant region to eliminate high error-prone region at the end of reads. These preprocessed reads were split into MID groups according to 12nt barcodes.

MID Sub-Cluster Generating and Filtering:

For each MID group, a quality threshold clustering was used to group reads derived from a common ancestor RNA molecule and separate reads derived from distinct RNAs as described in Example 4. Briefly, a Levenshtein distance of 15% of the read length was used as the threshold. For each sub-group, a consensus sequence was built based on the average nucleotide at each position, weighted by the quality score. In the case that there were only two reads in an MID sub-group, they were only considered useful reads if both were identical. Each MID sub-group is equivalent to an RNA molecule. Next, all of the identical consensus sequences were merged to form unique consensus sequences. Further, filtering of unique consensus sequences was applied after sub-cluster generation by (a) removing non-functional TCR sequences and (b) removing sequences with lower MID counts that are one Levenshtein distance away from the other. Then, for each unique consensus sequence, MID sub-clusters were removed if their reads are less than 20% of maximum read count based on the fitting of two negative binomial distribution (FIG. 35).

Theoretical Percentage of MIDs that Need Sub-Clustering:

The process of MID labeling was modeled as a Poisson distribution. Given the total number of MIDs being M and the number of target molecules being N, the probability that a unique MID will occur k time(s) is:

$${P}_{k} = \frac{{( \frac{N}{M} )}^{k}}{k !} \times {e}^{- \frac{N}{M}} \qquad ( 1 )$$

Thus, P₀ and P₁ are the probability that a MID will be tagged 0 and 1 time respectively and the percentage of MIDs that need sub-clustering, F(k>1), is given by:

$$F ( k > 1 ) = \frac{[ 1 - {e}^{- \frac{N}{M}} - \frac{N}{M} \times {e}^{- \frac{N}{M}} ]}{1 - {e}^{- \frac{N}{M}}} \qquad ( 2 )$$

With over 16 million MID combinations from 12 random nucleotides, when the number of target molecules, N is less than 5,000,000, equation (2) is an approximate linear function (FIG. 27B).

Diversity Coverage and RNA Copy Number Simulation:

The estimation of diversity will be affected by the initial RNA input (percentage of initial RNA used to construct the sequencing library). A statistical model was used to estimate the diversity coverage for the naïve T cells we sorted based on RNA sampling depth.

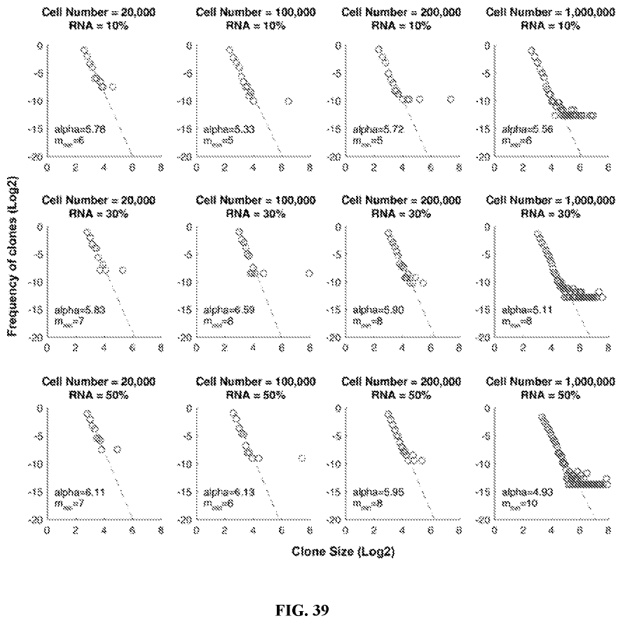

For N observed RNA molecules, there are K different RNA clones. The RNA molecule copy number of each clone is mᵢ (iϵ(1, K)), whose sum equals N. After fitting the data, mᵢ follows a power law distribution (FIG. 39):

mᵢ=m×xᵢ (3)

f(xᵢ)=(α−1)xᵢ^{-α},(α>1) (4)

(m is the RNA molecule copy number per cell, which is a constant across all T cells FIG. 29C). represents the cell numbers of each clone, which follows a power law distribution (Mora et al., 2016), and the parameter a was fitted with an algorithm combining maximum-likelihood fitting and goodness-of-fit test based on Kolmogorov-Smirnov statistic (Caluset et al., 2009). ‘fit_power_law’ function in R package igraph was applied (Csardi et al., 2006).

Specifically, the RNA molecule distribution (FIG. 39) was fitted with equation (5):

$$f ( {m}_{i} ) = ( \frac{α - 1}{{m}_{m i n}} ) {( \frac{{m}_{i}}{{m}_{m i n}} )}^{- α} , ( α > 1 ) \qquad ( 5 )$$

Since ‘m’ is a constant (see FIG. 29C), the alpha in equation (4) and (5) should be equal. The distribution was fitted across all libraries on log-log scale, and the average slope was taken as a in the above model).

When n RNA molecules are sampled from this population, the expected detected diversity, E(D), can be calculated as the following:

$$E ( D \vert m , {x}_{i} ) = K - \frac{{\sum }_{i = 1}^{K} ( \begin{matrix}N - m \times {x}_{i} \\ n\end{matrix} )}{( \begin{matrix}N \\ n\end{matrix} )} , {x}_{i} = ( {x}_{1} , {x}_{2} , … , {x}_{K} ) \qquad ( 6 )$$

And xᵢ can be sampled from the fitted power law distribution.

Then, the percentage of the RNA diversity coverage, P(D), can be estimated as:

$$P ( D \vert m , {x}_{i} ) = \frac{E ( D \vert m , {x}_{i} )}{K} \qquad ( 7 )$$

The diversity coverage of unique CDR3s was scaled to the estimated diversity coverage with 90% RNA input, D_{obs}. Equation (8) was used to get estimated m:

$${min}_{m} {\sum }_{i} {( P ( {D}_{i} \vert m , {x}_{i} ) - {D}_{obs} )}^{2} , m ∈ \{ 1 , 2 , … \} \qquad ( 8 )$$

Statistical Analysis:

Mann-Whitney U test was used to calculate the significance of copy number difference between pairs in naïve, effector, effector memory and central memory CD8⁺ T cells and p values was adjusted with Benjamini-Hochberg procedure. Adjusted p-value that was less than 0.05 was considered significant.

Expected Number of Identical RNA Molecules Tagged with Same MID:

When there are N different MIDs, the probability of RNA molecule B's MID shares RNA molecule A's MID is 1/N. Let the number of identical RNA molecules be n, then the probability that RNA molecule A's MID is shared is:

$$1 - {( 1 - \frac{1}{N} )}^{n - 1} \qquad ( 1 )$$

Based on equation (1), the expected number of identical RNA molecules tagged with same MID, E(n) is:

$$E ( n ) = n \times ( 1 - {( 1 - \frac{1}{N} )}^{n - 1} ) \qquad ( 2 )$$

### Example 7—Rapid HIV Progression is Associated with Extensive Ongoing Somatic Hypermutation

RPs are Defined by a Rapid Decline in CD4 Count:

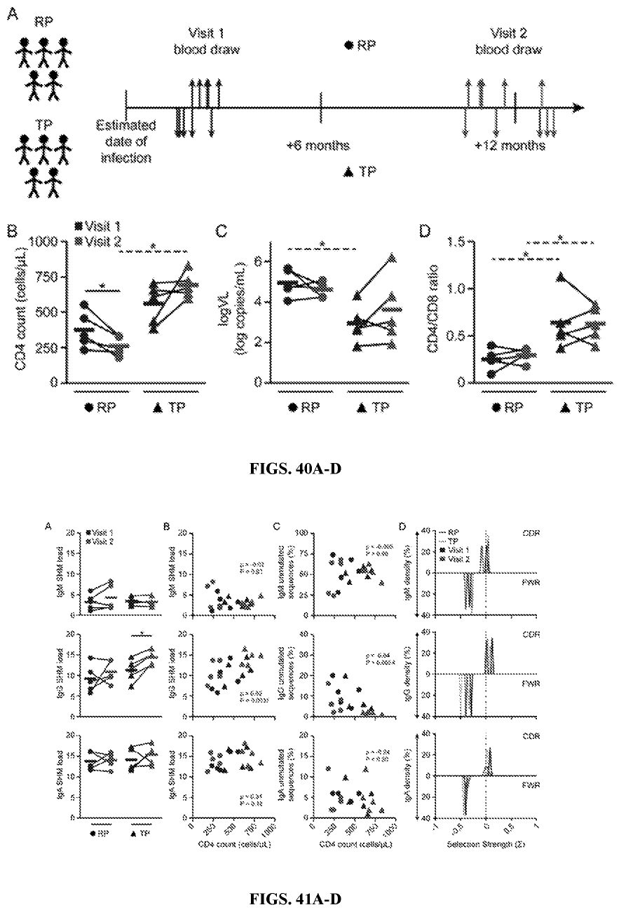

Isolated PBMCs were isolated from 10 HIV-infected individuals (5 RPs, 5 TPs) at two timepoints: the first visit occurring 1-3 months after infection and the second visit occurring around 1 year after infection (FIG. 40A and Table 16). RPs experience a dramatic reduction in peripheral CD4 counts, dropping below 350 cells/pt within the first year of infection, while TPs maintain normal CD4 counts of greater than 500 cells/pt for at least 2 years. Between visit 1 and visit 2, RPs exhibited uniform depletion of peripheral CD4⁺ T cells, while TPs' CD4 counts remain unchanged or even increased (FIG. 40B). The RP group was associated with a higher viral load at the early timepoint, but the decreasing CD4 count was not accompanied by an increasing viral load (FIG. 40C). RPs have lower CD4: CD8 ratios, a measure that is associated with T cell activation and poor prognosis in ART-treated HIV patients (Serrano-Villar et al., 2013; Serrano-Villar et al., 2014), than TPs across both timepoints (FIG. 40D).

Disease Severity Correlates with Diminished IgG SHM Load:

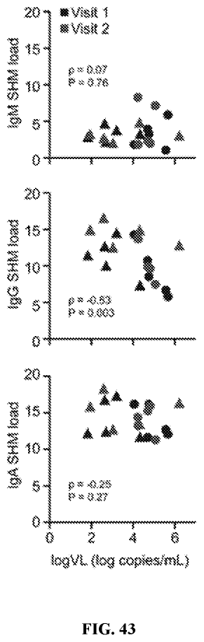

Despite the increased initial viral load and rapid loss of CD4⁺ T cells, collectively, RPs do not differ from TPs in overall SHM loads in the 3 major isotypes (FIG. 41A). In fact, on the bulk level, SHM loads within the RPs are not significantly altered between the two timepoints. Only IgG in TPs displays significantly more SHMs upon visit 2 (FIG. 41A, middle panel). Considering the occurrence of hypergammaglobulinemia in HIV patients and the dominance of the IgG1 subclass in HIV-specific antibodies (Tomaras and Haynes, 2009), it is likely that this overall increase in IgG SHMs is HIV-driven. The SHM load of IgG antibodies, but not IgM or IgA, is inversely correlated with disease severity (FIGS. 41B and 43). Higher CD4 count (FIG. 41B, middle panel) and lower viral load (FIG. 43, middle panel) both correlate with higher average IgG mutations. For the subset of subjects with available data (N=2 RPs and 2 TPs, 8 total samples), these IgG mutations were inversely correlated with the percent of CD8⁺ T cells expressing the activation marker CD38 (FIG. 44), suggesting that general immune activation could be linked to the reduced IgG SHM load observed in patients with more severe disease.

TABLE 16
Cohort Summary.

| Individ- | | | Visit 1 Age | Visit 1 Days | Visit 2 Days |
|---|---|---|---|---|---|
| ual | Group | Sex | (years) | Post-infection | Post-infection |
| R1 | RP | M | 27 | 76 | 332 |
| R2 | RP | M | 23 | 87 | 321 |
| R3 | RP | M | 22 | 69 | 335 |
| R4 | RP | M | 26 | 77 | 390 |
| R5 | RP | M | 17 | 62 | 334 |
| T1 | TP | M | 22 | 80 | 347 |
| T2 | TP | M | 22 | 50 | 395 |
| T3 | TP | M | 25 | 48 | 388 |
| T4 | TP | M | 22 | 54 | 401 |
| T5 | TP | M | 18 | 52 | 318 |

Chronic immune activation is a key factor in HIV infection (Deeks et al., 2004; Hazenberg et al., 2003). There is evidence that hyperactive naive B cells and/or CD27⁻ atypical memory B cells contribute to the increased secretion of IgG antibodies in HIV patients (De Milito et al., 2004). These subsets of B cells have undergone fewer divisions and harbor fewer SHM than classical memory B cells in these patients (Moir et al., 2008). The overall lower IgG SHM load with more severe disease could be caused by class-switching of these lowly mutated classes of B cells upon aberrant activation and/or defective germinal center T cell help. To test the first possibility, the percentage of unmutated sequences were compared to the CD4 counts within the cohort. Consistent with the hypothesis that recently activated and class-switched naive B cells contribute to the observed reduction of IgG SHM load with disease severity, the fraction of unmutated IgG, but not IgM or IgA, correlated with decreasing CD4 count (FIG. 41C) and increasing viral load (FIG. 45A). However, these unmutated sequences do not fully account for the trend, as the average number of mutations in IgG, but not IgM or IgA, still negatively correlated with disease severity after excluding unmutated sequences (FIGS. 45B and 45C). It is possible that a large, diverse CD4⁺ T cell receptor repertoire contributes to efficiently inducing SHM in the global antibody repertoire.

To test the second part of the hypothesis, BASELINe (Yaari et al., 2012) analysis was performed to assess the degree of antigen selection pressure as a measure of germinal center CD4⁺ T cell help (FIG. 41D). BASELINe compares the observed frequency of amino acid-changing (replacement) mutations to the expected frequency for random mutations. Evolving higher affinity antibodies necessitates replacement mutations, as the amino acid sequence ultimately determines the binding properties. Thus, if a higher affinity antibody is positively selected to proliferate, the replacement mutation that drives the higher affinity would be overrepresented in the resulting B cell progenies. A higher-than-random frequency of replacement mutations indicates the presence of antigen selection. Conversely, a lower-than-random frequency of replacement mutations indicates negative selection. Replacement mutations in the framework region (FWR) can disrupt proper antibody folding, so negative selection strength was expected and observed in the FWR of antibodies of all isotypes (FIG. 41D, bottom half of each panel, and Table 17). The complementary determining region (CDR) governs antibody binding properties. Slight positive selection was observed in the IgG antibodies during the first visit that was reduced upon visit 2 for both groups (FIG. 41D, top half of middle panel, and Table 17). The positive selection at the early timepoint could be caused by well-selected anti-HIV memory B cells during the early stages of acute infection. To put this selection into perspective, recent studies found strong selection strength (Σ>0.5) in the CDRs of B cells from the central nervous systems of multiple sclerosis patients (Stern et al., 2014) and neutral or negative (Σ≤0) selection strength in the CDRs of B cells from donors up to 4 weeks after receiving influenza vaccination (Laserson et al., 2014). Thus, this average level of Σ=0.1 in the IgG antibodies at visit 1 represents weak but significant selection. Indeed, HIV-specific IgG antibodies have been detected just 2 weeks post-infection and steadily rise over the next month (Tomaras et al., 2008). Despite the reduced CD4 count in RPs, no major differences were detected in selection strength between the two groups on the global level.

Longitudinally Tracked Clonal Lineages Mutate Dramatically in RPs with Impaired Selection:

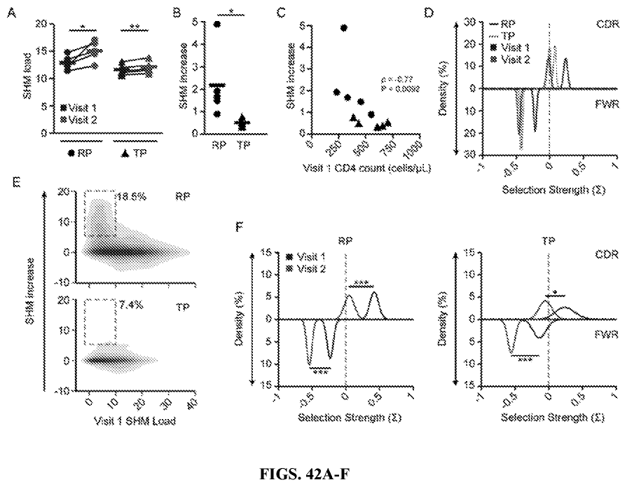

It was next sought to track the evolution of antibody sequences over time. The sequences were combined from both visits and formed clonal lineages on the basis of the same V and J gene usage and 90% similarity within the CDR3, as previously described (Wendel et al., 2017). Here, clonal lineages were isolated that contained sequences derived from both visits and compared the SHM properties of the visit 1 sequences to their visit 2 relatives. Both RPs and TPs harbor significantly more SHMs in their visit 2 sequences (FIG. 42A). These two-timepoint lineages, which already contain over 10 SHMs on average at the first visit, continue to mutate further. Surprisingly, despite fewer peripheral CD4⁺ T cells, RPs induce significantly more SHM over this time period (FIG. 42B). This increase in SHM within these two-timepoint lineages counterintuitively correlated with disease severity (FIGS. 42C and 46), though this could possibly be linked to the expansion of HIV-specific TFH cells in chronically infected lymph nodes (Lindqvist et al., 2012).

BASELINe analysis revealed that the initial mutations at visit 1 were strongly selected in RPs but only weakly selected in TPs (FIG. 42D, curves in top half, and Table 18). Unlike the influenza vaccination experiment that did not detect positive selection, the consistent availability of antigen and ongoing infection, particularly in the case of RPs with high viral load at visit 1 (FIG. 1C), could contribute to this stronger selection strength. However, the positive antigen selection strength completely disappeared by visit 2 (FIG. 42D, pink curves in top half). The de novo mutations that arise in visit 2, particularly in RPs, occur in the absence of antigen selection. These mutations may result from polyclonal activation in an extrafollicular T-independent manner, or they could be affected by dysfunctional TFH cells.

The differential mutation increase observed between RPs and TPs within these two-timepoint lineages stems from RP lineages with few mutations at visit 1 (≤10 SHM) undergoing a burst of SHM upon visit 2, increasing by upwards of 5-20 mutations (FIG. 42E). Further analyzing these actively mutating lineages revealed that the visit 1 sequences in these lineages were especially strongly selected, particularly in RPs (FIG. 42F). Analyzing lineages spanning the two timepoints allowed us to dissect the selection at the early stages of disease and after the infection has been established. B cells which have not had time to accumulate many mutations are initially well selected, but by visit 2, when the SHMs have increased, the selection is attenuated (FIG. 42F). However, most broadly neutralizing HIV antibodies are highly mutated and take years to develop (Wu et al., 2011). If multiple specific mutations must accumulate before an appreciable effect can be made on binding affinity, it is unlikely that these have occurred in the first year of infection. It is possible that these initial mutations reach a local energy minimum such that most replacement mutations reduce binding affinity, leading to an accumulation of silent mutations and reduction of the positive selection signal. Another possibility involves viral escape mutations disrupting affinity maturation. Additionally, the disruption of germinal center formation during early-stage infection has been reported and could contribute to diminished antigen selection (Levesque et al., 2009). The data suggest that RPs experience not only accelerated disease progression, but also an accelerated immune response. However, without outside intervention, the RP immune system ultimately loses this arms race.

In summary, antibody repertoire sequencing techniques were utilized to elucidate the antibody response to HIV infection in an underappreciated class of HIV-responders: RPs. On the global repertoire level, RPs are similar to TPs, though more severe disease progression was associated with a reduction in IgG SHM load, likely due to a combination of polyclonal activation and class-switching of activated naive B cells and poor SHM induction. Global IgG antibodies show signs of weak antigen selection at visit 1, but these signs disappear 1 year post-infection. Two-timepoint lineage analysis enabled direct detection of clonal lineage evolution between the 2 visits. These lineages continued to readily mutate in RPs, but the initial signs of strong antigen selection in the visit 1-derived sequences were lost by visit 2. Despite strong initial selection and the ability to further mutate, RPs fail to generate protective antibodies and experience a rapid decline in CD4 counts. Understanding the mechanism behind the loss of antigen selection pressure could be used for the design of an HIV vaccine.

### Example 8—Materials and Methods

Study design and cohort: Whole blood from 5 RPs and 5 TPs was obtained from treatment-naive HIV patients in the early stages of infection and one year post-infection. CD4 and CD8 counts were determine by FACSCalibur (Becton Dickinson, USA) and analyzed automatically using the MultiSET software (BD Biosciences). Viral loads were determined by a commercial HIV RNA quantitative detection assay, COBAS AmpliPrep/COBAS TaqMan HIV-1 Test (Roche, Germany), with a detection limit of 40 copies/mL in plasma. Infection date was estimated by Fiebig classification. Ficoll density gradient centrifugation was performed to isolate PBMCs for antibody repertoire sequencing.

Antibody Repertoire Sequencing:

Antibody repertoire sequencing library preparation and data processing were performed as previously described (Wendel et al., 2017). Briefly, up to 5 million PBMCs were lysed in RLT lysis buffer supplemented with 1%-beta-mercaptoethanol. RNA purification was performed using Qiagen AllPrep DNA/RNA purification kit following the manufacture's protocol. 30% of total RNA was used for reverse transcription utilizing a 12N molecular identifier (MID) fused to isotype-specific primers followed by 2 sequential PCR amplification steps. PCR products were gel purified and quantified via Agilent Tapestation 2000. Pooled libraries were sequenced via Miseq 2×250PE.

Raw sequencing reads were processed through MIDCIRS (Wendel et al., 2017) to group sequences with the same MID together. MID groups were further clustered with a 85% sequence similarity threshold to form subgroups, and consensus sequences (equivalent to RNA molecules) were generated within subgroups. Identical consensus sequences were merged to yield unique consensus sequences, or unique RNA molecules.

Unique RNA molecules were aligned to IMGT database set of human V-, D-, and J-gene alleles, and mismatches between the template and sequence of interest were tallied as SHMs, omitting the CDR3.

Selection Strength Analysis:

BASELINe (Yaari et al., 2012) was used to assess the strength of antigen selection pressure applied upon the antibody repertoire. As amino acid-replacing mutations are necessary to grant higher binding affinit, positive selection during affinity maturation leads to an enrichment of replacement mutations. BASELINe relates the observed replacement mutation frequency to that expected for a random mutation. A higher than expected frequency of replacement mutations is indicative of positive selection, as expected in the CDRs, while a lower than expected frequency is indicative of negative selection, as expected in the FWR, where replacement mutations can disrupt proper antibody folding.

To compare between progressor groups, probability density functions (pdf) for each subject were initially calculated, CDR and FWR separately. Then, the pdfs for the subjects belonging to the same group (RP or TP) were convoluted. To compare between sequences from lineages lowly mutated at visit 1 that increase in SHM load by visit 2, lineages with a visit 1 average SHM load of 10 or less that increased by 5 or more SHM at visit 2 were isolated. Visit 1 and visit 2-derived sequences were segregated. Selection strength pdfs for each unique sequence within each lineage of the corresponding visit were first convoluted, and then the resulted pdfs for each lineage for each subject were convoluted, and then finally the pdfs for subjects belonging to the same group were convoluted.

Clonal Lineage Formation and Two-Timepoint Analysis:

Unique sequences were clustered into clonal lineages as previously described (Wendel et al., 2017) with some modifications. Sequences from both visits were pooled together, and sequences with the same V- and J-gene alleles and 90% similarity on the CDR3 nucleotide sequence were clustered into clonal lineages. Lineages containing sequences derived from both visits were isolated to track the evolution of the antibody sequences over time. Within the two-timepoint lineages, visit 1- and visit 2-derived sequences were segregated and analyzed.

TABLE 17
Bulk repertoire antigen selection strength statistics.

| | RP visit 1 | RP visit 2 | TP visit 1 | TP visit 2 | |
|---|---|---|---|---|---|
| RP visit 1 | | <0.0001 | 0.0956 | 0.0669 | IgM |
| RP visit 2 | <0.0001 | | <0.0001 | <0.0001 | |
| TP visit 1 | 0.0012 | <0.0001 | | 0.4537 | |
| TP visit 2 | 0.0099 | <0.0001 | 0.1714 | | |
| RP visit 1 | | <0.0001 | 0.0242 | <0.0001 | IgG |
| RP visit 2 | <0.0001 | | <0.0001 | 0.1347 | |
| TP visit 1 | 0.0017 | <0.0001 | | 0.0011 | |
| TP visit 2 | <0.0001 | <0.0001 | <0.0001 | | |
| RP visit 1 | | 0.0616 | 0.4237 | 0.0023 | IgA |
| RP visit 2 | 0.2060 | | 0.0091 | 0.4244 | |
| TP visit 1 | 0.2453 | 0.3790 | | 0.0342 | |
| TP visit 2 | 0.0047 | 0.0153 | 0.0047 | | |

P-values between the BASELINe-generated antigen selection strength curves from FIG. 41D, split by isotype: IgM (top), IgG (middle), and IgA (bottom), for CDR (upper right half) and FWR (bottom left half), calculated as previously described (Yaari et al., 2012).

TABLE 18
Two-timepoint lineage selection strength statistics.

| | RP visit 1 | RP visit 2 | TP visit 1 | TP visit 2 |
|---|---|---|---|---|
| RP visit 1 | | <0.0001 | <0.0001 | <0.0001 |
| RP visit 2 | <0.0001 | | 0.0039 | 0.3393 |
| TP visit 1 | <0.0001 | 0.0412 | | 0.0034 |
| TP visit 2 | <0.0001 | 0.1607 | 0.1894 | |

P-values between the BASELINe-generated antigen selection strength curves from FIG. 3D for CDR (upper right half) and FWR (bottom left half), calculated as previously described (Yaari et al., 2012).

Statistics:

Significance tests were used as indicated in the figure legends. Two-tailed paired t test was used to determine significance for parameters compared between visits for matched subjects. Two-tailed Whitney Mann U test was used when comparing between progressor groups. Spearman's Rho was used to test correlations with disease severity. Selection strength significance was calculated as previously described (Yaari et al., 2012). Briefly, the P-value was determined by the probability that a random value from the pdf is higher than a random value from another pdf.

### Example 9—the Receptor Repertoire and Functional Profile of Follicular T Cells in Human HIV-Infected Lymph Nodes

HIV Infected LNs Contain Clonally Expanded GC T_{FH} Cells:

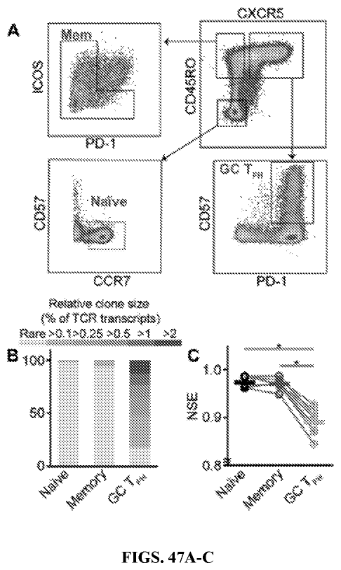

LNs from untreated HIV⁺ patients contain a high frequency of T_{FH} cells, but the mechanism that drives expansion of T_{FH} cells remains unclear. The enrichment of HIV antigens and the highly pro-inflammatory milieu in the LNs could lead to antigen-driven and/or bystander T cell expansion. To address whether proliferation of T_{FH} cells is antigen-dependent, it was tested whether HIV induces selective proliferation of certain T cell clones. GC T_{FH} cells were focused on because the frequency of these cells becomes greatly increased during chronic HIV infection. To identify GC T_{FH} cells, memory CD4⁺ T cells were selected that express T_{FH} cell markers CXCR5 and PD-1. CD57 is a glycan carbohydrate epitope expressed by T_{FH} cells in the GC, and this marker was used to further demarcate the GC subset. Naïve CD4⁺ T cells were identified by CD45RO⁻CXCR5⁻CD57⁻CCR7⁺ expression, and memory CD4⁺ T cells were CD45RO⁺CXCR5⁻PD-1⁻ICOS⁻ (FIG. 47A). 1,464 to 15,000 naïve, memory, and GC T_{FH} cells were sorted from freshly thawed LN samples and analyzed the TCR sequences of these subsets using a molecular identifier (MID)-based approach to increase the accuracy of repertoire sequencing. Because the variability of TCR sequences is encoded in the complementarity determining region 3 (CDR3) region, the number of transcripts detected were used for a particular CDR3 sequence to define TCR clone size. On average 11,839 TCR transcripts were detected for each sample. Unique TCR frequencies range from 1 in 37,129 (0.003%) for the rarest clones to 250 in 2,498 (˜10%) for the most expanded clone. To compare the degree of relative clonal expansion, TCR frequency was categorized into 6 groups, ranging from rare (<0.1%) to >2%, according to the clone size relative to the total TCR transcripts detected in that sample. As expected, the TCR repertoire of naïve CD4⁺ T cells was composed mostly of rare clones. In contrast, the TCR repertoire of GC T_{FH} cells had a much higher fraction of TCRs occupied by abundant clones (>0.1%) compared to naïve and memory CD4⁺ T cells (FIG. 47B, FIG. 50). The degree of TCR clonal expansion was quantified by normalized Shannon entropy (NSE). Consistent with the hypothesis that the increase in GC T_{FH} cell frequency is due to selective proliferation of certain T cell clones, GC T_{FH} cells had a lower NSE score compared to naive and memory cells (FIG. 47C). Taken together, the data demonstrated a notable expansion of clone size in GC T_{FH} cell populations.

TCRs from GC T_{FH} cells exhibit signatures of antigen-driven clonal convergence: Next, to test whether clonal expansion in GC T_{FH} cells from HIV-infected LNs was antigen-driven, the TCR sequences were analyzed for evidence of convergence to the same amino acid sequence from distinct nucleotide sequences. Unlike B cells, which can undergo somatic hypermutation, the TCR sequence of a naïve T cell is determined during maturation in the thymus and remains fixed throughout the lifespans of the T cell and its progeny. Thus, with the exception of clones that express 2 TCR α or β sequences, distinct TCR nucleotide sequences necessarily arise from distinct naïve T cells. However, multiple nucleotide sequences of different TCRs may encode the same amino acid sequence. These degenerate TCR sequences are typically rare, and the presence of these sequences suggests antigen selection pressure that favors certain TCR motifs that recognize particular antigen(s). Thus, having highly abundant CDR3 amino acid sequences that are encoded by multiple distinct nucleotide sequences indicates preferential expansion of T cells with that specificity.

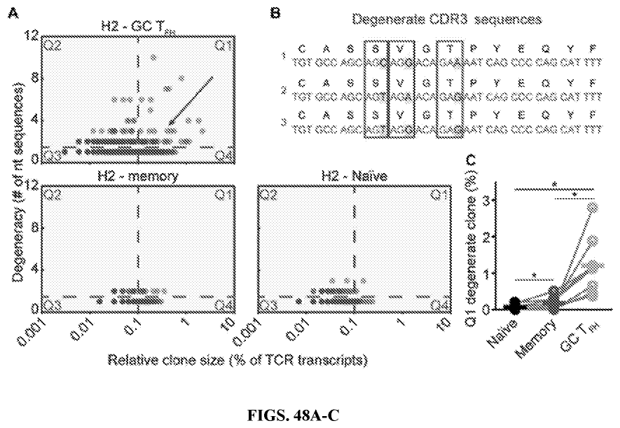

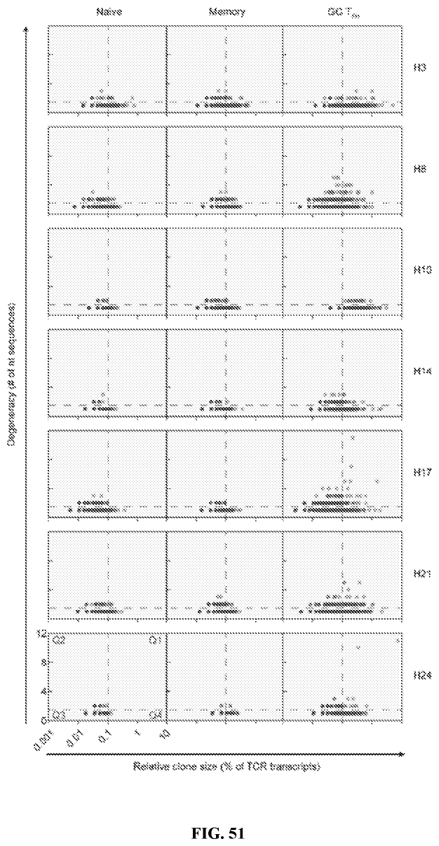

On the other hand, it would not be expected that multiple nucleotide sequences converge on the amino acid level in the absence of strong antigen-driven selection. Following this logic, the TCR nucleotide sequences were translated into amino acid sequences and tallied the number of different nucleotide sequences that encode each CDR3 amino acid sequence. These CDR3 amino acid sequences can be broken into 4 quadrants based on the level of degeneracy and frequency in the repertoire (FIG. 48A and FIG. 51). Q1 contained highly expanded amino acid CDR3 sequences that are encoded by 2 or more nucleotide sequences. These degenerate, abundant clones likely arose from strong antigen-driven selection and proliferation. Q2 contained low frequency amino acid CDR3 sequences that are also encoded by 2 or more nucleotide sequences. Degenerate clones can stochastically arise in the repertoire, but these are typically rare as reflected by the low frequency of non-clonally expanded sequences in Q2. Q3 contained amino acid CDR3 sequences that showed neither clonal expansion nor amino acid convergence and make up the majority of the repertoire. Q4 contained expanded amino acid CDR3 sequences derived from a single nucleotide sequence and are therefore non-degenerate. This TCR degeneracy analysis revealed a significant degree of antigen-driven clonal convergence in GC T_{FH} cells compared to naïve and memory T cells (FIG. 48B-C). Together with the NSE decrease in GC T_{FH} cells, these data provided further evidence that antigen-driven clonal expansion was preserved in GC T_{FH} cells.

HIV Promotes Selective Expansion of HIV-Reactive T_{FH} Cells:

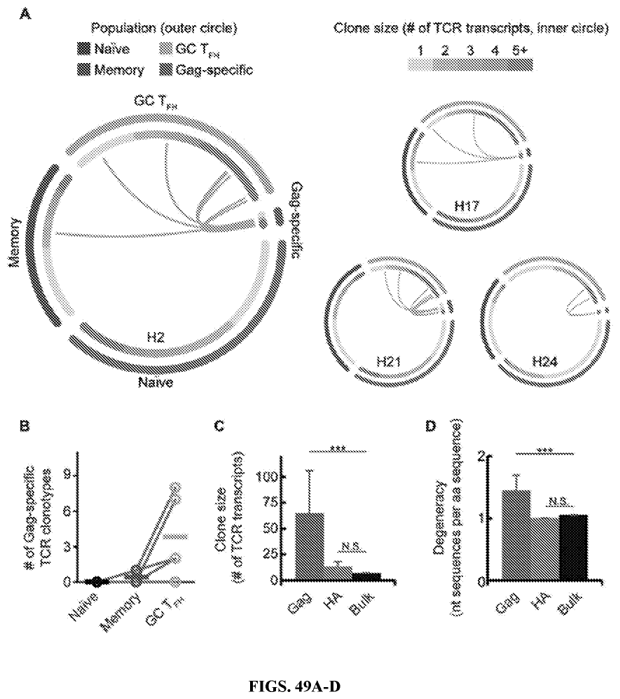

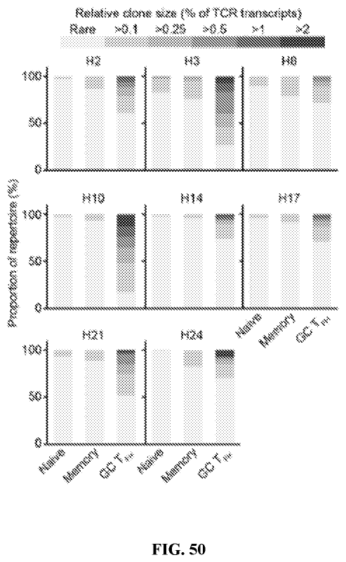

To determine if clonally expanded and/or convergently selected TCRs include HIV-specific sequences, approximately 2-3 million thawed LN cells were cultured with an HIV-1 consensus B Gag peptide pool for 3-4 weeks, then restimulated with the same peptide pool for 4 hours to identify antigen-specific T cells by CD40L and CD69 upregulation. LN cells were also stimulated with an overlapping set of hemagglutinin (HA) peptides from influenza virus (A/California/7/2009) as a non-HIV control. TCRs from CD40L⁺CD69⁺ Gag- or HA-reactive T cells were used to generate a reference TCR panel. These antigen-specific TCR sequences were mapped onto our bulk T cell sequencing data from freshly thawed LN cells to determine which sequences were Gag- or HA-specific. Common sequences shared between naïve, memory, or GC T_{FH} cells were shown as connecting lines on circos plots (FIG. 49A).

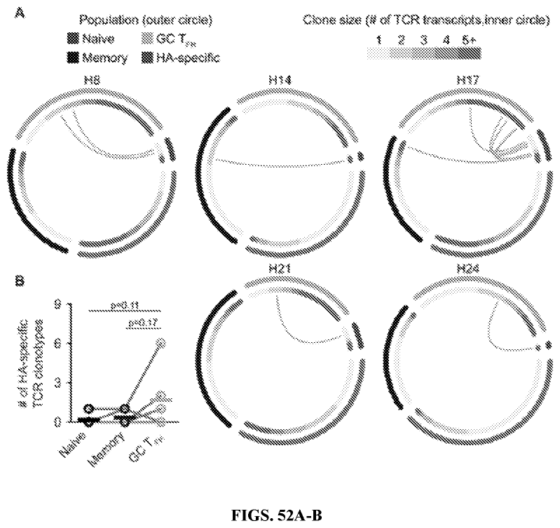

Several Gag-specific TCR sequences were found in the GC T_{FH} (0 to 7 clones) population. Though there were not enough data points to reach significance, the overlapping between Gag-specific TCR sequences was minimal in memory T cells (0 or 1 clones), and no Gag-specific sequences were found in the naïve T cell population (FIG. 49B). A similar trend of enrichment of antigen-specific clones in the GC T_{FH} phenotype was also observed for HA-specific TCR sequences (FIG. 52). This is unsurprising, as these individuals have likely been exposed to influenza infection and/or vaccinated against HA in the past. However, analysis of combined TCR sequencing data from all individuals clearly showed that these Gag-specific GC T_{FH} cells, but not the HA-specific clones, were highly expanded compared to the bulk GC T_{FH} cells of unknown specificity (FIG. 49C). Translating these antigen-specific TCR sequences into amino acid sequences showed that the Gag-specific TCR sequences within the GC T_{FH} population, but not the HA-specific sequences, have a significantly higher degree of coding degeneracy (FIG. 49D). Thus, the Gag-specific GC T_{FH} cells were preferentially expanded and degenerate. Collectively, these data indicate that Gag-specific T_{FH} cells respond to antigen stimulation and become selectively expanded in the LNs.

### Example 10—Materials and Methods

Study Design:

The goal of the study was to define T_{FH} cell diversity in primary human LNs. The HIV⁺ cohort was composed of 36 individuals. LNs were obtained from the excision of palpable cervical LNs for clinical diagnostic workup and after written informed consent was obtained. HC LNs included two samples from individuals undergoing clinically indicated bowel resection for benign polypectomy, samples from iliac region of nine transplant donors, and one cervical sample combined from 5 autopsy donors. Sample sizes were not pre-specified and were dictated by the availability of the samples, which were collected over four years.

CyTOF Staining and Data Analyses:

Cryopreserved cells were thawed and stained with metal-conjugated antibody panel, following a 5 hour stimulation with PMA and ionomycin in the presence monensin and Brefeldin A. Antibody stained cells were mixed with normalization beads and acquired on CyTOF 2. Bead standards were used to normalize CyTOF runs with the Matlab-based Nolan lab normalizer. Data analyses were performed using Cytobank and “cytofkit” package in R.

TCRβ Sequencing and Analyses:

TCR sequences from single cells were obtained by a series of three nested PCR reactions as previously described. TCR junctional region analysis was performed using IMGT/V-Quest. For bulk cell analyses, TCR library generation and raw sequence processing were performed using MIDs.

Statistical Methods:

Assessment of normality was performed using D'Agostino-Pearson test. Pearson or Spearman correlation was used depending on the normality of the data to measure the degree of association. The best-fitting line was calculated using least squares fit regression. Statistical comparisons were performed using two-tailed Student's t-test or Wilcoxon signed-rank test, using a p-value of <0.05 as a cutoff to determine statistical significance. Multiple-way comparisons were corrected using Holm-Sidak method. Statistical analyses were performed using GraphPad Prism.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Bernard et al, Anal. Biochem., 273: 221-228, 1999.
Bolotin et al., European journal of immunology 42, 3073-3083, 2012.
Brezinschek et al., 1995.
Cosstick, et al., Nucleic Acids Research 18(4):829-35, 1990.
DeKosky et al., Nature biotechnology 31, 166-169, 2013.
Georgiou et al., Nature biotechnology 32, 158-168, 2014.
Islam et al. Nat. Methods, 2014.
Jack and Wabl 1988.
Jiang et al., Proceedings of the National Academy of Sciences of the United States of America 108, 5348-5353, 2011.
Jiang et al., Science translational medicine 5, 171ra119, 2013.
Kivioja, T. et al. Nat. Methods, 9: 72-74, 2012.
Loman et al., 2012.
Michaeli et al., Front Immunol 3, 386, 2012.
Peet, Annu Rev. Ecol. Syst. 5:285, 1974.
PrabhuDas et al., Nature immunology 12, 189-194, 2011.
Ridings et al., Clinical and experimental immunology 108, 366-374, 1997.
Robins et al., Current opinion in immunology 25, 646-652, 2013.
Sambrook, Fritsch and Maniatis, MOLECULAR CLONING: A LABORATORY MANUAL, 2nd edition (1989).
Schroeder et al., Blood 98, 2745-2751, 2001.
Shugay et al., Nature methods, 2014.
Tibshirani et al. P.N.A.S. 99:6567-6572, 2002.
Vander Heiden et al., Bioinformatics, 2014.
Vollmers et al., Proceedings of the National Academy of Sciences of the United States of America 110, 13463-13468, 2013.
Weinstein et al., Science 324, 807-810, 2009.
Yaari et al., Nucleic acids research 40, e134, 2012.
Zhu et al., Proceedings of the National Academy of Sciences of the United States of America 110, 6470-6475, 2013.
U.S. Pat. No. 5,994,076
U.S. Pat. No. 7,435,572
U.S. Pat. No. 8,053,192
U.S. Patent Publication No. 2013/0274117
International Patent Publication No. WO 2012/142213
International Patent Publication No. WO05/068656

What is claimed is:

1. A method of amplifying variable immune sequences comprising:
(a) producing cDNA from a plurality of RNA molecules using barcoded oligonucleotides, wherein the barcoded oligonucleotides comprise a molecular identifier (MID) and a gene-specific primer, thereby generating a plurality of MID-tagged cDNAs; and
(b) amplifying the MID-tagged cDNAs using nested PCR, thereby producing a plurality of MID-tagged variable immune sequences.

2. The method of claim 1, wherein the gene-specific primer hybridizes to the constant region of an immunological receptor.

3. The method of claim 2, wherein the immunological receptor is an immunoglobulin, T cell receptor (TCR), major histocompatibility receptor, NK cell receptor, complement receptor, Fc receptor or fragment thereof.

4. The method of claim 2, wherein the constant region is an immunoglobulin heavy chain or immunoglobulin light chain.

5. The method of claim 2, wherein the constant region is a TCR α chain or TCR β chain.

6. The method of claim 4, wherein the gene-specific primer comprises SEQ ID NO:1 (AAGACCGATGGGCCCTTG), SEQ ID NO:2 (GAAGACCTTGGGGCTGGT), SEQ ID NO:3 (GGGAATTCTCACAGGAGACG), SEQ ID NO:4 (GAAGACGGATGGGCTCTGT), or SEQ ID NO:5 (GGGTGTCTGCACCCTGATA).

7. The method of claim 5, wherein gene-specific primer is SEQ ID NO:6 (GACCTCGGGTGGGAACAC) or SEQ ID NO:7 (GGTACACGGCAGGGTCAG).

8. The method of claim 1, wherein the plurality of MID-tagged variable immune sequences are further defined as nucleic acids which encode for the variable region of an immunoglobulin, T cell receptor (TCR), major histocompatibility receptor, NK cell receptor, complement receptor, Fc receptor or fragment thereof.

9. The method of claim 1, further comprising isolating a plurality of RNA molecules from a sample prior to step (a).

10. The method of claim 9, wherein the sample is blood, lymph, sputum, or tissue.

11. The method of claim 9, wherein the sample is a blood sample.

12. The method of claim 9, wherein the sample comprises peripheral blood mononuclear cells, B cells, T cells, or plasmablasts.

13. The method of claim 9, wherein the samples comprises 1,000 to 10,000,000 cells.

14. The method of claim 9, wherein the sample comprises less than 1,000 cells.

15. The method of claim 9, wherein the sample comprises more than 10,000,000 cells.

16. The method of claim 9, wherein the sample is obtained from a subject having an autoimmune disease, an infectious disease, or cancer.

17. The method of claim 16, wherein the sample is obtained from a transplant recipient or a vaccine recipient.

18. The method of claim 9, wherein the sample is obtained from a subject being treated with an immunosuppressive therapy.

19. The method of claim 1, wherein the MID comprises 8-16 nucleotides.

20. The method of claim 1, wherein the MID comprises 9 nucleotides.

21. The method of claim 1, wherein the MID comprises 12 nucleotides.

22. The method of claim 1, further comprising digesting the barcoded oligonucleotides with an enzyme prior to step (b).

23. The method of claim 22, wherein the enzyme is exonuclease I.

24. The method of claim 1, wherein steps (a) and (b) are performed in the same reaction tube.

25. The method of claim 1, wherein the cDNA of step (a) is not subjected to a purification prior to step (b).

26. The method of claim 1, wherein there is no purification of cDNA by size exclusion chromatography.

27. The method of claim 1, wherein the nested PCR comprises using a first set of primers specific to the leader region of an immunoglobulin or TCR.

28. The method of claim 27, wherein the first set of primers specific to the leader region of an immunoglobulin or TCR are selected from the primers listed in Table 1.

29. The method of claim 9, further comprising sequencing the plurality of MID-tagged immune variable sequences to obtain sequencing reads and analyzing the sequencing reads to determine the immune repertoire of the sample.

30. The method of claim 29, wherein analyzing comprises performing clustering data analysis.

31. The method of claim 30, wherein clustering data analysis comprises merging paired-end raw reads, identifying immunological receptor reads, and grouping sequence reads with identical MIDs.

32. The method of claim 31, further comprising applying a threshold clustering process to cluster reads with identical MIDs into subgroups.

33. The method of claim 32, wherein the clustering threshold is 1 to 20% of the read length.

34. The method of claim 32, wherein the clustering threshold is 4 to 6% of the read length.

35. The method of claim 32, wherein the clustering threshold is 14 to 15% of the read length.

36. The method of claim 32, further comprising building a consensus sequence for each cluster to produce a collection of consensus sequences.

37. The method of claim 36, wherein the collection of consensus sequences is used to determine the diversity and/or abundance of the immune repertoire.

38. The method of claim 37, further comprising calculating the sequencing error rate.

39. The method of claim 38, wherein the error rate is less than 0.005%.

40. The method of claim 38, wherein the error rate is less than 0.004%.

41. The method of any one of claims 31-40, further comprising counting RNA molecule copy number of the immune sequences.

42. The method of claim 41, wherein the immune sequences are TCRs.

43. The method of claim 41, wherein the counting is based on input cell number, percentage of RNA input, and sequencing depth.

44. The method of claim 41, wherein counting comprises performing digital PCR.

45. The method of claim 44, wherein performing digital PCR comprises using primers of Table 15.

46. The method of claim 42, wherein TCR RNA molecule copy number is determined for a single cell.

47. The method of claim 46, wherein single cell counting comprises fitting distribution of reads under each MID sub-group into two binomial distributions.

48. A method for monitoring T cell clonal expansion in a subject comprising:
(a) obtaining a population of T cells from the subject;
(b) determining the TCR sequence by the method of any one of claims 1-47; and
(c) quantifying T cell clonal expansion.

49. The method of claim 48, wherein the T cells are effector T cells.

50. The method of claim 48, wherein the subject has a viral infection.

51. The method of claim 48, wherein the viral infection is CMV.

52. The method of claim 48, wherein the subject has cancer, an infectious disease, or autoimmune disease.

53. The method of claim 48, wherein the sample subject is a transplant or vaccine recipient.

54. The method of claim 52 or 53, further comprising using T cell expansion quantification to predict response to a treatment or vaccine.

55. A method of producing a cDNA library for immune repertoire analysis comprising:
(a) obtaining a plurality of RNA molecules;
(b) hybridizing the plurality of RNA molecules to oligo(dT)-containing primers;
(c) performing reverse transcription using template switching oligonucleotides comprising a molecular identifier (MID) and a poly-uracil region, thereby generating a plurality of cDNAs; and
(d) PCR amplifying the plurality of cDNAs, thereby producing a cDNA library for immune repertoire analysis.

56. The method of claim 55, wherein the poly-uracil region comprises 2, 3, 4, 5, or 6 uracils.

57. The method of claim 55, further comprising contacting the template switching oligonucleotides with uracil-specific excision reagent (USER) enzyme prior to step (d), thereby degrading the template switching oligonucleotides.

58. The method of claim 55, wherein steps (c) and (d) comprise performing rapid amplification of cDNA ends (RACE).

59. The method of claim 55, wherein obtaining in step (a) comprises isolating a plurality of RNA molecules from a sample.

60. The method of claim 59, wherein the sample is blood, lymph, sputum, or tissue.

61. The method of claim 59, wherein the sample is a blood sample.

62. The method of claim 59, wherein the sample comprises peripheral blood mononuclear cells, B cells, T cells, or plasmablasts.

63. The method of claim 59, wherein the sample comprises 1,000 to 1,000,000 cells.

64. The method of claim 59, wherein the sample comprises less than 1,000 cells.

65. The method of claim 59, wherein the sample comprises less than 100 cells.

66. The method of claim 59, further comprising the addition of carrier RNA to the cells.

67. The method of claim 59, wherein the sample is obtained from a subject having an autoimmune disease, an infectious disease or cancer, or a transplant recipient.

68. The method of claim 59, wherein the sample is obtained from a subject being treated with an immunosuppressive therapy.

69. The method of claim 55, wherein the MID comprises 8-16 nucleotides.

70. The method of claim 55, wherein the MID comprises 9 nucleotides.

71. The method of claim 55, wherein the MID comprises 12 nucleotides.

72. The method of claim 55, wherein steps (b) to (d) are performed in a single reaction tube.

73. The method of claim 55, wherein the cDNA of step (c) is not subjected to a purification prior to step (d).

74. The method of claim 55, further comprising performing immune repertoire analysis.

75. The method of claim 74, wherein performing immune repertoire analysis comprises performing whole transcriptome sequencing of the cDNA library.

76. The method of claim 74, wherein performing immune repertoire analysis comprises immunoglobulin and/or TCR amplification prior to sequencing of the cDNA library.

77. The method of claim 75, further comprising performing clustering data analysis.

78. The method of claim 77, wherein clustering data analysis comprises merging paired-end raw reads, identifying immunological receptor reads, and grouping sequence reads with identical MIDs.

79. The method of claim 78, further comprising applying a threshold clustering process to cluster reads with identical MIDs into subgroups.

80. The method of claim 79, wherein the clustering threshold is 1 to 20% of the read length.

81. The method of claim 79, wherein the clustering threshold is 4 to 6% of the read length.

82. The method of claim 79, wherein the clustering threshold is 14 to 15% of the read length.

83. The method of claim 79, further comprising building a consensus sequence for each cluster to produce a collection of consensus sequences.

84. The method of claim 83, wherein the collection of consensus sequences is used to determine the diversity of the immune repertoire.

85. The method of claim 84, further comprising calculating the sequencing error rate.

86. The method of claim 85, wherein the error rate is less than 0.005%.

87. The method of claim 85, wherein the error rate is less than 0.004%.

88. A composition comprising T cell primers listed in Table 1.

89. The composition of claim 88, wherein the T cells primer are further defined as single cell TCR sequencing primers, bulk TCR repertoire sequencing primers, or single cell TCR with single cell RNA-sequencing primer.
